(19) United States
(12) Patent Application Publication
(10) Pub. No.: US 2024/0132913 A1
(43) Pub. Date: Apr. 25, 2024

(54) MODIFIED ORTHOPOXVIRUS VECTORS

(71) Applicants: OTTAWA HOSPITAL RESEARCH INSTITUTE, Ottawa (CA); TURNSTONE BIOLOGICS CORP., La Jolla, CA (US)

(72) Inventors: John C. Bell, Ottawa (CA); Fabrice Le Boeuf, Gatineau (CA); Michael S. Huh, Gatineau (CA); Matthew Y. Tang, Orleans (CA); Adrian Pelin, Ottawa (CA); Brian Andrew Keller, Ottawa (CA); Caroline J. Breitbach, Somerville, MA (US); Michael F. Burgess, Chester, NJ (US); Steven H. Bernstein, Lawrenceville, NJ (US)

(73) Assignees: OTTAWA HOSPITAL RESEARCH INSTITUTE, Ottawa (CA); TURNSTONE BIOLOGICS CORP., La Jolla, CA (US)

(21) Appl. No.: 18/461,241

(22) Filed: Sep. 4, 2023

Related U.S. Application Data
(62) Division of application No. 16/959,632, filed on Jul. 1, 2020, now Pat. No. 11,802,292.
(60) Provisional application No. 62/784,371, filed on Dec. 21, 2018.
(60) Provisional application No. 62/614,349, filed on Jan. 5, 2018.

(51) Int. Cl. C12N 15/86; A61K 35/768; C07K 14/545; C12N 7/00; C12N 9/22

(52) U.S. Cl. CPC C12N 15/86; A61K 35/768; C07K 14/545; C12N 7/00; C12N 9/22; C12N 2710/24121; C12N 2710/24132; C12Y 306/01023

(57) ABSTRACT

The disclosure relates to modified orthopoxvirus vectors, as well as methods of using the same for the treatment of various cancers. The disclosure provides modified orthopoxvirus vectors that exhibit various beneficial therapeutic activities, including enhanced oncolytic activity, spread of infection, immune evasion, tumor persistence, capacity for incorporation of exogenous DNA sequences and safety. The viruses we have discovered are also amenable to large scale manufacturing protocols.

### FIELD

The invention relates to the field of immunotherapy, e.g., for the treatment of cell proliferation disorders, such as cancers. Particularly, the invention relates to genetically modified orthopoxviruses, as well as methods of making and using the same.

### BACKGROUND

The immune system may be stimulated to identify tumor cells and target them for destruction. Immunotherapy employing oncolytic orthopoxviruses is a rapidly evolving area in cancer research. New approaches are needed to engineer and/or enhance tumor-selectivity for oncolytic viruses in order to maximize efficiency and safety. This selectivity is especially important when potentially toxic therapeutic agents or genes are added to the viruses.

Although the use of orthopoxviruses as clinical oncolytic vectors is a promising paradigm for cancer treatment, due to toxicity, such as pox lesions in patients, and immunosuppressive side effects, most current clinical candidates have shown only modest clinical success. There exists a need for methods to engineer orthopoxviruses that exhibit more robust virus replication, cancer cell killing, and spreading from the point of infection. The present invention addresses this need and provides a solution to selectivity and safety limitations by employing a modified vaccinia virus.

### SUMMARY

The present disclosure describes the use of orthopoxviruses for the treatment of cancer. In particular, the disclosure is based in part on the surprisingly enhanced oncolytic activity, spread of infection, and safety results engendered when a orthopoxvirus is genetically modified to contain deletions in one or more, or all, of the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, K ORF A, K ORF B, B ORF E, B ORF F, B ORF G, B21R, B22R, B23R, B24R, B25R, B26R, B27R, B28R, and B29R. Genetically modified orthopoxviruses, such as vaccinia viruses (e.g., Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1 viruses) that exhibit mutations in one or more, or all, of these genes may exhibit an array of beneficial features, such as improved oncolytic ability, replication in tumors, infectivity, immune evasion, tumor persistence, capacity for incorporation of exogenous DNA sequences, and/or amenability for large scale manufacturing. The present disclosure describes orthopox viruses further genetically modified to contain deletions in the B8R gene. In various embodiments disclosed below, the invention may or may not include a deletion of the B8R gene. In various embodiments, the modified orthopoxvirus expresses at least one of three transgenes: IL-12-TM, FLT3-L and anti-CLTA4 antibody.

In a first aspect, the invention features a nucleic acid that includes a recombinant orthopoxvirus genome, wherein the recombinant orthopoxvirus genome has a deletion of at least 2, 3, 4, 5, 6, 7, 8, 9, 10, 11, 12, 13, 14, 15, 16, 17, 18, 19, 20, 21 or 22 genes, each independently selected from the group consisting of C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R. In some embodiments, the deletion includes each of C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R genes. In any one of the embodiments, disclosed herein, the recombinant orthopoxvirus genome may further include a deletion of the B8R gene.

In another aspect, the invention features a nucleic acid that includes a recombinant orthopoxvirus genome, wherein the recombinant orthopoxvirus genome has a deletion of at least 1 gene selected from the group consisting of B14R, B16R, B17L, B18R, B19R, and B20R. In some embodiments, the deletion includes at least 2, 3, 4, or 5 genes, each independently selected from the group consisting of B14R, B16R, B17L, B18R, B19R, and B20R. In some embodiments, the deletion includes each of B14R, B16R, B17L, B18R, B19R, and B20R. In any one of the embodiments, disclosed herein, the recombinant orthopoxvirus genome may further include a B8R deletion.

In another aspect, the invention features a nucleic acid that includes a recombinant orthopoxvirus genome, wherein the recombinant orthopoxvirus genome has a deletion of at least 1 gene selected from the group consisting of C2L, C1L, N1L, N2L, M1L, M2L, K1L, K2L, K3L, K4L, K5L, K6L, K7R, F1L, F2L, and F3L. In some embodiments, the deletion includes at least 2, 3, 4, 5, 6, 7, 8, 9, 10, 11, 12, 13, 14, or 15 genes, each independently selected from the group consisting of C2L, C1L, N1L, N2L, M1L, M2L, K1L, K2L, K3L, K4L, K5L, K6L, K7R, F1L, F2L, and F3L. In some embodiments, the deletion includes each of C2L, C1L, N1L, N2L, M1L, M2L, K1L, K2L, K3L, K4L, K5L, K6L, K7R, F1L, F2L, and F3L. In any one of the embodiments, disclosed herein, the recombinant orthopoxvirus genome may further include a B8R deletion.

In another aspect, the invention features a nucleic acid that includes a recombinant orthopoxvirus genome, wherein the recombinant orthopoxvirus genome has a deletion of at least 1 gene that encodes a caspase-9 inhibitor.

In some embodiments, the recombinant orthopoxvirus genome has a deletion of at least 1 gene that encodes a caspase-9 inhibitor. In some embodiments, the gene that encodes a caspase-9 inhibitor is F1L.

In another aspect, the invention features a nucleic acid that includes a recombinant orthopoxvirus genome, wherein the recombinant orthopoxvirus genome has a deletion of at least 1 gene that encodes a BCL-2 inhibitor.

In some embodiments, the recombinant orthopoxvirus genome has a deletion of at least 1 gene that encodes a BCL-2 inhibitor. In some embodiments, the gene that encodes a BCL-2 inhibitor is N1L.

In another aspect, the invention features a nucleic acid that includes a recombinant orthopoxvirus genome, wherein the recombinant orthopoxvirus genome has a deletion of at least 1 gene that encodes a dUTPase.

In some embodiments, the recombinant orthopoxvirus genome has a deletion of at least 1 gene that encodes a dUTPase. In some embodiments, the gene that encodes a dUTPase is F2L.

In another aspect, the invention features a nucleic acid that includes a recombinant orthopoxvirus genome, wherein the recombinant orthopoxvirus genome has a deletion of at least 1 gene that encodes a IFN-alpha/beta-receptor-like secreted glycoprotein.

In some embodiments, the recombinant orthopoxvirus genome has a deletion of at least 1 gene that encodes a IFN-alpha/beta-receptor-like secreted glycoprotein. In some embodiments, the gene that encodes a IFN-alpha/beta-receptor-like secreted glycoprotein is B19R.

In another aspect, the invention features a nucleic acid that includes a recombinant orthopoxvirus genome, wherein the recombinant orthopoxvirus genome has a deletion of at least 1 gene that encodes an IL-1-beta-inhibitor.

In some embodiments, the recombinant orthopoxvirus genome has a deletion of at least 1 gene that encodes an IL-1-beta-inhibitor. In some embodiments, the gene that encodes an IL-1-beta-inhibitor is B16R.

In another aspect, the invention features a nucleic acid that includes a recombinant orthopoxvirus genome, wherein the recombinant orthopoxvirus genome has a deletion of at least 1 gene that encodes a phospholipase-D.

In some embodiments, the recombinant orthopoxvirus genome has a deletion of at least 1 gene that encodes a phospholipase-D. In some embodiments, the gene that encodes a phospholipase-D is K4L.

In another aspect, the invention features a nucleic acid that includes a recombinant orthopoxvirus genome, wherein the recombinant orthopoxvirus genome has a deletion of at least 1 gene that encodes a PKR inhibitor.

In some embodiments, the recombinant orthopoxvirus genome has a deletion of at least 1 gene that encodes a PKR inhibitor. In some embodiments, the gene that encodes a PKR inhibitor is K3L.

In another aspect, the invention features a nucleic acid that includes a recombinant orthopoxvirus genome, wherein the recombinant orthopoxvirus genome has a deletion of at least 1 gene that encodes a serine protease inhibitor.

In some embodiments, the recombinant orthopoxvirus genome has a deletion of at least 1 gene that encodes a serine protease inhibitor. In some embodiments, the gene that encodes a serine protease inhibitor is K2L.

In another aspect, the invention features a nucleic acid that includes a recombinant orthopoxvirus genome, wherein the recombinant orthopoxvirus genome has a deletion of at least 1 gene that encodes a TLR signaling inhibitor. In some embodiments, the recombinant orthopoxvirus genome has a deletion of at least 1 gene that encodes a TLR signaling inhibitor. In some embodiments, the gene that encodes a TLR signaling inhibitor is N2L.

In another aspect, the invention features a nucleic acid that includes a recombinant orthopoxvirus genome, wherein the recombinant orthopoxvirus genome has a deletion of at least 1 gene that encodes a kelch-like protein. In some embodiments, the recombinant orthopoxvirus genome has a deletion of at least 1 gene that encodes a kelch-like protein. In some embodiments, the recombinant orthopoxvirus genome has a deletion of at least 2 genes that each encodes a kelch-like protein. In some embodiments, the genes that encode a kelch-like protein are, independently, selected from the group consisting of F3L and C2L.

In another aspect, the invention features a nucleic acid that includes a recombinant orthopoxvirus genome, wherein the recombinant orthopoxvirus genome has a deletion of at least 1 gene that encodes a monoglyceride lipase.

In some embodiments, the recombinant orthopoxvirus genome has a deletion of at least 1 or 2 genes that encodes a monoglyceride lipase. In some embodiments, the genes that encode a monoglyceride lipase are, independently, selected from the group consisting of K5L and K6L.

In another aspect, the invention features a nucleic acid that includes a recombinant orthopoxvirus genome, wherein the recombinant orthopoxvirus genome has a deletion of at least 1, 2 or 3 genes that encodes an NF-κB inhibitor. In some embodiments, the genes that encode an NF-κB inhibitor are, independently selected from the group consisting of K7R, K1L, and M2L.

In another aspect, the invention features a nucleic acid that includes a recombinant orthopoxvirus genome, wherein the recombinant orthopoxvirus genome has a deletion of at least 1, 2, or 3 genes that encodes an Ankyrin repeat protein. In some embodiments, the genes that encode an Ankyrin repeat protein are, independently, selected from the group consisting of B18R, B20R, and M1L.

In another aspect, the invention features a nucleic acid that includes a recombinant orthopoxvirus genome, wherein the recombinant orthopoxvirus genome has a deletion of at least 1, 2 or 3 genes each independently selected from the group consisting of B15R, B17R, and B14R.

In another aspect, the invention features a nucleic acid that includes a recombinant orthopoxvirus genome, wherein the recombinant orthopoxvirus genome has a deletion of at least 1, 2, 3, or 4, gene selected from the group consisting of K ORF A, K ORF B, B ORF E, B ORF F, and B ORF G. In any one of the embodiments, disclosed herein, the recombinant orthopoxvirus genome further includes a B8R deletion.

In some embodiments, the recombinant orthopoxvirus genome has a deletion of at least 1 gene selected from the group of inverted terminal repeat (ITR) genes consisting of B21R, B22R, B23R, B24R, B25R, B26R, B27R, B28R, and B29R. In some embodiments, the deletion includes at least 2, 3, 4, 5, 6, 7, or 8 genes, each independently selected from the group of ITR genes consisting of B21R, B22R, B23R, B24R, B25R, B26R, B27R, B28R, and B29R. In some embodiments, the deletion includes each of B21R, B22R, B23R, B24R, B25R, B26R, B27R, B28R, and B29R. In any one of the embodiments, disclosed herein, the recombinant orthopoxvirus genome may further include a B8R deletion.

In some embodiments, the vaccinia virus is a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1. In some embodiments, the vaccinia virus is a strain selected from the group consisting of Copenhagen, Western Reserve, Tian Tan, Wyeth, and Lister. In some embodiments, the vaccinia virus is a Copenhagen strain vaccinia virus.

In some embodiments, one or more, or all, of the deletions is a deletion of the entire polynucleotide encoding the corresponding gene. In some embodiments, one or more, or all, of the deletions is a deletion of a portion of the polynucleotide encoding the corresponding gene, such that the deletion is sufficient to render the gene nonfunctional, e.g., upon introduction into a host cell.

In some embodiments, the nucleic acid further includes a transgene encoding a tumor-associated antigen. In some embodiments, the tumor-associated antigen is a tumor-associated antigen listed in any one of Tables 3-30 herein. In some embodiments, the tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK. In some embodiments, the tumor-associated antigen includes MAGE-A3, or one or more fragments thereof. In some embodiments, the tumor-associated antigen includes NY-ESO-1, or one or more fragments thereof. In some embodiments, the tumor-associated antigen includes one or more human papillomavirus (HPV) proteins, or fragments thereof. In some embodiments, the tumor-associated antigen includes (i) E6 and E7 proteins, or fragments thereof, of HPV16 and (ii) E6 and E7 proteins, or fragments thereof, of HPV18. In some embodiments, the tumor-associated antigen includes brachyury or one or more fragments thereof. In some embodiments, the tumor-associated antigen includes prostatic acid phosphatase, or one or more fragments thereof.

In some embodiments, the nucleic acid further includes a transgene encoding an immune checkpoint inhibitor. In some embodiments, the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In some embodiments, the immune checkpoint inhibitor is an anti-PD1 antibody or antigen-binding fragment thereof or an anti-CTLA-4 antibody or antigen-binding fragment thereof. In some embodiments, the immune checkpoint inhibitor is an anti-PD1 antibody or antigen-binding fragment thereof. In some embodiments, the immune checkpoint inhibitor is an anti-CTLA-4 antibody or antigen-binding fragment thereof.

Antibodies or antigen-binding fragments thereof described herein may be full-length antibodies or antibody fragments, such as a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv). In some embodiments, the antibody or antigen-binding fragment thereof contains two or more CDRs covalently bound to one another, e.g., by an amide bond, a thioether bond, a carbon-carbon bond, or a disulfide bridge, or by a linker, such as a linker described herein. In some embodiments, the antibody or antigen-binding fragment thereof is a single-chain polypeptide. In some embodiments, the antibody or antigen-binding fragment thereof has an isotype selected from the group consisting of IgG, IgA, IgM, IgD, and IgE.

In some embodiments, the nucleic acid further includes a transgene encoding an interleukin. In some embodiments, the interleukin (IL) is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In some embodiments, the interleukin is selected from the group consisting of IL-12 p35, IL-12 p40, and IL-12 p70. In some embodiments, the interleukin is membrane-bound.

In some embodiments, the nucleic acid further includes a transgene encoding an interferon. In some embodiments, the interferon is selected from the group consisting of IFN-alpha, IFN-beta, IFN-delta, IFN-epsilon, IFN-tau, IFN-omega, IFN-zeta, and IFN-gamma.

In some embodiments, the nucleic acid further includes a transgene encoding a TNF superfamily member protein. In some embodiments, the TNF superfamily member protein is selected from the group consisting of TRAIL, Fas ligand, LIGHT (TNFSF-14), TNF-alpha, and 4-1BB ligand.

In some embodiments, the nucleic acid further includes a transgene encoding a cytokine. In some embodiments, the cytokine selected from the group consisting of GM-CSF, FMS-like tyrosine kinase 3 ligand (Flt3 ligand), CD40 ligand, anti-TGF-beta, anti-VEGF-R2, and guanyl adenylate cyclase (cGAS). In some embodiments, the cytokine is Flt3 ligand.

In another aspect, the invention features a recombinant orthopoxvirus vector that has a deletion of at least 2, 3, 4, 5, 6, 7, 8, 9, 10, 11, 12, 13, 14, 15, 16, 17, 18, 19, 20, 21 or 22 genes, each independently selected from the group consisting of C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R. In some embodiments, the deletion includes each of C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R. In any one of the embodiments, disclosed herein, the recombinant orthopoxvirus genome may further include a B8R deletion.

In another aspect, the invention features a recombinant orthopoxvirus vector that includes a recombinant orthopoxvirus genome, wherein the recombinant orthopoxvirus genome has a deletion of at least 1 gene selected from the group consisting of B14R, B16R, B17L, B18R, B19R, and B20R. In some embodiments, the deletion includes at least 2, 3, 4, or 5 genes, each independently selected from the group consisting of B14R, B16R, B17L, B18R, B19R, and B20R. In some embodiments, the deletion includes each of B14R, B16R, B17L, B18R, B19R, and B20R. In any one of the embodiments, disclosed herein, the recombinant orthopoxvirus genome may further include a B8R deletion.

In another aspect, the invention features a recombinant orthopoxvirus vector that includes a recombinant orthopoxvirus genome, wherein the recombinant orthopoxvirus genome has a deletion of at least 1 gene selected from the group consisting of C2L, C1L, N1L, N2L, M1L, M2L, K1L, K2L, K3L, K4L, K5L, K6L, K7R, F1L, F2L, and F3L. In any one of the embodiments, disclosed herein, the recombinant orthopoxvirus genome may further include a B8R deletion.

In some embodiments, the recombinant orthopoxvirus vector has a deletion of at least 1 gene selected from the group consisting of C2L, C1L, N1L, N2L, M1L, K1L, K2L, K3L, K4L, K7R, and F2L. In some embodiments, the deletion includes at least 2, 3, 4, 5, 6, 7, 8, 9, 10, 11, 12, 13, 14, 15, or 16 genes, each independently selected from the group consisting of C2L, C1L, N1L, N2L, M1L, M2L, K1L, K2L, K3L, K4L, K5L, K6L, K7R, F1L, F2L, and F3L. In some embodiments, the deletion includes each of C2L, C1L, N1L, N2L, M1L, M2L, K1L, K2L, K3L, K4L, K5L, K6L, K7R, F1L, F2L, and F3. In any one of the embodiments, disclosed herein, the recombinant orthopoxvirus genome may further include a B8R deletion.

In another aspect, the invention features a recombinant orthopoxvirus vector that has a deletion of at least 1 gene that encodes a caspase-9 inhibitor.

In some embodiments, the recombinant orthopoxvirus genome has a deletion of at least 1 gene that encodes a caspase-9 inhibitor. In some embodiments, the gene that encodes a caspase-9 inhibitor is F1L.

In another aspect, the invention features a recombinant orthopoxvirus vector has a deletion of at least 1 gene that encodes a BCL-2 inhibitor.

In some embodiments, the recombinant orthopoxvirus genome has a deletion of at least 1 gene that encodes a BCL-2 inhibitor. In some embodiments, the gene that encodes a BCL-2 inhibitor is N1L.

In another aspect, the invention features a recombinant orthopoxvirus vector has a deletion of at least 1 gene that encodes a dUTPase.

In some embodiments, the recombinant orthopoxvirus genome has a deletion of at least 1 gene that encodes a dUTPase. In some embodiments, the gene that encodes a dUTPase is F2L.

In another aspect, the invention features a recombinant orthopoxvirus vector has a deletion of at least 1 gene that encodes a IFN-alpha/beta-receptor-like secreted glycoprotein.

In some embodiments, the recombinant orthopoxvirus genome has a deletion of at least 1 gene that encodes a IFN-alpha/beta-receptor-like secreted glycoprotein. In some embodiments, the gene that encodes a IFN-alpha/beta-receptor-like secreted glycoprotein is B19R.

In another aspect, the invention features a recombinant orthopoxvirus vector has a deletion of at least 1 gene that encodes an IL-1-beta-inhibitor.

In some embodiments, the recombinant orthopoxvirus genome has a deletion of at least 1 gene that encodes an IL-1-beta-inhibitor. In some embodiments, the gene that encodes an IL-1-beta-inhibitor is B16R.

In another aspect, the invention features a recombinant orthopoxvirus vector has a deletion of at least 1 gene that encodes a phospholipase-D.

In some embodiments, the recombinant orthopoxvirus genome has a deletion of at least 1 gene that encodes a phospholipase-D. In some embodiments, the gene that encodes a phospholipase-D is K4L.

In another aspect, the invention features a recombinant orthopoxvirus vector has a deletion of at least 1 gene that encodes a PKR inhibitor.

In some embodiments, the recombinant orthopoxvirus genome has a deletion of at least 1 gene that encodes a PKR inhibitor. In some embodiments, the gene that encodes a PKR inhibitor is K3L.

In another aspect, the invention features a recombinant orthopoxvirus vector has a deletion of at least 1 gene that encodes a serine protease inhibitor.

In some embodiments, the recombinant orthopoxvirus genome has a deletion of at least 1 gene that encodes a serine protease inhibitor. In some embodiments, the gene that encodes a serine protease inhibitor is K2L.

In another aspect, the invention features a recombinant orthopoxvirus vector has a deletion of at least 1 gene that encodes a TLR signaling inhibitor.

In some embodiments, the recombinant orthopoxvirus genome has a deletion of at least 1 gene that encodes a TLR signaling inhibitor. In some embodiments, the gene that encodes a TLR signaling inhibitor is N2L.

In another aspect, the invention features a recombinant orthopoxvirus vector has a deletion of at least 1 gene that encodes a kelch-like protein.

In some embodiments, the recombinant orthopoxvirus genome has a deletion of at least 1 or 2 genes that encodes a kelch-like protein. In some embodiments, the genes that encode a kelch-like protein are, independently, selected from the group consisting of F3L and C2L.

In another aspect, the invention features a recombinant orthopoxvirus vector has a deletion of at least 1 gene that encodes a monoglyceride lipase.

In some embodiments, the recombinant orthopoxvirus genome has a deletion of at least 1 gene that encodes a monoglyceride lipase. In some embodiments, the recombinant orthopoxvirus genome has a deletion of at least 2 genes that each encodes a monoglyceride lipase. In some embodiments, the genes that encode a monoglyceride lipase are, independently, selected from the group consisting of K5L and K6L.

In another aspect, the invention features a recombinant orthopoxvirus vector has a deletion of at least 1 gene that encodes an NF-κB inhibitor.

In some embodiments, the recombinant orthopoxvirus genome has a deletion of at least 1, 2, or 3 genes that encodes an NF-κB inhibitor. In some embodiments, the genes that encode an NF-κB inhibitor are, independently, selected from the group consisting of K7R, K1L, and M2L.

In another aspect, the invention features a recombinant orthopoxvirus vector has a deletion of at least 1 gene that encodes an Ankyrin repeat protein.

In some embodiments, the recombinant orthopoxvirus genome has a deletion of at least 1 gene that encodes an Ankyrin repeat protein. In some embodiments, the recombinant orthopoxvirus genome has a deletion of at least 2 genes that each encodes an Ankyrin repeat protein. In some embodiments, the recombinant orthopoxvirus genome has a deletion of at least 3 genes that each encodes an Ankyrin repeat protein. In some embodiments, the genes that encode an Ankyrin repeat protein are, independently, selected from the group consisting of B18R, B20R, and M1L.

In another aspect, the invention features a recombinant orthopoxvirus vector has a deletion of at least 1 gene selected from the group consisting of B15R, B17R, and B14R.

In some embodiments, the recombinant orthopoxvirus genome has a deletion of at least 1 gene selected from the group consisting of B15R, B17R, and B14R. In some embodiments, the recombinant orthopoxvirus genome has a deletion of at least 2 genes selected from the group consisting of B15R, B17R, and B14R. In some embodiments, the recombinant orthopoxvirus genome has a deletion of at least 3 genes selected from the group consisting of B15R, B17R, and B14R. In any one of the embodiments, disclosed herein, the recombinant orthopoxvirus genome may further include a B8R deletion.

In another aspect, the invention features a recombinant orthopoxvirus vector that includes a recombinant orthopoxvirus genome, wherein the recombinant orthopoxvirus genome has a deletion of at least 1 gene selected from the group consisting of K ORF A, K ORF B, B ORF E, B ORF F, and B ORF G.

In some embodiments, the recombinant orthopoxvirus vector has a deletion of at least 1 gene selected from the group consisting of K ORF A, K ORF B, B ORF E, B ORF F, and B ORF G. In some embodiments, the vector has a deletion of at least 2, 3, or 4 genes selected from the group consisting of K ORF A, K ORF B, B ORF E, B ORF F, and B ORF G. In some embodiments, the deletion includes each of K ORF A, K ORF B, B ORF E, B ORF F, and B ORF G. In any one of the embodiments, disclosed herein, the recombinant orthopoxvirus genome may further include a B8R deletion.

In some embodiments, the recombinant orthopoxvirus vector has a deletion of at least 1 gene selected from the group of ITR genes consisting of B21R, B22R, B23R, B24R, B25R, B26R, B27R, B28R, and B29R. In some embodiments, the deletion includes at least 2, 3, 4, 5, 6, 7 or 8 genes, each independently selected from the group of ITR genes consisting of B21R, B22R, B23R, B24R, B25R, B26R, B27R, B28R, and B29R. In some embodiments, the deletion includes each of B21R, B22R, B23R, B24R, B25R, B26R, B27R, B28R, and B29R. In any one of the embodiments, disclosed herein, the recombinant orthopoxvirus genome may further include a B8R deletion.

In some embodiments, the orthopoxvirus is a vaccinia virus.

In some embodiments, the vaccinia virus is a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1. In some embodiments, the vaccinia virus is a strain selected from the group consisting of Copenhagen, Western Reserve, Tian Tan, Wyeth, and Lister. In some embodiments, the vaccinia virus is a Copenhagen strain vaccinia virus.

In some embodiments, one or more, or all, of the deletions is a deletion of the entire polynucleotide encoding the corresponding gene. In some embodiments, one or more, or all, of the deletions is a deletion of a portion of the polynucleotide encoding the corresponding gene, such that the deletion is sufficient to render the gene nonfunctional, e.g., upon introduction into a host cell.

In some embodiments, the vector further includes a transgene encoding a tumor-associated antigen. In some embodiments, the tumor-associated antigen is a tumor-associated antigen listed in any one of Tables 3-30 herein. In some embodiments, the tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MC SP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK. In some embodiments, the tumor-associated antigen includes MAGE-A3, or one or more fragments thereof. In some embodiments, the tumor-associated antigen includes NY-ESO-1, or one or more fragments thereof. In some embodiments, the tumor-associated antigen includes one or more human papillomavirus (HPV) proteins, or fragments thereof. In some embodiments, the tumor-associated antigen includes (i) E6 and E7 proteins, or fragments thereof, of HPV16 and (ii) E6 and E7 proteins, or fragments thereof, of HPV18. In some embodiments, the tumor-associated antigen includes brachyury or one or more fragments thereof. In some embodiments, the tumor-associated antigen includes prostatic acid phosphatase, or one or more fragments thereof.

In some embodiments, the vector further includes a transgene encoding an immune checkpoint inhibitor. In some embodiments, the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In some embodiments, the immune checkpoint inhibitor is an anti-PD1 antibody or antigen-binding fragment thereof or an anti-CTLA-4 antibody or antigen-binding fragment thereof. In some embodiments, the immune checkpoint inhibitor is an anti-PD1 antibody or antigen-binding fragment thereof. In some embodiments, the immune checkpoint inhibitor is an anti-CTLA-4 antibody or antigen-binding fragment thereof.

As described above, antibodies or antigen-binding fragments thereof described herein may be full-length antibodies or antibody fragments, such as a monoclonal antibody or antigen-binding fragment thereof, a polyclonal antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv). In some embodiments, the antibody or antigen-binding fragment thereof contains two or more CDRs covalently bound to one another, e.g., by an amide bond, a thioether bond, a carbon-carbon bond, or a disulfide bridge, or by a linker, such as a linker described herein. In some embodiments, the antibody or antigen-binding fragment thereof is a single-chain polypeptide. In some embodiments, the antibody or antigen-binding fragment thereof has an isotype selected from the group consisting of IgG, IgA, IgM, IgD, and IgE.

In some embodiments, the vector further includes a transgene encoding an interleukin. In some embodiments, the interleukin (IL) is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In some embodiments, the interleukin is selected from the group consisting of IL-12 p35, IL-12 p40, and IL-12 p70. In some embodiments, the interleukin is membrane-bound.

In some embodiments, the vector further includes a transgene encoding an interferon. In some embodiments, the interferon is selected from the group consisting of IFN-alpha, IFN-beta, IFN-delta, IFN-epsilon, IFN-tau, IFN-omega, IFN-zeta, and IFN-gamma.

In some embodiments, the vector further includes a transgene encoding a TNF superfamily member protein. In some embodiments, the TNF superfamily member protein is selected from the group consisting of TRAIL, Fas ligand, LIGHT (TNFSF-14), TNF-alpha, and 4-1BB ligand.

In some embodiments, the vector further includes a transgene encoding a cytokine. In some embodiments, the cytokine selected from the group consisting of GM-CSF, FMS-like tyrosine kinase 3 ligand (Flt3 ligand), CD40 ligand, anti-TGF-beta, anti-VEGF-R2, and guanyl adenylate cyclase (cGAS). In some embodiments, the cytokine is Flt3 ligand.

In some embodiments, upon contacting a population of mammalian cells (e.g., human cells, such as human cancer cells) with the nucleic acid or the recombinant orthopoxvirus vector, the cells exhibit increased syncytia formation relative to a population of mammalian cells of the same type contacted with a form of the orthopoxvirus vector that does not include the deletions, as assessed, for instance, by visual inspection using microscopy techniques described herein or known in the art.

In some embodiments, upon contacting a population of mammalian cells (e.g., human cells, such as human cancer cells) with the nucleic acid or the recombinant orthopoxvirus vector, the cells exhibit increased spreading of the orthopoxvirus vector relative to a population of mammalian cells of the same type contacted with a form of the orthopoxvirus vector that does not include the deletions, as assessed, for instance, using plaque-forming assays described herein or known in the art.

In some embodiments, the nucleic acid or the recombinant orthopoxvirus vector exerts an increased cytotoxic effect on a population of mammalian cells (e.g., human cells, such as human cancer cells) relative to that of a form of the orthopoxvirus vector that does not include the deletions, as assessed, for instance, using cell death assays descried herein or known in the art.

In some embodiments, the mammalian cells are from a cell line selected from the group consisting of U2OS, 293, 293T, Vero, HeLa, A549, BHK, BSC40, CHO, OVCAR-8, 786-0, NCI-H23, U251, SF-295, T-47D, SKMEL2, BT-549, SK-MEL-28, MDA-MB-231, SK-OV-3, MCF7, M14, SF-268, CAKI-1, HPAV, OVCAR-4, HCT15, K-562, and HCT-116.

In another aspect, the invention features a packaging cell line that contains the nucleic acid or the recombinant orthopoxvirus vector of any of the aspects or embodiments described herein.

In another aspect, the invention features a method of treating cancer in a mammalian patient by administering a therapeutically effective amount of the nucleic acid or the recombinant orthopoxvirus vector to the patient.

In some embodiments, the mammalian patient is a human patient.

In some embodiments, the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In some embodiments, the cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

In some embodiments, the method further includes administering to the patient an immune checkpoint inhibitor. In some embodiments, the immune checkpoint inhibitor is selected from a group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In some embodiments, the immune checkpoint inhibitor is an anti-PD1 antibody or antigen-binding fragment thereof or an anti-CTLA-4 antibody or antigen-binding fragment thereof. In some embodiments, the immune checkpoint inhibitor is an anti-PD1 antibody or antigen-binding fragment thereof. In some embodiments, the immune checkpoint inhibitor is an anti-CTLA-4 antibody or antigen-binding fragment thereof.

In some embodiments, the method further includes administering to the patient an interleukin. In some embodiments, the interleukin is selected from a group consisting of IL-1 alpha, IL-1 beta, IL-2, IL-4, IL-7, IL-10, IL-12 p35, IL-12 p40, IL-12 p70, IL-15, IL-18, IL-21, and IL-23. In some embodiments, the interleukin is selected from a group consisting of IL-12 p35, IL-12 p40, and IL-12 p70. In some embodiments, the interleukin is membrane-bound.

In some embodiments, the method further includes administering to the patient an interferon. In some embodiments, the interferon is selected from a group consisting of IFN-alpha, IFN-beta, IFN-delta, IFN-epsilon, IFN-tau, IFN-omega, IFN-zeta, and IFN-gamma.

In some embodiments, the method further includes administering to the patient a TNF superfamily member protein. In some embodiments, the TNF superfamily member protein is selected from a group consisting of TRAIL, Fas ligand, LIGHT (TNFSF-14), TNF-alpha, and 4-1BB ligand.

In some embodiments, the method further includes administering to the patient a cytokine. In some embodiments, the cytokine is selected from a group consisting of GM-CSF, Flt3 ligand, CD40 ligand, anti-TGF-beta, anti-VEGF-R2, and cGAS (guanyl adenylate cyclase).

In another aspect, the invention features a kit containing the nucleic acid or vector of any of the aspects or embodiments described herein and a package insert instructing a user of the kit to express the nucleic acid or vector in a host cell.

In another aspect, the invention features a kit containing the nucleic acid or recombinant orthopoxvirus vector of any of the aspects or embodiments described herein and a package insert instructing a user to administer a therapeutically effective amount of the nucleic acid or recombinant orthopoxvirus vector to a mammalian patient (e.g., a human patient) having cancer, thereby treating the cancer.

### Definitions

As used herein, the term “about” refers to a value that is no more than 10% above or below the value being described. For example, the term “about 5 nM” indicates a range of from 4.5 nM to 5.5 nM.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rlgG, and scFv fragments. Moreover, unless otherwise indicated, the term “monoclonal antibody” (mAb) is meant to include both intact molecules, as well as, antibody fragments (such as, for example, Fab and F(ab′)2 fragments) that are capable of specifically binding to a target protein. Fab and F(ab′)2 fragments lack the Fc fragment of an intact antibody, clear more rapidly from the circulation of the animal, and may have less non-specific tissue binding than an intact antibody (see Wahl et al., J. Nucl. Med. 24:316, 1 983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)2, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment (Ward et al., Nature 341:544-546, 1 989), which consists of a VH domain; (vii) a dAb which consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “bispecific antibodies” refers to monoclonal, often human or humanized antibodies that have binding specificities for at least two different antigens.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations.

As used herein, the term “chimeric” antibody refers to an antibody having variable sequences derived from an immunoglobulin of one source organism, such as rat or mouse, and constant regions derived from an immunoglobulin of a different organism (e.g., a human). Methods for producing chimeric antibodies are known in the art. See, e.g., Morrison, 1985, Science 229(4719): 1202-7; Oi et al., 1986, BioTechniques 4:214-221; Gillies et al., 1985, J. Immunol. Methods 125:191-202; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest (National Institute of Health, Bethesda, Md. 1987; incorporated herein by reference).

As used herein, numbering of immunoglobulin amino acid residues is done according to the immunoglobulin amino acid residue numbering system of Kabat et al, unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below. From this table it is appreciated that the conservative amino acid families include (i) G, A, V, L and I; (ii) D and E; (iii) C, Sand T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

As used herein, the terms “delete,” “deletion,” and the like refer to modifications to a gene or a regulatory element associated therewith or operatively linked thereto (e.g., a transcription factor-binding site, such as a promoter or enhancer element) that remove the gene or otherwise render the gene nonfunctional. Exemplary deletions, as described herein, include the removal of the entirety of a nucleic acid encoding a gene of interest, from the start codon to the stop codon of the target gene. Other examples of deletions as described herein include the removal of a portion of the nucleic acid encoding the target gene (e.g., one or more codons, or a portion thereof, such as a single nucleotide deletion) such that, upon expression of the partially-deleted target gene, the product is nonfunctional or less functional then a wild-type form of the target gene. Exemplary deletions as described herein include the removal of all or a portion of the regulatory element(s) associated with a gene of interest, such as all or a portion of the promoter and/or enhancer nucleic acids that regulate expression of the target gene.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference).

As used herein, a “dual variable domain immunoglobulin” (“DVD-lg”) refers to an antibody that combines the target-binding variable domains of two monoclonal antibodies via linkers to create a tetravalent, dual-targeting single agent. (Gu et al., Meth. Enzymol., 502:25-41, 2012; incorporated by reference herein).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1 983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:21 0 (1986); incorporated herein by reference. Heterospecific antibodies can include Fc mutations that enforce correct chain association in multi-specific antibodies, as described by Klein et al, mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos. 5,530,101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, lgG-(scFv)2, DVD-1g, (scFv)2-(scFv)2-Fc and (scFv)2-Fc-(scFv)2. In case of lgG-(scFv)2, the scFv can be attached to either the N-terminal or the C-terminal end of either the heavy chain or the light chain. Exemplary multi-specific molecules have been reviewed by Kontermann, 2012, mAbs 4(2):182-197, Yazaki et al., 2013, Protein Engineering, Design & Selection 26(3):1 87-1 93, and Grote et al., 2012, in Proetzel & Ebersbach (eds.), Antibody Methods and Protocols, Methods in Molecular Biology vol. 901, chapter 16:247-263; incorporated herein by reference. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (ONASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a 5 position in the candidate sequence is occupied by the same amino acid residue as the corresponding position in the reference sequence, then the molecules are identical at that position.

As used herein, the term “primatized antibody” refers to an antibody comprising framework regions from primate-derived antibodies and other regions, such as CDRs and constant regions, from antibodies of a non-primate source. Methods for producing primatized antibodies are known in the art. See e.g., U.S. Pat. Nos. 5,658,570; 5,681,722; and 5,693,780; incorporated herein by reference.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the terms “regulatory element” and the like refer to promoters, enhancers, and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, C A, 1990); incorporated herein by reference.

As used herein, the terms “subject” and “patient” refer to an organism that receives treatment for a particular disease or condition as described herein (such as cancer or an infectious disease). Examples of subjects and patients include mammals, such as humans, receiving treatment for diseases or conditions, for example, cell proliferation disorders, such as cancer.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of the invention can be modified such that they vary in amino acid sequence from the antibody molecule from which they were derived. For example, in some embodiments, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen may bind to the antigen with a K D of less than 100 nM. For example, an antibody or antigen-binding fragment thereof that specifically binds to an antigen may bind to the antigen with a K D of up to 100 nM (e.g., between 1 pM and 100 nM). An antibody or antigen-binding fragment thereof that does not exhibit specific binding to a particular antigen or epitope thereof may exhibit a K D of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder, such as the progression of a cell proliferation disorder, such as cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors of the invention may contain one or more additional sequence elements used for the expression of proteins and/or the integration of these polynucleotide sequences into the genome of a host cell, such as a mammalian cell (e.g., a human cell). Exemplary vectors that can be used for the expression of antibodies and antibody fragments described herein include plasmids that contain regulatory sequences, such as promoter and enhancer regions, which direct gene transcription. Vectors may contain nucleic acids that modulate the rate of translation of a target gene or that improve the stability or nuclear export of the mRNA that results from gene transcription. These sequence elements may include, e.g., 5′ and 3′ untranslated regions, an internal ribosomal entry site (IRES), and polyadenylation signal site in order to direct efficient transcription of the gene carried on the expression vector. The vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VII” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 1 50,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

### Gene Definitions

As used herein, “C2L” refers to a orthopoxvirus gene, such as a gene that encodes a kelch-like protein. Non-limiting examples of protein sequences encoding the C2L gene are listed in tables 31-35 below. The term “C2L” may also include fragments or variants of the proteins listed in the tables below, or homologous genes from another orthopoxvirus strain.

As used herein, “C1L” refers to a orthopoxvirus gene. Non-limiting examples of protein sequences encoding the C2L gene are listed in tables 31-35 below. The term “C1L” may also include fragments or variants of the proteins listed in the tables below, or homologous genes from another orthopoxvirus strain.

As used herein, “N1L” refers to a orthopoxvirus gene, such as a gene that encodes a BCL-2 inhibitor. Non-limiting examples of protein sequences encoding the N1L gene are listed in tables 31-35 below. The term “N1L” may also include fragments or variants of the proteins listed in the tables below, or homologous genes from another orthopoxvirus strain.

As used herein, “N2L” refers to a orthopoxvirus gene, such as a gene that encodes a TLR signaling inhibitor. Non-limiting examples of protein sequences encoding the N2L gene are listed in tables 31-35 below. The term “N2L” may also include fragments or variants of the proteins listed in the tables below, or homologous genes from another orthopoxvirus strain.

As used herein, “M1L” refers to a orthopoxvirus gene, such as a gene that encodes an Ankyrin repeat protein. Non-limiting examples of protein sequences encoding the M1L gene are listed in tables 31-35 below. The term “M1L” may also include fragments or variants of the proteins listed in the tables below, or homologous genes from another orthopoxvirus strain.

As used herein, “M2L” refers to a orthopoxvirus gene, such as a gene that encodes an NF-κB inhibitor. Non-limiting examples of protein sequences encoding the M2L gene are listed in tables 31-35 below. The term “M2L” may also include fragments or variants of the proteins listed in the tables below, or homologous genes from another orthopoxvirus strain.

As used herein, “K1L” refers to a orthopoxvirus gene, such as a gene that encodes an NF-κB inhibitor. Non-limiting examples of protein sequences encoding the K1L gene are listed in tables 31-35 below. The term “K1L” may also include fragments or variants of the proteins listed in the tables below, or homologous genes from another orthopoxvirus strain.

As used herein, “K2L” refers to a orthopoxvirus gene, such as a gene that encodes an Ankyrin repeat protein. Non-limiting examples of protein sequences encoding the K2L gene are listed in tables 31-35 below. The term “K2L” may also include fragments or variants of the proteins listed in the tables below, or homologous genes from another orthopoxvirus strain.

As used herein, “K3L” refers to a orthopoxvirus gene, such as a gene that encodes a PKR inhibitor. Non-limiting examples of protein sequences encoding the K3L gene are listed in tables 31-35 below. The term “K3L” may also include fragments or variants of the proteins listed in the tables below, or homologous genes from another orthopoxvirus strain.

As used herein, “K4L” refers to a orthopoxvirus gene, such as a gene that encodes a phospholipase-D. Non-limiting examples of protein sequences encoding the K4L gene are listed in tables 31-35 below. The term “K4L” may also include fragments or variants of the proteins listed in the tables below, or homologous genes from another orthopoxvirus strain.

As used herein, “K5L” refers to a orthopoxvirus gene, such as a gene that encodes a monoglyceride lipase. Non-limiting examples of protein sequences encoding the K5L gene are listed in tables 31-35 below. The term “K5L” may also include fragments or variants of the proteins listed in the tables below, or homologous genes from another orthopoxvirus strain.

As used herein, “K6L” refers to a orthopoxvirus gene, such as a gene that encodes a monoglyceride lipase. Non-limiting examples of protein sequences encoding the K6L gene are listed in tables 31-35 below. The term “K6L” may also include fragments or variants of the proteins listed in the tables below, or homologous genes from another orthopoxvirus strain.

As used herein, “K7R” refers to a orthopoxvirus gene, such as a gene that encodes an NF-κB inhibitor. Non-limiting examples of protein sequences encoding the K7R gene are listed in tables 31-35 below. The term “K7R” may also include fragments or variants of the proteins listed in the tables below, or homologous genes from another orthopoxvirus strain.

As used herein, “F1L” refers to a orthopoxvirus gene, such as a gene that encodes a caspase-9 inhibitor. Non-limiting examples of protein sequences encoding the F1L gene are listed in tables 31-35 below. The term “F1L” may also include fragments or variants of the proteins listed in the tables below, or homologous genes from another orthopoxvirus strain.

As used herein, “F2L” refers to a orthopoxvirus gene, such as a gene that encodes a dUTPase. Non-limiting examples of protein sequences encoding the F2L gene are listed in tables 31-35 below. The term “F2L” may also include fragments or variants of the proteins listed in the tables below, or homologous genes from another orthopoxvirus strain.

As used herein, “F3L” refers to a orthopoxvirus gene, such as a gene that encodes a kelch-like protein. Non-limiting examples of protein sequences encoding the F3L gene are listed in tables 31-35 below. The term “F1L” may also include fragments or variants of the proteins listed in the tables below, or homologous genes from another orthopoxvirus strain.

As used herein, “B14R” refers to a orthopoxvirus gene. Non-limiting examples of protein sequences encoding the B14R gene are listed in tables 36-40 below. The term “B14R” may also include fragments or variants of the proteins listed in the tables below, or homologous genes from another orthopoxvirus strain.

As used herein, “B15R” refers to a orthopoxvirus gene. Non-limiting examples of protein sequences encoding the B15R gene are listed in tables 36-40 below. The term “B15R” may also include fragments or variants of the proteins listed in the tables below, or homologous genes from another orthopoxvirus strain.

As used herein, “B16R” refers to a orthopoxvirus gene, such as a gene that encodes a IL-1-beta inhibitor. Non-limiting examples of protein sequences encoding the B16R gene are listed in tables 31-35 below. The term “B16R” may also include fragments or variants of the proteins listed in the tables below, or homologous genes from another orthopoxvirus strain.

As used herein, “B17L” refers to a orthopoxvirus gene. Non-limiting examples of protein sequences encoding the B17L gene are listed in tables 36-40 below. The term “B17L” may also include fragments or variants of the proteins listed in the tables below, or homologous genes from another orthopoxvirus strain.

As used herein, “B18R” refers to a orthopoxvirus gene, such as a gene that encodes an Ankyrin repeat protein. Non-limiting examples of protein sequences encoding the B18R gene are listed in tables 36-40 below. The term “B18R” may also include fragments or variants of the proteins listed in the tables below, or homologous genes from another orthopoxvirus strain.

As used herein, “B19R” refers to a orthopoxvirus gene, such as a gene that encodes a IFN-alpha-beta-receptor-like secreted glycoprotein. Non-limiting examples of protein sequences encoding the B19R gene are listed in tables 36-40 below. The term “B19R” may also include fragments or variants of the proteins listed in the tables below, or homologous genes from another orthopoxvirus strain.

As used herein, “B20R” refers to a orthopoxvirus gene, such as a gene that encodes an Ankyrin repeat protein. Non-limiting examples of protein sequences encoding the B20R gene are listed in tables 36-40 below. The term “B20R” may also include fragments or variants of the proteins listed in the tables below, or homologous genes from another orthopoxvirus strain.

As used herein, “B8R” refers to a orthopoxvirus gene, such as a gene that encodes a secreted protein with homology to the gamma interferon (IFN-7). A nonlimiting example of a protein sequence encoded by an exemplary B8R gene in a Copenhagen strain of the vaccinia virus is given in UniProtKB database entry P21004 and is reproduced below:

(SEQ ID NO: 209)
MRYIIILAVLFINSIHAKITSYKFESVNFDSKIEWTGDGLYNISLKNYGI
KTWQTMYTNVPEGTYDISAFPKNDFVSFWVKFEQGDYKVEEYCTGLCVEV
KIGPPTVTLTEYDDHINLYIEHPYATRGSKKIPIYKRGDMCDIYLLYTAN
FTFGDSEEPVTYDIDDYDCTSTGCSIDFATTEKVCVTAQGATEGFLEKIT
PWSSEVCLTPKKNVYTCAIRKEDVPNFKDKMARVIKRKFNKQSQSYLTKF
LGSTSNDVTTFLSMLNLTKYS

The term “B8R” may also include fragments or variants of the proteins listed above, or homologous genes from another orthopoxvirus strain. Variants include without limitation those sequences having 85 percent or greater identity to the sequences disclosed herein.

### BRIEF DESCRIPTION OF THE FIGURES

FIG. 1 shows the phylogenetic analysis of 59 poxvirus strains, including the Orthopoxvirus virus strains.

FIG. 2 shows the abundance of different viral strains after passaging 5 Vaccinia viruses in different tumor types.

FIG. 3 shows the ability to replicate in various different patient tumor cores of Vaccinia wild-type strains.

FIG. 4 shows plaque size measurements of different Vaccinia wild-type strains.

FIG. 5A shows the number of TTAA sites across 1 kb regions in Vaccinia Copenhagen genome.

FIG. 5B shows the frequency of Transposon Insertions across Vaccinia Copenhagen genome. Each dot represents a transposon knockout of a particular gene. The position of the dot on the y-axis is determined by the frequency of the knockout.

FIG. 5C shows Poxvirus gene conservation in 59 viruses. Higher conservation indicates the gene is present in a larger amount of species.

FIG. 6 shows the frequency of various transposon knockouts after passaging in permissive cancer cells.

FIG. 7 shows plaque size measurements of purified transposons.

FIG. 8 shows the genomic structure of a 5p deletion (CopMD5p) and a 3p deletion (CopMD3p). CopMD5p and CopMD3p were crossed to generate CopMD5p3p.

FIG. 9 shows a heatmap showing cancer cell death following infection with either Copenhagen or CopMD5p3p at various doses.

FIG. 10 shows the growth curves of Copenhagen and CopMD5p3p replication in 4 different cancer cell lines.

FIG. 11 shows the ability of Copenhagen and CopMD5p3p to replicate in patient ex vivo samples as shown by tittering.

FIG. 12 shows that the modified CopMD5p3p virus forms different plaques than the parental virus. CopMD5p3p plaques are much clearer in the middle and we can see syncytia (cell fusion).

FIG. 13 shows CopMD5p3p induces syncytia (cell fusion) in 786-O cells.

FIG. 14 shows that CopMD5p3p is able to control tumour growth similarly to Copenhagen wild-type but does not cause weight loss.

FIG. 15 shows that CopMD5p3p does not cause pox lesion formation when compared to two other Vaccinia strains (Copenhagen and Wyeth) harboring the oncolytic knockout of thymidine kinase.

FIG. 16 shows the IVIS bio-distribution of Vaccinia after systemic administration in nude CD-1 mice. Luciferase encoding CopMD5p3p (TK KO) is tumor specific and does not replicate in off target tissues.

FIG. 17 shows the bio-distribution of Vaccinia after systemic administration. CopMD5p3p replicates similarly to other oncolytic Vaccinia in the tumour but replicates less in off target tissues/organs.

FIG. 18 shows the immunogenicity of Vaccinia in Human PBMCs. The ability of CopMD5p3p to induce human innate immune cell activation is stronger than that of wild-type Copenhagen. Data was acquired through flow cytometric analysis.

FIG. 19 shows the immunogenicity of Vaccinia in Mouse Splenocytes. The ability of CopMD5p3p to induce mouse innate immune cell activation is stronger than that of Copenhagen. Data was acquired through flow cytometric analysis.

FIG. 20 shows the immunogenicity of Vaccinia in Human cells. The ability of CopMD5p3p to activate NF-kB immune transcription factor is stronger than that of Copenhagen or VVdd but similar to that of MG-1. Data shown are western blots.

FIG. 21 shows the synergy with immune checkpoint inhibitor Anti-CTLA-4 (100 μg) in an aggressive melanoma model (B16-F10). In vivo efficacy measured by survival in an immune competent murine model treated with Vaccinia and Immune Checkpoint Inhibitors Anti-CTLA4.

FIG. 22 shows the synergy with immune checkpoint inhibitor Anti-CTLA4 (100 μg). In vivo efficacy measured by tumor growth (top row) and survival (bottom row) in an immune competent murine model treated with Vaccinia and Immune Checkpoint Inhibitor Anti-CTLA4. CopMD5p3p (left column) is compared to oncolytic Copenhagen TK KO (right column).

FIG. 23 shows the synergy with immune checkpoint inhibitor Anti-PD1 (100 μg). In vivo efficacy measured by tumor growth (top row) and survival (bottom row) in an immune competent murine model treated with Vaccinia and Immune Checkpoint Inhibitor Anti-PD1. CopMD5p3p (left column) is compared to oncolytic Copenhagen TK KO (right column).

FIG. 24 shows the synergy with immune checkpoint inhibitor Anti-PD1 (25 μg) and Anti-CTLA-4 (25 μg). In vivo efficacy measured by tumor growth (top row) and survival (bottom row) in an immune competent murine model treated with Vaccinia and Immune Checkpoint Inhibitors Anti-PD1 and Anti-CTLA4. CopMD5p3p (left column) is compared to oncolytic Copenhagen TK KO (right column).

FIG. 25 shows a schematic representation of the homologous recombination targeting strategy employed to generate denovo 5p (left) and 3p (right) major deletions in various vaccinia strains.

FIG. 26 shows the ability of wild-type Copenhagen vaccinia virus and several modified Copenhagen vaccinia virions to proliferate in various cell lines.

FIG. 27 shows the cytotoxic effects of wild-type Copenhagen vaccinia virus and several modified Copenhagen vaccinia virions on various cell lines, as assessed by coomassie blue (upper panel) and an Alamar Blue assay (lower panel). The order of strains listed for each cell line along the x-axis of the chart shown in the lower panel is as follows: from left to right, CopMD5p, CopMD5p3p, CopMD3p, and CopWT.

FIG. 28 shows the distribution of wild-type Copenhagen vaccinia virus and several modified Copenhagen vaccinia virions upon administration to mice.

FIG. 29 shows the ability of wild-type Copenhagen vaccinia virus and several modified Copenhagen vaccinia virions to activate Natural Killer (NK) cells and stimulate an immune response.

FIG. 30 shows the ability of wild-type Copenhagen vaccinia virus and several modified Copenhagen vaccinia virions to enhance NK cell-mediated degranulation against HT29 cells, a measure of NK cell activity and stimulate an immune response.

FIG. 31 shows the ability of wild-type Copenhagen vaccinia virus and several modified Copenhagen vaccinia virions to prime T-cells to initiate an anti-tumor immune response.

FIG. 32 shows the ability of wild-type Copenhagen vaccinia virus and several modified Copenhagen vaccinia virions to spread to distant locations from the initial point of infection.

FIG. 33 shows the ability of wild-type Copenhagen vaccinia virus and several modified Copenhagen vaccinia virions to form plaques, a measure of viral proliferation.

FIG. 34 shows the ability of wild-type Copenhagen vaccinia virus and several modified Copenhagen vaccinia virions to form plaques in 786-O cells.

FIG. 35 shows the percentage of genes deleted in CopMD5p3p in various poxvirus genomes.

FIG. 36 shows infection of normal versus cancer cell lines of SKV-B8R+ virus.

FIG. 37 shows SKV-B8R+ does not impair interferon signaling.

FIG. 38 shows SKV (CopMD5p3-B8R−) has similar efficacy in tumour control compared to SKV-B8R+.

FIG. 39 shows SKV engineered to express 2 immunotherapeutic transgenes and an antibody.

FIG. 40 shows SKV expressing murine IL-12 p35 membrane bound has greater efficacy in controlling murine tumours.

FIG. 41 shows major double deletions engineered in various vaccinia strains enhance cancer cell killing in vitro.

FIG. 42 shows the phenotypic characterization of HeLa cells infected with various vaccinia strains.

FIG. 43 shows 5p3p vaccinia strains do not induce weight loss compared to wildtype strains.

FIG. 44 shows 5p3p vaccinia strains do not induce pox lesions compared to wildtype strains.

### DETAILED DESCRIPTION

The present invention features genetically modified orthopoxviruses, such as vaccinia viruses (e.g. Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1 viruses), as well as the use of the same for the treatment of various cancers. The invention is based in part on the surprising discovery that orthopoxviruses, such as Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1 viruses, exhibit markedly improved oncolytic activity, replication in tumors, infectivity, immune evasion, tumor persistence, capacity for incorporation of exogenous DNA sequences, and amenability for large scale manufacturing when the viruses are engineered to contain deletions in one or more, or all, of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, K ORF A, K ORF B, B ORF E, B ORF F, B ORF G, B21R, B22R, B23R, B24R, B25R, B26R, B27R, B28R, and B29R genes. In various embodiments of the invention, the modified orthopox viruses contain a deletion of the B8R gene. While inactive in mice, the B8R gene neutralizes antiviral activity of human IFN-γ. In various embodiments, at least one transgene is subsequently inserted into locus of the B8R gene (now deleted) through a homologous recombination targeting strategy. In various embodiments, the modified orthopoxvirus expresses at least one of three transgenes: IL-12-TM, FLT3-L and anti-CLTA4 antibody.

The orthopoxviruses described herein can be administered to a patient, such as a mammalian patient (e.g., a human patient) to treat a variety of cell proliferation disorders, including a wide range of cancers. The sections that follow describe orthopoxviruses and genetic modifications thereto, as well as methods of producing and propagating genetically modified orthopoxviruses and techniques for administering the same to a patient.

### Poxvirus

Generally, a poxvirus viral particle is oval or brick-shaped, measuring some 200-400 nm long. The external surface is ridged in parallel rows, sometimes arranged helically. Such particles are extremely complex, containing over 100 distinct proteins. The extracellular forms contain two membranes (EEV: extracellular enveloped virions), whereas intracellular particles only have an inner membrane (IMV: intracellular mature virions). The outer surface is composed of lipid and protein that surrounds the core, which is composed of a tightly compressed nucleoprotein. Antigenically, poxviruses are also very complex, inducing both specific and cross-reacting antibodies. There are at least ten enzymes present in the particle, mostly concerned with nucleic acid metabolism/genome replication.

The genome of the wild-type poxvirus is linear double-stranded DNA of 130-300 Kbp. The ends of the genome have a terminal hairpin loop with several tandem repeat sequences. Several poxvirus genomes have been sequenced, with most of the essential genes being located in the central part of the genome, while non-essential genes are located at the ends. There are about 250 genes in the poxvirus genome. Replication takes place in the cytoplasm, as the virus is sufficiently complex to have acquired all the functions necessary for genome replication. There is some contribution by the cell, but the nature of this contribution is not clear. However, even though poxvirus gene expression and genome replication occur in enucleated cells, maturation is blocked, indicating some role by the cell.

Once into the cell cytoplasm, gene expression is carried out by viral enzymes associated with the core. Expression is divided into 2 phases: early genes: which represent about of 50% genome, and are expressed before genome replication, and late genes, which are expressed after genome replication. The temporal control of expression is provided by the late promoters, which are dependent on DNA replication for activity. Genome replication is believed to involve self-priming, leading to the formation of high molecular weight concatemers, which are subsequently cleaved and repaired to make virus genomes. Viral assembly occurs in the cytoskeleton and probably involves interactions with the cytoskeletal proteins (e.g., actin-binding proteins). Inclusions form in the cytoplasm that mature into virus particles. Cell to cell spread may provide an alternative mechanism for spread of infection. Overall, replication of this large, complex virus is rather quick, taking just 12 hours on average. At least nine different poxviruses cause disease in humans, but variola virus and vaccinia are the best known. Variola strains are divided into variola major (25-30% fatalities) and variola minor (same symptoms but less than 1% death rate). Infection with both viruses occurs naturally by the respiratory route and is systemic, producing a variety of symptoms, but most notably with variola characteristic pustules and scarring of the skin.

### Vaccinia Virus as a Species of Orthopoxvirus

Vaccinia virus is a large, complex enveloped virus having a linear double-stranded DNA genome of about 190K by and encoding for approximately 250 genes. Vaccinia is well-known for its role as a vaccine that eradicated smallpox. Post-eradication of smallpox, scientists have been exploring the use of vaccinia as a tool for delivering genes into biological tissues (gene therapy and genetic engineering). Vaccinia virus is unique among DNA viruses as it replicates only in the cytoplasm of the host cell. Therefore, the large genome is required to code for various enzymes and proteins needed for viral DNA replication. During replication, vaccinia produces several infectious forms, which differ in their outer membranes: the intracellular mature virion (IMV), the intracellular enveloped virion (IEV), the cell-associated enveloped virion (CEV), and the extracellular enveloped virion (EEV). IMV is the most abundant infectious form and is thought to be responsible for spread between hosts. On the other hand, the CEV is believed to play a role in cell-to-cell spread and the EEV is thought to be important for long-range dissemination within the host organism.

Vaccinia virus is closely related to the virus that causes cowpox. The precise origin of vaccinia is unknown, but the most common view is that vaccinia virus, cowpox virus, and variola virus (the causative agent for smallpox) were all derived from a common ancestral virus. There is also speculation that vaccinia virus was originally isolated from horses. A vaccinia virus infection is mild and typically asymptomatic in healthy individuals, but it may cause a mild rash and fever, with an extremely low rate of fatality. An immune response generated against a vaccinia virus infection protects that person against a lethal smallpox infection. For this reason, vaccinia virus was used as a live-virus vaccine against smallpox. The vaccinia virus vaccine is safe because it does not contain the smallpox virus, but occasionally certain complications and/or vaccine adverse effects may arise, especially if the vaccine is immunocompromised.

Exemplary strains of the vaccinia virus include, but are not limited to, Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1.

### Thymidine Kinase Mutants

Several current clinical studies testing vaccinia virus as an oncolytic virus harbor deletions in the viral Thymidine Kinase (TK) gene. This deletion attenuates the virus, rendering the virus dependent upon the activity of cellular thymidine kinase for DNA replication and, thus, viral propagation. Cellular thymidine kinase is expressed at a low level in most normal tissues and at elevated levels in many cancer cells. Through metabolic targeting, TK-viruses can grow in cells that have a high metabolic rate (e.g., healthy cells or tumor cells) and will not grow well in cells that have low levels of thymidine kinase. Since there exist quiescent tumour cells (e.g., cancer stem cells), TK-viruses are likely compromised in their ability to kill this population of cancer cells just as chemotherapy is largely ineffective. The modified viral vectors described in this disclosure retains virus synthetic machinery (including TK) and may propagate in quiescent cancer cells. The viral modifications of this disclosure may allow the virus to be highly selective without deleting TK or other DNA metabolizing enzymes (e.g., ribonucleotide reductase) and could be more effective in tumors with a low metabolic rate.

### Virus Propagation

The present invention features poxviruses, including those constructed with one or more gene deletions compared to wild-type, such that the virus exhibits desirable properties for use against cancer cells, while being less toxic or non-toxic to non-cancer cells. This section summarizes various protocols, by way of example, for producing recombinant poxviruses described herein, such as methods for generating mutated viruses through the use of recombinant DNA technology.

For example, to generate mutations in the poxvirus genome, native and modified polypeptides may be encoded by a nucleic acid molecule comprised in a vector. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1994, both incorporated herein by reference. In addition to encoding a modified polypeptide such as modified gelonin, a vector may encode non-modified polypeptide sequences such as a tag or targeting molecule.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors or viruses (which does not qualify as a vector if it expresses no exogenous polypeptides). A host cell may be “transfected” or “transformed,” which refers to a process by which exogenous nucleic acid, such as a modified protein-encoding sequence, is transferred or introduced into the host cell. A transformed cell includes the primary subject cell and its progeny. Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KCB, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris. Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Genetic Modifications to the Orthopoxvirus Genome

### Methods of Genetic Modification.

Methods for the insertion or deletion of nucleic acids from a target genome include those described herein and known in the art. One such method that can be used for incorporating polynucleotides encoding target genes into a target genome involves the use of transposons. Transposons are polynucleotides that encode transposase enzymes and contain a polynucleotide sequence or gene of interest flanked by 5′ and 3′ excision sites. Once a transposon has been delivered to a target nucleic acid (e.g., in a host cell), expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the target genome by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the target genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US2005/0112764), the disclosures of each of which are incorporated herein by reference.

Additional methods for nucleic acid delivery to effect expression of compositions of the present invention are believed to include virtually any method by which a nucleic acid (e.g., DNA, including viral and non-viral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### CopMD5p, CopMD3p, and CopMD5p3p Deletions.

In various embodiments, various genes are deleted to enhance the oncolytic activity of the orthopoxvirus. Most of the deletions described herein are either involved in blocking a host response to viral infection or otherwise have an unknown function. In various embodiments, at least one of the genes depicted in Table 2 are deleted from the recombinant orthopoxvirus genome. In various embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of the genes depicted in Table 2 are deleted from the recombinant orthopox genome. In various embodiments, all of the genes depicted in Table 2 are deleted from the recombinant orthopoxvirus genome. Three exemplary embodiments of the present invention, CopMD5p, CopMD3p and CopMD5p3p, are described herein. Depicted in Table 2 below are clusters of deleted genes and their function in CopMD5p, CopMD3p, and CopMD5p3p virus. In various embodiments, where two copies of an ITR exist, only the right ITR of the genome is deleted and the left ITR remains intact. Deletions are confirmed by whole genome sequencing.

TABLE 2
Deleted genes in Orthopoxviruses

| Name | Category | Function | Virus Deletions | |
|---|---|---|---|---|
| C2L | Host interaction | Inhibits NFkB | CopMD5p | CopMD5p3p |
| C1L | Unknown | Unknown | | |
| N1L | Host interaction | Inhibits NFkB and Apoptosis | | |
| N2L | Host interaction | Inhibits IRF3 | | |
| M1L | Unknown | Unknown | | |
| M2L | Host interaction | Inhibits NFkB and Apoptosis | | |
| K1L | Host interaction | Inhibits PKR and NF-kB | | |
| K2L | Host interaction | Prevents cell fusion | | |
| K3L | Host interaction | Inhibits PKR | | |
| K4L | DNA replication | DNA modifying nuclease | | |
| K5L | Pseudogene | Pseudogene | | |
| K6L | Pseudogene | Pseudogene | | |
| K7R | Host interaction | Inhibits NFkB and IRF3 | | |
| F1L | Host interaction | Inhibits Apoptosis | | |
| F2L | DNA replication | Deoxyuridine triphosphatase | | |
| F3L | Host interaction | Virulence factor | | |
| B14R | Pseudogene | Pseudogene | CopMD3p | |
| B15R | Unknown | Unknown | | |
| B16R | Host interaction | IL-1-beta-inhibitor | | |
| B17L | Unknown | Unknown | | |
| B18R | Unknown | Ankyrin-like | | |
| B19R | Host interaction | Secreted IFNa sequestor | | |
| B20R | Unknown | Ankyrin-like | | |
| B21R-ITR* | Unknown | Unknown | | |
| B22R-ITR* | Unknown | Unknown | | |
| B23R-ITR* | Unknown | Unknown | | |
| B24R-ITR* | Unknown | Unknown | | |
| B25R-ITR* | Unknown | Unknown | | |
| B26R-ITR* | Unknown | Unknown | | |
| B27R-ITR* | Unknown | Unknown | | |
| B28R-ITR* | Pseudogene | TNF-a receptor | | |
| B29R-ITR* | Host interaction | Secreted CC-chemokine sequestor | | |

### B8R Gene Deletions.

In various embodiments, the orthopox viruses are further genetically modified to contain deletions in the B8R gene. The vaccinia virus B8R gene encodes a secreted protein with homology to gamma interferon receptor (IFN-γ). In vitro, the B8R protein binds to and neutralizes the antiviral activity of several species of gamma interferon including human and rat gamma interferon; it does not, however, bind significantly to murine IFN-γ. Deleting the B8R gene prevents the impairment of IFN-γ in humans. Deletion of the B8R gene results in enhanced safety without a concomitant reduction in immunogenicity.

### Transgene Insertions

In various embodiments, additional transgenes may be inserted into the vector. In various embodiments, one, two or three transgenes are inserted into the locus of the deleted B8R gene. In some strains, in addition to the transgene(s) present at the site of the B8R deletion, the strain also has, at least one transgene is inserted into an additional locus on the orthopox virus that is not the locus of the deleted B8R gene. In various embodiments, at least one transgene is inserted into boundaries of the 5p deletions, at least one transgene is inserted into the boundaries of the 3p deletions or both. In various, embodiments at least three, four, five or more transgenes are inserted into the modified orthopox virus genome.

In various embodiments, the recombinant orthopoxvirus vector can include at least one transgene encoding an immune checkpoint inhibitor. Exemplary immune checkpoint inhibitors for expression by the orthopoxvirus of the compositions and methods of the invention include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.

In various embodiments, the recombinant orthopoxvirus vector can include at least one transgene encoding at least one interleukin protein. Exemplary interleukin proteins for expression by the orthopoxvirus of the compositions and methods of the invention include but are not limited to IL-1 alpha, IL-1 beta, IL-2, IL-4, IL-7, IL-10, IL-12 p35, IL-12 p40, IL-12 p70, IL-15, IL-18, IL-21, and IL-23.

In various embodiments, recombinant orthopoxvirus vector can include a transgene encoding an interferon. Exemplary interferons for expression by the orthopoxvirus of the compositions and methods of the invention include but are not limited to IFN-alpha, IFN-beta, IFN-delta, IFN-epsilon, IFN-tau, IFN-omega, IFN-zeta, and IFN-gamma.

In various embodiments, the recombinant orthopoxvirus vector can include a transgene encoding a TNF superfamily member protein. Exemplary TNF superfamily member proteins for expression by the orthopoxvirus of the compositions and methods of the invention include but are not limited to TRAIL, Fas ligand, LIGHT (TNFSF-14), TNF-alpha, and 4-1BB ligand.

In various embodiments, the recombinant orthopoxvirus vector can include a transgene encoding a cytokine. Exemplary cytokines for expression by the orthopoxvirus of the compositions and methods of the invention include but are not limited to GM-CSF, Flt3 ligand, CD40 ligand, anti-TGF-beta, anti-VEGF-R2, and cGAS (guanyl adenylate cyclase).

In various embodiments, the recombinant orthopoxvirus vector can include a transgene encoding a tumor-associated antigen. Exemplary tumor-associated antigens for expression by the orthopoxvirus of the compositions and methods of the invention include but are not limited to CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, NTRK, MAGE-A3, NY-ESO-1, one or more human papillomavirus (HPV) proteins, E6 and E7 proteins of HPV16, E6 and E7 proteins of HPV18, brachyury, or prostatic acid phosphatase, or one or more fragments thereof. Additional examples of tumor-associated antigens for use in conjunction with the compositions and methods described herein include, but are not limited to, those listed in tables 3-30.

### Methods of Treatment

### Pharmaceutical Composition, Administration, and Doses

Therapeutic compositions containing recombinant orthopoxvirus vectors of the invention can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

To induce oncolysis, kill cells, inhibit growth, inhibit metastases, decrease tumor size and otherwise reverse or reduce the malignant phenotype of tumor cells, using the methods and compositions of the present invention, one may contact a tumor with the modified orthopoxvirus, e.g., by administration of the orthopoxvirus to a patient having cancer by way of, for instance, one or more of the routes of administration described herein. The route of administration may vary with the location and nature of the cancer, and may include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation.

The term “intravascular” is understood to refer to delivery into the vasculature of a patient, meaning into, within, or in a vessel or vessels of the patient. In certain embodiments, the administration is into a vessel considered to be a vein (intravenous), while in others administration is into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Intratumoral injection, or injection directly into the tumor vasculature is specifically contemplated for discrete, solid, accessible tumors. Local, regional or systemic administration also may be appropriate. The viral particles may advantageously be contacted by administering multiple injections to the tumor, spaced, for example, at approximately 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature. Such continuous perfusion may take place, for example, for a period of from about 1-2 hours, to about 2-6 hours, to about 6-12 hours, or about 12-24 hours following the initiation of treatment. Generally, the dose of the therapeutic composition via continuous perfusion may be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary, and often depend on tumor type, tumor location, disease progression, and health and age of the patient. Certain types of tumor will require more aggressive treatment, while at the same time, certain patients cannot tolerate more taxing protocols. The clinician will be best suited to make such decisions based on the known efficacy and toxicity (if any) of the therapeutic formulations. In certain embodiments, the tumor being treated may not, at least initially, be resectable. Treatments with the therapeutic agent of the disclosure may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses may range from 10³, 10⁴, 10⁵, 10⁶, 10⁷, 10⁸, 10⁹, 10¹⁰, 10¹¹, 10¹², to 10¹³ pfu and higher. Additionally or alternatively, depending on the kind of virus and the titer attainable, one may deliver 1 to 100, 10 to 50, 100-1000, or up to about or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp), including all values and ranges there between, to the tumor or tumor site.

Another method of delivery of the recombinant orthopoxvirus genome disclosed herein to cancer or tumor cells may be via intratumoral injection. However, the pharmaceutical compositions disclosed herein may alternatively be administered parenterally, intravenously, intradermally, intramuscularly, transdermally or even intraperitoneally as described in U.S. Pat. Nos. 5,543,158; 5,641,515 and 5,399,363 (each specifically incorporated herein by reference in its entirety). Injection of nucleic acid constructs may be delivered by syringe or any other method used for injection of a solution, as long as the expression construct can pass through the particular gauge of needle required for injection. An exemplary needleless injection system that may be used for the administration of recombinant orthopoxviruses described herein is exemplified in U.S. Pat. No. 5,846,233. This system features a nozzle defining an ampule chamber for holding the solution and an energy device for pushing the solution out of the nozzle to the site of delivery. Another exemplary syringe system is one that permits multiple injections of predetermined quantities of a solution precisely at any depth (U.S. Pat. No. 5,846,225).

Mixtures of the viral particles or nucleic acids described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form may be sterile and may be fluid to the extent that easy syringability exists. It may be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### Cancer

The recombinant orthopoxvirus disclosed herein can be administered to a mammalian subject, such as a human, suffering from a cell proliferation disorder, such as cancer, e.g., to kill cancer cells directly by oncolysis and/or to enhance the effectiveness of the adaptive immune response against the target cancer cells. In some embodiments, the cell proliferation disorder is a cancer, such as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer. In particular cases, the cell proliferation disorder may be a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenström macroglobulinemia.

A physician having ordinary skill in the art can readily determine an effective amount of the recombinant orthopoxvirus vector for administration to a mammalian subject (e.g., a human) in need thereof. For example, a physician may start prescribing doses of recombinant orthopoxvirus vector at levels lower than that required in order to achieve the desired therapeutic effect and gradually increase the dosage until the desired effect is achieved. Alternatively, a physician may begin a treatment regimen by administering a dose of recombinant orthopoxvirus vector and subsequently administer progressively lower doses until a therapeutic effect is achieved (e.g., a reduction in the volume of one or more tumors). In general, a suitable daily dose of a recombinant orthopoxvirus vector of the invention will be an amount of the recombinant orthopoxvirus vector which is the lowest dose effective to produce a therapeutic effect. A daily dose of a therapeutic composition of the recombinant orthopoxvirus vector of the invention may be administered as a single dose or as two, three, four, five, six or more doses administered separately at appropriate intervals throughout the day, week, month, or year, optionally, in unit dosage forms. While it is possible for the recombinant orthopoxvirus vector of the invention to be administered alone, it may also be administered as a pharmaceutical formulation in combination with excipients, carriers, and optionally, additional therapeutic agents.

Recombinant orthopoxvirus vectors of the invention can be monitored for their ability to attenuate the progression of a cell proliferation disease, such as cancer, by any of a variety of methods known in the art. For instance, a physician may monitor the response of a mammalian subject (e.g., a human) to treatment with recombinant orthopoxvirus vector of the invention by analyzing the volume of one or more tumors in the patient. Alternatively, a physician may monitor the responsiveness of a subject (e.g., a human) t to treatment with recombinant orthopoxvirus vector of the invention by analyzing the T-reg cell population in the lymph of a particular subject. For instance, a physician may withdraw a sample from a mammalian subject (e.g., a human) and determine the quantity or density of cancer cells using established procedures, such as fluorescence activated cell sorting. A finding that the quantity of cancer cells in the sample has decreased (e.g., by 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99%, or more) relative to the quantity of cancer cells in a sample obtained from the subject prior to administration of the recombinant orthopoxvirus may be an indication that the orthopoxvirus administration is effectively treating the cancer.

### Combination Therapy

In various embodiments, the recombinant orthopoxvirus may be co-administered with other cancer therapeutics. Furthermore, in various embodiments, the recombinant orthopoxviruses described herein are administered in conjunction with other cancer treatment therapies, e.g., radiotherapy, chemotherapy, surgery, and/or immunotherapy. In some aspects of this invention, the recombinant orthopoxvirus described herein are administered in conjunction with checkpoint inhibitors. In various embodiments, the recombinant orthopoxvirus may be administered in conjunction with treatment with another immunoncology product. The recombinant orthopoxviruses of the present invention and other therapies or therapeutic agents can be administered simultaneously or sequentially by the same or different routes of administration. The determination of the identity and amount of therapeutic agent(s) for use in the methods of the present invention can be readily made by ordinarily skilled medical practitioners using standard techniques known in the art.

The recombinant orthopoxvirus vectors described herein may be administered with one or more additional agents, such as an immune checkpoint inhibitor. For instance, the recombinant orthopoxvirus vector can be administered simultaneously with, admixed with, or administered separately from an immune checkpoint inhibitor. Exemplary immune checkpoint inhibitors for use in conjunction with the compositions and methods of the invention include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.

Additionally or alternatively, a vector of the invention can be administered simultaneously with, admixed with, or administered separately from an interleukin (IL). For instance, the recombinant orthopoxvirus vector can be administered simultaneously with, admixed with, or administered separately from an interleukin. Exemplary interleukins for use in conjunction with the compositions and methods of the invention include but are not limited to IL-1 alpha, IL-1 beta, IL-2, IL-4, IL-7, IL-10, IL-12 p35, IL-12 p40, IL-12 p70, IL-15, IL-18, IL-21, and IL-23.

Additionally or alternatively, a vector of the invention can be administered simultaneously with, admixed with, or administered separately from an interferon. For instance, the recombinant orthopoxvirus vector can be administered simultaneously with, admixed with, or administered separately from an interferon. Exemplary interferons for use in conjunction with the compositions and methods of the invention include but are not limited to IFN-alpha, IFN-beta, IFN-delta, IFN-epsilon, IFN-tau, IFN-omega, IFN-zeta, and IFN-gamma.

Additionally or alternatively, a vector of the invention can be administered simultaneously with, admixed with, or administered separately from a TNF superfamily member protein. For instance, the recombinant orthopoxvirus vector can be administered simultaneously with, admixed with, or administered separately from a TNF superfamily member protein. Exemplary TNF superfamily member proteins for use in conjunction with the compositions and methods of the invention include but are not limited to TRAIL, Fas ligand, LIGHT (TNFSF-14), TNF-alpha, and 4-1BB ligand.

Additionally or alternatively, a vector of the invention can be administered simultaneously with, admixed with, or administered separately from a cytokine. For instance, the recombinant orthopoxvirus vector can be administered simultaneously with, admixed with, or administered separately from a cytokine. Exemplary cytokines for use in conjunction with the compositions and methods of the invention includes but are not limited to GM-CSF, Flt3 ligand, CD40 ligand, anti-TGF-beta, anti-VEGF-R2, and cGAS (guanyl adenylate cyclase).

TABLE 3
Ovarian cancer

| | Tumor-associated | Reported immunogenic | |
|---|---|---|---|
| No. | antigen | epitopes | Sources |
| 1 | Kallikrein 4 | FLGYLILGV; | Wilkinson et al. Cancer Immunol. |
| | | SVSESDTIRSISIAS; | Immunother. 61(2): 169-79 (2012). |
| | | LLANGRMPTVLQCVN; | Hural et al. J. Immunol. 169(1): 557- |
| | | and | 65 (2002). |
| | | RMPTVLQCVNVSVVS | |
| 2 | PBF | CTACRWKKACQR | Tsukahara et al. Cancer Res. |
| | | | 64(15): 5442-8 (2004). |
| 3 | PRAME | VLDGLDVLL; | Kessler et al. J. Exp. Med. |
| | | SLYSFPEPEA; | 193(1): 73-88 (2001). |
| | | ALYVDSLFFL; | Ikeda et al. Immunity 6(2): 199-208 |
| | | SLLQHLIGL; | (1997). |
| | | and | |
| | | LYVDSLFFL | |
| 4 | WT1 | TSEKRPFMCAY; | Asemissen et al. Clin. Cancer Res. |
| | | CMTWNQMNL; | 12(24): 7476-82 (2006) |
| | | LSHLQMHSRKH; | Ohminami et al. Blood. 95(1): 286- |
| | | KRYFKLSHLQMHSRKH; | 93 (2000). |
| | | and | Guo et al. Blood. 106(4): 1415-8 |
| | | KRYFKLSHLQMHSRKH | 2005). |
| | | | Lin et al. J. Immunother. 36(3): 159- |
| | | | 70 (2013). |
| | | | Fujiki et al. J. Immunother. |
| | | | 30(3): 282-93 (2007). |
| 5 | HSDL1 | CYMEAVAL | Wick et al. Clin. Cancer Res. |
| | | | 20(5): 1125-34 (2014). |
| 6 | Mesothelin | SLLFLLFSL | Hassan et al. Appl. |
| | | VLPLTVAEV | Immunohistochem. Mol. Morphol. |
| | | ALQGGGPPY | 13(3): 243-7 (2005). |
| | | LYPKARLAF | Thomas et al J Exp Med. 2004 Aug |
| | | AFLPWHRLF | 2; 200(3): 297-306. |
| 7 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAE | Knights et al. Cancer Immunol |
| | | L-ARRSLAQ | Immunother. 58(3): 325-38 (2009). |
| | | EFYLAMPFATPM | Jager et al. Cancer Immun. 2: 12 |
| | | PGVLLKEFTVSGNILTIR | (2002). |
| | | L-TAADHR | Zeng et al. Proc Natl Acad Sci USA. |
| | | RLLEFYLAMPFA | 98(7): 3964-9 (2001). |
| | | QGAMLAAQERRVPRAA | Mandic et al. J Immunol. |
| | | E-VPR | 174(3): 1751-9 (2005). |
| | | PFATPMEAELARR | Chen et al. Proc Natl Acad Sci USA. |
| | | PGVLLKEFTVSGNILTIR | 101(25): 9363-8 (2004). |
| | | LT | Ayyoub et al. Clin Cancer Res. |
| | | VLLKEFTVSG | 16(18): 4607-15 (2010). |
| | | AADHRQLQLSISSCLQQL | Slager et al. J Immunol. |
| | | LKEFTVSGNILTIRL | 172(8): 5095-102 (2004). |
| | | PGVLLKEFTVSGNILTIR | Mizote et al. Vaccine. 28(32): 5338- |
| | | L-TAADHR | 46 (2010). |
| | | LLEFYLAMPFATPMEAE | Jager et al. J Exp Med. 191(4): 625- |
| | | L-ARRSLAQ | 30 (2000). |
| | | KEFTVSGNILT | Zarour et al. Cancer Res. |
| | | LLEFYLAMPFATPM | 60(17): 4946-52 (2000). |
| | | AGATGGRGPRGAGA | Zeng et at. J Immunol. 165(2): 1153- |
| | | | 9(2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 8 | CEA | TYYRPGVNLSLSC | Galanis et al. Cancer Res. 70(3): 875- |
| | | EIIYPNASLLIQN | 82 (2010). |
| | | YACFVSNLATGRNNS | Bast et al. Am. J. Obstet. Gynecol. |
| | | LWWVNNQSLPVSP | 149(5): 553-9 (1984). |
| | | LWWVNNQSLPVSP | Crosti et al. J Immunol. |
| | | LWWVNNQSLPVSP | 176(8): 5093-9 (2006). |
| | | EIIYPNASLLIQN | Kobayashi et al. Clin Cancer Res. |
| | | NSIVKSITVSASG | 8(10): 3219-25 (2002). |
| | | KTWGQYWQV | Campi et al. Cancer Res. |
| | | (A)MLGTHTMEV | 63(23): 8481-6 (2003). |
| | | ITDQVPFSV | Bakker et al. Int J Cancer. 62(1): 97- |
| | | YLEPGPVTA | 102 (1995). |
| | | LLDGTATLRL | Tsai et al. J Immunol. 158(4): 1796- |
| | | VLYRYGSFSV | 802 (1997). |
| | | SLADTNSLAV | Kawakami et al. J Immunol. |
| | | RLMKQDFSV | 154(8): 3961-8 (1995). |
| | | RLPRIFCSC | Cox et al. Science. 264(5159): 716-9 |
| | | LIYRRRLMK | (1994). |
| | | ALLAVGATK | Kawakami et al. J Immunol. |
| | | IALNFPGSQK | 154(8): 3961-8 (1995). |
| | | RSYVPLAHR | Kawakami et al. J Immunol. |
| | | | 161(12): 6985-92 (1998). |
| | | | Skipper et al. J Immunol. |
| | | | 157(11): 5027-33 (1996). |
| | | | Michaux et al. J Immunol. |
| | | | 192(4): 1962-71 (2014). |
| 9 | p53 | VVPCEPPEV | Hung et al. Immunol. Rev. 222: 43- |
| | | | 69 (2008). |
| 10 | Her2/Neu | HLYQGCQVV | Nakatsuka et al. Mod. Pathol. |
| | | YLVPQQGFFC | 19(6): 804-814 (2006). |
| | | PLQPEQLQV | Pils et al. Br. J Cancer 96(3): 485-91 |
| | | TLEEITGYL | (2007). |
| | | ALIHHNTHL | Scardino et al. Eur J Immunol. |
| | | PLTSIISAV | 31(11): 3261-70 (2001). |
| | | VLRENTSPK | Scardino et al. J Immunol. |
| | | TYLPTNASL | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 11 | EpCAM | RYQLDPKFI | Spizzo et al. Gynecol. Oncol. |
| | | | 103(2): 483-8 (2006). |
| | | | Tajima et al. Tissue Antigens. |
| | | | 64(6): 650-9 (2004). |
| 12 | CA125 | ILFTINFTI | Bast et al. Cancer 116(12): 2850- |
| | | VLFTINFTI | 2853 (2010). |
| | | TLNFTITNL | |
| | | VLQGLLKPL | |
| | | VLQGLLRPV | |
| | | RLDPKSPGV | |
| | | QLYWELSKL | |
| | | KLTRGIVEL | |
| | | QLTNGITEL | |
| | | QLTHNITEL | |
| | | TLDRNSLYV | |
| 13 | Folate | FLLSLALML | Bagnoli et al. Gynecol. Oncol. |
| | receptor α | NLGPWIQQV | 88: S140-4 (2003). |
| | | | Pampeno et al. (2016) High-ranking |
| | | | In Silico epitopes [determined by 3 |
| | | | algorithms: BISMAS, IEDB, |
| | | | RANKPEP] unpublished |
| 14 | Sperm | ILDSSEEDK | Chiriva-Inernati et al. J. |
| | protein 17 | | Immunother. 31(8): 693-703 (2008). |
| 15 | TADG-12 | YLPKSWTIQV | Bellone et al. Cancer 115(4): 800-11 |
| | | WIHEQMERDLKT | (2009). |
| | | | Underwood et al. BBA Mol. Basis of |
| | | | Disease. 1502(3): 337-350 (2000). |
| 16 | MUC-16 | ILFTINFTI | Chekmasova et al. Clin. Cancer Res. |
| | | VLFTINFTI | 16(14): 3594-606 (2010). |
| | | TLNFTITNL | |
| | | VLQGLLKPL | |
| | | VLQGLLRPV | |
| | | RLDPKSPGV | |
| | | QLYWELSKL | |
| | | KLTRGIVEL | |
| | | QLTNGITEL | |
| | | QLTHNITEL | |
| | | TLDRNSLYV | |
| 17 | L1CAM | LLANAYIYV | Hong et al. J. Immunother. 37(2): 93- |
| | | YLLCKAFGA | 104 (2014). |
| | | KLSPYVHYT | Pampeno et al. (2016) High-ranking |
| | | | In Silico epitopes [determined by 3 |
| | | | algorithms: BISMAS, IEDB, |
| | | | RANKPEP] unpublished |
| 18 | Mannan-MUC-1 | PDTRPAPGSTAPPAHGV | Loveland et al. Clin. Cancer Res. |
| | | TSA | 12(3 Pt 1): 869-77 (2006). |
| | | STAPPVHNV | Godelaine et al. Cancer Immunol |
| | | LLLLTVLTV | Immunother. 56(6): 753-9 (2007). |
| | | PGSTAPPAHGVT | Ma et al. Int J Cancer. 129(10): 2427- |
| | | | 34 (2011). |
| | | | Wen et al. Cancer Sci. 102(8): 1455- |
| | | | 61 (2011). |
| | | | Jerome et al. J Immunol. |
| | | | 151(3): 1654-62 (1993). |
| | | | Brossart et al. Blood. 93(12): 4309- |
| | | | 17 (1999). |
| | | | Hiltbold et al. Cancer Res. |
| | | | 58(22): 5066-70 (1998). |
| 19 | HERV-K-MEL | MLAVISCAV | Schiavetti et al. Cancer Res. |
| | | | 62(19): 5510-6 (2002). |
| 20 | KK-LC-1 | RQKRILVNL | Fukuyama et al. Cancer Res. |
| | | | 66(9): 4922-8 (2006). |
| 21 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(19 |
| | | | Pt 1): 6047-57 (2004). |
| 22 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAA | Sun et al. Cancer Immunol |
| | | EVP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 23 | MAGE-A4 | EVDPASNTY | Kobayashi et al. Tissue Antigens. |
| | | GVYDGREHTV | 62(5): 426-32 (2003). |
| | | NYKRCFPVI | Duffour et al. Eur J Immunol. |
| | | SESLICMIF | 29(10): 3329-37 (1999). |
| | | | Miyahara et al. Clin Cancer Res. |
| | | | 11(15): 5581-9 (2005). |
| | | | Ottaviani et al. Cancer Immunol |
| | | | Immunother. 55(7): 867-72 (2006) |
| | | | Zang et al. Tissue Antigens. |
| | | | 60(5): 365-71 (2002). |
| 24 | Sp17 | ILDSSEEDK | Chiriva-Internati et al. Int J Cancer. |
| | | | 107(5): 863-5 (2003). |
| 25 | SSX-4 | INKTSGPKRGKHAWTHR | Ayyoub et al. Clin Immunol. |
| | | LRE | 114(1): 70-8 (2005). |
| | | YFSKKEWEKMKSSEKIV | Valmori et al. Clin Cancer Res. |
| | | YVY | 12(2): 398-404 (2006). |
| | | MKLNYEVMTKLGFKVT | |
| | | LPPF | |
| | | KHAWTHRLRERKQLVV | |
| | | YEEI | |
| | | LGFKVTLPPFMRSKRAA | |
| | | DFH | |
| | | KSSEKIVYVYMKLNYEV | |
| | | MTK | |
| | | KHAWTHRLRERKQLVV | |
| | | YEEI | |
| 26 | TAG-1 | SLGWLFLLL | Adair et al. J Immunother. 31(1): 7- |
| | | LSRLSNRLL | 17 (2008). |
| 27 | TAG-2 | LSRLSNRLL | Adair et al. J Immunother. 31(1): 7- |
| | | | 17 (2008). |

TABLE 4
Breast cancer

| | Tumor- | | |
|---|---|---|---|
| | associated | Reported immunogenic | |
| No. | antigen | epitopes | Sources |
| 1 | ENAH(hMena) | TMNGSKSPV | Di Modugno et al. Int. J. Cancer. |
| | | | 109(6): 909-18 (2004). |
| 2 | mammaglobin-A | PLLENVISK | Jaramillo et al. Int. J. Cancer. |
| | | | 102(5): 499-506 (2002). |
| 3 | NY-BR-1 | SLSKILDTV | Wang et al. Cancer Res. 66(13): 6826- |
| | | | 33 (2006). |
| 4 | EpCAM | RYQLDPKFI | Gastl et al. Lancet 356(9246): 1981-2 |
| | | | (2000). |
| | | | Tajima, 2004 |
| 5 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. 132(2): 345- |
| | | APRGPHGGAASGL | 54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. 132(2): 345- |
| | | SLLMWITQCFLPVF | 54 (2013). |
| | | LLEFYLAMPFATPMEAE | Knights et al. Cancer Immunol |
| | | L-ARRSLAQ | Immunother. 58(3): 325-38 (2009). |
| | | EFYLAMPFATPM | Jäger et al. Cancer Immun. 2: 12 |
| | | PGVLLICEFTVSGNILTIR | (2002). |
| | | L-TAADHR | Zeng et al. Proc Natl Acad Sci USA. |
| | | RLLEFYLAMPFA | 98(7): 3964-9 (2001). |
| | | QGAMLAAQERRVPRAA | Mandic et al. J Immunol. 174(3): 1751- |
| | | E-VPR | 9 (2005). |
| | | PFATPMEAELARR | Chen et al. Proc Natl Acad Sci USA. |
| | | PGVLLKEFTVSGNILTIR | 101(25): 9363-8 (2004). |
| | | LT | Ayyoub et al. Clin Cancer Res. |
| | | VLLKEFTVSG | 16(18): 4607-15 (2010). |
| | | AADHRQLQLSISSCLQQL | Slager et al. J Immunol. 172(8): 5095- |
| | | LKEFTVSGNILTIRL | 102 (2004). |
| | | PGVLLKEFTVSGNILTIR | Mizote et al. Vaccine. 28(32): 5338- |
| | | L-TAADHR | 46 (2010). |
| | | LLEFYLAMPFATPMEAE | Jager et al. J Exp Med. 191(4): 625- |
| | | L-ARRSLAQ | 30 (2000). |
| | | KEFTVSGNILT | Zarour et al. Cancer Res. 60(17): 4946- |
| | | LLEFYLAMPFATPM | 52 (2000). |
| | | AGATGGRGPRGAGA | Zeng et al. J Immunol. 165(2): 1153- |
| | | | 9 (2000). |
| | | | Bioley et al. Clin Cancer Res. |
| | | | 15(13): 4467-74 (2009) |
| | | | Zarour et al. Cancer Res. 62(1): 213-8 |
| | | | (2002). |
| | | | Hasegawa et al. Clin Cancer Res. |
| | | | 12(6): 1921-7 (2006). |
| 6 | BAGE-1 | AARAVFLAL | Boel et al. Immunity. 2(2): 167- |
| | | | 75 (1995). |
| 7 | HERV-K- | MLAVISCAV | Schiavetti et al. Cancer Res. |
| | MEL | | 62(19): 5510-6 (2002). |
| 8 | KK-LC-1 | RQKRILVNL | Fukuyama et al. Cancer Res. |
| | | | 66(9): 4922-8 (2006). |
| 9 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 Pt |
| | | | 1): 6047-57 (2004). |
| 10 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAA | Sun et al. Cancer Immunol |
| | | EVP-R | Immunother. 55(6): 644-52 (2006). |
| | | AADHRQLQLSISSCLQQL | Slager et al. Cancer Gene Ther. |
| | | CLSRRPWICRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. 172(8): 5095- |
| | | | 102 (2004). |
| | | | Jager et al. J Exp Med. 191(4): 625- |
| | | | 30 (2000). |
| | | | Slager et al. J Immunol. 170(3): 1490- |
| | | | 7(2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 11 | MAGE-A1 | EADPTGHSY | Traversari et al. J Exp Med. |
| | | KVLEYVIKV | 176(5): 1453-7 (1992). |
| | | SLFRAVITK | Ottaviani et al. Cancer Immunol |
| | | EVYDGREHSA | Immunother. 54(12): 1214-20 (2005). |
| | | RVRFFFPSL | Pascolo et al. Cancer Res. |
| | | EADPTGHSY | 61(10): 4072-7 (2001). |
| | | REPVTKAEML | Chaux et al. J Immunol. 163(5): 2928- |
| | | KEADPTGHSY | 36 (1999). |
| | | DPARYEFLW | Luiten et al. Tissue Anitgens. |
| | | ITKKVADLVGF | 55(2): 49-52 (2000). |
| | | SAFPTTINF | Luiten et al. Tissue Antigens. |
| | | SAYGEPRKL | 56(1): 77-81 (2000). |
| | | RVRFFFPSL | Tanzarella et al. Cancer Res. |
| | | TSCILESLFRAVITK | 59(11): 2668-74 (1999). |
| | | PRALAETSYVKVLEY | Stroobant et al. Eur J Immunol. |
| | | FLLLKYRAREPVTKAE | 42(6): 1417-28 (2012). |
| | | EYVIKVSARVRF | Corbière et al. Tissue Antigens. |
| | | | 63(5): 453-7 (2004). |
| | | | Goodyear et al. Cancer Immunol |
| | | | Immunother. 60(12): 1751-61 (2011). |
| | | | van der Bruggen et al. Eur J Immunol. |
| | | | 24(9): 2134-40 (1994). |
| | | | Wang et al. Cancer Immunol |
| | | | Immunother. 56(6): 807-18 (2007). |
| | | | Chaux et al. J Exp Med. 189(5): 767-78 |
| | | | (1999). |
| | | | Chaux et al. Eur J Immunol. 31(6): |
| | | | 1910-6 (2001). |
| 12 | MAGE-A2 | YLQLVFGIEV | Kawashima et al. Hum Immunol. |
| | | EYLQLVFGI | 59(1): 1-14 (1998). |
| | | REPVTKAEML | Tahara et al. Clin Cancer Res. |
| | | EGDCAPEEK | 5(8): 2236-41 (1999). |
| | | LLKYRAREPVTKAE | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. 89(5): 767-78 |
| | | | (1999). |
| 13 | mucink | PDTRPAPGSTAPPAHGV | Jerome et al. J Immunol. 151(3): 1654- |
| | | TSA | 62 (1993). |
| 14 | Sp17 | ILDSSEEDK | Chiriva-Internati et al. Int J Cancer. |
| | | | 107(5): 863-5 (2003). |
| 15 | SSX-2 | KASEKIFYV | Ayyoub et al. J Immunol. |
| | | EKIQKAFDDIAKYFSK | 168(4): 1717-22 (2002). |
| | | FGRLQGISPKI | Ayyoub et al. J Immunol. |
| | | WEKMKASEKIFYVYMK | 172(11): 7206-11 (2004). |
| | | RK | Neumann et al. Cancer Immunol |
| | | KIFYVYMKRKYEAMT | Immunother. 60(9): 1333-46 (2011). |
| | | KIFYVYMKRKYEAM | Ayyoub et al. Clin Immunol. |
| | | | 114(1): 70-8 (2005). |
| | | | Neumann et al. Int J Cancer. |
| | | | 112(4): 661-8 (2004). |
| | | | Ayyoub et al. J Clin Invest. |
| | | | 113(8): 1225-33 (2004). |
| 16 | TAG-1 | SLGWLFLLL | Adair et al. J Immunother. 31(1): 7-17 |
| | | LSRLSNRLL | (2008). |
| 17 | TAG-2 | LSRLSNRLL | Adair et al. J Immunother. 31(1): 7-17 |
| | | | (2008). |
| 18 | TRAG-3 | CEFHACWPAFTVLGE | Janjic et al. J Immunol. 177(4): 2717- |
| | | | 27 (2006). |
| 19 | Her2/Neu | HLYQGCQVV | Nakatsuka et al. Mod. Pathol. |
| | | YLVPQQGFFC | 19(6): 804-814 (2006). |
| | | PLQPEQLQV | Pils et al. Br. J. Cancer 96(3): 485-91 |
| | | TLEEITGYL | (2007). |
| | | ALIHHNTHL | Scardino et al. Eur J Immunol. |
| | | PLTSIISAV | 31(11): 3261-70 (2001). |
| | | VLRENTSPK | Scardino et al. J Immunol. |
| | | TYLPTNASL | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 20 | c-myc | | Reuschenbach et al. Cancer Immunol. |
| | | | Immunother. 58: 1535-1544 (2009) |
| 21 | cyclin B1 | | Reuschenbach et al. Cancer Immunol. |
| | | | Immunother. 58: 1535-1544 (2009) |
| 22 | MUC1 | | Reuschenbach et al. Cancer Immunol. |
| | | | Immunother. 58: 1535-1544 (2009) |
| 23 | p53 | VVPCEPPEV | Hung et al. Immunol. Rev. 222:43-69 |
| | | | (2008). |
| | | | http://cancerimmunity.org/peptide/mutations/ |
| 24 | p62 | | Reuschenbach et al. Cancer Immunol. |
| | | | Immunother. 58: 1535-1544 (2009) |
| 25 | Survivin | | Reuschenbach et al. Cancer Immunol. |
| | | | Immunother. 58: 1535-1544 (2009) |

TABLE 5
Testicular cancer

| | Tumor- | | |
|---|---|---|---|
| | associated | Reported immunogenic | |
| No. | antigen | epitopes | Sources |
| 1 | CD45 | KFLDALISL | Tomita et al. Cancer Sci. |
| | | | 102(4): 697-705 (2011). |
| 2 | DKK1 | ALGGHPLLGV | Qian et al. Blood. (5): 1587-94 |
| | | | (2007). |
| 3 | PRAME | VLDGLDVLL, | Kessler et al. J Exp Med. |
| | | SLYSFPEPEA, | 193(1): 73-88 (2001). |
| | | ALYVDSLFFL, | Ikeda et al. Immunity 6(2): 199- |
| | | SLLQHLIGL, | 208 (1997). |
| | | LYVDSLFFL | |
| 4 | RU2AS | LPRWPPPQL | Van Den Eynde et al. J. Exp. |
| | | | Med. 190(12): 1793-800 (1999). |
| 5 | Telomerase | ILAKFLHWL; | Vonderheide et al. Immunity |
| | | RLVDDFLLV; | 10(6): 673-9 (1999). |
| | | RPGLLGASVLGLDDI; | Miney et al. Proc. Natl. Acad. |
| | | and | Sci. U.S.A. 97(9): 4796-801 |
| | | LTDLQPYMRQFVAHL | (2000). |
| | | | Schroers et al. Cancer Res. |
| | | | 62(9): 2600-5 (2002). |
| | | | Schroers et al. Clin. Cancer Res. |
| | | | 9(13): 4743-55 (2003). |

TABLE 6
Pancreatic cancer

| | Tumor- | | |
|---|---|---|---|
| | associated | Reported immunogenic | |
| No. | antigen | epitopes | Sources |
| 1 | ENAH (hMena) | TMNGSKSPV | Di Modugno et al. Int. J. |
| | | | Cancer. 109(6): 909-18 |
| | | | (2004). |
| 2 | PBF | CTACRWKKACQR | Tsukahara et al. Cancer Res. |
| | | | 64(15): 5442-8 (2004). |
| 3 | K-ras | VVVGAVGVG | Gjertsen et al. Int. J. Cancer. |
| | | | 72(5): 784-90 (1997). |
| 4 | Mesothelin | SLLFLLFSL | Le et al. Clin. Cancer Res. |
| | | VLPLTVAEV | 18(3): 858-68 (2012). |
| | | ALQGGGPPY | Hassan et al. Appl. |
| | | LYPKARLAF | Immunohistochem. Mol. |
| | | AFLPWHRLF | Morphol. 13(3): 243-7 (2005). |
| | | | Thomas et al J Exp Med. |
| | | | 2004 Aug. 2; 200(3): 297-306. |
| 5 | mucink | PDTRPAPGSTAPPAHGVTSA | Jerome et al. J Immunol. |
| | | | 151(3): 1654-62 (1993). |

TABLE 7
Liver cancer

| | Tumor- | | |
|---|---|---|---|
| | associated | Reported immunogenic | |
| No. | antigen | epitopes | Sources |
| 1 | G250/ | HLSTAFAR; | Vissers et al. Cancer Res. 59(21): 5554-9 |
| | MN/ | KIFGSLAFL; | (1999). |
| | CAIX | IISAVVGIL; | Fisk et al. J Exp Med. 181(6): 2109-17 |
| | | ALCRWGLLL; | (1995). |
| | | ILHNGAYSL; | Brossart et al. Cancer Res. 58(4): 732-6 |
| | | RLLQETELV; | (1998). |
| | | VVKGVVFGI; and | Kawashima et al. Hum Immunol. |
| | | YMIMVKCWMI | 59(1): 1-14 (1998). |
| | | | Rongcun et al. J Immunol. 163(2): 1037- |
| | | | 44 (1999). |
| 2 | Hepsin | SLLSGDWVL; | Guo et al. Scand J Immunol. 78(3): 248- |
| | | GLQLGVQAV; | 57 (2013). |
| | | and | |
| | | PLTEYIQPV | |
| 3 | Intestinal | SPRWWPTCL | Ronsin et al. J Immunol. 163(1): 483-90 |
| | carboxyl | | (1999). |
| | esterase | | |
| 4 | alpha- | GVALQTMKQ; | Butterfield et al. Cancer Res. |
| | foetoprotein | FMNICFIYEI; | 59(13): 3134-42 (1999). |
| | | and QLAVSVILRV | Pichard et al. J Immunother. 31(3): 246- |
| | | | 53 (2008) |
| | | | Alisa et al. Clin. Cancer Res. |
| | | | 11(18): 6686-94 (2005). |
| 5 | M-CSF | LPAVVGLSPGEQEY | Probst-Kepper et al. J Exp Med. |
| | | | 193(10): 1189-98 (2001). |
| 6 | PBF | CTACRWKKACQR | Tsukahara et al. Cancer Res. |
| | | | 64(15): 5442-8 (2004). |
| 7 | PSMA | NYARTEDFF | Horiguchi et al. Clin Cancer Res. |
| | | | 8(12): 3885-92 (2002). |
| 8 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. 60(16): 4499- |
| | | YLAMPFATPME | 506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. 132(2): 345- |
| | | APRGPHGGAASGL | 54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. 132(2): 345- |
| | | SLLMWITQCFLPVF | 54 (2013). |
| | | LLEFYLAMPFATPMEAEL- | Knights et al. Cancer Immunol |
| | | ARRSLAQ | Immunother. 58(3): 325-38 (2009). |
| | | EFYLAMPFATPM | Jäger et al. Cancer Immun. 2: 12 (2002). |
| | | PGVLLKEFTVSGNILTIRL- | Zeng et al. Proc Natl Acad Sci USA. |
| | | TAADHR | 98(7): 3964-9 (2001). |
| | | RLLEFYLAMPFA | Mandic et al. J Immunol. 174(3): 1751- |
| | | QGAMLAAQERRVPRAAE- | 9 (2005). |
| | | VPR | Chen et al. Proc Natl Acad Sci USA. |
| | | PFATPMEAELARR | 101(25): 9363-8 (2004). |
| | | PGVLLKEFTVSGNILTIRLT | Ayyoub et al. Clin Cancer Res. |
| | | VLLKEFTVSG | 16(18): 4607-15 (2010). |
| | | AADHRQLQLSISSCLQQL | Slager et al. J Immunol. 172(8): 5095- |
| | | LKEFTVSGNILTIRL | 102 (2004). |
| | | PGVLLKEFTVSGNILTIRL- | Mizote et al. Vaccine. 28(32): 5338- |
| | | TAADHR | 46 (2010). |
| | | LLEFYLAMPFATPMEAEL- | Jager et al. J Exp Med. 191(4): 625- |
| | | ARRSLAQ | 30 (2000). |
| | | KEFTVSGNILT | Zarour et al. Cancer Res. 60(17): 4946- |
| | | LLEFYLAMPFATPM | 52 (2000). |
| | | AGATGGRGPRGAGA | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213-8 |
| | | | (2002). |
| | | | Hasegawa et al. Clin Cancer Res. |
| | | | 12(6): 1921-7 (2006). |
| 9 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol Immunother. |
| | | VP-R | 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Stager et al. J Immunol. 172(8): 5095- |
| | | | 102 (2004). |
| | | | Jager et al. J Exp Med. 191(4): 625- |
| | | | 30 (2000). |
| | | | Stager et al. J Immunol. 170(3): 1490- |
| | | | 7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 10 | HERV-K- | MLAVISCAV | Schiavetti et al. Cancer Res. |
| | MEL | | 62(19): 5510-6 (2002). |
| 11 | KK-LC-1 | RQKRILVNL | Fukuyama et al. Cancer Res. |
| | | | 66(9): 4922-8 (2006). |
| 12 | KM-HN-1 | NYNNFYRFL | Fukuyama et al. Cancer Res. |
| | | | 66(9): 4922-8 (2006). |
| | | EYSKECLKEF | Monji et al. Clin Cancer Res. 10(18 Pt |
| | | | 1): 6047-57 (2004). |
| | | EYLSLSDKI | |
| 13 | Sp17 | ILDSSEEDK | Chiriva-Internati et al. Int J Cancer. |
| | | | 107(5): 863-5 (2003). |
| 14 | c-myc | | Reuschenbach et al. Cancer Immunol. |
| | | | Immunother. 58: 1535-1544 (2009) |
| 15 | cyclin B1 | | Reuschenbach et al. Cancer Immunol. |
| | | | Immunother. 58: 1535-1544 (2009) |
| 16 | p53 | VVPCEPPEV | Hung et al. Immunol. Rev. 222:43-69 |
| | | | (2008). |
| | | | http://cancerimmunity.org/peptide/mutations/ |
| 17 | p62 | | Reuschenbach et al. Cancer Immunol. |
| | | | Immunother. 58: 1535-1544 (2009) |
| 18 | Survivin | | Reuschenbach et al. Cancer Immunol. |
| | | | Immunother. 58: 1535-1544 (2009) |

TABLE 8
Colorectal cancer

| | Tumor- | | |
|---|---|---|---|
| | associated | Reported immunogenic | |
| No. | antigen | epitopes | Sources |
| 1 | ENAH (hMena) | TMNGSKSPV | Di Modugno et al. Int. J Cancer. |
| | | | 109(6): 909-18 (2004). |
| 2 | Intestinal | SPRWWPTCL | Ronsin et al. J Immunol. 163(1): 483- |
| | carboxyl | | 90 (1999). |
| | esterase | | |
| 3 | CASP-5 | FLIIWQNTM | Schwitalle et al. Cancer Immun. 4: 14 |
| | | | (2004). |
| 4 | COA-1 | TLYQDDTLTLQAAG | Maccalli et al. Cancer Res. |
| | | | 63(20): 6735-43 (2003). |
| 5 | OGT | SLYKFSPFPL | Ripberger. J Clin Immunol. 23(5): 415- |
| | | | 23 (2003). |
| 6 | OS-9 | KELEGILLL | Vigneron et al. Cancer Immun. 2: 9 |
| | | | (2002). |
| 7 | TGF-betaRII | RLSSCVPVA | Linnebacher et al. Int. J. Cancer. |
| | | | 93(1): 6-11 (2001). |
| 8 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. 132(2): 345- |
| | | APRGPHGGAASGL | 54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. 132(2): 345- |
| | | SLLMWITQCFLPVF | 54 (2013). |
| | | LLEFYLAMPFATPMEAE | Knights et al. Cancer Immunol |
| | | L-ARRSLAQ | Immunother. 58(3): 325-38 (2009). |
| | | EFYLAMPFATPM | Jäger et al. Cancer Immun. 2:12 |
| | | PGVLLKEFTVSGNILTLR | (2002). |
| | | L-TAADHR | Zeng et al. Proc Natl Acad Sci USA. |
| | | RLLEFYLAMPFA | 98(7): 3964-9 (2001). |
| | | QGAMLAAQERRVPRAA | Mandic et al. J Immunol. 174(3): 1751- |
| | | E-VPR | 9 (2005). |
| | | PFATPMEAELARR | Chen et al. Proc Natl Acad Sci USA. |
| | | PGVLLKEFTVSGNILTIR | 101(25): 9363-8 (2004). |
| | | LT | Ayyoub et al. Clin Cancer Res. |
| | | VLLKEFTVSG | 16(18): 4607-15 (2010). |
| | | AADHRQLQLSISSCLQQL | Slager et al. J Immunol. 172(8): 5095- |
| | | LKEFTVSGNILTIRL | 102 (2004). |
| | | PGVLLKEFTVSGNILTIR | Mizote et al. Vaccine. 28(32): 5338- |
| | | L-TAADHR | 46 (2010). |
| | | LLEFYLAMPFATPMEAE | Jager et al. J Exp Med. 191(4): 625- |
| | | L-ARRSLAQ | 30 (2000). |
| | | KEFTVSGNILT | Zarour et al. Cancer Res. 60(17): 4946- |
| | | LLEFYLAMPFATPM | 52 (2000). |
| | | AGATGGRGPRGAGA | Zeng et al. J Immunol. 165(2): 1153- |
| | | | 9 (2000). |
| | | | Bioley et al. Clin Cancer Res. |
| | | | 15 (13): 4467-74 (2009). |
| | | | Zarour et al. Cancer Res. 62(1): 213-8 |
| | | | (2002). |
| | | | Hasegawa et al. Clin Cancer Res. |
| | | | 12(6): 1921-7 (2006). |
| 9 | CEA | TYYRPGVNLSLSC | Duffy, Clin. Chem. 47(4): 624-30 |
| | | EIIYPNASLLIQN | (2001). |
| | | YACFVSNLATGRNNS | Parkhurst et al. Mol. Ther. 19(3): 620-6 |
| | | LWWVNNQSLPVSP | (2011). |
| | | LWWVNNQSLPVSP | Galanis et al. Cancer Res. 70(3): 875- |
| | | LWWVNNQSLPVSP | 82 (2010). |
| | | EIIYPNASLLIQN | Bast et al. Am. J. Obstet. Gynecol. |
| | | NSIVKSITVSASG | 149(5): 553-9 (1984). |
| | | KTWGQYWQV | Crosti et al. J Immunol. 176(8): 5093-9 |
| | | (A)MLGTHTMEV | (2006). |
| | | ITDQVPFSV | Kobayashi et al. Clin Cancer Res. |
| | | YLEPGPVTA | 8(10): 3219-25 (2002). |
| | | LLDGTATLRL | Campi et al. Cancer Res. 63(23): 8481- |
| | | VLYRYGSFSV | 6 (2003). |
| | | SLADTNSLAV | Bakker et at. Int J Cancer. 62(1): 97- |
| | | RLMKQDFSV | 102 (1995). |
| | | RLPRIFCSC | Tsai et al. J Immunol. 158(4): 1796- |
| | | LIYRRRLMK | 802 (1997). |
| | | ALLAVGATK | Kawakami et al. J Immunol. |
| | | IALNFPGSQK | 154(8): 3961-8 (1995). |
| | | RSYVPLAHR | Cox et al. Science. 264(5159): 716-9 |
| | | | (1994). |
| | | | Kawakami et al. J Immunol. |
| | | | 154(8): 3961-8 (1995). |
| | | | Kawakami et al. J Immunol. |
| | | | 161(12): 6985-92 (1998). |
| | | | Skipper et al. J Immunol. |
| | | | 157(11): 5027-33 (1996). |
| | | | Michaux et al. J Immunol. |
| | | | 192(4): 1962-71 (2014). |
| 10 | HERV-K- | MLAVISCAV | Schiavetti et al. Cancer Res. |
| | MEL | | 62(19): 5510-6 (2002). |
| 11 | KK-LC-1 | RQKRILVNL | Fukuyama et al. Cancer Res. |
| | | | 66(9): 4922-8 (2006). |
| 12 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 Pt |
| | | | 1): 6047-57 (2004). |
| 13 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAA | Sun et at. Cancer Immunol |
| | | EVP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. 172(8): 5095- |
| | | | 102 (2004). |
| | | | Jager et al. J Exp Med. 191(4): 625- |
| | | | 30 (2000). |
| | | | Slager et al. J Immunol. 170(3): 1490- |
| | | | 7(2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 14 | MAGE-A2 | YLQLVFGIEV | Kawashima et al. Hum Immunol. |
| | | EYLQLVFGI | 59(1): 1-14 (1998). |
| | | REPVTKAEML | Tahara et al. Clin Cancer Res. |
| | | EGDCAPEEK | 5(8): 2236-41 (1999). |
| | | LLKYRAREPVTKAE | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. 89(5): 767-78 |
| | | | (1999). |
| 15 | Sp17 | ILDSSEEDK | Chiriva-Internati et al. Int J Cancer. |
| | | | 107(5): 863-5 (2003). |
| 16 | TAG-1 | SLGWLFLLL | Adair et al. J Immunother. 31(1): 7-17 |
| | | LSRLSNRLL | (2008). |
| 17 | TAG-2 | LSRLSNRLL | Adair et al. J Immunother. 31(1): 7-17 |
| | | | (2008). |
| 18 | c-myc | | Reuschenbach et al. Cancer Immunol. |
| | | | Immunother. 58: 1535-1544 (2009) |
| 19 | cyclin B1 | | Reuschenbach et al. Cancer Immunol. |
| | | | Immunother. 58: 1535-1544 (2009) |
| 20 | MUC1 | | Reuschenbach et al. Cancer Imnumol. |
| | | | Immunother. 58: 1535-1544 (2009) |
| 21 | p53 | VVPCEPPEV | Hung et al. Immunol. Rev. 222:43-69 |
| | | | (2008). |
| | | | http://cancerimmunity.org/peptide/mutations/ |
| 22 | p62 | | Reuschenbach et al. Cancer Immunol. |
| | | | Immunother. 58: 1535-1544 (2009) |
| 23 | Survivin | | Reuschenbach et al. Cancer Immunol. |
| | | | Immunother. 58: 1535-1544 (2009) |
| 24 | gp70 | | Castle et al., BMC Genomics 15: 190 |
| | | | (2014) |

TABLE 9
Thyroid cancer

| | Tumor- | | |
|---|---|---|---|
| | associated | Reported immunogenic | |
| No. | antigen | epitopes | Sources |
| 1 | CALCA | VLLQAGSLHA | El Hage et al. Proc. Natl. |
| | | | Acad. Sci. U.S.A. |
| | | | 105(29): 10119-24 (2008). |
| 2 | NY-ESO-1 | HLA-A2-restricted peptide | Jager et al. Proc. Natl. Acad. |
| | | p157-165 (SLLMWITQC), | Scie. U.S.A. 103(39): 14453-8 |
| | | HLA-Cw3- | (2006). |
| | | restricted p92-100 (LAMP- | Gnjatic et al. PNAS |
| | | FATPM) and | Sep. 26, 2000 vol. 97 |
| | | HLA-Cw6-restricted p80-88 | no. 20 p. 10919 |
| | | (ARGPESRLL) | Jager et al. J Exp Med. |
| | | SLLMWITQC | 187(2): 265-70 (1998). |
| | | MLMAQEALAFL | Chen et al. J Immunol. |
| | | YLAMPFATPME | 165(2): 948-55 (2000). |
| | | ASGPGGGAPR | Valmori et al. Cancer Res. |
| | | LAAQERRVPR | 60(16): 4499-506 (2000). |
| | | TVSGNILTIR | Aarnoudse et al. Int J Cancer. |
| | | APRGPHGGAASGL | 82(3): 442-8 (1999). |
| | | MPFATPMEAEL | Eikawa et al. Int J Cancer. |
| | | KEFTVSGNILTI | 132(2): 345-54 (2013). |
| | | MPFATPMEA | Wang et al. J Immunol. |
| | | FATPMEAEL | 161(7): 3598-606 (1998). |
| | | FATPMEAELAR | Matsuzaki et al. Cancer |
| | | LAMPFATPM | Immunol Immunother. |
| | | ARGPESRLL | 57(8)1185-95 (2008). |
| | | SLLMWITQCFLPVF | Ebert et al. Cancer Res. |
| | | LLEFYLAMPFATPMEAEL- | 69(3): 1046-54 (2009). |
| | | ARRSLAQ | Eikawa et al. Int J Cancer. |
| | | EFYLAMPFATPM | 132(2): 345-54 (2013). |
| | | PGVLLKEFTVSGNILTIRL- | Knights et al. Cancer |
| | | TAADIAR | Immunol Immunother. |
| | | RLLEFYLAMPFA | 58(3): 325-38 (2009). |
| | | QGAMLAAQERRVPRAAE- | Jäger et al. Cancer Immun. |
| | | VPR | 2: 12 (2002). |
| | | PFATPMEAELARR | Zeng et al. Proc Natl Acad Sci |
| | | PGVLLKEFTVSGNILTIRLT | USA. 98(7): 3964-9 (2001). |
| | | VLLKEFTVSG | Mandic et al. J Immunol. |
| | | AADHRQLQLSISSCLQQL | 174(3): 1751-9 (2005). |
| | | LKEFTVSGNILTIRL | Chen et al. Proc Natl Acad |
| | | PGVLLKEFTVSGNILTIRL- | Sci USA. 101(25): 9363- |
| | | TAADHR | 8 (2004). |
| | | LLEFYLAMPFATPMEAEL- | Ayyoub et al. Clin Cancer |
| | | ARRSLAQ | Res. 16(18): 4607-15 (2010). |
| | | KEFTVSGNILT | Slager et al. J Immunol. |
| | | LLEFYLAMPFATPM | 172(8): 5095-102 (2004). |
| | | AGATGGRGPRGAGA | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 3 | HERV-K-MEL | MLAVISCAV | Schiavetti et al. Cancer Res. |
| | | | 62(19): 5510-6 (2002). |
| 4 | KK-LC-1 | RQKRILVNL | Fukuyama et al. Cancer Res. |
| | | | 66(9): 4922-8 (2006). |
| 5 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. |
| | | | 10(18 Pt 1): 6047-57 (2004). |
| 6 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. |
| | | SLLMWITQCFLPVF | 161(7): 3598-606 (1998). |
| | | QGAMLAAQERRVPRAAEVP-R | Sun et al. Cancer Immunol |
| | | AADHRQLQLSISSCLQQL | Immunother. 55(6): 644-52 |
| | | CLSRRPWKRSWSAGSCPG- | (2006). |
| | | MPHL | Slager et al. Cancer Gene |
| | | ILSRDAAPLPRPG | Ther. 11(3): 227-36 (2004). |
| | | AGATGGRGPRGAGA | Zeng et al. Proc Natl Acad Sci |
| | | | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 7 | Sp17 | ILDSSEEDK | Chiriva-Internati et al. Int J |
| | | | Cancer. 107(5): 863-5 (2003). |

TABLE 10
Lung cancer

| | Tumor- | Reported | |
|---|---|---|---|
| | associated | immunogenic | |
| No. | antigen | epitopes | Sources |
| 1 | CD274 | LLNAFTVTV | Munir et al. Cancer Res. 73(6): 1764-76 |
| | | | (2013). |
| 2 | mdm-2 | VLFYLGQY | Asai et al. Cancer Immun. 2: 3 (2002). |
| 3 | alpha- | FIASNGVKLV | Echchakir et al. Cancer Res. |
| | actinin-4 | | 61(10): 4078-83 (2001). |
| 4 | Elongation | ETVSEQSNV | Hogan et al. Cancer Res. 58(22): 5144- |
| | factor 2 | | 50 (1998). |
| | (squamous | | |
| | cell | | |
| | carcinoma of | | |
| | the lung) | | |
| 5 | ME1(non- | FLDEFMEGV | Karanikas et al. Cancer Res. |
| | small cell | | 61(9): 3718-24 (2001). |
| | lung | | |
| | carcinoma) | | |
| 6 | NFYC | QQITKTEV | Takenoyama et al. Int. J Cancer. |
| | (squamous | | 118(8): 1992-7 (2006). |
| | cell | | |
| | carcinoma of | | |
| | the lung) | | |
| 7 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. 132(2): |
| | | APRGPHGGAASGL | 345-54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. 132(2): |
| | | SLLMWITQCFLPVF | 345-54 (2013). |
| | | LLEFYLAMPFATPMEAE | Knights et al. Cancer Immunol |
| | | L-ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAA | Mandic et al. J Immunol. 174(3): 1751- |
| | | E-VPR | 9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. 172(8): 5095- |
| | | PGVLLKEFTVSGNILTIRL | 102 (2004). |
| | | -TAADHR | Mizote et al. Vaccine. 28(32): 5338- |
| | | LLEFYLAMPFATPMEAE | 46 (2010). |
| | | L-ARRSLAQ | Jager et al. J Exp Med. 191(4): 625- |
| | | KEFTVSGNILT | 30 (2000). |
| | | LLEFYLAMPFATPM | Zarour et al. Cancer Res. 60(17): 4946- |
| | | AGATGGRGPRGAGA | 52 (2000). |
| | | | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213-8 |
| | | | (2002). |
| | | | Hasegawa et al. Clin Cancer Res. |
| | | | 12(6): 1921-7 (2006). |
| 8 | GAGE-1, 2, 8 | YRPRPRRY | Van den Eynde et al. J Exp Med. |
| | | | 182(3): 689-98 (1995). |
| 9 | HERV-K- | MLAVISCAV | Schiavetti et al. Cancer Res. |
| | MEL | | 62(19): 5510-6 (2002). |
| 10 | KK-LC-1 | RQKRILVNL | Fukuyama et al. Cancer Res. |
| | | | 66(9): 4922-8 (2006). |
| 11 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 Pt |
| | | | 1): 6047-57 (2004). |
| 12 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAA | Sun et al. Cancer Immunol |
| | | EVP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. 172(8): 5095- |
| | | | 102 (2004). |
| | | | Jager et al. J Exp Med. 191(4): 625- |
| | | | 30 (2000). |
| | | | Slager et al. J Immunol. 170(3): 1490- |
| | | | 7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 13 | MAGE-A2 | YLQLVFGIEV | Kawashima et al. Hum Immunol. |
| | | EYLQLVFGI | 59(1): 1-14 (1998). |
| | | REPVTKAEML | Tahara et al. Clin Cancer Res. |
| | | EGDCAPEEK | 5(8): 2236-41 (1999). |
| | | LLKYRAREPVTKAE | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. 89(5): 767-78 |
| | | | (1999). |
| 14 | MAGE-A6 | MVKISGGPR | Zorn et al. Eur J Immunol. 29(2): 602-7 |
| | (squamous | EVDPIGHVY | (1999). |
| | cell lung | REPVTKAEML | Benlalam et al. J Immunol. |
| | carcinoma) | EGDCAPEEK | 171(11): 6283-9 (2003). |
| | | ISGGPRISY | Tanzarella et al. Cancer Res. |
| | | LLKYRAREPVTKAE | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. 189(5): 767- |
| | | | 78 (1999). |
| 15 | Sp17 | ILDSSEEDK | Chiriva-Internati et al. Int J Cancer. |
| | | | 107(5): 863-5 (2003). |
| 16 | TAG-1 | SLGWLFLLL | Adair et al. J Immunother. 31(1): 7-17 |
| | | LSRLSNRLL | (2008). |
| 17 | TAG-2 | LSRLSNRLL | Adair et al. J Immunother. 31(1): 7-17 |
| | | | (2008). |
| 18 | TRAG-3 | CEFHACWPAFTVLGE | Janjic et al. J Immunol. 177(4): 2717-27 |
| | | | (2006). |
| 19 | XAGE- | RQKKIRIQL | Ohue et al. Int J Cancer. 131(5): E649- |
| | 1b/GAGED2a | HLGSRQKKIRIQLRSQ | 58 (2012). |
| | (non-small | CATWKVICKSCISQTPG | Shimono et al. Int J Oncol. 30(4): 835- |
| | cell lung | | 40 (2007). |
| | cancer) | | |
| 20 | c-myc | | Reuschenbach et al. Cancer Immunol. |
| | | | Immunother. 58: 1535-1544 (2009) |
| 21 | cyclin B1 | | Reuschenbach et al. Cancer Immunol. |
| | | | Immunother. 58: 1535-1544 (2009) |
| 22 | Her2/Neu | HLYQGCQVV | Nakatsuka et al. Mod. Pathol. |
| | | YLVPQQGFFC | 19(6): 804-814 (2006). |
| | | PLQPEQLQV | Pils et al. Br. J. Cancer 96(3): 485-91 |
| | | TLEEITGYL | (2007). |
| | | ALIHHNTHL | Scardino et al. Eur J Immunol. |
| | | PLTSIISAV | 31(11): 3261-70 (2001). |
| | | VLRENTSPK | Scardino et al. J Immunol. |
| | | TYLPTNASL | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 23 | MUC1 | | Reuschenbach et al. Cancer Immunol. |
| | | | Immunother. 58: 1535-1544 (2009) |
| 24 | p53 | VVPCEPPEV | Hung et al. Immunol. Rev. 222: 43-69 |
| | | | (2008). |
| | | | http://cancerimmunity.org/peptide/muta- |
| | | | tions/ |
| 25 | p62 | | Reuschenbach et al. Cancer Immunol. |
| | | | Immunother. 58: 1535-1544 (2009) |
| 26 | Survivin | | Reuschenbach et al. Cancer Immunol. |
| | | | Immunother. 58: 1535-1544 (2009) |

TABLE 11
Prostate cancer

| | Tumor- | Reported | |
|---|---|---|---|
| | associated | immunogenic | |
| No. | antigen | epitopes | Sources |
| 1 | DKK1 | ALGGHPLLGV | Qian et al. Blood. |
| | | | 110(5): 1587-94 (2007). |
| 2 | ENAH (hMena) | TMNGSKSPV | Di Modugno et al. Int. J. |
| | | | Cancer. 109(6): 909-18 |
| | | | (2004). |
| 3 | Kallikrein 4 | FLGYLILGV; | Wilkinson et al. Cancer |
| | | SVSESDTIRSISIAS; | Immunol Immunother. |
| | | LLANGRMPTVLQCVN; | 61(2): 169-79 (2012). |
| | | and | Hural et al. J. Immunol. |
| | | RMPTVLQCVNVSVVS | 169(1): 557-65 (2002). |
| 4 | PSMA | NYARTEDFF | Horiguchi et al. Clin Cancer |
| | | | Res. 8(12): 3885-92 (2002). |
| 5 | STEAP1 | MIAVFLPIV and | Rodeberg et al. Clin. Cancer |
| | | HQQYFYKIPILVINK | Res. 11(12): 4545-52 (2005). |
| | | | Kobayashi et al. Cancer |
| | | | Res. 67(11): 5498-504 |
| | | | (2007). |
| 6 | PAP | FLFLLFFWL; | Olson et al. Cancer |
| | | TLMSAMTNL; | Immunol Immunother. |
| | | and | 59(6): 943-53 (2010). |
| | | ALDVYNGLL | |
| 7 | PSA (prostate | FLTPKKLQCV and | Correale et al. J Natl. |
| | carcinoma) | VISNDVCAQV | Cancer Inst. 89(4): 293-300 |
| | | | (1997). |
| 8 | NY-ESO-1 | HLA-A2-restricted peptide | Jager et al. Proc. Natl. Acad. |
| | | p157-165 (SLLMWITQC), | Scie. U.S.A. 103(39): 14453- |
| | | HLA-Cw3- | 8 (2006). |
| | | restricted p92-100 (LAMP- | Gnjatic et al. PNAS |
| | | FATPM) and | Sep. 26, 2000 vol. 97 |
| | | HLA-Cw6-restricted p80-88 | no. 20 p. 10919 |
| | | (ARGPESRLL) | Jager et al. J Exp Med. |
| | | SLLMWITQC | 187(2): 265-70 (1998). |
| | | MLMAQEALAFL | Chen et al. J Immunol. |
| | | YLAMPFATPME | 165(2): 948-55 (2000). |
| | | ASGPGGGAPR | Valmori et al. Cancer Res. |
| | | LAAQERRVPR | 60(16): 4499-506 (2000). |
| | | TVSGNILTIR | Aarnoudse et al. Int J |
| | | APRGPHGGAASGL | Cancer. 82(3): 442-8 (1999). |
| | | MPFATPMEAEL | Eikawa et al. Int J Cancer. |
| | | KEFTVSGNILTI | 132(2): 345-54 (2013). |
| | | MPFATPMEA | Wang et al. J Immunol. |
| | | FATPMEAEL | 161(7): 3598-606(1998). |
| | | FATPMEAELAR | Matsuzaki et al. Cancer |
| | | LAMPFATPM | Immunol Immunother. |
| | | ARGPESRLL | 57(8)1185-95 (2008). |
| | | SLLMWITQCFLPVF | Ebert et al. Cancer Res. |
| | | LLEFYLAMPFATPMEAEL- | 69(3): 1046-54 (2009). |
| | | ARRSLAQ | Eikawa et al. Int J Cancer. |
| | | EFYLAMPFATPM | 132(2): 345-54 (2013). |
| | | PGVLLKEFTVSGNILTLRL- | Knights et al. Cancer |
| | | TAADHR) | Immunol Immunother. |
| | | RLLEFYLAMPFA | 58(3): 325-38 (2009). |
| | | QGAMLAAQERRVPRAAE- | Jäger et al. Cancer Immun. |
| | | VPR | 2: 12 (2002). |
| | | PFATPMEAELARR | Zeng et al. Proc Natl Acad |
| | | PGVLLKEFTVSGNILTIRLT | Sci USA. 98(7): 3964- |
| | | VLLKEFTVSG | 9 (2001). |
| | | AADHRQLQLSISSCLQQL | Mandic et al. J Immunol. |
| | | LKEFTVSGNILTIRL | 174(3): 1751-9 (2005). |
| | | PGVLLICEFTVSGNILTIRL- | Chen et al. Proc Natl Acad |
| | | TAADHR | Sci USA. 101(25): 9363- |
| | | LLEFYLAMPFATPMEAEL- | 8 (2004). |
| | | ARRSLAQ | Ayyoub et al. Clin Cancer |
| | | KEFTVSGNILT | Res. 16(18): 4607-15 (2010). |
| | | LLEFYLAMPFATPM | Slager et al. J Immunol. |
| | | AGATGGRGPRGAGA | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer |
| | | | Res. 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 9 | BAGE-1 (non- | AARAVFLAL | Boel et al. Immunity. |
| | small cell lung | | 2(2): 167-75 (1995). |
| | carcinoma) | | |
| 10 | GAGE-1, 2, 8 | YRPRPRRY | Van den Eynde et al. J Exp |
| | (non-small cell | | Med. 182(3): 689-98 (1995). |
| | lunch carcinoma) | | |
| 11 | GAGE-3, 4, 5, 6, 7 | YYWPRPRRY | De Backer et al. Cancer |
| | (lung squamous | | Res. 59(13): 3157-65 (1999). |
| | cell carcinoma | | |
| | and lung | | |
| | adenocarcinoma) | | |
| 12 | HERV-K-MEL | MLAVISCAV | Schiavetti et al. Cancer Res. |
| | | | 62(19): 5510-6 (2002). |
| 13 | KK-LC-1 | RQKRILVNL | Fukuyama et al. Cancer Res. |
| | | | 66(9): 4922-8 (2006). |
| 14 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer |
| | | | Res. 10(18 Pt 1): 6047-57 |
| | | | (2004). |
| 15 | LAGE-1 | MLMAQEALAFL | Aarnoudse et al. Int J |
| | | SLLMWITQC | Cancer. 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. |
| | | SLLMWITQCFLPVF | 161(7): 3598-606 (1998). |
| | | QGAMLAAQERRVPRAAEVP-R | Sun et al. Cancer Immunol |
| | | AADHRQLQLSISSCLQQL | Immunother. 55(6): 644-52 |
| | | CLSRRPWKRSWSAGSCPG- | (2006). |
| | | MPHL | Slager et al. Cancer Gene |
| | | ILSRDAAPLPRPG | Ther. 11(3): 227-36 (2004). |
| | | AGATGGRGPRGAGA | Zeng et al. Proc Natl Acad |
| | | | Sci USA. 98(7): 3964- |
| | | | 9(2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 16 | Sp17 | ILDSSEEDK | Chiriva-Internati et al. Int J |
| | | | Cancer. 107(5): 863-5 |
| | | | (2003). |

TABLE 12
Kidney cancer

| | Tumor- | Reported | |
|---|---|---|---|
| | associated | immunogenic | |
| No. | antigen | epitopes | Sources |
| 1 | FGF5 | NTYASPRFK | Hanada et al. Nature. |
| | | | 427(6971): 252-6 (2004). |
| 2 | Hepsin | SLLSGDWVL; | Guo et al. Scand J Immunol. |
| | | GLQLGVQAV; | 78(3): 248-57 (2013). |
| | | and | |
| | | PLTEYIQPV | |
| 3 | Intestinal | SPRWWPTCL | Ronsin et al. J Immunol. |
| | carboxyl | | 163(1): 483-90 (1999). |
| | esterase | | |
| 4 | M-CSF | LPAVVGLSPGEQEY | Probst-Kepper et al. J Exp |
| | | | Med. 193(10): 1189-98 (2001). |
| 5 | RU2AS | LPRWPPPQL | Van Den Eynde et al. J. Exp. |
| | | | Med. 190(12): 1793-800 |
| | | | (1999). |
| 6 | hsp70-2 (renal | SLFEGIDIYT | Gaudin et al. J. Immunol. |
| | cell carcinoma) | | 162(3): 1730-8 (1999). |
| 7 | Mannan-MUC-1 | PDTRPAPGSTAPPAHGVTSA | Loveland et al. Clin. Cancer |
| | (renal cell | STAPPVHNV | Res. 12(3 Pt 1): 869-77 (2006). |
| | carcinoma) | LLLLTVLTV | Loveland et al. Clin. Cancer |
| | | PGSTAPPAHGVT | Res. 12(3 Pt 1): 869-77 (2006). |
| | | | Godelaine et al. Cancer |
| | | | Immunol Immunother. |
| | | | 56(6): 753-9 (2007). |
| | | | Ma et al. Int J Cancer. |
| | | | 129(10): 2427-34 (2011). |
| | | | Wen et al. Cancer Sci. |
| | | | 102(8): 1455-61 (2011). |
| | | | Jerome et al. J Immunol. |
| | | | 151(3): 1654-62 (1993). |
| | | | Brossart et al. Blood. |
| | | | 93(12): 4309-17 (1999). |
| | | | Hiltbold et al. Cancer Res. |
| | | | 58(22): 5066-70 (1998). |
| 8 | MAGE-A9 (renal | ALSVMGVYV | Oehlrich et al. Int J Cancer. |
| | cell carcinoma) | | 117(2): 256-64 (2005). |

TABLE 13
Melanoma

| | Tumor- | Reported | |
|---|---|---|---|
| | associated | immunogenic | |
| No. | antigen | epitopes | Sources |
| 1 | Hepsin | SLLSGDWVL; | Guo et at. Scand J Immunol. |
| | | GLQLGVQA; | 78(3): 248-57 (2013). |
| | | and | |
| | | PLTEYIQPV | |
| 2 | ARTC1 | YSVYFNLPADTIYTN | Wang et al J Immunol. 174(5): 2661- |
| | | | 70 (2005). |
| 3 | B-RAF | EDLTVKIGDFGLATEKSR | Sharkey et at. Cancer Res. |
| | | WSGSHQFEQLS | 64(5): 1595-9 (2004). |
| 4 | beta-catenin | SYLDSGIHF | Robbins et al. J. Exp. Med. |
| | | | 183(3): 1185-92 (1996). |
| 5 | Cdc27 | FSWAMDLDPKGA | Wang et al. Science. |
| | | | 284(5418): 1351-4 (1999). |
| 6 | CDK4 | ACDPHSGHFV | Wölfel et al. Science. |
| | | | 269(5228): 1281-4 (1995). |
| 7 | CDK12 | CILGKLFTK | Robbins et al. Nat Med. 19(6): 747- |
| | | | 52. (2013). |
| 8 | CDKN2A | AVCPWTWLR | Huang et al. J Immunol. |
| | | | 172(10): 6057-64 (2004). |
| 9 | CLPP | ILDKVLVHL | Corbière et al. Cancer Res. |
| | | | 71(4): 1253-62 (2011). |
| 10 | CSNK1A1 | GLFGDIYLA | Robbins et al. Nat Med. 19(6): 747- |
| | | | 52 (2013). |
| 11 | FN1 | MIFEKHGFRRTTPP | Wang et al. J Exp Med. |
| | | | 195(11): 1397-406 (2003). |
| 12 | GAS7 | SLADEAEVYL | Robbins, et al. Nat Med. 19(6): 747- |
| | | | 52 (2013). |
| 13 | GPNMB | TLDWLLQTPK | Lennerz et al. Proc. Natl. Acad. Sci. |
| | | | U.S.A. 102(44): 16013-8 (2005). |
| 14 | HAUS3 | ILNAMIAKI | Robbins et al. Nat Med. 19(6): 747- |
| | | | 52 (2013). |
| 15 | LDLR- | WRRAPAPGA | Wang et al. J Exp Med. |
| | fucosyltransferase | and | 189(10): 1659-68 (1999). |
| | | PVTWRRAPA | |
| 16 | MART2 | FLEGNEVGKTY | Kawakami et al. J Immunol. |
| | | | 166(4): 2871-7 (2001). |
| 17 | MATN | KTLTSVFQK | Robbins et al. Nat Med. 19(6): 747- |
| | | | 52 (2013). |
| 18 | MUM-1 | EEKLIVVLF | Coulie et al. Proc. Natl. Acad. Sci. |
| | | | U.S.A. 92(17): 7976-80 (1995). |
| 19 | MUM-2 | SELFRSGLDSY | Chiari et al. Cancer Res. |
| | | and | 59(22): 5785-92 (1999). |
| | | FRSGLDSYV | |
| 20 | MUM-3 | EAFIQPITR | Baurain et al. J. Immunol. |
| | | | 164(11): 6057-66 (2000). |
| 21 | neo-PAP | RVIKNSIRLTL | Topalian et al. Cancer Res. |
| | | | 62(19): 5505-9 (2002). |
| 22 | Myosin class I | KINKNPKYK | Zorn, et al. Eur. J. Immunol. |
| | | | 29(2): 592-601 (1999). |
| 23 | PPP1R3B | YTDFHCQYV | Robbins et al. Nat Med. 19(6): 747- |
| | | | 52 (2013). |
| | | | Lu et al. J Immunol. 190(12): 6034- |
| | | | 42 (2013). |
| 24 | PRDX5 | LLLDDLLVSI | Sensi et al. Cancer Res. 65(2): 632- |
| | | | 40 (2005). |
| 25 | PTPRK | PYYFAAELPPRNLPEP | Novellino et al. J. Immunol. |
| | | | 170(12): 6363-70 (2003). |
| 26 | N-ras | ILDTAGREEY | Linard et al. J. Immunol. |
| | | | 168(9): 4802-8 (2002). |
| 27 | RBAF600 | RPHVPESAF | Lennerz et al. Proc. Natl. Acad. Sci. |
| | | | U.S.A. 102(44): 16013-8 (2005). |
| 28 | SIRT2 | KIFSEVTLK | Lennerz et al. Proc. Natl. Acad. Sci. |
| | | | U.S.A. 102(44): 16013-8 (2005). |
| 29 | SNRPD1 | SHETVIIEL | Lennerz et al. Proc. Natl. Acad. Sci. |
| | | | U.S.A. 102(44): 16013-8 (2005). |
| 30 | Triosephosphate | GELIGILNAAKVPAD | Pieper et al. J Exp Med. 189(5): 757- |
| | isomerase | | 66 (1999). |
| 31 | OA1 | LYSACFWWL | Touloukian et al. J. Immunol. |
| | | | 170(3): 1579-85 (2003). |
| 32 | RAB38/NY-MEL-1 | VLHWDPETV | Walton et al. J Immunol. |
| | | | 177(11): 8212-8 (2006). |
| 33 | TRP-1/gp75 | MSLQRQFLR; | Touloukian et al. Cancer Res. |
| | | ISPNSVFSQWRVVCDSLE | 62(18): 5144-7 (2002). |
| | | DY; | Robbins et al. J. Immunol. |
| | | SLPYWNFATG; | (10): 6036-47 (2002). |
| | | and | Osen et al. PLoS One. 5(11): e14137 |
| | | SQWRVVCDSLEDYDT | (2010). |
| 34 | TRP-2 | SVYDFFVWL; | Parkhurst et al. Cancer Res. |
| | | TLDSQVMSL; | 58(21): 4895-901 (1998). |
| | | LLGPGRPYR; | Noppen et al. Int. J. Cancer. |
| | | ANDPIFVVL; | 87(2): 241-6 (2000). |
| | | QCTEVRADTRPWSGP; | Wang et al. J. Exp. Med. |
| | | and | 1184(6): 2207-16 (1996). |
| | | ALPYWNFATG | Wang et al. J. Immunol. 160(2): 890- |
| | | | 7(1998). |
| | | | Castelli et al. J. Immunol. |
| | | | 162(3): 1739-48 (1999). |
| | | | Paschen et al. Clin. Cancer Res. |
| | | | (14): 5241-7 (2005). |
| | | | Robbins et al. J. Immunol. |
| | | | 169(10): 6036-47 (2002). |
| 35 | tyrosinase | KCDICTDEY; | Kittlesen et al. J. Immunol. |
| | | SSDYVIPIGTY; | 160(5): 2099-106 (1998). |
| | | MLLAVLYCL; | Kawakami et al. J. Immunol. |
| | | CLLWSFQTSA; | (12): 6985-92 (1998). |
| | | YMDGTMSQV; | Wölfel et al. Eur. J. Immunol. |
| | | AFLPWHRLF; | 24(3): 759-64 (1994). |
| | | IYMDGTADFSF; | Riley et al. J. Immunother. |
| | | QCSGNFMGF; | 24(3): 212-20 (2001). |
| | | TPRLPSSADVEF; | Skipper et al. J. Exp. Med. |
| | | LPSSADVEF; | 183(2): 527-34 (1996). |
| | | LHHAFVDSIF; | Kang et al. J. Immunol. |
| | | SEIWRDIDF; | 155(3): 1343-8 (1995). |
| | | QNILLSNAPLGPQFP; | Dalet et al. Proc. Natl. Acad. Sci. |
| | | SYLQDSDPDSFQD; | U.S.A. 108(29): E323-31 (2011) |
| | | and | Lennerz et al. Proc. Natl. Acad. Sci. |
| | | FLLHHAFVDSIFEQWLQR | U.S.A. 102(44): 16013-8 (2005). |
| | | HRP | Benlalam et al. J. Immunol. |
| | | | 171(11): 6283-9 (2003). |
| | | | Morel et al. Int. J. Cancer. |
| | | | 83(6): 755-9 (1999). |
| | | | Brichard et al. Eur. J. Immunol. |
| | | | 26(1): 224-30 (1996). |
| | | | Topalian et al. J. Exp. Med. |
| | | | (5): 1965-71 (1996). |
| | | | Kobayashi et al. Cancer Res. |
| | | | 58(2): 296-301 (1998). |
| 36 | Melan-A/MART-1 | YTTAEEAAGIGILTVILGV | Meng et al. J. Immunother. 23: 525- |
| | | LLLIGCWYCRR | 534(2011) |
| 37 | gp100/Pmel17 | ALNFPGSQK | El Hage et al. Proc. Natl. Acad. Sci. |
| | | ALNFPGSQK | U.S.A. 105(29): 10119-24 (2008). |
| | | VYFFLPDHL | Kawashima et al. Hum Immunol. |
| | | RTKQLYPEW | 59(1): 1-14 (1998). |
| | | HTMEVTVYHR | Robbins et al. J Immunol. |
| | | SSPGCQPPA | 159(1): 303-8 (1997). |
| | | VPLDCVLYRY | Sensi et al. Tissue Antigens. |
| | | LPHSSSHWL | 59(4): 273-9 (2002). |
| | | SNDGPTLI | Lennerz et al. Proc Natl Acad Sci |
| | | GRAMLGTHTMEVTVY | USA. 102(44): 16013-8 (2005). |
| | | WNRQLYPEWTEAQRLD | Benlalam et al. J Immunol. |
| | | TTEWVETTARELPIPEPE | 171(11): 6283-9 (2003). |
| | | TGRAMLGTHTMEVTVYH | Vigneron et al. Tissue Antigens. |
| | | GRAMLGTHTMEVTVY | 65(2): 156-62 (2005). |
| | | | Castelli et al. J Immunol. |
| | | | 162(3): 1739-48 (1999). |
| | | | Touloukian et al. J Immunol. |
| | | | 164(7): 3535-42 (2000). |
| | | | Parkhurst et al. J Immunother. |
| | | | 27(2): 79-91 (2004). |
| | | | Lapointe et al. J Immunol. |
| | | | 167(8): 4758-64 (2001). |
| | | | Kobayashi et al. Cancer Res. |
| | | | 61(12): 4773-8 (2001). |
| 38 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9(2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 39 | BAGE-1 | AARAVFLAL | Boel et al. Immunity. 2(2): 167- |
| | | | 75 (1995). |
| 40 | GAGE-1, 2, 8 | YRPRPRRY | Van den Eynde et al. J Exp Med. |
| | | | 182(3): 689-98 (1995). |
| 41 | GAGE-3, 4, 5, 6, 7 | YYWPRPRRY | De Backer et al. Cancer Res. |
| | (cutaneous | | 59(13): 3157-65 (1999). |
| | melanoma) | | |
| 42 | GnTVf | VLPDVFIRC(V) | Guilloux et al. J Exp Med. |
| | | | 183(3): 1173-83 (1996). |
| 43 | HERV-K-MEL | MLAVISCAV | Schiavetti et al. Cancer Res. |
| | | | 62(19): 5510-6 (2002). |
| 44 | KK-LC-1 | RQKRILVNL | Fukuyama et al. Cancer Res. |
| | | | 66(9): 4922-8 (2006). |
| 45 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 46 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Stager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 47 | LY6K | RYCNLEGPPI | Suda et al. Cancer Sci. 98(11): 1803- |
| | | KWTEPYCVIAAVKIFPRF | 8 (2007). |
| | | FMV-AKQ | Tomita et al. Oncoimmunology. |
| | | KCCKIRYCNLEGPPINSSVF | 3: e28100 (2014). |
| 48 | MAGE-A1 | EADPTGHSY | Traversari et al. J Exp Med. |
| | | KVLEYVIKV | 176(5): 1453-7 (1992). |
| | | SLFRAVITK | Ottaviani et al. Cancer Immunol |
| | | EVYDGREHSA | Immunother. 54(12): 1214-20 (2005). |
| | | RVRFFFPSL | Pascolo et al. Cancer Res. |
| | | EADPTGHSY | 61(10): 4072-7 (2001). |
| | | REPVTKAEML | Chaux et al. J Immunol. |
| | | KEADPTGHSY | 163(5): 2928-36 (1999). |
| | | DPARYEFLW | Luiten et al. Tissue Antigens. |
| | | ITKKVADLVGF | 55(2): 149-52 (2000). |
| | | SAFPTTINF | Luiten et al. Tissue Antigens. |
| | | SAYGEPRKL | 56(1): 77-81 (2000). |
| | | RVRFFFPSL | Tanzarella et al. Cancer Res. |
| | | TSCILESLFRAVITK | 59(11): 2668-74 (1999). |
| | | PRALAETSYVKVLEY | Stroobant et al. Eur J Immunol. |
| | | FLLIKYRAREPVTKAE | 42(6): 1417-28 (2012). |
| | | EYVIKVSARVRF | Corbière et al. Tissue Antigens. |
| | | | 63(5): 453-7 (2004). |
| | | | Goodyear et al. Cancer Immunol |
| | | | Immunother. 60(12): 1751-61 (2011). |
| | | | van der Bruggen et al. Eur J |
| | | | Immunol. 24(9): 2134-40 (1994). |
| | | | Wang et al. Cancer Immunol |
| | | | Immunother. 56(6): 807-18 (2007). |
| | | | Chaux et al. J Exp Med. 189(5): 767- |
| | | | 78 (1999). |
| | | | Chaux et al. Eur J Immunol. |
| | | | 31(6): 1910-6 (2001). |
| 49 | MAGE-A6 | MVKISGGPR | Zorn et al. Eur J Immunol. |
| | | EVDPIGHVY | 29(2): 602-7 (1999). |
| | | REPVTKAEML | Benlalam et al. J Immunol. |
| | | EGDCAPEEK | 171(11): 6283-9 (2003). |
| | | ISGGPRISY | Tanzarella et al. Cancer Res. |
| | | LLKYRAREPVTKAE | 59(11): 2668-74 (1999). |
| | | | Breckpot et al. J Immunol. |
| | | | 172(4): 2232-7 (2004). |
| | | | Vantomme et al. Cancer Immun. |
| | | | 3:17 (2003). |
| | | | Chaux et al. J Exp Med. 189(5): 767- |
| | | | 78 (1999). |
| 50 | MAGE-A10 | GLYDGMEHL | Huang et al. J Immunol. |
| | | DPARYEFLW | 162(11): 6849-54 (1999). |
| | | | Chaux et al. J Immunol. |
| | | | 163(5): 2928-36 (1999). |
| 51 | MAGE-A12 | FLWGPRALV | van der Bruggen et al. Eur J |
| | | VRIGHLYIL | Immunol. 24(12): 3038-43 (1994). |
| | | EGDCAPEEK | Heidecker et al. J Immunol. |
| | | REPFTKAEMLGSVIR | 164(11): 6041-5 (2000). |
| | | AELVHFLLLKYRAR | Panelli et al. J Immunol. |
| | | | 164(8): 4382-92 (2000). |
| | | | Breckpot et al. J Immunol. |
| | | | 172(4): 2232-7 (2004). |
| | | | Wang et al. Cancer Immunol |
| | | | Immunother. 56(6): 807-18 (2007). |
| | | | Chaux et al. J Exp Med. 189(5): 767- |
| | | | 78 (1999). |
| 52 | MAGE-C2 | LLFGLALIEV | Ma et al. Int J Cancer. 109(5): 698- |
| | | ALKDVEERV | 702 (2004). |
| | | SESIKKKVL | Godelaine et al. Cancer Immunol |
| | | ASSTLYLVF | Immunother. 56(6): 753-9 (2007). |
| | | SSTLYLVFSPSSFST | Ma et al. Int J Cancer. 129(10): 2427- |
| | | | 34 (2011). |
| | | | Wen et al. Cancer Sci. 102(8): 1455- |
| | | | 61 (2011). |
| 53 | NA88-A | QGQHFLQKV | Moreau-Aubry et al. J Exp Med. |
| | | | 191(9): 1617-24 (2000). |
| 54 | Sp17 | ILDSSEEDK | Chiriva-Internati et al. Int J Cancer. |
| | | | 107(5): 863-5 (2003). |
| 55 | SSX-2 | KASEKIFYV | Ayyoub et al. J Immunol. |
| | | EKIQKAFDDIAKYFSK | 168(4): 1717-22 (2002). |
| | | FGRLQGISPKI | Ayyoub et al. J Immunol. |
| | | WEKMKASEKIFYVYMKRK | 172(11): 7206-11 (2004). |
| | | KIFYVYMKRKYEAMT | Neumann et al. Cancer Immunol |
| | | KIFYVYMKRKYEAM | Immunother. 60(9): 1333-46 (2011). |
| | | | Ayyoub et al. Clin Immunol. |
| | | | 114(1): 70-8 (2005). |
| | | | Neumann et al. Int J Cancer. |
| | | | 112(4): 661-8 (2004). |
| | | | Ayyoub et al. J Clin Invest. |
| | | | 113(8): 1225-33 (2004). |
| 56 | SSX-4 | INKTSGPKRGKHAWTHR | Ayyoub et al. J Immunol. |
| | | LRE | 174(8): 5092-9 (2005). |
| | | YFSKKEWEKMKSSEKIV | Valmori et al. Clin Cancer Res. |
| | | YVY | 12(2): 398-404 (2006). |
| | | MKLNYEVMTKLGFKVTL | |
| | | PPF | |
| | | KHAWTHRLRERKQLVV | |
| | | YEEI | |
| | | LGFKVTLPPFMRSKRAA | |
| | | DFH | |
| | | KSSEKIVYVYMKLNYEV | |
| | | MTK | |
| | | KHAWTHRLRERKQLVV | |
| | | YEEI | |
| 57 | TRAG-3 | CEFHACWPAFTVLGE | Janjic et al. J Immunol. 177(4): 2717- |
| | | | 27 (2006). |
| 58 | TRP2-INT2g | EVISCKLIKR | Lupetti et al. J Exp Med. |
| | | | 188(6): 1005-16 (1998). |
| 59 | pgk | | Morgan et al., J. Immunol. |
| | | | 171: 3287-3295 (2003) |

TABLE 14
Squamous cell carcinoma

| | Tumor- | Reported | |
|---|---|---|---|
| | associated | immunogenic | |
| No. | antigen | epitopes | Sources |
| 1 | CASP-8 | FPSDSWCYF | Mandruzzato et al. J. Exp. Med. |
| | | | 186(5): 785-93 (1997). |
| 2 | p53 | VVPCEPPEV | Ito et al. Int. J. Cancer. |
| | | | 120(12): 2618-24 (2007). |
| 3 | SAGE | LYATVIHDI | Miyahara et al. Clin Cancer Res. |
| | | | 11(15): 5581-9 (2005). |

TABLE 15
Chronic myeloid leukemia

| | Tumor- | | |
|---|---|---|---|
| | associated | Reported immunogenic | |
| No. | antigen | epitopes | Sources |
| 1 | BCR-ABL | SSKALQRPV; | Yotnda et al. J. Clin. Invest. |
| | | GFKQSSKAL; | 101(10): 2290-6 (1998). |
| | | ATGFKQSSKALQRPVAS; | Bosch et al. Blood. 88(9): 3522-7 |
| | | and | (1996). |
| | | ATGFKQSSKALQRPVAS | Makita et al. Leukemia. |
| | | | 16(12): 2400-7 (2002). |
| 2 | dek-can | TMKQICKKEIRRLHQY | Makita et al. Leukemia. |
| | | | 16(12): 2400-7 (2002). |
| 3 | EFTUD2 | KILDAVVAQK | Lennerz et al. Proc. Natl. Acad. |
| | | | Sci. U.S.A. 102(44): 16013-8 |
| | | | (2005). |
| 4 | GAGE-3, 4, 5, | YYWPRPRRY | De Backer et al. Cancer Res. |
| | 6, 7 | | 59(13): 3157-65 (1999). |

TABLE 16
Acute lymphoblastic leukemia

| | Tumor- | | |
|---|---|---|---|
| | associated | Reported immunogenic | |
| No. | antigen | epitopes | Sources |
| 1 | ETV6-AML1 | RIAECILGM | Yotnda et al. J. Clin. Invest. |
| | | and | (2): 455-62 (1998). |
| | | IGRIAECILGMNPSR | Yun et al. Tissue Antigens. |
| | | | 54(2): 153-61 (1999). |
| 2 | GAGE-3, 4, 5, | YYWPRPRRY | De Backer et al. Cancer Res. |
| | 6, 7 | | 59(13): 3157-65 (1999). |

TABLE 17
Acute myelogenous leukemia

| | Tumor- | | |
|---|---|---|---|
| | associated | Reported immunogenic | |
| No. | antigen | epitopes | Sources |
| 1 | FLT3-ITD | YVDFREYEYY | Graf et al. Blood. 109(7): 2985-8 |
| | | | (2007). |
| 2 | Cyclin-A1 | FLDRFLSCM | Ochsenreither et al. Blood. |
| | | and | 119(23): 5492-501 (2012). |
| | | SLIAAAAFCLA | |
| 3 | GAGE-3, 4, 5, | YYWPRPRRY | De Backer et al. Cancer Res. |
| | 6, 7 | | 59(13): 3157-65 (1999). |

TABLE 18
Chronic lymphocytic leukemia

| | Tumor- | | |
|---|---|---|---|
| | associated | Reported immunogenic | |
| No. | antigen | epitopes | Sources |
| 1 | FNDC3B | VVMSWAPPV | Rajasagi et al. Blood. 124(3): 453- |
| | | | 62 (2014). |
| 2 | GAGE-3, 4, 5, | YYWPRPRRY | De Backer et al. Cancer Res. |
| | 6, 7 | | 59(13): 3157-65 (1999). |

TABLE 19
Promyelocytic leukemia

| | Tumor- | | |
|---|---|---|---|
| | associated | Reported immunogenic | |
| No. | antigen | epitopes | Sources |
| 1 | pml-RARalpha | NSNHVASGAGEAAIETQS | Gambacorti-Passerini et al. Blood. |
| | | SSSEEIV | 81(5): 1369-75 (1993). |
| 2 | GAGE-3, 4, 5, | YYWPRPRRY | De Backer et al. Cancer Res. |
| | 6, 7 | | 59(13): 3157-65 (1999). |

TABLE 20
Multiple myeloma

| | Tumor- | | |
|---|---|---|---|
| | associated | Reported immunogenic | |
| No. | antigen | epitopes | Sources |
| 1 | MAGE-C1 | ILFGISLREV | Anderson et al. Cancer Immunol |
| | | KVVEFLAML | Immunother. 60(7): 985-97 (2011). |
| | | SSALLSIFQSSPE | Nuber et al. Proc Natl Acad Sci USA. |
| | | SFSYTLLSL | 107(34): 15187-92 (2010). |
| | | VSSFFSYTL | |
| 2 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9(2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 3 | LAGE-1 | MLMAQEALAFL | Aarnoudse et al. Int J Cancer. |
| | | SLMMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 4 | HERV-K-MEL | MLAVISCAV | Schiavetti et al. Cancer Res. |
| | | | 62(19): 5510-6 (2002). |
| 5 | KK-LC-1 | RQKRILVNL | Fukuyama et al. Cancer Res. |
| | | | 66(9): 4922-8 (2006). |
| 6 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 7 | Sp17 | ILDSSEEDK | Chiriva-Internati et al. Int J Cancer. |
| | | | 107(5): 863-5 (2003). |

TABLE 21
B-cell lymphoma

| | Tumor- | Reported | |
|---|---|---|---|
| | associated | immunogenic | |
| No. | antigen | epitopes | Sources |
| 1 | D393-CD20 | KPLFRRMSSLELVIA | Vauchy et al. Int |
| | | | J Cancer. 137(1): |
| | | | 116-26 (2015). |

TABLE 22
Bladder carcinoma

| | Tumor- | | |
|---|---|---|---|
| | associated | Reported immunogenic | |
| No. | antigen | epitopes | Sources |
| 1 | BAGE-1 | AARAVFLAL | Boel et al. Immunity. 2(2): 167- |
| | | | 75 (1995). |
| 2 | GAGE-1, 2, 8 | YRPRPRRY | Van den Eynde et al. J Exp Med. |
| | | | 182(3): 689-98 (1995). |
| 3 | GAGE-3, 4, 5, 6, 7 | YYWPRPRRY | De Backer et al. Cancer Res. |
| | | | 59(13): 3157-65 (1999). |
| 4 | MAGE-A4 | EVDPASNTY | Kobayashi et al. Tissue Antigens. |
| | (transitional cell | GVYDGREHTV | 62(5): 426-32 (2003). |
| | carcinoma of | NYKRCFPVI | Duffour et al. Eur J Immunol. |
| | urinary bladder) | SESLKMIF | 29(10): 3329-37 (1999). |
| | | | Miyahara et al. Clin Cancer Res. |
| | | | 11(15): 5581-9 (2005). |
| | | | Ottaviani et al. Cancer Immunol |
| | | | Immunother. 55(7): 867-72 (2006). |
| | | | Zhang et al. Tissue Antigens. |
| | | | 60(5): 365-71 (2002). |
| 5 | MAGE-A6 | MVKISGGPR | Zorn et al. Eur J Immunol. |
| | | EVDPIGHVY | 29(2): 602-7 (1999). |
| | | REPVTKAEML | Benlalam et al. J Immunol. |
| | | EGDCAPEEK | 171(11): 6283-9 (2003). |
| | | ISGGPRISY | Tanzarella et al. Cancer Res. |
| | | LLKYRAREPVTKAE | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. 189(5): 767- |
| | | | 78 (1999). |
| 6 | SAGE | LYATVIHDI | Miyahara et al. Clin Cancer Res. |
| | | | 11(15): 5581-9 (2005). |
| 7 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. 161(7): 3598- |
| | | KEFTVSGNILTI | 606 (1998). |
| | | MPFATPMEA | Matusuzki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 8 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 9 | HERV-K-MEL | MLAVISCAV | Schiavetti et al. Cancer Res. |
| | | | 62(19): 5510-6 (2002). |
| 10 | KK-LC-1 | RQKRILVNL | Fukuyama et al. Cancer Res. |
| | | | 66(9): 4922-8 (2006). |
| 11 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 12 | SP17 | ILDSSEEDK | Chiriva-Internati et al. Int J Cancer. |
| | | | 107(5): 863-5 (2003). |

TABLE 23
Head and neck cancer

| | Tumor- | | |
|---|---|---|---|
| | associated | Reported immunogenic | |
| No. | antigen | epitopes | Sources |
| 1 | BAGE-1 (head and | AARAVFLAL | Boel et al. Immunity. 2(2): 167- |
| | neck squamous cell | | 75 (1995). |
| | carcinoma) | | |
| 2 | GAGE-1, 2, 8 | YRPRPRRY | Van den Eynde et al. J Exp Med. |
| | | | 182(3): 689-98 (1995). |
| 3 | GAGE-3, 4, 5, 6, 7 | YYWPRPRRY | De Backer et al. Cancer Res. |
| | | | 59(13): 3157-65 (1999). |
| 4 | LY6K | RYCNLEGPPI | Suda et al. Cancer Sci. 98(11): 1803- |
| | | KWTEPYCVIAAVKIFPRF | 8 (2007). |
| | | FMV-AKQ | Tomita et al. Oncoimmunology. |
| | | KCCKIRYCNLEGPPINSSVF | 3: e28100 (2014). |
| 5 | MAGE-A3 (head | EVDPIGHLY | Gaugler et al. J Exp Med. |
| | and neck squamous | FLWGPRALV | 179(3): 921-30 (1994). |
| | cell carcinoma) | KVAELVHFL | van der Bruggen et al. Eur J |
| | | TFPDLESEF | Immunol. 24(12): 3038-43 (1994). |
| | | VAELVHFLL | Kawashima et al. Hum Immunol. |
| | | MEVDPIGHLY | 59(1): 1-14 (1998). |
| | | EVDPIGHLY | Oiso et al. Int J Cancer. 81(3): 387- |
| | | REPVTKAEML | 94 (1999). |
| | | AELVHFLLL | Miyagawa et al. Oncology. 70(1): 54- |
| | | MEVDPIGHLY | 62 (2006). |
| | | WQYFFPVIF | Bilsborough et al. Tissue Antigens. |
| | | EGDCAPEEK | 60(1): 16-24 (2002). |
| | | KKLLTQHFVQENYLEY | Schultz et al. Tissue Antigens. |
| | | RKVAELVHFLLLKYR | 57(2): 103-9 (2001). |
| | | KKLLTQHFVQENYLEY | Tanzarella et al. Cancer Res. |
| | | ACYEFLWGPRALVETS | 59(11): 2668-74 (1999). |
| | | RKVAELVHFLLLKYR | Schultz et al. J Exp Med. |
| | | VIFSKASSSLQL | 195(4): 391-9 (2002). |
| | | VFGIELMEVDPIGHL | Herman et al. Immunogenetics. |
| | | GDNQIMPKAGLLIIV | 43(6): 377-83 (1996). |
| | | TSYVKVLHHMVKISG | Russo et al. Proc Natl Acad Sci USA. |
| | | RKVAELVHFLLLKYRA | 97(5): 2185-90 (2000). |
| | | FLLLKYRAREPVTKAE | Breckpot et al. J Immunol. |
| | | | 172(4): 2232-7 (2004). |
| | | | Schultz et al. Cancer Res. |
| | | | 60(22): 6272-5 (2000). |
| | | | Cesson et al. Cancer Immunol |
| | | | Immunother. 60(1): 23-35 (2011). |
| | | | Schultz et al. J Immunol. |
| | | | 172(2): 1304-10 (2004). |
| | | | Zhang et al. J Immunol. 171(1): 219- |
| | | | 25 (2003). |
| | | | Cesson et al. Cancer Immunol |
| | | | Immunother. 60(1): 23-35 (2010). |
| | | | Kobayashi et al. Cancer Res. |
| | | | 61(12): 4773-8 (2001). |
| | | | Cesson et al. Cancer Immunol |
| | | | Immunother. 60(1): 23-35 (2011). |
| | | | Consogno et al. Blood. 101(3): 1038- |
| | | | 44 (2003). |
| | | | Manici et al. J Exp Med. 189(5): 871- |
| | | | 6 (1999). |
| | | | Chaux et al. J Exp Med. 189(5): 767- |
| | | | 78 (1999). |
| 6 | MAGE-A6 | MVKISGGPR | Zorn et al. Eur J Immunol. |
| | | EVDPIGHVY | 29(2): 602-7 (1999). |
| | | REPVTKAEML | Benlalam et al. J Immunol. |
| | | EGDCAPEEK | 171(11): 6283-9 (2003). |
| | | ISGGPRISY | Tanzarella et al. Cancer Res. |
| | | LLKYRAREPVTKAE | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. 189(5): 767- |
| | | | 78 (1999). |
| 7 | SAGE | LYATVIHDI | Miyahara et al. Clin Cancer Res. |
| | | | 11(15): 5581-9 (2005). |

TABLE 24
Esophageal cancer

| | Tumor- | | |
|---|---|---|---|
| | associated | Reported immunogenic | |
| No. | antigen | epitopes | Sources |
| 1 | GAGE-3, 4, 5, 6, 7 | YYWPRPRRY | De Backer et al. Cancer Res. |
| | (Esophageal | | 59(13): 3157-65 (1999). |
| | squamous cell | | |
| | carcinoma and | | |
| | esophageal | | |
| | adenocarcinoma) | | |
| 2 | MAGE-A2 | YLQLVFGIEV | Kawashima et al. Hum Immunol. |
| | | EYLQLVFGI | 59(1): 1-14 (1998). |
| | | REPVTKAEML | Tahara et al. Clin Cancer Res. |
| | | EGDCAPEEK | 5(8): 2236-41 (1999). |
| | | LLKYRAREPVTKAE | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. 189(5): 767- |
| | | | 78 (1999). |
| 3 | MAGE-A6 | MVKISGGPR | Zorn et al. Eur J Immunol. |
| | | EVDPIGHVY | 29(2): 602-7 (1999). |
| | | REPVTKAEML | Benlalam et al. J Immunol. |
| | | EGDCAPEEK | 171(11): 6283-9 (2003). |
| | | ISGGPRISY | Tanzarella et al. Cancer Res. |
| | | LLKYRAREPVTKAE | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. 189(5): 767- |
| | | | 78 (1999). |
| 4 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int. J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol 161(7): 3598- |
| | | KEFTVSGNILTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 5 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 6 | HERV-K-MEL | MLAVISCAV | Schiavetti et al. Cancer Res. |
| | | | 62(19): 5510-6 (2002). |
| 7 | KK-LC-1 | RQKRILVNL | Fukuyama et al. Cancer Res. |
| | | | 66(9): 4922-8 (2006). |
| 8 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 9 | Sp17 | ILDSSEEDK | Chiriva-Internati et al. Int J Cancer. |
| | | | 107(5): 863-5 (2003). |

TABLE 25
Brain cancer

| | Tumor- | | |
|---|---|---|---|
| | associated | Reported immunogenic | |
| No. | antigen | epitopes | Sources |
| 1 | TAG-1 | SLGWLFLLL | Adair et al. J Immunother. 31(1): 7- |
| | | LSRLSNRLL | 17 (2008). |
| 2 | TAG-2 | LSRLSNRLL | Adair et al. J Immunother. 31(1): 7- |
| | | | 17 (2008). |

TABLE 26
Pharynx cancer

| | Tumor- | | |
|---|---|---|---|
| | associated | Reported immunogenic | |
| No. | antigen | epitopes | Sources |
| 1 | TAG-1 | SLGWLFLLL | Adair et al. J Immunother. 31(1): 7- |
| | | LSRLSNRLL | 17 (2008). |
| 2 | TAG-2 | LSRLSNRLL | Adair et al. J Immunother. 31(1): 7- |
| | | | 17 (2008). |

TABLE 27
Tumors of the tongue

| | Tumor- | | |
|---|---|---|---|
| | associated | Reported immunogenic | |
| No. | antigen | epitopes | Sources |
| 1 | TAG-1 | SLGWLFLLL | Adair et al. J Immunother. 31(1): 7- |
| | | LSRLSNRLL | 17 (2008). |
| 2 | TAG-2 | LSRLSNRLL | Adair et al. J Immunother. 31(1): 7- |
| | | | 17 (2008). |

TABLE 28
Synovial cell sarcoma

| | Tumor- | | |
|---|---|---|---|
| | associated | Reported immunogenic | |
| No. | antigen | epitopes | Sources |
| 1 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. |
| | | SLLMWITQCFLPVF | 161(7): 3598-606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 2 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 |
| | | (LAMP-FATPM) | p. 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. |
| | | KEFTVSGNILTI | 161(7): 3598-606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Immunother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 3 | HERV-K-MEL | MLAVISCAV | Schiavetti et al. Cancer Res. |
| | | | 62(19): 5510-6 (2002). |
| 4 | KK-LC-1 | RQKRILVNL | Fukuyama et al. Cancer Res. |
| | | | 66(9): 4922-8 (2006). |
| 5 | KM-HN-1 | NYNNFYRFL | Fukuyama et al. Cancer Res. |
| | | EYSKECLKEF | 66(9): 4922-8 (2006). |
| | | EYLSLSDKI | |
| 6 | Sp17 | ILDSSEEDK | Chiriva-Internati et al. Int J Cancer. |
| | | | 107(5): 863-5 (2003). |

TABLE 29
Neuroblastoma

| | Tumor- | | |
|---|---|---|---|
| | associated | Reported immunogenic | |
| No. | antigen | epitopes | Sources |
| 1 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 2 | NY-ESO-1 | HLA-A2-restricted peptide | Jager et al. Proc. Natl. Acad. Scie. |
| | | p157-165 (SLLMWITQC), | U.S.A. 103(39): 14453-8 (2006). |
| | | HLA- | Gnjatic et al. PNAS |
| | | Cw3-restricted p92-100 | Sep. 26, 2000 vol. 97 no. 20 p. |
| | | (LAMP-FATPM) | 10919 |
| | | and HLA-Cw6- | Jager et al. J Exp Med. 187(2): 265- |
| | | restricted p80-88 | 70 (1998). |
| | | (ARGPESRLL) | Chen et al. J Immunol. 165(2): 948- |
| | | SLLMWITQC | 55 (2000). |
| | | MLMAQEALAFL | Valmori et al. Cancer Res. |
| | | YLAMPFATPME | 60(16): 4499-506 (2000). |
| | | ASGPGGGAPR | Aarnoudse et al. Int J Cancer. |
| | | LAAQERRVPR | 82(3): 442-8 (1999). |
| | | TVSGNILTIR | Eikawa et al. Int J Cancer. |
| | | APRGPHGGAASGL | 132(2): 345-54 (2013). |
| | | MPFATPMEAEL | Wang et al. J Immunol. 161(7): 3598- |
| | | KEFTVSGNLLTI | 606 (1998). |
| | | MPFATPMEA | Matsuzaki et al. Cancer Immunol |
| | | FATPMEAEL | Immunother. 57(8)1185-95 (2008). |
| | | FATPMEAELAR | Ebert et al. Cancer Res. 69(3): 1046- |
| | | LAMPFATPM | 54 (2009). |
| | | ARGPESRLL | Eikawa et al. Int J Cancer. |
| | | SLLMWITQCFLPVF | 132(2): 345-54 (2013). |
| | | LLEFYLAMPFATPMEAEL | Knights et al. Cancer Immunol |
| | | -ARRSLAQ | Imununother. 58(3): 325-38 (2009). |
| | | EFYLAMPFATPM | Jäger et al. Cancer Immun. 2: 12 |
| | | PGVLLKEFTVSGNILTIRL | (2002). |
| | | -TAADHR | Zeng et al. Proc Natl Acad Sci USA. |
| | | RLLEFYLAMPFA | 98(7): 3964-9 (2001). |
| | | QGAMLAAQERRVPRAAE | Mandic et al. J Immunol. |
| | | -VPR | 174(3): 1751-9 (2005). |
| | | PFATPMEAELARR | Chen et al. Proc Natl Acad Sci USA. |
| | | PGVLLKEFTVSGNILTIRLT | 101(25): 9363-8 (2004). |
| | | VLLKEFTVSG | Ayyoub et al. Clin Cancer Res. |
| | | AADHRQLQLSISSCLQQL | 16(18): 4607-15 (2010). |
| | | LKEFTVSGNILTIRL | Slager et al. J Immunol. |
| | | PGVLLKEFTVSGNILTIRL | 172(8): 5095-102 (2004). |
| | | -TAADHR | Mizote et al. Vaccine. 28(32): 5338- |
| | | LLEFYLAMPFATPMEAEL | 46 (2010). |
| | | -ARRSLAQ | Jager et al. J Exp Med. 191(4): 625- |
| | | KEFTVSGNILT | 30 (2000). |
| | | LLEFYLAMPFATPM | Zarour et al. Cancer Res. |
| | | AGATGGRGPRGAGA | 60(17): 4946-52 (2000). |
| | | | Zeng et al. J Immunol. 165(2): 1153- |
| | | | 9 (2000). |
| | | | Bioley et al. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 3 | HERV-K-MEL | MLAVISCAV | Schiavetti et al. Cancer Res. |
| | | | 62(19): 5510-6 (2002). |
| 4 | KK-LC-1 | RQKRILVNL | Fukuyama et al. Cancer Res. |
| | | | 66(9): 4922-8 (2006). |
| 5 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 6 | Sp17 | ILDSSEEDK | Chiriva-Internati et al. Int J Cancer. |
| | | | 107(5): 863-5 (2003). |

TABLE 30
Uterine cancer

| | Tumor-associated | Reported immunogenic | |
|---|---|---|---|
| No. | antigen | epitopes | Sources |
| 1 | LAGE-1 | MLMAQEALAFL | Aarnoudse et al. Int J Cancer. |
| | | SLLMWITQC | 82(3): 442-8 (1999). |
| | | LAAQERRVPR | Rimoldi et al. J Immunol. |
| | | ELVRRILSR | 165(12): 7253-61 (2000). |
| | | APRGVRMAV | Wang et al. J Immunol. 161(7): 3598- |
| | | SLLMWITQCFLPVF | 606 (1998). |
| | | QGAMLAAQERRVPRAAE | Sun et al. Cancer Immunol |
| | | VP-R | Immunother. 55(6): 644-52 (2006). |
| | | AADHRQLQLSISSCLQQL | Slager et al. Cancer Gene Ther. |
| | | CLSRRPWKRSWSAGSCP | 11(3): 227-36 (2004). |
| | | G-MPHL | Zeng et al. Proc Natl Acad Sci USA. |
| | | ILSRDAAPLPRPG | 98(7): 3964-9 (2001). |
| | | AGATGGRGPRGAGA | Slager et al. J Immunol. |
| | | | 172(8): 5095-102 (2004). |
| | | | Jager et al. J Exp Med. 191(4): 625- |
| | | | 30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 2 | NY-ESO-1 | HLA-A2-restricted peptide | Jager et at. Proc. Natl. Acad. Scie. |
| | | p157-165 (SLLMWITQC), | U.S.A. 103(39): 14453-8 (2006). |
| | | HLA-Cw3-restricted p92- | Gnjatic et al. PNAS |
| | | 100 (LAMP-FATPM) and | Sep. 26, 2000 vol. 97 no. 20 p. |
| | | HLA-Cw6-restricted p80-88 | 10919 |
| | | (ARGPESRLL) | Jager et al. J Exp Med. 187(2): 265- |
| | | SLLMWITQC | 70 (1998). |
| | | MLMAQEALAFL | Chen et al. J Immunol. 165(2): 948- |
| | | YLAMPFATPME | 55 (2000). |
| | | ASGPGGGAPR | Valmori et al. Cancer Res. |
| | | LAAQERRVPR | 60(16): 4499-506 (2000). |
| | | TVSGNILTIR | Aarnoudse et al. Int J Cancer. |
| | | APRGPHGGAASGL | 82(3): 442-8 (1999). |
| | | MPFATPMEAEL | Eikawa et al. Int J Cancer. |
| | | KEFTVSGNILTI | 132(2): 345-54 (2013). |
| | | MPFATPMEA | Wang et al. J Immunol. 161(7): 3598- |
| | | FATPMEAEL | 606 (1998). |
| | | FATPMEAELAR | Matsuzaki et al. Cancer Immunol |
| | | LAMPFATPM | Immunother. 57(8)1185-95 (2008). |
| | | ARGPESRLL | Ebert et al. Cancer Res. 69(3): 1046- |
| | | SLLMWITQCFLPVF | 54 (2009). |
| | | LLEFYLAMPFATPMEAEL- | Eikawa et al. Int J Cancer. |
| | | ARRSLAQ | 132(2): 345-54 (2013). |
| | | EFYLAMPFATPM | Knights et al. Cancer Immunol |
| | | PGVLLKEFTVSGNILTIRL- | Immunother. 58(3): 325-38 (2009). |
| | | TAADHR | Jäger et al. Cancer Immun. 2: 12 |
| | | RLLEFYLAMPFA | (2002). |
| | | QGAMLAAQERRVPRAAE- | Zeng et al. Proc Natl Acad Sci USA. |
| | | VPR | 98(7): 3964-9 (2001). |
| | | PFATPMEAELARR | Mandic et al. J Immunol. |
| | | PGVLLKEFTVSGNILTIRLT | 174(3): 1751-9 (2005). |
| | | VLLKEFTVSG | Chen et al. Proc Natl Acad Sci USA. |
| | | AADHRQLQLSISSCLQQL | 101(25): 9363-8 (2004). |
| | | LKEFTVSGNILTIRL | Ayyoub et al. Clin Cancer Res. |
| | | PGVLLKEFTVSGNILTIRL- | 16(18): 4607-15 (2010). |
| | | TAADHR | Slager et al. J Immunol. |
| | | LLEFYLAMPFATPMEAEL- | 172(8): 5095-102 (2004). |
| | | ARRSLAQ | Mizote et al. Vaccine. 28(32): 5338- |
| | | KEFTVSGNILT | 46 (2010). |
| | | LLEFYLAMPFATPM | Jager et al. J Exp Med. 191(4): 625- |
| | | AGATGGRGPRGAGA | 30 (2000). |
| | | | Zarour et al. Cancer Res. |
| | | | 60(17): 4946-52 (2000). |
| | | | Zeng et al. J Immunol. 165(2): 1153- |
| | | | 9 (2000). |
| | | | Bioley et at. Clin Cancer Res. |
| | | | 15(13): 4467-74 (2009). |
| | | | Zarour et al. Cancer Res. 62(1): 213- |
| | | | 8 (2002). |
| | | | Hasegawa et al. Clin Cancer Res. |
| | | | 12(6): 1921-7 (2006). |
| 3 | HERV-K-MEL | MLAVISCAV | Schiavetti et al. Cancer Res. |
| | | | 62(19): 5510-6 (2002). |
| 4 | KK-LC-1 | RQKRILVNL | Fukuyama et al. Cancer Res. |
| | | | 66(9): 4922-8 (2006). |
| 5 | KM-HN-1 | NYNNFYRFL | Fukuyama et al. Cancer Res. |
| | | EYSKECLKEF | 66(9): 4922-8 (2006). |
| | | EYLSLSDKI | Monji et al. Clin Cancer Res. 10(18 |
| | | | Pt 1): 6047-57 (2004). |
| 6 | Sp17 | ILDSSEEDK (SEQ ID NO: | Chiriva-Internati et al. Int J Cancer. |
| | | 299) | 107(5): 863-5 (2003). |

### Gene Alignment

An exemplary alignment of select orthopoxvirus genes is shown below. Various genes of 5 vaccinia virus strains, Copenhagen (“cop”), Western Reserver (“WR”), Tian Tan (“Tian”), Wyeth, and Lister, align as follows:

C2L
CLUSTAL O(1.2.4) multiple sequence alignment

| | | |
|---|---|---|
| cop | MESVIFSINGEIIQVNKEIITASPYNFFKRIQDHHLKDEAIILNGINYHAFESLLDYIRW | 60 |
| WR | MESVIFSINGEIIQVNKEIITASPYNFFKRIQDHHLKDEAIILNGINYHAFESILDYIRW | 60 |
| Tian | MESVIFSINGEIIQVNKEIITASPYNFFKRIQDHHLKDEAIILNGINYHAFESLLDYIRW | 60 |
| Wyeth | MESVTFSINGEIIQVNKEIITASPYNFFKRIQEHHINDEVIILNGINYHAFESLLDYMRW | 60 |
| Lister | MESVIFSINGEIIQVNKEIITASPYNFFKRIQDHHLKDEAIILNGINYHAFESLLDYMRW | 60 |
| | **** ***************************:**::**.*****************:** | |
| cop | KKINITINNVEMILVAAIIIDVPPVVDLCVKTMIHNINSTNCIRMFNFSKRYGIKKLYNA | 120 |
| WR | KKINITINNVEMILVAAIIIDVPPVVDLCVKTMIHNINSTNCIRMFNFSKRYGIKKLYNA | 120 |
| Tian | KKINITINNVEMILVAAIIIDVPPVVDLCVKTMIHNINSTNCIRMFNFSKQYGIKKLYNA | 120 |
| Wyeth | KKINITINNVEMILVAAVIIDVTPVVDLCVKTMIHNINSTNCIRMFNESKRYGIKKLYNA | 120 |
| Lister | KKINITINNVEMILVAAIIIDVPPVVDLCVKTMIHNINFTNCIRMFNFSKRYGIKKLYNA | 120 |
| | *****************:**** *************** ***********:********* | |
| cop | SMSEIINNITAVTSDPEFGKLSKDELTTILSHENVNVNHEDVTAMILLKWIHKNPNDVDI | 180 |
| WR | SMSEIINNITAVTSDPEFGKLSKDELTTILSHENVNVNHEDVTAMILLKWIHKNPNDVDI | 180 |
| Tian | SMSEIINNITAVTSDPEFGKLSKDELTTILSHEDVNVNHEDVTAMILLKWIHKNPNDVDI | 180 |
| Wyeth | SMSEIINNITAVTSDPEFGKLSKDELTTILSHEDVNVNHEDVTAMILLKWIHKNPNDVDI | 180 |
| Lister | SMSEIINNITAVTSDPEFGKLSKDELTTILSHEDVNVNHEDVTAMILLKWIHKNPNDVDI | 180 |
| | *********************************:************************** | |
| cop | INILHPKFMTNTMRNAISLLGLTISKSTKPVTRNGIKHNIVVIKNSDYISTITHYSPRTE | 240 |
| WR | INILHPKFMTNTMRNAISLLGLTISKSTKPVTRNGIKHNIVVIKNSDYISTITHYSPRTE | 240 |
| Tian | INILHPKFMTNTMRNAISLLGLTISKSTKPVTRNGIKHNIVVIKNSDYISTITHYSPRTE | 240 |
| Wyeth | INILHPKFMTNTMRNAISLLGLTISKSTKPVTRNGIKHNIVVIKNSDYISTITHYSPRTE | 240 |
| Lister | INILHPKFMTNTMRNAISLLGLTISKSTKPVTRNGIKHNIVVIKNSDYISTITHYSPRTE | 240 |
| | ************************************************************ | |
| cop | YWTIVGNTDRQFYNANVLHNCLYIIGGMINNRHVYSVSRVDLETKKWKTVTNMSSLKSEV | 300 |
| WR | YWTIVGNTDRQFYNANVLHNCLYIIGGMINNRHVYSVSRVDLETKKWKTVTNMSSLKSEV | 300 |
| Tian | YWTIVGNTDRQFYNANVLHNCLYIIGGMINNRHVYSVSRVDLETKKWKTVTNMSSLKSEV | 300 |
| Wyeth | YWTIVGNTDRQFYNANVLHNCLYIIGGMINNRHVYSVSRVDLETKKWKTVTNMSSLKSEV | 300 |
| Lister | YWTIVGNTDRQFYNANVLHNCLYIIGGMINNRHVYSVSRVDLKTKKWKTVTNMSSLKSEV | 300 |
| | ******************************************:***************** | |
| cop | STCVNDGKLYVIGGLEFSISTGVAEYLKHGTSKWIRLPNLITPRYSGASVFVNDDIYVMG | 360 |
| WR | STCVNDGKLYVIGGLEFSISTGVAEYLKHGTSKWIRLPNLITPRYSGASVFVNDDIYVMG | 360 |
| Tian | STCVNNGKLYVIGGLEFSISTGVAEYLKHGTSKWIRLPNLITPRYSGASVFVNDDIYVMG | 360 |
| Wyeth | STCVNNGKLYVIGGLEFSISTGVAEYLKHGTSKWIRLPNLITPRYSGASVFVNDDIYVMG | 360 |
| Lister | STCVNDGKLYVIGGLEFSISTGVAEYLKHGTSKWIRLPNLITPRYSGASVFVNDDIYVMG | 360 |
| | *****:****************************************************** | |
| cop | GVYTTYEKYVVLNDVECFTKNRWIKKSPMPRHHSIVYAVEYDGDIYVITGITHETRNYLY | 420 |
| WR | GVYTTYEKYVVLNDVECFTKNRWIKKSPMPRHHSIVYAVEYDGDIYVITGITHETRNYLY | 420 |
| Tian | GVYTTYEKYVVLNDVECFTKNRWIKKSPMPRHHSIVYAVEYDGDIYVITGITHETRNYLY | 420 |
| Wyeth | GVYTTYEKYVVLNDVECFTKNRWIKKSPMPRHHSIVYAVEYDGDIYAITGITHETRNYLY | 420 |
| Lister | GVYTTYEKYVVLNDVECFTKNRWIKKSPMPRHHSIVYAVEYDGDIYVITGITHETRNYLY | 420 |
| | **********************************************.************* | |
| cop | KYIVKEDKWIELYMYFNHVGKMFVCSCGDYILIIADAKYEYYPKSNTWNLFDMSTRNIEY | 480 |
| WR | KYIVKEDKWIELYMYFNHVGKMFVCSCGDYILIIADAKYEYYPKSNTWNLFDMSTRNIEY | 480 |
| Tian | KYIVKEDKWIELYMYFNHVGKMFVCSCGDYILIIADAKYEYYPKSNTWNLFDMSTRNIEY | 480 |
| Wyeth | KYIVKEDKWIELYMYFNHVGKMFVCSCGDYILIIADAKYEYYPKSNTWNLFDMSTRNIEY | 480 |
| Lister | KYIVKEDKWIELYMYFNHVGKMFVCSCGDYILIIADAKYEYYPKSNTWNLFDMSTRNIEY | 480 |
| | ************************************************************ | |
| cop | YDMFTKDETPKCNVTHKSLPSFLSNCEKQFLQ | 512 |
| WR | YDMFTKDETPKCNVTHKSLPSFLSNCEKQFLQ | 512 |
| Tian | YDMFTKDETPKCNVTHKSLPSFLSNCEKQFLQ | 512 |
| Wyeth | YDMFTKDET------HKSLPSFLSNCEKQFLQ | 506 |
| Lister | YDMFTKDETPKCNVTHKSLPSFLSNCEKQFLQ | 512 |
| | ********* ***************** | |

C1L
CLUSTAL O(1.2.4) multiple sequence alignment

| | | |
|---|---|---|
| Cop | MVKNNKI-----SNSCRMIMSTNPNNILMRHLKNLTDDEFKCIIHRSSDFLYLSDSDYTS | 55 |
| WR | MVKNNKIQKNKISNSCRMIMSTDPNNILMRHLKNLTDDEFKCIIHRSSDFLYLSDSDYTS | 60 |
| Tian | MVKNNKI-----SNSCRMIMSTDPNNILMRHLKNLTDDEFKCIIHRSSDFLYLSDSDYTS | 55 |
| Wyeth | MVKNNKI-----SNSCRMIMSTNPNNILMRHLKNLTDDEFKCIIHRSSDFLYLSDRDYTS | 55 |
| Lister | MVKNNKI-----SNSCRMIMSTNPNNILMRHLKNLTDDEFKCIIHRSSDFLYLSDSDYTS | 55 |
| | ******* ************************************************ | |
| Cop | ITKETLVSEIVEEYPDDCNKILAIIFLVLDKDIDVDIETKLKPKPAVRFAILDKMTEDIK | 115 |
| WR | ITKETLVSEIVEEYPDDCNKILAIIFLVLDKDIDVDIKTKLKPKPAVRFAILDKMTEDIK | 120 |
| Tian | ITKETLVSEIVEEYPDDCNKILAIIFLVLDKDIDVDIKTKLKPKPAVRFAILDKMTEDIK | 115 |
| Wyeth | ITKETLVSEIVEEYPDDCNKILAIIFLVLDKDIDVDIKTKLKPKPAVRFAILDKMTEDIK | 115 |
| Lister | ITKETLVSEIVEEYPDDCNKILAIIFLVLDKDIDVDIETKLKPKPAVRFAILDKMTADIK | 115 |
| | *************************************:****************** *** | |
| Cop | LTDLVRHYFRYIEQDIPLGPLFKKIDSYRTRAINKYSKELGLATEYFNKYGHLMFYTLPI | 175 |
| WR | LTDLVRHYFRYIEQDIPLGPLFKKIDSYRTRAINKYSKELGLATEYFNKYGHLMFYTLPI | 180 |
| Tian | LTDLVRHYFRYIEQDIPLGPLFKKIDSYRTRAINKYSKELGLATEYFNKYGHLMFYTLPI | 175 |
| Wyeth | LTDLVRHYFRYIEQDIPLGPLFKKIDSYRTRAINKYSKELGLATEYFNKYGHLMFYTLPI | 175 |
| Lister | LTDLVRHYFRYIEQDIPLGPLFKKIDSYRTRAINRYSKELGLATEYFNKYGHLMFYTLPI | 175 |
| | ************************************************************ | |
| Cop | PYNRFFCRNSIGFLAVLSPTIGHVKAFYKFIEYVSIDDRRKFKKELMSK | 224 |
| WR | PYNRFFCRNSIGFLAVLSPTIGHVKAFYKFIEYVSIDDRRKFKKELMSK | 229 |
| Tian | PYNRFFCRNSIGFLAVLSPTIGHVKAFYKFIEYVSIDDRRKFKKELMSK | 224 |
| Wyeth | PYNRFFCRNSIGFLAVLSPTIGHVKAFYKFIEYVSIDDRRKFKKELMSK | 224 |
| Lister | PYNRFFCRNSIGFLAVLSPTIGHVKAFYRFIEYVSIDDRRKFKKELMSK | 224 |
| | ************************************************* | |

N1L
CLUSTAL O(1.2.4) multiple sequence alignment

| | | |
|---|---|---|
| Cop | MRTLLIRYILWRNDNDQTYYNDDFKKLMLLDELVDDGDVCTLIKNMRMTLSDGPLLDRLN | 60 |
| WR | MRTLLIRYILWRNDNDQTYYNDNFKKLMLLDELVDDGDVCTLIKNMRMTLSDGPLLDRLN | 60 |
| Tian | MRTLLIRYILWRNDNDQTYYNDDFKKLMLLDELVDDGDVCTLIKNMRMTLSDGPLLDRLN | 60 |
| Wyeth | MRTLLIRYILWRNDNDQTYYNDDFKKLMLLDELVDDGDVCTLIKNMRMTLSDGPLLDRLN | 60 |
| Lister | MRTLLIRYILWRNDNDQTYYNDDFKKLMLLDELVDDGDVCTLIKNMRMTLSDGPLLDRLN | 60 |
| | **********************:************************************* | |
| Cop | QPVNNIEDAKRMIAISAKVARDIGERSEIRWEESFTILFRMIETYFDDLMIDLYGEK | 117 |
| WR | QPVNNIEDAKRMIAISARVARDIGERSEIRWEESFTILFRMIETYFDDLMIDLYGEK | 117 |
| Tian | QPVNNIEDAKRMIAISAKVARDIGERSEIRWEESFTILFRMIETYFDDLMIDLYGEK | 117 |
| Wyeth | QPVNNIEDAKRMIAISARVARDIGERSEIRWEESFTILFRMIETYFDDLMIDLYGEK | 117 |
| Lister | QPVNNIEDAKRMIAISAKVARDIGERSEIRWEESFTILFRMIETYFDDLMIDLYGEK | 117 |
| | ********************************************************* | |

N2L
CLUSTAL O(1.2.4) multiple sequence alignment

| | | |
|---|---|---|
| Cop | MTSSAMDNNEPKVLEMVYDATILPEGSSMDPNIMDCINRHINMCIQRTYSSSIIAILNRF | 60 |
| WR | MTSSANDNNEPKVLEMVYDATILPEGSSMDPNIMDCINRHINMCIQRTYSSSIIAILDRF | 60 |
| Tian | MTSSAMDNNEPKVLEMVYDATILPEGSSMDPYIMDCINRHINMCIQRTYSSSIIAILDRF | 60 |
| Wyeth | MTSSAMDNNEPKVLEMVYDATILPEGSSMDPNIIDCINRHINMCIQRTYSSSIIAILDRF | 60 |
| Lister | MTSSAMDNNEPKVLEMVYDATILPEGSSMDPNIMDCINRHINMCIQRTYSSSIIAILDRF | 60 |
| | ******************************* *:***********************:** | |
| Cop | LTMNKDELNNTQCHIIKEFMTYEQMAIDHYGEYVNAILYQIRKRPNQHHTIDLFKKIKRT | 120 |
| WR | LMMNKDELNNTQCHIIKEFMTYEQMAIDHYGGYVNAILYQIRKRPNQHHTIDLFKRIKRT | 120 |
| Tian | LMMNKDELNNTQCHIIKNL----------------------------------------- | 79 |
| Wyeth | LTMNKDELNNTQCHIIKEFMTYEQMAIDHYGGYVNAILYQIRKRPNQHHTIDLFKKIKRT | 120 |
| Lister | LTMNRDELNNTQCHIIKEFMTYEQMAIDHYGEYVNAILYQIRKRPNQHHTIDLFKKIKRT | 120 |
| | * ***************:: | |
| Cop | PYDTFKVDPVEFVKKVIGFVSILNKYKPVYSYVLYENVLYDEFKCFINYVETKYF | 175 |
| WR | RYDTFKVDPVEFVKKVIGFVSILNKYKPVYSYVLYENVLYDEFKCFINYVETKYF | 175 |
| Tian | ------------------------------------------------------- | |
| Wyeth | RYDTFKVDPVEFVKKVIGFVSILNKYKPVYSYVLYENVLYDEFKCFIDYVETKYF | 175 |
| Lister | RYDTFKVDPVEFVKKVIGFVSILNKYKPVYSYVLYENVLYDEFKCFIDYVETKYF | 175 |

M1L
CLUSTAL O(1.2.4) multiple sequence alignment

| | | |
|---|---|---|
| Cop | MIFVIESKLLQIYRNRNRNINFYTTMDNIMSAEYYLSLYAKYNSKNLDVFRNMLQAIEPS | 60 |
| WR | MIFVIESKLLQIYRNRNRNINFYTTMDNIMSAEYYLSLYAKYNSKNLDVFRNMLQAIEPS | 60 |
| Tian | MIFVIESKLLQIYRNRNRNINFYTTMDNIMSAEYYLSLYAKYNSKNLDVFRNMLQAIEPS | 60 |
| Wyeth | MIFVIESKLLQIYRNRNRNINFYTTMDNIMSAEYYLSLYAKYNSKNLDVFRNMLQAIEPS | 60 |
| Lister | MIFVIESKLLQIYRN--RNINFYTTMDNIMSAEYYLSLYAKYNSKNLDVFRNMLQAIEPS | 58 |
| | *************** ******************************************* | |
| Cop | GNNYHILHAYCGIKGLDERFVEELLHRGYSPNETDDDGNYPLHIASKINNNRIVAMLLTH | 120 |
| WR | GNNYHILHAYCGIKGLDERFVEELLHRGYSPNETDDDGNYPLHIASKINNNRIVAMLLTH | 120 |
| Tian | GNNYHILHAYCGIKGLDERFVEELLHRGYSPNETDDDGNYPLHIASKINNNRIVAMLLTH | 120 |
| Wyeth | GNNYHILHAYCGIKGLDERFVEELLHRGYSPNETDDDGNYPLHIASKINNNRIVAMLLTH | 120 |
| Lister | GNNYHILHAYCGIKGLDERFVEELLHRGYSPNETDDDGNYPLHIASKINNNRIVAMLLTH | 118 |
| | ************************************************************ | |
| Cop | GADPNACDKHNKTPLYYLSGTDDEVIERINLLVQYGAKINNSVDEEGCGPLLACTDPSER | 180 |
| WR | GADPNACDKHNKTPLYYLSGTDDEVIERINLLVQYGAKINNSVDEEGCGPLLACTDPSER | 180 |
| Tian | GADPNACDKHNKTPLYYLSGTDDEVIERINLLVQYGAKINNSVDEEGCGPLLACTDPSER | 180 |
| Wyeth | GADPNACDKHNKTPLYYLSGTDDEVIERINLLVQYGAKINN------------------- | 161 |
| Lister | GADPNACDKQHKTPLYYLSGTDDEVIERINLLVQYGAKINNSVDEEGCGPLLACTDPSER | 178 |
| | *********::****************************** | |
| Cop | VFKKIMSIGFEARIVDKFGKNHIHRHLMSDNPKASTISWMMKLGISPSKPDHDGNTPLHI | 240 |
| WR | VFKKIMSIGFEARIVDKFGKNHIHRHLMSDNPKASTISWMMKLGISPSKPDHDGNTPLHI | 240 |
| Tian | VFKKIMSIGFEARIVDKFGKNHIHRHLMSDNPKASTISWMMKLGISPSKPDHDGNTPLHI | 240 |
| Wyeth | ------------------------------------------------------------ | |
| Lister | VFKKIMSIGFEARIVDKFGKNHIHRHLMSDNPKASTISWMMKLGISPSKPDHDGNTPLHI | 238 |
| Cop | VCSKTVKNVDIIDLLLPSTDVNKQNKFGDSPLTLLIKTLSPAHLINKLLSTSNVITDQTV | 300 |
| WR | VCSKTVKNVDIIDLLLPSTDVNKQNKFGDSPLTLLIKTLSPAHLINKLLSTSNVITDQTV | 300 |
| Tian | VCSKTVKNVDIIDLLLPSTDVNKQNKFGDSPLTLLIKTLSPAHLINKLLSTSNVITDQTV | 300 |
| Wyeth | ------------------------------------------------------------ | |
| Lister | VCSKTVKNVDIIDLLLPSTDVNKQNKFGDSPLTLLIKTLSPAHLINKLLSTSNVITDQTV | 298 |
| Cop | NICIFYDRDDVLEIINDKGKQYDSTDFKMAVEVGSIRCVKYLLDNDIICEDAMYYAVLSE | 360 |
| WR | NICIFYDRDDVLEIINDKGKQYDSTDFKMAVEVGSIRCVKYLLDNDIICEDAMYYAVLSE | 360 |
| Tian | NICIFYDRDDVLEIINDKGKQYDSTDFKMAVEVGSIRCVKYLLDNDIICEDAMYYAVLSE | 360 |
| Wyeth | ------------------------------------------------------------ | |
| Lister | NICIFYDRDDVLEIINDKGKQYDSTDFKMAVEVGSIRCVKYLLDNDIICEDAMYYAVLSE | 358 |
| Cop | YETMVDYLLFNHFSVDSVVNGHTCMSECVRLNNPVILSKLMLHNPTSETMYLTMKAIEKD | 420 |
| WR | YETMVDYLLFNHFSVDFVVNGHTCMSECVRLNNPVILSKLMLHNPTSETMYLTMKAIEKD | 420 |
| Tian | YETMVDYLLFNHFSVDFVVNGHTCMSECVRLNNPVILSKLMLHNLTSETMYLTMKAIEKD | 420 |
| Wyeth | ------------------------------------------------------------ | |
| Lister | YETMVDYLLENHFSVDSVVNGHTCMSECVRLNNPVILSKLMLHNPTSETMYLTMKAIEKD | 418 |
| Cop | KLDKSIIIPFIAYFVLMHPDFCKNRRYFTSYKRFVTDYVHEGVSYEVFDDYF | 472 |
| WR | RLDKSIIIPFIAYFVLMHPDFCKNRRYFTSYKRFVTDYVHEGVSYEVFDDYF | 472 |
| Tian | RLDKSIIIPFIAYFVLMHPDFCKNRRYFTSYKRFVTDYVHEGVSYEVFDDYF | 472 |
| Wyeth | ---------------------------------------------------- | |
| Lister | RLDKSIIIPFIAYFVLMHPDFCKNRRYFTSYKRFVTDYVHEGVSYEVFDDYF | 470 |

M2L
CLUSTAL O(1.2.4) multiple sequence alignment

| | | |
|---|---|---|
| Cop | MVYKLVLLFCIASLGYSVEYKNTICPPRQDYRYWYFAAELTIGVNYDINSTIIGECHMSE | 60 |
| WR | MVYKLVLLFCIASLGYSVEYKNTICPPRQDYRYWYFAAELTIGVNYDINSTIIGECHMSE | 60 |
| Tian | ------------------------MSSSTRLPVLVLAAELTIGVNYDINSTIIGECHMSE | 36 |
| Wyeth | MVYKLVLLFCIASLGYSVEYKNTICPPRQDYRYWYFAAELTIGVNYDINSTIIGECHMSE | 60 |
| Lister | MVYKLVLLFCIASLGYSVEYKNTICPPRQDYRYWYFAAELTIGVNYDINSTIIGECHMSE | 60 |
| | :************************ | |
| Cop | SYIDRNANIVLIGYGLEINMTIMDTDQREVAAAEGVGKDNKLSVLLFTTQRLDKVHHNIS | 120 |
| WR | SYIDRNANIVLTGYGLEINNTIMDTDQRFVAAAEGVGKDNKLSVLLFTTQRLDKVHHNIS | 120 |
| Tian | SYIDRNANIVLTGYGLEINMTIMDTDQRFVAAAEGVGKDNKLSVLLFTTQRLDKVHHNIS | 96 |
| Wyeth | SYIDRNANIVLTGYGLEINMTIMDTDQRFVAAAEGVGKDNKLSVLLFTTQRLDKVHHNIS | 120 |
| Lister | SYIDRNANIVLTGYGLEINMTIMDTDQRFVAAAEGVGKDNKLSVLLFTTQRLDKVHHNIS | 120 |
| | ************************************************************ | |
| Cop | VTITCMEMNCGTTKYDSDLPESIHKSSSCDITINGSCVTCVNLETDPTKINPHYLHPKDK | 180 |
| WR | VTITCMEMNCGTTKYDSDLPESIHKSSSCDITINGSCVTCVNLETDPTKINPHYLHPKDK | 180 |
| Tian | VTITCMEMNCGTTKYDSDLPESIHKSSSCDITINGSCVTCVNLETDPTKINPHYLHPKDK | 156 |
| Wyeth | VTITCMEMNCGTTKYDSDLPESIHKSSSCDITINGSCVTCVNLETDPTKINPHYLHPKDK | 180 |
| Lister | VTITCMEMNCGTTKYDSDLPESIHKSSSCDITINGSCVTCVNLETDPTKINPHYLHPKDK | 180 |
| | ************************************************************ | |
| Cop | YLYHNSEYGMRGSYGVTFIDELNQCLLDIKELSYDICYRE | 220 |
| WR | YLYHNSEYSMRGSYGVTFIDELNQCLLDIKELSYDICYRE | 220 |
| Tian | YLYHNSEYGMRGSYGVTFIDELNQCLLDIKELSYDICYRE | 196 |
| Wyeth | YLYHNSEYGMRGSYGVTFIDELNQCLLDIKELSYDICYRE | 220 |
| Lister | YLYHNSEYGMRGSYGVTFIDELNQCLLDIKELSYDICYRE | 220 |
| | ********.******************************* | |

K1L
CLUSTAL O(1.2.4) multiple sequence alignment

| | | |
|---|---|---|
| Cop | MDLSRINTWKSKQLKSFLSSKDTFKADVHGHSALYYAIADNNVRLVCTLLNAGALKNLLE | 60 |
| WR | MDLSRINTWKSKQLKSFLSSKDAFKADVHGHSALYYAIADNNVRLVCTLLNAGALKNLLE | 60 |
| Tian | MDLSRINTWKSKQLKSFLSSKDTFKADVHGHSALYYAIADNNVRLVCTLLNSGALKNLLE | 60 |
| Wyeth | MDLSRINTWKSKQLKSFLSSKDTFKADVHGHSALYYAIADNNVRLVCTLLNAGALKNLLE | 60 |
| Lister | MDLSRINTWKSKQLKSFLSSKDAFKADINGHSALYYAIADNNVRLVCTLLNAGALKNLLE | 60 |
| | **********************:****::**********************:******** | |
| Cop | NEFPLHQAATLEDTKIVKILLFSGMDDSQFDDKGNTALYYAVDSGNMQTVKLFVKKNWRL | 120 |
| WR | NEFPLHQAATLEDTKIVKILLFSGLDDSQFDDKGNTALYYAVDSGNMQTVKLFVKKNWRL | 120 |
| Tian | NEFPLHQAATLEDTKIVKILLFSGLDDSQFDDKGNTALYYAVDSGNMQTVKLFVKKNWRL | 120 |
| Wyeth | NEFPLHQAATLEDTKIVKILLFSGLDDSQFDDKGNTALYYAVDSGNMQTVKLFVKKNWRL | 120 |
| Lister | NEFPLHQAATLEDTKIVKILLFSGLDDSQFDDKGNTALYYAVDSGNMQTVKLFVKKNWRL | 120 |
| | ************************:*********************************** | |
| Cop | MFYGKTGWKTSFYHAVMLNDVSIVSYFLSEIPSTFDLAILLSCIHTTIKNGHVDMMILLL | 180 |
| WR | MFYGKTGWETSFYHAVMLNDVSIVSYFLSEIPSTFDLAILLSCIHITIKNGHVDMMILLL | 180 |
| Tian | MFYGKTGWKTSFYHAVMLNDVSIVSYFLSEIPSTFDLAILLSCIHITIKNGHVDMMILLL | 180 |
| Wyeth | MFYGKTGWKTSFYHAVMLNDVSIVSYFLSEIPSTFDLAILLSCIHITIKNGHVDMMILLL | 180 |
| Lister | MFYGKTGWKTSFYHAVMLNDVSIVSYFLSEIPSTFDLAILLSCIHITIKNGHVDMMILLL | 180 |
| | ********************************************* ************** | |
| Cop | DYMTSTNTNNSLLFIPDIKLAIDNKDIEMLQALFKYDINIYSVNLENVLLDDAEITKMII | 240 |
| WR | DYMTSTNTNNSLLFIPDIKLAIDNKDIEMLQALFKYDINIYSANLENVLLDDAEIAKMII | 240 |
| Tian | DYMTVDKHQ--------------------------------------------------- | 189 |
| Wyeth | DYMTSTNTNNSLLFIPDIKLAIDNKDIEMLQALFKYDINIYSANLENVLLDDAEIAKMII | 240 |
| Lister | DYMTSTNTNNSLLFIPDIKLAIDNKDIEMIQALFKYDINIYSANLENVLLDDAEIAKMII | 240 |
| | **** : : | |
| Cop | EKHVEYKSDSYTKDLDIVKNNKLDEIISKNKELRLMYVNCVKKN | 284 |
| WR | EKHVEYKSDSYTKDLDIVKNNKLDEIISKNKELRLMYVNCVKKN | 284 |
| Tian | -------------------------------------------- | |
| Wyeth | EKHVEYKSDSYTKDLDIVKNNKLDEIISKNKELRLMYVNCVKKN | 284 |
| Lister | EKHVEYKSDSYTKDLDIVKNNKLDEIISKNKELKLMYVNCVKKN | 284 |

K2L
CLUSTAL O(1.2.4) multiple sequence alignment

| | | |
|---|---|---|
| Cop | MIALLILSLTCSVSTYRLQGFTNAGIVAYKNIQDDNIVFSPFGYSFSMFMSLLPASGNTR | 60 |
| WR | MIALLILSLTCSVSTYRLQGFTNAGIVAYKNIQDDNIVFSPFGYSFSMFMSLLPASGNTR | 60 |
| Tian | MIALLILSLACSASAYRLQGFTNAGIVAYKNIQDDNIVFSPFGYSFSMFMSLLPASGNTR | 60 |
| Wyeth | MIALLILSLTCSVSTYRLQGFTNAGIVAYKNIQDDNIVFSPFGYSFSMFMSLLPASGNTR | 60 |
| Lister | ------------------------------------------------------------ | |
| Cop | IELLKTMDLRKRDLGPAFTELISGLAKLKTSKYTYTDLTYQSFVDNTVCIKPLYYQQYHR | 120 |
| WR | IELLKTMDLRKRDLGPAFTELISGLAKLKTSKYTYTDLTYQSFVDNTVCIKPSYYQQYHR | 120 |
| Tian | IELLKTMDLRKRDLGPAFTELISGLAKLKTSKYTYTDLTYQSFVDNTVCIKPSYYQQYHR | 120 |
| Wyeth | IELLKTMDLRKRDLGPAFTELISGLAKLKTSKYTYTDLTYQSFVDNTVCIKPSYYQQYHR | 120 |
| Lister | IELLKTMDLRKRDLGPAFTELISGLAKLKTSKYTYTDLTYQSFVDNTVCIKPSYYQQYHR | 60 |
| | **************************************************** ******* | |
| Cop | FGLYRLNFRRDAVNKINSIVERRSGMSNVVDSNMLDNNTLWAIINTIYFKGTWQYPFDIT | 180 |
| WR | FGLYRLNFRRDAVNKINSIVERRSGMSNVVDSNMLDNNTLWAIINTIYFKGIWQYPFDIT | 180 |
| Tian | FGLYRLNFRRDAVNKINSIVERRSGMSNVVDSNMLDNNTLWAIINTIYFKGTWQYPFDIT | 180 |
| Wyeth | -----LNFRRDAVNKINSIVERRSGMSNVVDSNMLDNNTLWAIINTIYFKGIWQYPFDIT | 175 |
| Lister | FGLYRLNFRRDAVNKINSIVERRSGMSNVVDSNMLDNNTLWAIINTIYFKGIWQYPFDIT | 120 |
| | ********************************************** ******** | |
| Cop | KTRNASFTNKYGTKTVPMMNVVTKLQGNTITIDDEEYDMVRLPYKDANISMYLAIGDNMT | 240 |
| WR | KTRNASFTNKYGTKTVPMMNVVTKLQGNTITIDDEEYDMVRLPYKDANISMYLAIGDNMT | 240 |
| Tian | KTRNASFTNKYGTKTVPMMNVVTKLQGNTITIDDEEYDMVRLPYKDANISMYLAIGDNMT | 240 |
| Wyeth | KTRNASFTNKYGTKTVPMMNVVTKLQGNTITIDDKEYDMVRLPYKDANISMYLAIGDNMT | 235 |
| Lister | KTRNASFTNKYGTKTVPMMNVVTKLQGNTITIDDEEYDMVRLPYKDANISMYLAIGDNMT | 180 |
| | **********************************:************************* | |
| Cop | HFTDSITAAKLDYWSFQLGNKVYNLKLPKFSIENKRDIKSIAEMMAPSMFNPDNASFKHM | 300 |
| WR | HFTDSITAAKLDYWSFQLGNKVYNLKLPKFSIENKRDIKSIAEMMAPSMENPDNASFKHM | 300 |
| Tian | HFTDSITAA-KDYWSFQLGNKVYNLKLPKFSIENKRDIKSIAEMMAPSMFNPDNASFKHM | 299 |
| Wyeth | HFTDSITAAKLDYWSFQLGNKVYNLKLPKFSIENKRDIKSIAEMMAPSMFNPDNASFKHM | 295 |
| Lister | HFTDSITAAKLDYWSSQLGNKVYNLKLPKFSIENKRDIKSIAEMMAPSMFNPDNASFKHM | 240 |
| | ********* **** ******************************************** | |
| Cop | TRDPLYIYKMFQNAKIDVDEQGTVAEASTIMVATARSSPEKLEFNTPFVFIIRHDITGFI | 360 |
| WR | TRDPLYIYKMFQNAKIDVDEQGTVARASTIMVATARSSPEKLEFNTPFVFIIRHDITGFI | 360 |
| Tian | TRDPLYIYKMFQNAKIDVDEQGTVAEASTIMVATARSSPEELEFNTPFVFIIRHDITGFI | 359 |
| Wyeth | TRDPLYIYKMFQNAKIDVDEQGTVAEASTIMVATARSSPEKLEFNTPFVFIIRHDITGFI | 355 |
| Lister | TRDPLYIYKMFQNAKIDVDEQGTVAEASTIMVATARSSPEKLEFNTPFVFIIRHDITGFI | 300 |
| | ****************************************:******************* | |
| Cop | LFMGKVESP | 369 |
| WR | LFMGKVESP | 369 |
| Tian | LFMGKVESP | 368 |
| Wyeth | LFMGKVESP | 364 |
| Lister | LFMGKVESP | 309 |
| | ********* | |

K ORF A
CLUSTAL O(1.2.4) multiple sequence alignment

| | | |
|---|---|---|
| Cop | MGHIITYCQVHTNISILIRKAHHIIFFVIDCDCISLQFSNYVHHGNRFRTVLISKTSIAC | 60 |
| Tian | MGHIITYCQVHTNISILIRKAYHIIFFVIDCDCISLQFSNYVHHGNRFRTVLISKTSIAC | 60 |
| | *********************:************************************** | |
| Cop | FSDIKRILPCTFKIYSINDCP | 81 |
| Tian | FSDIKRILPCTFKIYSINDCP | 81 |
| | ********************* | |

K3L
CLUSTAL O(1.2.4) multiple sequence alignment

| | | |
|---|---|---|
| Cop | MLAFCYSLPNAGDVIKGRVYEKDYALYIYLFDYPHSEAILAESVKMHMDRYVEYRDKLVG | 60 |
| WR | MLAFCYSLPNAGDVIKGRVYEKDYALYIYLFDYPHFEAILAESVKMHMDRYVEYRDKLVG | 60 |
| Tian | MLAFCYSLPNAGDVIKGRVYEKDYALYIYLFDYPHSEAILAESVKMHMDRYVEYRDKLVG | 60 |
| Wyeth | MLAFCYSLPNAGDVIKGRVYENDYALYIYLFDYPHFEAILAESVKMHMDRYVEYRDKLVG | 60 |
| Lister | MLAFCYSLPNAGDVIKGRVYENDYALYIYLFDYPHSEAILAESVKMHMDRYVEYRDKLVG | 60 |
| | *********************:************* ************************ | |
| Cop | KTVKVKVIRVDYTKGYIDVNYKRMCRHQ | 88 |
| WR | KTVKVKVIRVDYTKGYIDVNYKRMCRHQ | 88 |
| Tian | KTVKVKVIRVDYTKGYIDVNYKRMCRHQ | 88 |
| Wyeth | KTVKVKVIRVDYTKGYIDVNYKRMCRHQ | 88 |
| Lister | KTVKVKVIRVDYTKGYIDVNYKRMCRHQ | 88 |
| | **************************** | |

K4L
CLUSTAL O(1.2.4) multiple sequence alignment

| | | |
|---|---|---|
| Cop | MNPDNTIAVITETIPIGMQFDKVYLSTFNMWREILSNTTKTLDISSFYWSLSDEVGTNFG | 60 |
| WR | MNPDNTIAVITETIPIGMQFDKVYLSTFNMWREILSNTTKTLDISSFYWSLSDEVGTNFG | 60 |
| Wyeth | MNPDNTIAVITETIPIGMQFDKVYLSTFNMWREILSNTTKTLDISSFYWSLSDEVGTNFG | 60 |
| Lister | MNPDNTIAVITETIPIGMQFDKVYLSTFNMWREILSNTTKTLDISSFYWSLSDEVGTNFG | 60 |
| | ************************************************************ | |
| Cop | TIILNEIVQLPKRGVRVRVAVNKSNKPLKDVERLQMAGVEVRYIDITNILGGVLHTKFWI | 120 |
| WR | TIILNKIVQLPKRGVRVRVAVNKSNKPLKDVERLQMAGVEVRYIDITNILGGVLHTKFWI | 120 |
| Wyeth | TIILNEIVQLPKRGVRVRVAVNKSNKPLKDVERLQMAGVEVRYIDITNILGGVLHTKFWI | 120 |
| Lister | TIILNEIVQLPKRGVRVRVAVNKSNKPLKDVERLQMAGVEVRYIDITNILGGVLHTKFWI | 120 |
| | *****:****************************************************** | |
| Cop | SDNTHIYLGSANMDWRSLTQVKELGIAIFNNRNLAADLTQIFEVYWYLGVNNLPYNWKNF | 180 |
| WR | SDNTHIYLGSANMDWRSLTQVKELGIAIFNNRNLAADLTQIFEVYWYLGVNNLPYNWKNF | 180 |
| Wyeth | SDNTHIYLGSANMDWRSLTQVKELGIAIFNNRNLAADLIQIFEVYWYLGVNNLPYNWKNF | 180 |
| Lister | SDNTHIYLGSANMDWRSLTQVKELGIAIFNNRNLAADLIQIFEVYWYLGVNNLPYNWKNF | 180 |
| | ************************************************************ | |
| Cop | YPSYYNTDHPLSINVSGVPHSVFIASAPQQLCTMERTNDLTALLSCIRNASKFVYVSVMN | 240 |
| WR | YPSYYNTDHPLSINVSGVPHSVFIASAPQQLCTMERTNDLTALLSCIRNASKFVYVSVMN | 240 |
| Wyeth | YPSYYNTDHPLSINVSGVPHSVFIASAPQQLCTMERTNDLTALLSCIRNASKFVYVSVMN | 240 |
| Lister | YPSYYNTDHPLSINVSGVPHSVFIASAPQQLCTMERTNDLTALLSCIRNASKFVYVSVMN | 240 |
| | ************************************************************ | |
| Cop | FIPIIYSKAGKILFWPYIEDELRRSAIDRQVSVKLLISCWQRSSFIMRNFLRSIAMLKSK | 300 |
| WR | FIPIIYSKAGNILFWPYIEDELRRAAIDRQVSVKLLISCWQRSSFIMRNFLRSIAMLKSK | 300 |
| Wyeth | FIPIIYSKAGKILFWPYIEDELRRSAIDRQVSVKLLISCWQRSSFIMRNFLRSIAMLKSK | 300 |
| Lister | FIPIIYSKAGKILFWPYIEDELRRSAIDRQVSVKLLISCWQRSSFIMRNFLRSIAMLKSK | 300 |
| | **********:*************:*********************************** | |
| Cop | NIDIEVKLFIVPDADPPIPYSRVNHAKYMVTDKTAYIGTSNWTGNYFTDTCGASINITPD | 360 |
| WR | NINIEVKLFIVPDADPPIPYSRVNEAKYMVTDKTAYIGTSNWTGNYFTDTCGASINITPD | 360 |
| Wyeth | NINIEVKLFIVPDADPPIPYSRVNHAKYMVTDKTAYIGTSNWTGNYFTDTCGASINITPD | 360 |
| Lister | NINIEVKLFIVPDADPPIPYSRVNHAKYMVTDKTAYIGTSNWTGNYFTDTCGASINITPD | 360 |
| | **:********************************************************* | |
| Cop | DGLGLRQQLEDIFMRDWNSKYSYELYDTSPTKRCKLLKNMKQCTNDIYCDEIQPEKEIPE | 420 |
| WR | DGLGLRQQLEDIFMRDWNSKYSYELYDTSPTKRCRLLKNMKQCTNDIYCDEIQPEKEIPE | 420 |
| Wyeth | DGLGLRQQLEDIFMRDWNSKYSYELYDTSPTKRCKLLKNMKQCTNDIYCDEIQPEKEIPE | 420 |
| Lister | DGLGLRQQLEDIFMRDWNSKYSYELYDTSPTKRCKLLKNMKQCTNDIYCDEIQPEKEIPE | 420 |
| | **********************************:************************* | 420 |
| Cop | YSLE | 424 |
| WR | YSLE | 424 |
| Wyeth | YSLE | 424 |
| Lister | YSLE | 424 |
| | **** | |

K5L
CLUSTAL O(1.2.4) multiple sequence alignment

| | | |
|---|---|---|
| Cop | -------------MGATISILASYDNPNLFTAMILMSPLVNADAVSRLNLLAAKLMGTIT | 47 |
| WR | MTLVQHVVTIKSTYWVIPWELASYDNPNLFTAMILMSPLVNADAVSKLNLLAAKINGTIT | 60 |
| Tian | MTLVQHVVTIKSTYWVIPWELASYDNPNLFTAMILMSPLVNADAVSKLNLLAAKLMGTIT | 60 |
| Wyeth | -------------MGATISILASYDNPNLFTAMILMSPLVNADAVSKLNLLAAKLMGTIT | 47 |
| Lister | ---------MGHSMGATISILASYDNPNLFTAMILMSPLVNADAVSRINLLAAKLMGTIT | 51 |
| | . **************************:************* | |
| Cop | PNAPVGKLCPESVSRDMDKVYKYQYDPLINHEKIKAGFASQVLKATNKVRKIISKINTPR | 107 |
| WR | LNAPVGKLCPESVSRDMDKVYKYQYDPLINHEKIKAGFASQVLKATNKVRKIISKINTPR | 120 |
| Tian | LNAPVGKLCPESVSRDMDKVYKYQYDPLINHEKIKAGFASQVLKATNKVRKIISKINTPR | 120 |
| Wyeth | PNAPVGKLCPESVSRDMDKVYKYQYDPLINHEKIKAGFASQVLKATNKVRKIISKINTPP | 107 |
| Lister | PNAPVGKLCPESVSRDMDKVYKYQYDPLINHEKIKAGFASQVLKATNKVRKIISKINTPP | 111 |
| | ********************************************************** | |
| Cop | LSYSREQTMRL-----VMFQVHIISCNMQIVIE--------------------------- | 135 |
| WR | LSYSREQTIRL-----AMF----------------------------------------- | 134 |
| Tian | LSYSREQTIRL-----AMF----------------------------------------- | 134 |
| Wyeth | TLILQGTNNEISDVLGAYYFMQHANCNREIKIYEGAKHHLHKETDEVKKSVMKEIETWIF | 167 |
| Lister | TLILQGTNNKISDVLGAYYFMQHANCNREIKIYEGAKHHLHKETDEVKKSVMKEIETWIF | 171 |
| | :..: .: | |
| Cop | ---- | |
| WR | ---- | |
| Tian | ---- | |
| Wyeth | NRVK | 171 |
| Lister | NRVK | 175 |

K6L
CLUSTAL O(1.2.4) multiple sequence alignment

| | | |
|---|---|---|
| Cop | MSANCMFNLDNDYIYWKPITYPKALVFISHGAGKHSGRYDELAENISSLGILVFSHDHIG | 60 |
| WR | MSANCMFNIDNDYIYWKPITYPKALVFISHGAGKHSGRYDELAENISSLGILVFSHDHIG | 60 |
| Wyeth | MSANCMFNLDNDYIYWKPITYPKALVFISHGAGKHSGRYDELAENISSLGILVFSHDHIG | 60 |
| Lister | MSANCMFNLDNDYIYWKPITYPKALVFISHGAGKHSGRYDELAENISSLGILVFSHDHIG | 60 |
| | ************************************************************ | |
| Cop | HGRSNGEKMMIDDFGTARGNY | 81 |
| WR | HGRSNGEKMMIDDFGTARGNY | 81 |
| Wyeth | HGRSNGEKMMIDDFGTARGNY | 81 |
| Lister | HGRSNGEKMMIDDFGTARGNY | 81 |
| | ********************* | |

K7R
CLUSTAL O(1.2.4) multiple sequence alignment

| | | |
|---|---|---|
| Cop | MATKLDYEDAVFYFVDDDKICSRDSIIDLIDEYITWRNHVIVFNKDITSCGRLYKELMKF | 60 |
| WR | MATKLDYEDAVFYFVDDDKICSRDSIIDLIDEYITWRNHVIVFNKDITSCGRLYKELMKF | 60 |
| Tian | MATKLDYEDAVFYFVDDDKICSRDSIIDLIDEYITWRNHVIVFNKDITSCGRLYKELMKF | 60 |
| Wyeth | MATKLDYEDAVFYFVDDDKICSRDSIIDLIDEYITWRNHVIVFNKDITSCGRLYKELMKF | 60 |
| Lister | MATKLDYEDAVFYFVDDDKICSRDSIIDLIDEYITWRNHVIVFNKDITSCGRLYKELMKF | 60 |
| | ************************************************************ | |
| Cop | DDVAIRYYGIDKINEIVEAMSEGDHYINFTKVHDQESLFATIGICAKITEHWGYKKISES | 120 |
| WR | DDVAIRYYGIDKINEIVEAMSEGDHYINFTKVHDQESLFATIGICAKITEHWGYKKISES | 120 |
| Tian | DDVAIRYYGIDKINEIVEAMSEGDHYINFTKVHDQESLFATIGICAKITEHWGYKKISES | 120 |
| Wyeth | DDVAIRYYGIDKINEIVEAMSEGDHYINFTKVHDQESLFATIGICAKITEHWGYKKISES | 120 |
| Lister | DDVAIRYYGIDKINEIVEAMSEGDHYINFTKVHDQESLFATIGICAKITEHWGYKKISES | 120 |
| | ************************************************************ | |
| Cop | RFQSLGNITDLMTDDNINILILFLEKKLN | 149 |
| WR | RFQSLGNITDLMTDDNINILILFLEKKLN | 149 |
| Tian | RFQSLGNITDLMTDDNINILILFLEKKLN | 149 |
| Wyeth | RFQSLGNITDLMTDDNINILILFLEKKLN | 149 |
| Lister | RFQSLGNITDLMTDDNINILILFLEKKLN | 149 |
| | ***************************** | |

F1L
CLUSTAL O(1.2.4) multiple sequence alignment

| | | |
|---|---|---|
| Cop | MLSMFMCNNIVDYVDDIDNGIVQDIEDEASNNVDHDYVYPLPENMVYRFDKSTNILDYLS | 60 |
| WR | MLSMFMCNNIVDYVDDIDNGIVQDIEDEASNNVDHDYVYPLPENMVYRFDKSTNILDYLS | 60 |
| Tian | MLSMFMCNNIVDYVDDIDNGIVQDIEDEASNNVDRDYVYPLPENMVYRFDKSTNILDYLS | 60 |
| Wyeth | MLSMFMCNNIVDYVDDIDNGIVQDIEDEASNNVDHDYVYPLPENMVYRFDKSTNILDYLS | 60 |
| Lister | MLSMFMCNNIVDYVDDIDNGIVQDIEDEASNNVDHDYVYPLPENMVYRFDKSTNILDYLS | 60 |
| | **********************************:************************* | |
| Cop | TERDHVMMAVRYYMSKQRLDDLYRQLPTKTRSYIDIINIYCDKVSNDYNRDMNIMYDMAS | 120 |
| WR | TERDHVMMAVRYYMSKQRLDDLYRQLPTKTRSYIDIINIYCDKVSNDYNRDMNIMYDMAS | 120 |
| Tian | TERDHVMMAVRYYMSKQRLDDLYRQLPTKTRSYIDIINIYCDKVSNDYNRDMNIMYDMAS | 120 |
| Wyeth | TERDHVMMAVRYYMSKQRLDDLYRQLPTKTRSYIDIINIYCDKVSNDYNRDMNIMYDMAS | 120 |
| Lister | TERDHVMMAVRYYMSKQRLDDLYRQLPTKTRSYIDIINIYCDKVSNDYNRDMNIMYDMAS | 120 |
| | ************************************************************ | |
| Cop | TKSFTVYDINNEVNTILMDNKGLGVRLATISFITELGRRCMNPVKTIKMFTLLSHTICDD | 180 |
| WR | TKSFTVYDINNEVNTILMDNKGLGVRLATISFITELGRRCMNPVETIKMFTLLSHTICDD | 180 |
| Tian | TKSFTVYDINNEVNTILMDNKGLGVRLATISFITELGRRUMNPVKTIKMFTLLSHTICDD | 180 |
| Wyeth | TKSFTVYDINNEVNTILMDNKGLGVRLATISFITKLGRRCMNPVKTIKMFTLLSHTICDD | 180 |
| Lister | TKSFTVYDINNEVNTILMDNKGLGVRLATISFITELGRRCMNPVKTIKMFTLLSHTICDD | 180 |
| | **********************************:*********:*************** | |
| Cop | CFVDYITDISPPDNTIPNTSTREYLKLIGITAIMFATYKTLKYMIG | 226 |
| WR | YFVDYITDISPPDNTIPNTSTREYLKLIGITAIMFATYKTLKYMIG | 226 |
| Tian | CFVDYITDISPPDNTIPNTSTREYLKLIGITAIMFATYKTLKYMIG | 226 |
| Wyeth | CFVDYITDISPPDNTIPNTSTREYLKLIGITAIMFATYKTLKYMIG | 226 |
| Lister | CFVDYITDISPPDNTIPNTSTREYLKLIGITAIMFATYKTLKYMIG | 226 |
| | ********************************************** | |

F2L
CLUSTAL O(1.2.4) multiple sequence alignment

| | | |
|---|---|---|
| Cop | MFNMNINSPVRFVKETNRAKSPTRQSPGAAGYDLYSAYDYTIPPGERQLIKTDISMSMPK | 60 |
| WR | MFNMNINSPVRFVKETNRAKSPTRQSPYAAGYDLYSAYDYTIPPGERQLIKTDISMSMPK | 60 |
| Tian | MFNMNINSPVRFVKETNRAKSPTRQSPGAAGYDLYSAYDYTIPPGERQLIKTDISMSMPK | 60 |
| Wyeth | MFNMNINSPVRFVKETNRAKSPTRQSPGAAGYDLYSAYDYTIPPGERQLIKTDISMSMPK | 60 |
| Lister | MFNMNINSPVRFVKETNRAKSPTRQSPGAAGYDLYSAYDYTIPPGERQLIKTDISMSMPK | 60 |
| | ************************************************************ | |
| Cop | ICYGRIAPRSGLSLKGIDIGGGVIDEDYRGNIGVILINNGKCTFNVNTGDRIAQLIYQRI | 120 |
| WR | FCYGRIAPRSGLSLKGIDIGGGVIDEDYRGNIGVILINNGKCTFNVNTGDRIAQLIYQRI | 120 |
| Tian | ICYGRIAPRSGLSLKGIDIGGGVIDEDYRGNIGVILINNGKCTFNVNTGDRIAQLIYQRI | 120 |
| Wyeth | ICYGRIAPRSGLSLKGIDIGGGVIDEDYRGNIGVILINNGKCTFNVNTGDRIAQLIYQRI | 120 |
| Lister | FCYGRIAPRSGLSLKGIDIGGGVIDEDYRGNIGVILINNGKCTFNVNTGDRIAQLIYQRI | 120 |
| | :*********************************************************** | |
| Cop | YYPELEEVQSLDSTNRGDQGFGSTGLR | 147 |
| WR | YYPELEEVQSLDSTNRGDQGFGSTGLR | 147 |
| Tian | YYPELEEVQSLDSTDRGDQGFGSTGLR | 147 |
| Wyeth | YYPELEEVQSLDSTNRGDQGFGSTGLR | 147 |
| Lister | YYPELEEVQSLDSTNRGDQGFGSTGLR | 147 |
| | **************:************ | |

F3L
CLUSTAL O(1.2.4) multiple sequence alignment

| | | |
|---|---|---|
| Cop | MPIFVNTVYCKNILALSMTKKFKTIIDAIGGNIIVNSTILKKLSPYFRTHLRQKYTKNKD | 60 |
| WR | MPIFTNTVYCKNILALSMTKKFKTIIDAIGGNIIVNSTILKKLSPYFRTHLRQKYTKNKD | 60 |
| Tian | MPIFVNTVYCKNILALSMTKKFKTIIDAIGGNIIVNSTILKKLSPYFRTHLRQKYTKNKD | 60 |
| Wyeth | MPIFVNTVYCKNILALSMTKKFKTIIDAIGGNIIVNSTILKKLSPYFRTHLRQKYTKNKD | 60 |
| Lister | MPIFVNTVYCKNILALSMTKKFRTIIDAIGGNSIVNSTILKKLSPYFRTHLRQKYTKNKD | 60 |
| | ************************************************************ | |
| Cop | PVTRVCLDLDIHSLTSIVIYSYTGKVYIDSHNVVNLLRASILTSVEFIIYTCINFILRDF | 120 |
| WR | PVTWVCLDLDIHSLTSIVIYSYTGKVYIDSHNVVNLLRASILTSVEFIIYTCINFILRDF | 120 |
| Tian | PVTRVCLDLDIHSLTSIVIYSYTGKVYIDSHNVVNLLRASILTSVEFIIYTCINFILRDF | 120 |
| Wyeth | PVTRVCLDLDIHSLTSIVIYSYTGKVYIDSHNVVNLLRASILTSVEFIIYTCINFILRDF | 120 |
| Lister | PVTRVCLDLDIHSLTSIVIYSYTGKVYIDSHNVVNLLRASILTSVEFIIYTCINFILRDF | 120 |
| | *** ******************************************************** | |
| Cop | RKEYCVECYMMGIEYGLSNLLCHTKNFIAKHFLELEDDIIDNFDYLSMKLILESDELNVP | 180 |
| WR | RKEYCVECYMMGIEYGLSNLLCHTKNFIAKHFLELEDDIIDNFDYLSMKLILESDELNVP | 180 |
| Tian | RKEYCVECYMMGIEYGLSNLLCHTKNFIAKHFLELEDDIIDNFDYLSMELILESDELNVP | 180 |
| Wyeth | RKEYCVECYMMGIEYGLSNLLCHTKNFIAKHFLELEDDIIDNFDYLSMKLILESDELNVP | 180 |
| Lister | RKEYCVECYMMGIEYGLSNLLCHTKNFIAKHFLELEDDIIDNFDYLSIKLILESDELNVP | 180 |
| | ***********************************************:************ | |
| Cop | DEDYVVDFVIKWYIKRRNKLGNLLLLIKNVIRSNYLSPRGINNVKWILDCTKIFHCDKQP | 240 |
| WR | DEDYVVDFVIKWYIKRRNKLGNLLLLIKNVIRSNYLSPRGINNVKWILDCTKIFHCDKQP | 240 |
| Tian | DEDYVVDFVIKWYIKRRNKLGNLLLLIKNVIRSNYLSPRGINNVKWILDCTKIFHCDKQP | 240 |
| Wyeth | DEDYVVDFVIKWYIKRRNKLGNLLLLIKNVIRSNYLSPRGINNVKWILDCTKIFHCDKQP | 240 |
| Lister | DEDYVVDFVIKWYIKRRNKLGNLLLLIKNVIRSNYLSPRGINNVKWILDCTKIFHCDKQP | 240 |
| | ************************************************************ | |
| Cop | RKSYKYPFIEYPMNMDQIIDIFHMCTSTHVGEVVYLIGGWMNNEIHNNAIAVNYISNNWI | 300 |
| WR | RKSYKYPFIEYPMNMDQIIDIFHMCTSTHVGEVVYLIGGWMNNEIHNNAIAVNYISNNWI | 300 |
| Tian | RKSYKYPFIEYPMNMDQIIDIFHMCTSTHVGEVVYLIGGWMNNEIHNNAIAVNYISNNWI | 300 |
| Wyeth | RKSYKYPFIEYPMNMDQIIDIFHMCTSTHVGEVVYLIGGWMNNEIHNNAIAVNYISNNWI | 300 |
| Lister | RKSYKYPFIEYPMNMDQIIDIFHMCTSTHVGEVVYLIGGWMNNEIHNNAIAVNYISNNWI | 300 |
| | ************************************************************ | |
| Cop | PIPPMNSPRLYATGIPANNKLYVVGGLPNPTSVERWFHGDAAWVNMPSLLKPRCNPAVAS | 360 |
| WR | PIPPMNSPRLYASGIPANNKLYVVGGLPNPTSVERWFHGDAAWVNMPSLLKPRCNPAVAS | 360 |
| Tian | PIPPMNSPRLYASGIPANNKLYVVGGLPNPTSVERWFHGDAAWVNMPSLLKPRCNPAVAS | 360 |
| Wyeth | PIPPMNSPRLYASGIPANNKLYVVGGLPNPTSVERWFHGDAAWVNMPSLLKPRCNPAVAS | 360 |
| Lister | PIPPMNSPRLYASGIPANNKLYVVGGLPNPTSVERWFHGDAAWVNMPSLLKPRCNPAVAS | 360 |
| | ************:*********************************************** | |
| Cop | INNVIYVMGGHSETDTTTEYLLPNHDQWQFGPSTYYPHYKSCALVFGRRLFLVGRNAEFY | 420 |
| WR | INNVIYVMGGHSETDTTTEYLLPNHDQWQFGPSTYYPHYKSCALVFGRRLFLVGRNAEFY | 420 |
| Tian | INNVIYVMGGHSETDTTTEYLLPNHDQWQFGPSTYYPHYKSCALVFGRRLFLVGRNAEFY | 420 |
| Wyeth | INNVIYVMGGHSETDTTTEYLLPNHDQWQFGPSTYYPHYKSCALVFGRRLFLVGRNAEFY | 420 |
| Lister | INNVIYVMGGHSETDTTTEYLLPNHDQWQFGPSTYYPHYKSCALVFGRRLFLVGRNAEFY | 420 |
| | ************************************************************ | |
| Cop | CESSNTWTLIDDPIYPRDNPELIIVDNKLLLIGGFYRGSYIDTIEVYNHHTYSWNIWDGK | 480 |
| WR | CESSNTWTLIDDPIYPRDNPELIIVDNKLLLIGGFYRESYIDTIEVYNHHTYSWNINDGK | 480 |
| Tian | CESSNTWTLIDDPIYPRDNPELIIVDNKLLLIGGFYRESYIDTIEVYNHHTYSWNINDGK | 480 |
| Wyeth | CESSNTWTLIDDPIYPRDNPELIIVDNKLLLIGGFYRESYIDTIEVYNHHTYSWNIWDGK | 480 |
| Lister | CESSNTWTLIDDPIYPRDNPELIIVDNKLLLIGGFYRESYIDTIEVYNHHTYSWNIWDGK | 480 |
| | ************************************* ********************** | |

B14R
CLUSTAL O(1.2.4) multiple sequence alignment

| | | |
|---|---|---|
| Cop | ------------------------------------------------------------ | |
| WR | MDIFREIASSMKGENVFISPASISSVLTILYYGANGSTAEQLSKYVEKEENMDKVSAQNI | 60 |
| Tian | ------------------------------------------------------------ | |
| Wyeth | ------------------------------------------------------------ | |
| Cop | ------------------------------------------------------------ | |
| WR | SFKSINKVYGRYSAVFKDSFLRKIGDKFQTVDFTDCRTIDAINKCVDIFTEGKINPLLDE | 120 |
| Tian | ------------------------------------------------------------ | |
| Wyeth | ------------------------------------------------------------ | |
| Cop | ---MNHCLLAISAVYFKAKWLTPFEKEFTSDYPFYVSPTEMVDVSMMSMYGELFNHASVK | 57 |
| WR | PLSPDTCLLAISAVYFKAKNLTPFEKEFTSDYPFYVSPTEMVDVSMNSMYGKAFNHASVK | 180 |
| Tian | ---MNHCLLAISAVYFKAKWLTPFEKEFTSDYPFYVSPTEMVDVSMMSMYGKAFNHASVK | 57 |
| Wyeth | ---MNHCLLAISAVYFKAKWLTPFEKEFTSDYPFYVSPTEMVDVSMMSMYGKAFNHASVK | 57 |
| | : *********************************************: ******* | |
| Cop | ESFGNFSIIELPYVGDTSMMVILPDKIDGLESIEQNLTDTNFKKWCNSLDAMFIDVHIPK | 117 |
| WR | ESFGNFSIIELPYVGDTSMMVILPDKIDGLESIEQNLTDTNFKKWCNSLEATFIDVHIPK | 240 |
| Tian | ESFGNFSIIELPYVGDTSMMVILPDKIDGLESIEQNLTDTNFKKWCDFMDAMFIDVHIPK | 117 |
| Wyeth | ESFGNFSIIELPYVGDTSMMVILPDKIDGLESIEQNLTDTNFKKWCDFMDAMFIDVHIPK | 117 |
| | **********************************************: ::* ******** | |
| Cop | FKVTGSYNLVDTLVKSGLTEVFGSTGDYSNMCNLDVSVDAMIHKTYIDVNEEYTEAAAAT | 177 |
| WR | FKVTGSYNLVDTLVKSGLTEVFGSTGDYSNMCNSDVSVDAMIHKTYIDVNEEYTEAAAAT | 300 |
| Tian | FKVTGSYNLVDTLVKSGLTEVFGSTGDYSNMCNLDVSVDAMIHKTYIDVNEEYTEAAAAT | 177 |
| Wyeth | FKVTGSYNLVDTLVKSGLTEVFGSTGDYSNMCNLDVSVDAMIHKTYIDVNEEYTFAAAAT | 177 |
| | ********************************* ************************** | |
| Cop | CALVSDCASTITNEFCVDHPFIYVIRHVDGKILFVGRYCSPTTNC | 222 |
| WR | CALVSDCASTITNEFCVDHPFIYVIRHVDGKILFVGRYCSPTTNC | 345 |
| Tian | CALVSDCASTITNEFCVDHPFIYVIRHVDGKILFVGRYCSPTTNC | 222 |
| Wyeth | CALVSDCASTVTNEFCADHPFIYVIRHVDGKILFVGRYCSPTTNC | 222 |
| | **********:*****.**************************** | |

B15R
CLUSTAL O(1.2.4) multiple sequence alignment

| | | |
|---|---|---|
| Cop | MTANFSTHVFSPQHCGCDRLTSIDDVKQCLTEYIYWSSYAYRNRQCAGQLYSTLLSFRDD | 60 |
| WR | MTANFSTHVFSPQHCGCDRLTSIDDVKQCLTEYIYWSSYAYANRQCAGQLYSTLLSFRDD | 60 |
| Tian | MTANFSTHVFSPQHCGCDRLTSIDDVKQCLTEYIYWSSYAYRNRQCAGQLYSTLLSFRDD | 60 |
| Wyeth | MTANFSTHVFSPQHCGCDRLTSIDDVKQCLTEYIYWSSYAYRNRQCAGQLYSTLLSFRDD | 60 |
| Lister | MTANFSTHVFSPQHCGCDRLTSIDDVKQCLTEYIYWSSYAYRNRQCAGQLYSTLLSFRDD | 60 |
| | **************************:********************************* | |
| Cop | AELVFIDIRELVKNMPWDDVKDCTEIIRCYIPDEQKTIREISAIIGLCAYAATYWGGEDH | 120 |
| WR | AELVFIDIRELVKNMPWDDVKDCAEIIRCYIPDEQKTIREISAIIGLCAYAATYWGGEDH | 120 |
| Tian | AELVFIDIRELVKNMEWDDVKDCTEIIRCYIPDEQKTIREISAIIGLCAYAATYWGGEDH | 120 |
| Wyeth | AELVFIDIRELVKHMPWDDVKDCAEIIRCYIPDEQKTIREISAIIGLCAYAATYWGGEDH | 120 |
| Lister | AELVFIDIRELVKNMPWDDVKDCTEIIRCYIPDEQKTIREISAIIGLCAYAATYWGGEDH | 120 |
| | *************:*********:************************************ | |
| Cop | PTSNSLNALFVMLEMLNYVDYNIIFRRMN | 149 |
| WR | PTSNSLNALFVMLEMLNYVDYNIIFRRMN | 149 |
| Tian | PTSNSLNALFVMLEMLNYVDYNIIFRRMN | 149 |
| Wyeth | PTSNSLNALFVMLEMLNYVDYNIIFRRMN | 149 |
| Lister | PTSNSLNALFVMLEMLNYVDYNIIFRRMN | 149 |
| | ***************************** | |

B ORF E
CLUSTAL O(1.2.4) multiple sequence alignment

| | | |
|---|---|---|
| Cop | MYNSSIHTPEYDVIIHVIEHLKHHKQCVQTVTSGMVFTSPVSSSICTKSDDGRNLSDGFL | 60 |
| Tian | MYNSSIHTPEYDVIIHVIEHLKHHKQCVQTVTSGMVFTSPVSSSICTKSDDGRNLSDGFL | 60 |
| | ************************************************************ | |
| Cop | LIRYITTDDFCTIFDIIPRHIFYQLANVDEH | 91 |
| Tian | LIRYITTDDFCTIFDIIPRHIFYQLANVDEH | 91 |
| | ******************************* | |

B16R
CLUSTAL O(1.2.4) multiple sequence alignment

| | | |
|---|---|---|
| Cop | MSILPVIFLPIFFYSSFVQTFNASECIDKGXYFASFMELENEPVILPCPQINTISSGYNI | 60 |
| WR | MSILPVIFLSIFFYSSFVQTFNAPECIDKGQYFASFMELENEPVILPCPQINTLSSGYNI | 60 |
| Tian | ------------------------------------MELENEPVILPCPQINTLSSGYNI | 24 |
| Wyeth | MSILPVIFLSIFFYSSFVQTFNASECIDKGQYFASFMELENEPVILPCPQINTLSSGYNI | 60 |
| Lister | MSILPVIFLPIFFYSSFVQTFNAPECIDKGQYFASFMELENEPVILPCPQINTLSSGYNI | 60 |
| | ************************ | |
| Cop | LDILWEKRGADNDRIIPIDNGSNMLILNPTQSDSGIYICITTNETYCDMMSLNLTIVSVS | 120 |
| WR | LDILWEKRGADNDRIIPIDNGSNMLILNPTQSDSGIYICITTNETYCDMMSLNLTIVSVS | 120 |
| Tian | LDILWEKAGADNDRIIPIDNGSNMLILNPTQSDSGIYICITTNETYCDMMSLNLTIVSVL | 84 |
| Wyeth | LDILWEKRGADNDRIIPIDNGSNMLILNPTQSDSGIYICITTNETYCDMMSLNLTIVSVS | 120 |
| Lister | LDILWEKRGADNDRIIPIDNGSNMLILNPTQSDSGIYICITTNETYCDMMSLNLTIVSVS | 120 |
| | ************************************************************ | |
| Cop | ESNIDFISYPQIVNERSTGEMVCPNINAFIASNVNADIIWSGHRRLRNKRLKQRTPGIIT | 180 |
| WR | ESNIDLISYPQIVNERSTGEMVCPNINAFIASNVNADIIWSGHRRLRNKRLKQRTPGIIT | 180 |
| Tian | ESNIDLISYPQIVNERSTGEMVCPNINAFIASNVNADIIWSGHRRLRNKRLKQRTPGIIT | 144 |
| Wyeth | ESNIDLISYPQIVNERSTGEMVCPNINAFIASNVNADIIWSGHRRLRNKRLKQRTPGIIT | 180 |
| Lister | ESNIDLISYPQIVNERSTGEMVCPNINAFIASNVNADIIWSGHRRLRNKRLKQRTPGIIT | 180 |
| | *****:****************************************************** | |
| Cop | IEDVRKNDAGYYTCVLEYIYGGKTYNVTRIVKLEVRDKIIHPTMQLPEGVVTSIGSNLTI | 240 |
| WR | IEDVRKNDAGYYTCVLEYIYGGKTYNVTRIVKLEVRDKIIPSTMQLPDGIVTSIGSNLTI | 240 |
| Tian | IEDVRKNDAGYYTCVLEYIYRGKTYNVTRIVKLEVRDKIIPSTMQLPDGIVTSIGSNLTI | 204 |
| Wyeth | IEDVRKNDAGYYTCVLEYIYGGKTYNVTRIVKLEVRDKIIPSTMQLPDGIVTSIGSNLTI | 240 |
| Lister | IEDVRKNDAGYYTCVLEYIYRGKTYNVTRIVKLEVRDKIIPSTMQLPDGIVTSIGSNLTI | 240 |
| | ******************** ******************* *****:*:********** | |
| Cop | ACRVSLRPPTTDADVFWISNGMYYEEDDGDGDGRISVANKIYMTDKRRVITSRLNINPVK | 300 |
| WR | ACRVSLRPPTTDADVFWISNGMYYEEDDGDGNGRISVANKIYMTDKRRVITSRLNINPVK | 300 |
| Tian | ACRVSLRPPTTDADVFWISNGMYYEEDDGDGNGRISVANKIYMTDKRRVITSRLNINPVK | 264 |
| Wyeth | ACRVSLRPPTTDTDVFWISNGMYYEEDDGDGDGRISVANKIYMIDKRRVITSRLNINPVK | 300 |
| Lister | ACRVSLRPPTTDADVFWISNGMYYEEDDGDGNGRISVANKIYMTDKRRVITSRLNINPVK | 300 |
| | ************:******************:**************************** | |
| Cop | EEDATTFTCMAFTIPSISKTVTVSIT | 326 |
| WR | EEDATTFTCMAFTIPSISKTVTVSIT | 326 |
| Tian | EEDATTFTCMAFTIPSISKTVTVSI- | 289 |
| Wyeth | EEDATTFTCMAFTIPSISKTVTVSIT | 326 |
| Lister | EEDATTFTCMAFTIPSISKTVTVSIT | 326 |
| | ************************* | |

B ORF F
CLUSTAL O(1.2.4) multiple sequence alignment

| | | |
|---|---|---|
| Cop | MVIIPGVRCLSLLFLRRRCPLHIISAFTLLAINALILGHTISPVDLSFTICGYEIKSIFD | 60 |
| Tian | MVIIPGVRCLSLLFLAARCPLHIISAFTLLAINALILGHTISPVDLSFTICGYEIRSIFD | 60 |
| | *******************************************************:**** | |
| Cop | SETDTIVKFNDIMSQ | 75 |
| Tian | SKTDTIVKFNDIMSQ | 75 |
| | *:************* | |

B17L
CLUSTAL O(1.2.4) multiple sequence alignment

| | | |
|---|---|---|
| Cop | MSRKFMQVYEYDREQYLDEFIEDRYNDSFITSPEYYSAEKYMCRYTTLNHNCVNVRRCAL | 60 |
| WR | MSRKFMQVYEYDREQYLDEFIEDRYNDSFITSPEYYSAEKYMCRYTTLNHNCINVRRCAL | 60 |
| Tian | MSRKFMQVYEYDREQYLDEFIEDRYNDSFITSPEYYSAEKYMCRYTTLNHNCVNVRRCAL | 60 |
| Wyeth | MSRKFMQVYEYDREQYLDEFIEDRYNDSFITSPEYYSAEKYMCRYTTLNHNCVNVRRCAL | 60 |
| Lister | MSRKFMQVYEYDREQYLDEFIEDRYNDSFITSPEYYSAEKYMCRYTTLNHNCINVRRCAL | 60 |
| | ****************************************************:******* | |
| Cop | DSKLLHDIITNCKIYNNIELVRATKFVYYLDLIKCNWVSKVGDSVLYPVIFITHTSTRNL | 120 |
| WR | DSKLLHDIITNCKIYNNIELVRATKFVYYLDLIKCNWVSKVGDSVLYPVIFITHTSTRNL | 120 |
| Tian | DSKLLHDIITNCKIYNNIELVRATKFVYYLDLIKCNWVSKVGDSVLYPVIFITHTSTRNL | 120 |
| Wyeth | DSKLLHDIITNCKIYNNIELVRATKFVYYLDLIKCNWVSKVGDSVLYPVIFITHTSTRNL | 120 |
| Lister | DSKLLHDIITNCKIYNNIELVRATKFVYYLDLIKCNWVSKVGDSVLYPVIFITHTSTRNL | 120 |
| | ************************************************************ | |
| Cop | DKVSVKTYKGVKVKKLNRCADHAIVINPFVKFKLTLPNKTSHAKVLVTFCKLRTDITPVE | 180 |
| WR | DKVSVKTYKGVKVKKLNRCADHAIVINPFVKFKLTLPNKTSHAKVLVTFCKLKTDITPVE | 180 |
| Tian | DKVSVKTYKGVKVKKLNRCADHAIVINPFVKFKLTLPNKTSHAKVLVTFCKLRTDITQIE | 180 |
| Wyeth | DKVSVKTYKGVKVKKLNRCADHAIVINPFVKFKLTLPNKTSHAKVLVTFCKLRTDITQIE | 180 |
| Lister | DKVSVKTYKGVKVKKLNRCADHAIVINPFVKFKLTLPNKTSHAKVLVTFCKLRTDITQIE | 180 |
| | ****************************************************:**** :* | |
| Cop | APLPGNVLVYTFPDINKRIPGYIHVNIEGCIDGMIYINSSKFACVLKLHRSMYRIPPFPI | 240 |
| WR | APLPGNVLVYTFPDINKRIPGYIHVNIEGCIDGMIYINSSKFACVLKLHRSMYRIPPFPI | 240 |
| Tian | APLSGNVLVYTFPNINKRIPGYIHVNIEGCIDGMIYINSSKFACVLKLHRSMYRIPPFPI | 240 |
| Wyeth | AFISGNVLVYTFPDINKRIPGYIHVNIEGCIDGMIYINSSKFACVLKLHRSMYRIPPFPI | 240 |
| Lister | APLSGNVLVYTFPDINKRIPGYIHVNIEGCIDGMIYINSSKFACVLKLHRSMYRIPPFPI | 240 |
| | *** *********:**********:*********************************** | |
| Cop | DICSCCSQYTNDDIEIPIHDLIKDVAIFKNKETVYYLKLNNKTIARFTYFNNIDTAITQE | 300 |
| WR | DICSCCSQYINYDIEIPIHDLIKDVAIFKNKETVYYLKLNNKTIARFTYFNNIDTAITQE | 300 |
| Tian | DICSCCSQYTNGDIEIPIHDLIKDVAIFKNKETVYYLKLNNKTIARFTYFNNIDTAITQE | 300 |
| Wyeth | DICSCCSQYTNDDIEIPIHDLIKDVAIFKNKETVYYLKLNNKTIARFTYFNNIDTAITQE | 300 |
| Lister | DICSCCSQYTNDDIEIPIHDLIKDVAIFKNKETVYYLKLNNKTIARFTYFNNIDTAITQE | 300 |
| | ********* * ************************************************ | |
| Cop | HEYVKIALGIVCKLMINNMHSIVGVNHSNTFVNCLLEDNV | 340 |
| WR | HEYVKIALGIVCKLMINNMHSIVGVNHSNTFVNCLLEDNV | 340 |
| Tian | HEYVKIALGIVCKLMINNMHSIVGVNRSNTFVNCLLEDNV | 340 |
| Wyeth | HEYVKIALGIVCKLMINNMHSIVGVNHSNTFVNCLLEDNV | 340 |
| Lister | HEYVKIALGIVCKLMINNMHSIVGVNHSNTFVNCLLEDNV | 340 |
| | **************************************** | |

B18R
CLUSTAL O(1.2.4) multiple sequence alignment

| | | |
|---|---|---|
| Cop | MSRRLIYVLNINRKSTHKIQENEIYTYFSHCNIDHTSTELDFVVKNYDLNRRQHVTGYTA | 60 |
| WR | MSRRLIYVLNINRESTHKIQENEIYTYFSHCNIDHTSTELDFVVKNYDLNRRQPVTGYTA | 60 |
| Tian | MSRRLIYVLNINRESTHKIQENEIYTYFSHCNIDHTSTELDFVVKNYDLNRRHPVTGYTA | 60 |
| Wyeth | MSRRLIYVLNINRESTHKIQENEIYTYFSHCNIDHTSTELDFVVKNYDLNRRQPVTGYTA | 60 |
| | *************:**************************************: ****** | |
| Cop | LHCYLYNNYFTNDVLKILLNHDVNVTMKTSSGRMPVYILLTRCCNISHDVVIDMIDKDKN | 120 |
| WR | LHCYLYNNYFTNDVLKILLNHGVDVTMKTSSGRMPVYILLTRCCNISHDVVIDMIDKDKN | 120 |
| Tian | LHCYLYNNYFTNDVLKILLNHGVDVTMKTSSGRMPVYILLTRCCNISHDVVIDMIDKDKN | 120 |
| Wyeth | LHCYLYNNYFTNDVLKILLNHGVDVTMKTSSGRMPVYILLTRCCNISHDVVIDMIDKDKN | 120 |
| | *********************.*:************************************ | |
| Cop | HLSHRDYSNLLLEYIKSRYMLLKEEDIDENIVSTLLDKGIDPNETQDGYTALHYYYLCLA | 180 |
| WR | HLLHRDYSNLLLEYIKSRYMLLKEEDIDENIVSTLLDKGIDPNFKQDGYTALHYYYLCLA | 180 |
| Tian | HLLHRDYSNLLLEYIKSRYMLLKEEDIDENIVSTLLDKGIDPNFKQDGYTALHYYYLCLA | 180 |
| Wyeth | HLSHRDYSNLLLEYIKSRYMLLKEEDIDENIVSTLLDKGIDPNFKQDGYTALHYYYLCLA | 180 |
| | ** ********************************************************* | |
| Cop | HVYKPGECRKPITIKKAKRIISLFIQHGANLNALDNCGNTPFHLYLSIEMCNNIHMTKML | 240 |
| WR | HVYKPGECRKPITIKKAKRIISLFIQHGANLNALDNCGNTPFHLYLSIEMCNNIHMTKML | 240 |
| Tian | HVYKPGECRKPITIKKAKRIISLFIQHGANLNALDNCGNTPFHLYLSIEMCNNIHMTKML | 240 |
| Wyeth | HVYKPGECRKPITIKKAKRIISLFIQHGANLNALDNCGNTPFHLYLSIEMCNNIHMTKML | 240 |
| | ************************************************************ | |
| Cop | LTFNPNFKICNNHGLTPILCYITSDYIQHDILVMLIHHYETNVGEMPIDERRMIVFEFIK | 300 |
| WR | LTFNPNFEICNNHGLTPILCYITSDYIQHDILVMLIHHYETNVGEMPIDERRIIVFEFIK | 300 |
| Tian | LTFNPNFKICNNHGLTPILCYITSDYIQHDILVMLIHHYETNVGEMPIDERRIIVFEFIK | 300 |
| Wyeth | LTFNPNFKICNNHGLTPILCYITSDYIQHDILVMLIHHYETNVGEMPIDERRIIVFEFIK | 300 |
| | *******:********************************************:******* | |
| Cop | TYSTRPADSITYLMNRFKNINIYTRYEGKTLLHVACEYNNTQVIDYLIRINGDINALTDN | 360 |
| WR | TYSTRPADSITYLMNRFKNIDIYTRYEGKTLLHVACEYNNTHVIDYLIRINGDINALTDN | 360 |
| Tian | TYSTRPADSITYLMNRFKNINIYTRYEGKTLLHVACEYNNTQVIDYLIRINGDINALTDN | 360 |
| Wyeth | TYSTRPADSITYLMNRFKNINIYTRYEGKTLLHVACEYNNTHVIDYLIRINGDINALTDN | 360 |
| | ********************:********************:****************** | |
| Cop | NKHATQLIIDNKENSPYTINCLLYILRYIVDKNVIRSLVDQLPSLPIFDIKSFEKFISYC | 420 |
| WR | NKHATQLIIDNKENSPYTINCLLYILRYIVDKNVIRSLVDQLPSLPIFDIKSFEKFISYC | 420 |
| Tian | NKHATQLIIDNKENSPYTINCLLYILRYIVDKNVIRSLVDOLPSLPIFDIKSFEKFISYC | 420 |
| Wyeth | NKHAIQLIIDNKENSPYTIDCLLYILRYIVDKNVIRSLVDQLPSLPIFDIKSFEKFISYC | 420 |
| | **** **************:**************************************** | |
| Cop | ILLDDTFYDRHVKNRDSKTYRYAFSKYMSFDKYDGIITKCHDETMLLKLSTVLDTTLYAV | 480 |
| WR | ILIDDTFYNRHVRNRDSKTYRYAFSKYMSFDKYDGIITKCHKETILLKLSTVLDTTLYAV | 480 |
| Tian | ILLDDTFYDRHVKNRNSKTYRYAFSKYMSFDKYDGIITKCHDETMLLKLSTVLDTTLYAV | 480 |
| Wyeth | ILLDDTFYNRHVRNRNSKTYRYAFSKYMSFDKYDGIITKCHDETMLLKLSTVLDTTLYAV | 480 |
| | ********:***:**:*************************.**:*************** | |
| Cop | LRCHNSRKLRRYLTELKKYNNDKSFKIYSNIMNERYLNVYYKDMYVSKVYDKLFPVFTDK | 540 |
| WR | LRCHNSKKLRRYLTELKKYNNDKSFKIYSNIMNERYLNVYYKDMYVSKVYDKLFPVFTDK | 540 |
| Tian | LRCHNSRKLRRYLTELKKYNNDKSFKIYSNIMNERYLNVYYKDMYVSKVYDKLFPVFTDK | 540 |
| Wyeth | LRCHNSKKLRRYLNELKKYNNDKSFKIYSNIMNERYLNVYYKDMYVSKVYDKLFPVFTDK | 540 |
| | ******:******.********************************************** | |
| Cop | NCLLTLLPSEIIYEILYMLTINDLYNISYPPTKV | 574 |
| WR | NCLLTILPSEIIYEILYMLTINDLYNISYPPTKV | 574 |
| Tian | NCLLTLLPSEIIYEILYMLTINDLYNISYPPTKV | 574 |
| Wyeth | NCLLTLLPSEIIYEILYMLTINDLYNISYPPTKV | 574 |
| | ********************************** | |

B19R
CLUSTAL O(1.2.4) multiple sequence alignment

| | | |
|---|---|---|
| Cop | MTMKMMVHIYFVSLSLLLLLFHSYAIDIENEITEFFNKMRDTLPAKDSKWLNPACMFGGT | 60 |
| WR | MTMKMMVHIYFVSL--LLLLFHSYAIDIENEITEFFNKMRDTIPAKDSKWLNPACMFGGT | 58 |
| Tian | MTMKMMVHIYFVSLSLLLLLFHSYAIDIENEITEFFNKMRDTLPAKDSKWLNPACMFGGT | 60 |
| Wyeth | MTMKMMVHIYFVSLSLLLLLFHSYAIDIENEITEFFNKMRDTLPAKDSKWLNPACMFGGT | 60 |
| | ************** ******************************************** | |
| Cop | MNDMATLGEPFSAKCPPIEDSLLSHRYKDYVVKWERLEKNRRRQVSNKRVKHGDLWIANY | 120 |
| WR | MNDIAALGEPFSAKCPPIEDSLLSHRYKDYVVKWERLEKNRRRQVSNKRVKHGDLWIANY | 118 |
| Tian | MNDIAALGEPFSAKCPPIEDSLLSHRYKDYVVKWERLEKNRRRQVSNKRVKHGDLWIANY | 120 |
| Wyeth | MNDIATLGEPFSAKCPPIEDSLLSHRYKDYVVKWERLEKNRRRQVSNKRVKHGDLWIANY | 120 |
| | ***:*:****************************************************** | |
| Cop | TSKFSNRRYLCTVTTKNGDCVQGIVRSHIKKPPSCIPKTYELGTHDKYGIDLYCGILYAK | 180 |
| WR | TSKFSNRRYLCTVTTKNGDCVQGIVRSHIRKPPSCIPKTYELGTHDKYGIDLYCGILYAK | 178 |
| Tian | TSKFSNRRYLCTVTTKNGDCVQGIVRSHIKKPPSCIPKTYELGTHDKYGIDLYCGILYAK | 180 |
| Wyeth | TSKFSNRRYLCTVTTKNGDCVQGIVRSHIRKPPSCIPKTYELGTHDKYGIDLYCGILYAK | 180 |
| | *****************************:****************************** | |
| Cop | HYNNITWYKDNKEINIDDIKYSQTGKELIIHNPELEDSGRYDCYVHYDDVRIKNDIVVSR | 240 |
| WR | HYNNITWYKDNKEINIDDIKYSQTGKELIIHNPELEDSGRYDCYVHYDDVRIKNDIVVSR | 238 |
| Tian | HYNNITWYKDNKEINIDDIKYSQTGKELIIHNPELEDSGRYDCYVHYDDVRIKNDIVVSR | 240 |
| Wyeth | HYNNITWYKDNKEINIDDIKYSQTGKKLIIHNPELEDSGRYDCYVHYDDVRIKNDIVVSR | 240 |
| | **************************:********************************* | |
| Cop | CKILTVIPSQDHRFKLILDPKINVTIGEPANITCTAVSTSLLIDDVLIEWENPSGWLIGF | 300 |
| WR | CKILTVIPSQDHRFKLILDPKINVTIGEPANITCTAVSTSLLIDDVLIEWENPSGWLIGF | 298 |
| Tian | CKILTVIPSQDHRFKLILDPKINVTIGEPANITCTAVSTSLLIDDVLIEWENPSGWLIGF | 300 |
| Wyeth | CKILVTIPSQDHRFKLKRNCGYASN---------------------------------- | 265 |
| | **************** : . | |
| Cop | DFDVYSVLTSRGGITEATLYFENVTEEYIGNTYKCRGHNYYFEKTLTTTVVLE | 353 |
| WR | DFDVYSVLTSRGGITEATLYFENVTEEYIGNTYKCRGHNYYFEKTLTTTVVLE | 351 |
| Tian | DFDVYSVLTSRGGITEATLYFENVTEEYIGNTYKCRGHNYYFEKTLTTTVVLE | 353 |
| Wyeth | ----------------------------------------------------- | |

B21R
CLUSTAL O(1.2.4) multiple sequence alignment

| | | |
|---|---|---|
| Cop | MSLESFIITTFNNNSSTNIDNMCHLYVKVCPSSLLFRLFVECCDINKLVEGTTPLHCYLM | 60 |
| Wyeth | MSLESFIITTFNNNSSTNIDNMCHLYVKVCPSSLLFRLFVECCDINKLVEGTTPLHCYLM | 60 |
| | ************************************************************ | |
| Cop | NEGFESSVLKNLLKEYVMNTFNVHDIHYTNI | 91 |
| Wyeth | NEGFESSVLKNLLKEYVMTSITQIFNS---- | 87 |
| | ******************.::. | |

B22R
CLUSTAL O(1.2.4) multiple sequence alignment

| | | |
|---|---|---|
| Cop | MISLSFLIHNPLKKWKLKPSISINGYRSTFTMAFPCAQFRPCHCHATKDSLNTVADVRHC | 60 |
| Wyeth | MISLSFLIHNPLKKWKLKPSISINGYRSTFTMAFPCAQFRPCHCHATKDSLNTVADVRHC | 60 |
| Lister | -------------------------------MASPCAKFRPCHCHATKDSLNTVADVRHC | 29 |
| | ** ***:********************** | |
| Cop | LTEYILWVSHRWTHRESAGSLYRLLISFRTDATELFGGELKDSLPWDNIDNCVEIIKCFI | 120 |
| Wyeth | LTEYILWVSHRWTHRETAGPLYRLLISFRTDATELEGGELKDSLPWDNIDNCVEIIKCFI | 120 |
| Lister | LTEYILWVSHRWTHRESAGSLYRLLISFRTDATELFGGELKDSLPWD---NCVEIIKCFI | 86 |
| | ****************:** *************************** ********** | |
| Cop | RNDSMKTAEELRAIIGLCTQSAIVSGRVFNDKYIDILLMLRKILNENDYLTLLDHIRTAK | 180 |
| Wyeth | RNDSMKTAEELRAIIGLCTQSAIVSGRVFNDKYIDILLMLRKILNENDYLTLLDHIRTAK | 180 |
| Lister | RNDSMKTAEELRAIIGLCTQSAIVSGRVFNDKYIDILLMLRKILNENDYLTLLDHIRTAK | 146 |
| | ************************************************************ | |
| Cop | Y | 181 |
| Wyeth | Y | 181 |
| Lister | Y | 147 |
| | * | |

B23R
CLUSTAL O(1.2.4) multiple sequence alignment

| | | |
|---|---|---|
| Cop | MIAFIIFREIGIISTRIAMDYCGRECTILCRLLDEDVTYKKIKLEIETCHNLSKHIDRRG | 60 |
| Wyeth | MIAFIIFREIGIISTRIAMDCT----CILCRLLDEDVTYKKIKLEIETCHNLSKHIDRRG | 56 |
| | ******************** ********************************* | |
| Cop | NNALHCYVSNKCDTDIKIVRLLLSRGVERLCRNNEGLTPLGAYSKHRYVKSQIVHLLISS | 120 |
| Wyeth | NNALHCYVFNKCDTDIKIVRLLLSRGVERLCRNNEGLTPLGVYSKHRYVKSQIVHLLISS | 116 |
| | ******** ********************************.****************** | |
| Cop | YSNSSNELKSNINDFDLSSDNIDLRLLKYLIVDKRIRPSKNTNYAINGLGLVDIYVTTPN | 180 |
| Wyeth | YSNSSNELKSNINDFDLSSDNIDLRLLKYLIVDKRIRPSKRTNYAINSLGLVDIYVTTPN | 176 |
| | ***********************************************.************ | |
| Cop | PRPEVLLWLLKSECYSTGYVFRTCMYNSDMCKNSLHYYISSHRESQSLSKDVIKCLINNN | 240 |
| Wyeth | PRPEVLLWLLKSECYSTGYVFRTCMYNSDMCKNSLHYYISSHRESQSLSKDVIKCLINNN | 236 |
| | ************************************************************ | |
| Cop | VSIHGRDEGGSLPIQYYWSFSTIDIEIVKLLLIKDVDTCRVYDVSPILEAYYLNKRFRVT | 300 |
| Wyeth | VSIHGRDEGGSLPIQYYWSFSTIDIEIVKLLLIKDVDTCRVYDVSPILEAYYLNKRFRVT | 296 |
| | ************************************************************ | |
| Cop | PYNVDMEIVNLLIERRHTLVDVMRSITSYDSREYNHYIIDNILKRFRQQDESIVQAMLIN | 360 |
| Wyeth | PYNVDMEIVNLLIERRHTLVDVMRSITSYDSREYNHYIIDNILKRFRQQDESIVQAMLIN | 356 |
| | ************************************************************ | |
| Cop | YLHYGDMVVRCMLDNGQQLSSARLLC | 386 |
| Wyeth | YLHYGDMVVRCMLDNGQQLSSARLLC | 382 |
| | ************************** | |

B24R
CLUSTAL O(1.2.4) multiple sequence alignment

| | | |
|---|---|---|
| Cop | MYGLILSRFNNCGYHCYETILIDVFDILSKYMDDIDMIDNENKTLLYYAVDVNNIQFAKR | 60 |
| Wyeth | MYGLILSRFNNCGYHCYETILIDVFDILSKYMDNIDMIDNENKTLLYYAVDVNNIQFAKR | 60 |
| | *********************************:************************** | |
| Cop | LLEYGASVTTSRSIINTAIQKSSYQRENKTRIVDLLLSYHPTLETMIDAFNRDIRYLYPE | 120 |
| Wyeth | LLEYGASVTTSRSIINTAIQKSSYRRENKTKLVDLLLSYHPTLETMIDAFNRDIRYLYPE | 120 |
| | ************************:*****::**************************** | |
| Cop | PLFACIRYALILDDDFPSKVSMISPVIIRN------------------------------ | 150 |
| Wyeth | PLFACIRYALILDDDFPSKVKYDISGRHKELKRYRVDINRMKNAYISGVSMFDILFKRSK | 180 |
| | ********************. :: | |
| Cop | ------------------ | |
| Wyeth | RHRLRYAKNPTSNGTKKN | 198 |

B25R
CLUSTAL O(1.2.4) multiple sequence alignment

| | | |
|---|---|---|
| Cop | MBRINITKKIYCSVFLFLFLFLSYISNYEKVNDEMYEMGEMDEIVSIVRDSMWYIPNVFM | 60 |
| WR | ----------------------------------------MDEIVRIVRDSMWYIPNVFM | 20 |
| Wyeth | MBRINITKKIYCSVFLF--LFLSYISNYEKVNDEMYEMGEMDEIVSIVRDSMWYIPNVFM | 58 |
| | ***** ************** | |
| Cop | DDGKNEGHVSVNNVCHMYFTFFDVDTSSHLFKLVIKHCDLNKRGNSPLHCYTMNTRFNPS | 120 |
| WR | DDGKNEGHVSVNNVCHMYFTFFDVDTSSHLFKLVIKHCDLNKRGNSPLHCYTMNTRFNPS | 80 |
| Wyeth | DDGKNEGHVSVNNVCMMYFTFFDVDTSSHLFKLVIKHCDLNKRGNSPLHCYTMNTRFNPS | 118 |
| | ************************************************************ | |
| Cop | VLKILLHHGMRNFDSKDEKGHHYLIHSLSIDNKIFDILTDTIDDFSKSSDLLLCYLRYKF | 180 |
| WR | VLKILLHHGMRNFDSKDEKGHHYQSITRSLIY---------------------------- | 112 |
| Wyeth | VLKILLHHGMRNFDSKD---DHYQSITRSLIY---------------------------- | 147 |
| | ***************** .** : *: | |
| Cop | NGSLNYYVLYKGSDPNCADEDELTSLHYYCKHISTFYKSNYYKLSHTKMRAEKRFIYAII | 240 |
| WR | ------------------------------------------------------------ | |
| Wyeth | ------------------------------------------------------------ | |
| Cop | DYGANINAVTHLPSTVYQT | 259 |
| WR | ------------------- | |
| Wyeth | ------------------- | |

B26R
CLUSTAL O(1.2.4) multiple sequence alignment

| | | |
|---|---|---|
| Cop | MEQTLTRLHTYLQQYTKHSPRVVYALLSRGYVIILIVHPSWNDCATGHILIMLLNWHEQK | 60 |
| WR | -------------------MLFYLEEPIRGYVIILIVHPSWNDCATGHILIMLLNWHEQK | 41 |
| Wyeth | MEQTLTRLHTYLQQYTKHSPRVVYALLSRGYVIILIVHPSWNDCATGHILIMLLNWHEQK | 60 |
| Lister | MEQTLTRLHTYLQQYTKHSPRVVYALLSRGYVIILIVHPSWNDCATGHILIMLLNWHEQK | 60 |
| | . ******************************** | |
| Cop | EEGQHLLYLFIKHNQGYTLNILRYLLDRFDIQKDEYIYRLSKL----------------- | 103 |
| WR | EEGQHLLYLFIKHNQGYTLNILRYLLDRFDIQKDEYYNTAFQNCNNNVASYIGYDINLPT | 101 |
| Wyeth | EEGQHLLYLFIKHNQGYTLNILRYLLDRFDIQKDEYYNTAFQNCNNNVASYIGYDINLPT | 120 |
| Lister | EEGQHLLYLFIKHNQGYTLNILRYLLDRFDIQKDEYYNTAFQNCNNNVASYIGYDINLPT | 120 |
| | ************************************ : | |
| Cop | -------- | |
| WR | KDGIRLGV | 109 |
| Wyeth | KDGIRLGV | 128 |
| Lister | KDGIRLGV | 128 |

B27R
CLUSTAL O(1.2.4) multiple sequence alignment

| | | |
|---|---|---|
| Cop | MLPHTSDTTSTFRLKTVFDLVFENRNIIYKADVVNDIIKHRLKVSLPMIKSLFYKMSEFS | 60 |
| WR | MLPHTSDTTSTFRLKTVFDLVFENRNIIYKADVVNDIIHHRLKVSLPMIKSLFYKMSLPT | 60 |
| Wyeth | MLPHTSDTTSTFRLKTVFDLVFENRNIIYKADVVNDIIHHRLK--VPMIKSLFYKMSEFS | 58 |
| Lister | MLPHTSDTTSTFRLKTVFDLVFENRNIIYKADVVNDIIHHRLKVSLPMIKSLFYKMSLPT | 60 |
| | ******************************************* :*********** : | |
| Cop | PYDDYYVKKILAYCLLRDESFAELHSKFCLNEDYKSVFMKNISFDKIDSIIVT | 113 |
| WR | TITT------------------------------------------------- | 64 |
| Wyeth | PYDDYYVKKILAYCLLRDESFAELHSKFCLNEDYKSVFMKNISFDKIDSIIVT | 111 |
| Lister | TITT------------------------------------------------- | 64 |

B28R
CLUSTAL O(1.2.4) multiple sequence alignment

| | | |
|---|---|---|
| Cop | MKSVLYSYILFLSCIIINGRDIAPHAPSDGKCKDNEYKRHNLCPGTYASALCDSKTNTQC | 60 |
| WR | MKSVLYSYILELSCIIINGRDIAPHAPSDGKCKDNEYKRHNLCPGTYASRLCDSKTNTQC | 60 |
| Wyeth | -----------------------MHHPMESVKTTN--TNAIICV---REHTLPDYANTQC | 32 |
| | * * :. . * .. :* .: . :**** | |
| Cop | TPCGSGTFTSRNNHLPACLSCNGRRDRVTLLTIESVNALPDIIVFSKDHPDARHVFPKQN | 120 |
| WR | TPCGSGTFTSRNNHLPACLSCNGRRDRVTRLTIESVNALPDIIVFSKDHPDARHVFPKQN | 120 |
| Wyeth | TPCGSGTFTSRNNHLPACLSCNGRRDRVTLLTIESVNALPDIIVFSKDHPDARHVFPKQN | 92 |
| | ***************************** ****************************** | |
| Cop | VE | 122 |
| WR | VE | 122 |
| Wyeth | V- | 93 |
| | * | |

C23L/B29R
CLUSTAL O(1.2.4) multiple sequence alignment

| | | |
|---|---|---|
| Cop | --------------MHVPASLQQSSSSSSSCTEEENKHHMGIDVIIKVTKQDQTPTNDKI | 46 |
| WR | --------------MHVPASLQQSSSSSSSCTEEENKHHMGIDVIIKVTKQDQTPTNDKI | 46 |
| Tian | --------------MHVPASLQQSSSSSSSCTEEENKHHMGIDVIIKVTKQDQTPTNDKI | 46 |
| Wyeth | --------------MHVPASLQQ---SSSSCTEEENKHHMGIDVIIKVTKQDQTPTNDKI | 43 |
| Lister | MKQYIVLACMCLAAAAMPASLQQSSSSSSSCTEEENKHHMGIDVIIKVTKQDQTPTNDKI | 60 |
| | :****** ********************************** | |
| Cop | CQSVTEITESESDPDPEVESEDDSTSVEDVDPPTTYYSIIGGGLRMNFGFTKCPQIKSIS | 106 |
| WR | CQSVTEITESESDPDPEVESEDDSTSVEDVDPPTTYYSIIGGGLRMNFGFTKCPQIKSIS | 106 |
| Tian | CQSVTEITESESDPDPEVESEDDSTSVEDVDPPTTYYSIIGGGLRMNFGFTKCPQIKSIS | 106 |
| Wyeth | CQSVTEITESESDPDPEVESEDDSTSVEDVDIPTTYYSIIGGGLRMNFGFTKCPQIKSIS | 103 |
| Lister | CQSVTEITESESDPDPEVESEDDSTSVEDVDPPTTYYSIIGGGLRMNFGFTKCPQIKSIS | 120 |
| | ******************************* **************************** | |
| Cop | ESADGNTVNARLSSVSPGQGKDSPAITREEALAMIKDCEVSIDIRCSEEEKDSDIKTHPV | 166 |
| WR | ESADGNTVNARLSSVSPGQGKDSPAITHEEALAMIKDCEVSIDIRCSEEEKDSDIKTHPV | 166 |
| Tian | ESADGNTVNARLSSVSPGQGKDSPAITHEEALAMIKDCEVSIDIRCSEEEKDSDIKTHPV | 166 |
| Wyeth | ESADGNTVNARLSSVSPGQGKDSPAITHEEALAMIKDCEVSIDIRCSEEEKDSDIKTHPV | 163 |
| Lister | ESADGNTVNARLSSVSPGQGKDSPAITHEEALAMIKDCEVSIDIRCSEEEKDSDIKTHPV | 180 |
| | ***************************:******************************** | |
| Cop | LGSNISHKKVSYEDIIGSTIVDTKCVKNLEFSVRIGDMCKESSELEVKDGFKYVDGSASE | 226 |
| WR | LGSNISHKKVSYEDIIGSTIVDTKCVKNLEFSVRIGDMCKESSELEVKDGFKYVDGSASE | 226 |
| Tian | LGSNISHKKVSYEDIIGSTIVDTKCVENLEFSVRIGDMCKESSELEVKDGFKYVDGSASE | 226 |
| Wyeth | LGSNISHKKVSYEDIIGSTIVDTKCVKNLEFSVRIGDMCKESSELEVKDGFKYVDGSASE | 223 |
| Lister | LGSNISHKKVSYEDIIGSTIVDTKCVKNLEFSVRIGDMCKESSELEVKDGFKYVDGSASE | 240 |
| | ************************************************************ | |
| Cop | GATDDTSLIDSTKLKACV | 244 |
| WR | GATDDTSLIDSTKLKACV | 244 |
| Tian | GATDDTSLIDSTKLKACV | 244 |
| Wyeth | GATDDTSLIDSTKLKACV | 241 |
| Lister | GATDDTSLIDSTKLKACV | 258 |
| | ****************** | |

### Assays for Measuring Virus Characteristics

Assays known in the art to measure the tumor spreading and virulence of a virus include but are not limited to measuring plaque size, syncytia formation, and/or comet assays (EEVs). Assays known in the art to measure the immunostimulatory activity of a virus include but are not limited to NK activation (measured in % CD69 expression), NK degranulation (measured in fold increase of CD107a), and/or T-cell priming assays. Assays known in the art to measure the selectivity of a virus include, but are not limited to, tail pox lesions, biodistribution, and/or body mass measurements.

### Examples of Proteins Encoded by Orthopoxvirus Genes

Exemplary proteins encoded by orthopoxvirus genes described in this disclosure are reproduced below in Tables 31-40. As used below, the term “location” refers to the location of the gene with respect to the deleted nucleic acids in exemplary orthopoxvirus vectors described herein. For various genes, amino acid sequence information and protein accession ID numbers are provided.

TABLE 31
Examples of proteins encoded by Copenhagen Vaccinia genes deleted in CopMD5p vector

| SEQ ID | | Protein | | |
|---|---|---|---|---|
| NO. | Gene | Accession ID | Amino Acid Sequence | Location |
| SEQ ID | C2L | AAA47999.1 | MESVIFSINGEIIQVNKEIITASPYNFFKRIQDHHLKD | Inside |
| NO: 23 | (26% 5′) | | EAIILNGINYHAFESLLDYIRWKKINITINNVEMILVA | Deletion |
| | | | AIIIDVPPVVDLCVKTMIHNINSTNCIRMFNFSKRYGI | |
| | | | KKLYNASMSEIINNITAVTSDPEFGKLSKDELTTILS | |
| | | | HENVNVNHEDVTAMILLKWIHKNPNDVDIINILHPK | |
| | | | FMTNTMRNAISLLGLTISKSTKPVTRNGIKHNIVVIK | |
| | | | NSDYISTITHYSPRTEYWTIVGNTDRQFYNANVLHN | |
| | | | CLYIIGGMINNRHVYSVSRVDLETICKWKTVTNMSS | |
| | | | LKSEVSTCVNDGKLYVIGGLEFSISTGVAEYLKHGT | |
| | | | SKWIRLPNLITPRYSGASVFVNDDIYVMGGVYTTYE | |
| | | | KYVVLNDVECFTKNRWIKKSPMPRHHSIVYAVEYD | |
| | | | GDIYVITGITHETRNYLYKYIVKEDKWIELYMYFNH | |
| | | | VGKMFVCSCGDYILIIADAKYEYYPKSNTWNLFDM | |
| | | | STRNIEYYDMFTKDETPKCNVTHKSLPSFLSNCEKQ | |
| | | | FLQ | |
| SEQ ID | C1L | AAA48000.1 | MVKNNKISNSCRMIMSTNPNNILMRHLKNLTDDEF | Inside |
| NO: 24 | | | KCIIHRSSDFLYLSDSDYTSITKETLVSEIVEEYPDDC | Deletion |
| | | | NKILAIIFLVLDKDIDVDIETKLKPKPAVRFAILDKM | |
| | | | TEDIKLTDLVRHYFRYIEQDIPLGPLFKKIDSYRTRAI | |
| | | | NKYSKELGLATEYFNKYGHLMFYTLPIPYNRFFCRN | |
| | | | SIGFLAVLSPTIGHVKAFYKFIEYVSIDDRRKFKKEL | |
| | | | MSK | |
| SEQ ID | N1L | AAA48001.1 | MRTLLIRYILWRNDNDQTYYNDDFKKLMLLDELVD | Inside |
| NO: 25 | | | DGDVCTLIKNMRMTLSDGPLLDRLNQPVNNIEDAK | Deletion |
| | | | RMIAISAKVARDIGERSEIRWEESFTILFRMIETYFDD | |
| | | | LMIDLYGEK | |
| SEQ ID | N2L | AAA48002.1 | MTSSAMDNNEPKVLEMVYDATILPEGSSMDPNIMD | Inside |
| NO: 26 | | | CINRHINMCIQRTYSSSIIAILNRFLTMNKDELNNTQ | Deletion |
| | | | CHIIKEFMTYEQMAIDHYGEYVNAILYQIRKRPNQH | |
| | | | HTIDLFKKIKRTPYDTFKVDPVEFVKKVIGFVSILNK | |
| | | | YKPVYSYVLYENVLYDEFKCFINYVETKYF | |
| SEQ ID | M1L | AAA48003.1 | MIFVIESKLLQIYRNRNRNINFYTTMDNIMSAEYYLS | Inside |
| NO: 27 | | | LYAKYNSKNLDVFRNMLQAIEPSGNNYHILHAYCG | Deletion |
| | | | IKGLDERFVEELLHRGYSPNETDDDGNYPLHIASKIN | |
| | | | NNRIVAMLLTHGADPNACDKHNKSVDTPLYYLSGTDDE | |
| | | | VIERINLLVQYGAKINNSVDEEGCGPLLACTDPSER | |
| | | | VFKKIMSIGFEARIVDKFGKNHIHRHLMSDNPKASTI | |
| | | | SWMMKLGISPSKPDHDGNTPLHIVCSKTVKNVDIID | |
| | | | LLLPSTDVNKQNKFGDSPLTLLIKTLSPAHLINKLLS | |
| | | | TSNVITDQTVNICIFYDRDDVLEIINDKGKQYDSTDF | |
| | | | KMAVEVGSIRCVKYLLDNDIICEDAMYYAVLSEYE | |
| | | | TMVDYLLFNHFSVDSVVNGHTCMSECVRLNNPVIL | |
| | | | SKLMLHNPTSETMYLTMKAIEKDKLDKSIIIPFIAYF | |
| | | | VLMHPDFCKNRRYFTSYKRFVTDYVHEGVSYEVFD | |
| | | | DYF | |
| SEQ ID | M2L | AAA48004.1 | MVYKLVLLFCIASLGYSVEYKNTICPPRQDYRYWY | Inside |
| NO: 28 | | | FAAELTIGVNYDINSTIIGECHMSESYIDRNANIVLTG | Deletion |
| | | | YGLEINMTIMDTDQRFVAAAEGVGKDNICLSVLLFT | |
| | | | TQRLDKVHHNISVTITCMEMNCGTTKYDSDLPESIH | |
| | | | KSSSCDITINGSCVTCVNLETDPTKINPHYLHPKDKY | |
| | | | LYHNSEYGMRGSYGVTFIDELNQCLLDIKELSYDIC | |
| | | | YRE | |
| SEQ ID | HR/K1L | AAA48005.1 | MDLSRINTWKSKQLKSFLSSKDTFKADVHGHSALY | Inside |
| NO: 29 | | | YAIADNNVRLVCTLLNAGALKNLLENEFPLHQAAT | Deletion |
| | | | LEDTKIVKILLFSGMDDSQFDDKGNTALYYAVDSG | |
| | | | NMQTVKLFVKKNWRLMFYGKTGWKTSFYHAVML | |
| | | | NDVSIVSYFLSEIPSTFDLAILLSCIHTTIKNGHVDMM | |
| | | | ILLLDYMTSTNTNNSLLFIPDIKLAIDNKDIEMLQAL | |
| | | | FKYDINIYSVNLENVLLDDAEITKMIIEKHVEYKSDS | |
| | | | YTKDLDIVKNNKLDEIISKNKELRLMYVNCVKKN | |
| SEQ ID | SPI- | AAA48006.1 | MIALLILSLTCSVSTYRLQGFTNAGIVAYKNIQDDNI | Inside |
| NO: 30 | 3/K2L | | VFSPFGYSFSMFMSLLPASGNTRIELLKTMDLRKRD | Deletion |
| | | | LGPAFTELISGLAKLKTSKYTYTDLTYQSFVDNTVCI | |
| | | | KPLYYQQYHRFGLYRLNFRRDAVNKINSIVERRSG | |
| | | | MSNVVDSNMLDNNTLWAIINTIYFKGTWQYPFDIT | |
| | | | KTRNASFTNKYGTKTVPMMNVVTKLQGNTITIDDE | |
| | | | EYDMVRLPYKDANISMYLAIGDNMTHFIDSITAAK | |
| | | | LDYWSFQLGNKVYNLKLPKFSIENKRDIKSIAEMM | |
| | | | APSMFNPDNASFKHMTRDPLYIYKMFQNAKIDVDE | |
| | | | QGTVAEASTIMVATARSSPEKLEFNTPFVFIIRHDITG | |
| | | | FILFMGKVESP | |
| SEQ ID | K ORF A | AAA48007.1 | MGHIITYCQVHTNISILIRKAHHIIFFVIDCDCISLQFS | Inside |
| NO: 31 | | | NYVHHGNRFRTVLISKTSIACFSDIKRILPCTFKIYSI | Deletion |
| | | | NDCP | |
| SEQ ID | K ORF B | AAA48008.1 | MGTVFVPYLLVKLALRVLVISNGYCHVPLKYIVLMI | Inside |
| NO: 32 | | | AHRVLLSSILESTTLDIPDLRSTIELILLTASRLICFNLY | Deletion |
| | | | RPNL | |
| SEQ ID | K ORF B | AAA48009.1 | MLAFCYSLPNAGDVIKGRVYEKDYALYIYLFDYPH | Inside |
| NO: 33 | | | SEAILAESVICMHMDRYVEYRDKLVGKTVKVKVIR | Deletion |
| | | | VDYTKGYIDVNYKRMCRHQ | |
| SEQ ID | K4L | AAA48010.1 | MNPDNTIAVITETIPIGMQFDKVYLSTFNMWREILSN | Inside |
| NO: 34 | | | TTKTLDISSFYWSLSDEVGTNFGTIILNEIVQLPKRG | Deletion |
| | | | VRVRVAVNKSNKPLKDVERLQMAGVEVRYIDITNI | |
| | | | LGGVLHTKFWISDNTHIYLGSANMDWRSLTQVKEL | |
| | | | GIAIFNNRNLAADLTQIFEVYWYLGVNNLPYNWKN | |
| | | | FYPSYYNTDHPLSINVSGVPHSVFIASAPQQLCTME | |
| | | | RTNDLTALLSCIRNASKFVYVSVMNFIPIIYSKAGKI | |
| | | | LFWPYIEDELRRSAIDRQVSVKLLISCWQRSSFIMRN | |
| | | | FLRSIAMLKSKNIDIEVKLFIVPDADPPIPYSRVNHAK | |
| | | | YMVTDKTAYIGTSNWTGNYFTDTCGASINITPDDGL | |
| | | | GLRQQLEDIFMRDWNSKYSYELYDTSPTKRCKLLK | |
| | | | NMKQCTNDIYCDEIQPEKEIPEYSLE | |
| SEQ ID | K5L | AAA48011.1 | MGATISILASYDNPNLFTAMILMSPLVNADAVSRLN | Inside |
| NO: 35 | | | LLAAKLMGTITPNAPVGKLCPESVSRDMDKVYKYQ | Deletion |
| | | | YDPLINHEKIKAGFASQVLKATNKVRKIISKINTPRL | |
| | | | SYSREQTMRLVMFQVHIISCNMQIVIE | |
| SEQ ID | K6L | AAA48012.1 | MSANCMFNLDNDYIYWKPITYPKALVFISHGAGKH | Inside |
| NO: 36 | | | SGRYDELAENISSLGILVFSHDHIGHGRSNGEKMMI | Deletion |
| | | | DDFGTARGNY | |
| SEQ ID | K7R | AAA48013.1 | MATKLDYEDAVFYFVDDDKICSRDSIIDLIDEYITW | Inside |
| NO: 37 | | | RNHVIVFNKDITSCGRLYKELMKFDDVAIRYYGIDK | Deletion |
| | | | INEIVEAMSEGDHYINFTKVHDQESLFATIGICAKITE | |
| | | | HWGYKKISESRFQSLGNITDLMTDDNINILILFLEKK | |
| | | | LN | |
| SEQ ID | F1L | AAA48014.1 | MLSMFMCNNIVDYVDDIDNGIVQDIEDEASNNVDH | Inside |
| NO: 38 | | | DYVYPLPENMVYRFDKSTNILDYLSTERDHVMMA | Deletion |
| | | | VRYYMSKQRLDDLYRQLPTKTRSYIDIINIYCDKVS | |
| | | | NDYNRDMNIMYDMASTKSFTVYDINNEVNTILMD | |
| | | | NKGLGVRLATISFITELGRRCMNPVKTIKMFTLLSHT | |
| | | | ICDDCFVDYITDISPPDNTIPNTSTREYLKLIGITAIMF | |
| | | | ATYKTLKYMIG | |
| SEQ ID | DUT/F2L | AAA48015.1 | MFNMNINSPVRFVKETNRAKSPTRQSPGAAGYDLY | Inside |
| NO: 39 | | | SAYDYTIPPGERQLIKTDISMSMPKICYGRIAPRSGLS | Deletion |
| | | | LKGIDIGGGVIDEDYRGNIGVILINNGKCTFNVNTGD | |
| | | | RIAQLIYQRIYYPELEEVQSLDSTNRGDQGFSTGLR | |
| SEQ ID | F3L | AAA48016.1 | MPIFVNTVYCKNILALSMTKKFKTIIDAIGGNIIVNST | Inside |
| NO: 40 | (75% 3′) | | ILKKLSPYFRTHLRQKYTKNKDPVTRVCLDLDIHSL | Deletion |
| | | | TSIVIYSYTGKVYIDSHNVVNLLRASILTSVEFITYTCI | |
| | | | NFILRDFRKEYCVECYMMGIEYGLSNLLCHTKNFIA | |
| | | | KHFLELEDDIIDNFDYLSMKLILESDELNVPDEDYV | |
| | | | VDFVIKWYIKRRNKLGNLLLLIKNVIRSNYLSPRGIN | |
| | | | NVKWILDCTKIFHCDKQPRKSYKYPFIEYPMNMDQI | |
| | | | IDIFHMCTSTHVGEVVVYLIGGWMNNEIHNNAIAVN | |
| | | | YISNNWIPIPPMNSPRLYATGIPANNKLYVVGGLPNP | |
| | | | TSVERWFHGDAAWVNMPSLLKPRCNPAVASINNVI | |
| | | | YVMGGHSETDTTTEYLLPNHDQWQFGPSTYYPHY | |
| | | | KSCALVFGRRLFLVGRNAEFYCESSNTWTLIDDPIY | |
| | | | PRDNPELIIVDNKLLLIGGFYRGSYIDTIEVYNHHTY | |
| | | | SWNIWDGK | |

TABLE 32
Examples of proteins encoded by Western Reserve Vaccinia genes equivalent
to those deleted in CopMD5p vector

| SEQ ID | | Protein | | |
|---|---|---|---|---|
| NO. | Gene | Accession ID | AA Sequence | Location |
| SEQ ID | VACWR026 | AAO89305.1 | MESVIFSINGEIIQVNKEIITASPYNFFKRIQDHHLKD | Inside |
| NO: 41 | (26% 5′) | | EAIILNGINYHAFESLLDYIRWKKINITINNVEMILVA | Deletion |
| | | | AIIIDVPPVVDLCVKTMIHNINSTNCIRMFNFSKRYGI | |
| | | | KKLYNASMSEIINNITAVTSDPEFGKLSKDELTTILS | |
| | | | HENVNVNHEDVTAMILLKWIHKNPNDVDIINILHPK | |
| | | | FMTNTMRNAISLLGLTISKSTKPVTRNGIKHNIVVIK | |
| | | | NSDYISTITHYSPRTEYWTIVGNTDRQFYNANVLHN | |
| | | | CLYIIGGMINNRHVYSVSRVDLETKKWKTVTNMSS | |
| | | | LKSEVSTCVNDGKLYVIGGLEFSISTGVAEYLKHGT | |
| | | | SKWIRLPNLITPRYSGASVFVNDDIYVMGGVYTTYE | |
| | | | KYVVLNDVECFTKNRWIKKSPMPRHHSIVYAVEYD | |
| | | | GDIYVITGITHETRNYLYKYIVKEDKWIELYMYFNH | |
| | | | VGKMFVCSCGDYILIIADAKYEYYPKSNTWNLFDM | |
| | | | STRNIEYYDMFTKDETPKCNVTHKSLPSFLSNCEKQ | |
| | | | FLQ | |
| SEQ ID | VACWR027 | AAO89306.1 | MVKNNKIQKNKISNSCRMIMSTDPNNILMRHLKNL | Inside |
| NO: 42 | | | TDDEFKCIIHRSSDFLYLSDSDYTSITKETLVSEIVEE | Deletion |
| | | | YPDDCNKILAIIFLVLDKDIDVDIKTKLKPKPAVRFAI | |
| | | | LDKMTEDIKLTDLVRHYFRYIEQDIPLGPLFKKIDSY | |
| | | | RTRAINKYSKELGLATEYFNKYGHLMFYTLPIPYNR | |
| | | | FFCRNSIGFLAVLSPTIGHVKAFYKFIEYVSIDDRRKF | |
| | | | KKELMSK | |
| SEQ ID | VACWR028 | AAO89307.1 | MRTLLIRYILWRNDNDQTYYNDNFKKLMLLDELVD | Inside |
| NO: 43 | | | DGDVCTLIKNMRMTLSDGPLLDRLNQPVNNIEDAK | Deletion |
| | | | RMIAISAKVARDIGERSEIRWEESFTILFRMIETYFDD | |
| | | | LMIDLYGEK | |
| SEQ ID | VACWR029 | AAO89308.1 | MTSSAMDNNEPKVLEMVYDATILPEGSSMDPNIMD | Inside |
| NO: 44 | | | CINRHINMCIQRTYSSSIIAILDRFLMMNKDELNNTQ | Deletion |
| | | | CHIIKEFMTYEQMAIDHYGGYVNAILYQIRKRPNQH | |
| | | | HTIDLFKRIKRTRYDTFKVDPVEFVKKVIGFVSILNK | |
| | | | YKPVYSYVLYENVLYDEFKCFINYVETKYF | |
| SEQ ID | VACWR030 | AAO89309.1 | MIFVIESKLLQIYRNRNRNINFYTTNIDNIMSAEYYLS | Inside |
| NO: 45 | | | LYAKYNSKNLDVFRNMLQAIEPSGNNYHILHAYCG | Deletion |
| | | | IKGLDERFVEELLHRGYSPNETDDDGNYPLHIASKIN | |
| | | | NNRIVAMLLTHGADPNACDKHNKTPLYYLSGTDDE | |
| | | | VIERINLLVQYGAKINNSVDEEGCGPLLACTDPSER | |
| | | | VFKKIMSIGFEARIVDKFGKNHIHRHLMSDNPKASTI | |
| | | | SWMMKLGISPSKPDHDGNTPLHIVCSKTVKNVDIID | |
| | | | LLLPSTDVNKQNKFGDSPLTLLIKTLSPAHLINKLLS | |
| | | | TSNVITDQTVNICIFYDRDDVLEIINDKGKQYDSTDF | |
| | | | KMAVEVGSIRCVKYLLDNDIICEDAMYYAVLSEYE | |
| | | | TMVDYLLFNHFSVDFVVNGHTCMSECVRLNNPVIL | |
| | | | SKLMLHNPTSETMYLTMKAIEKDRLDKSIIIPFIAYF | |
| | | | VLMHPDFCKNRRYFTSYKRFVTDYVHEGVSYEVFD | |
| | | | DYF | |
| SEQ ID | VACWR031 | AAO89310.1 | MVYKLVLLFCIASLGYSVEYKNTICPPRQDYRYWY | Inside |
| NO: 46 | | | FAAELTIGVNYDINSTIIGECHMSESYIDRNANIVLTG | Deletion |
| | | | YGLEINMTIMDTDQRFVAAAEGVGKDNKLSVLLFT | |
| | | | TQRLDKVHHNISVTITCMEMNCGTTKYDSDLPESIH | |
| | | | KSSSCDITINGSCVTCVNLETDPTKINPHYLHPKDKY | |
| | | | LYHNSEYSMRGSYGVTFIDELNQCLLDIKELSYDIC | |
| | | | YRE | |
| SEQ ID | VACWR032 | AAO89311.1 | MDLSRINTWKSKQLKSFLSSKDAFKADVHGHSALY | Inside |
| NO: 47 | | | YAIADNNVRLVCTLLNAGALKNLLENEFPLHQAAT | Deletion |
| | | | LEDTKIVKILLFSGLDDSQFDDKGNTALYYAVDSGN | |
| | | | MQTVKLFVKKNWRLMFYGKTGWKTSFYHAVMLN | |
| | | | DVSIVSYFLSEIPSTFDLAILLSCIHITIKNGHVDMMIL | |
| | | | LLDYMTSTNTNNSLLFIPDIKLAIDNICDIEMLQALFK | |
| | | | YDINIYSANLENVLLDDAEIAKMIIEKHVEYKSDSYT | |
| | | | KDLDIVKNNKLDEIISKNKELRLMYVNCVKKN | |
| SEQ ID | SPI-3 | AAO89312.1 | MIALLILSLTCSVSTYRLQGFTNAGIVAYKNIQDDNI | Inside |
| NO: 48 | | | VFSPFGYSFSMFMSLLPASGNTRIELLKTMDLRKRD | Deletion |
| | | | LGPAFTELISGLAKLKTSKYTYTDLTYQSFVDNTVCI | |
| | | | KPSYYQQYHRFGLYRLNFRRDAVNKINSIVERRSG | |
| | | | MSNVVDSNMLDNNTLWAIINTIYFKGIWQYPFDITK | |
| | | | TRNASFTNKYGTKTVPMMNVVTKLQGNTITIDDEE | |
| | | | YDMVRLPYKDANISMYLAIGDNMTHFTDSITAAKL | |
| | | | DYWSFQLGNKVYNLKLPKFSIENKRDIKSIAEMMAP | |
| | | | SMFNPDNASFKHMTRDPLYIYKMFQNAKIDVDEQG | |
| | | | TVAEASTIMVATARSSPEKLEFNTPFVFIIRHDITGFI | |
| | | | LFMGKVESP | |
| SEQ ID | VACWR034 | AAO89313.1 | MLAFCYSLPNAGDVIKGRVYEKDYALYIYLFDYPH | Inside |
| NO: 49 | | | FEAILAESVKMHMDRYVEYRDKLVGKTVKVKVIR | Deletion |
| | | | VDYTKGYIDVNYKRMCRHQ | |
| SEQ ID | VACWR035 | AAO89314.1 | MNPDNTIAVITETIPIGMQFDKVYLSTFNMWREILSN | Inside |
| NO: 50 | | | TTKTLDISSFYWSLSDEVGTNFGTIILNKIVQLPKRG | Deletion |
| | | | VRVRVAVNKSNKPLKDVERLQMAGVEVRYIDITNI | |
| | | | LGGVLHTKFWISDNTHIYLGSANMDWRSLTQVKEL | |
| | | | GIAIFNNRNLAADLTQIFEVYWYLGVNNLPYNWKN | |
| | | | FYPSYYNTDHPLSINVSGVPHSVFIASAPQQLCTME | |
| | | | RTNDLTALLSCIRNASKFVYVSVMNFIPIIYSKAGNI | |
| | | | LFWPYIEDELRRAAIDRQVSVKLLISCWQRSSFIMRN | |
| | | | FLRSIAMLKSKNINIEVKLFIVPDADPPIPYSRVNHAK | |
| | | | YMVTDKTAYIGTSNWTGNYFTDTCGASINITPDDGL | |
| | | | GLRQQLEDIFMRDWNSKYSYELYDTSPTKRCRLLK | |
| | | | NMKQCINDIYCDEIQPEKEIPEYSLE | |
| SEQ ID | VACWR036 | AAO89315.1 | MQHANCNREIKIYEGAKHHLHKETDEVKKSVMKEI | Outside |
| NO: 51 | | | ETWIFNRVK | Deletion |
| SEQ ID | VACWR037 | AAO89316.1 | MTLVQHVVTIKSTYWVIPWELASYDNPNLFTAMIL | Inside |
| NO: 52 | | | MSPLVNADAVSKLNLLAAKLMGTITLNAPVGKLCP | Deletion |
| | | | ESVSRDMDKVYKYQYDPLINHEKIKAGFASQVLKA | |
| | | | TNKVRKIISKINTPRLSYSREQTIRLAMF | |
| SEQ ID | VACWR038 | AAO89317.1 | MSANCMFNLDNDYIYWKPITYPKALVFISHGAGKH | Inside |
| NO: 53 | | | SGRYDELAENISSLGILVFSHDHIGHGRSNGEKMMI | Deletion |
| | | | DDFGTARGNY | |
| SEQ ID | VACWR039 | AAO89318.1 | MATKLDYEDAVFYFVDDDKICSRDSIIDLIDEYITW | Inside |
| NO: 54 | | | RNHVIVFNKDITSCGRLYKELMKFDDVAIRYYGIDK | Deletion |
| | | | INEIVEAMSEGDHYINFTKVHDQESLFATIGICAKITE | |
| | | | HWGYKKISESRFQSLGNITDLMTDDNINILILFLEKK | |
| | | | LN | |
| SEQ ID | VACWR040 | AAO89319.1 | MLSMFMCNNIVDYVDDIDNGIVQDIEDEASNNVDH | Inside |
| NO: 55 | | | DYVYPLPENMVYRFDKSTNILDYLSTERDHVMMA | Deletion |
| | | | VRYYMSKQRLDDLYRQLPTKTRSYIDIINIYCDKVS | |
| | | | NDYNRDMNIMYDMASTKSFTVYDINNEVNTILMD | |
| | | | NKGLGVRLATISFITELGRRCMNPVETIKMFTLLSHT | |
| | | | ICDDYFVDYITDISPPDNTIPNTSTREYLKLIGITAIMF | |
| | | | ATYKTLKYMIG | |
| SEQ ID | DUT | AAO89320.1 | MFNMNINSPVRFVKETNRAKSPTROQSPYAAGYDLY | Inside |
| NO: 56 | | | SAYDYTIPPGERQLIKTDISMSMPKFCYGRIAPRSGL | Deletion |
| | | | SLKGIDIGGGVIDEDYRGNIGVILINNGKCTFNVNTG | |
| | | | DRIAQLIYQRIYYPELEEVQSLDSTNRGDQGFGSTGLR | |
| SEQ ID | VACWR042 | AAO89321.1 | MPIFVNTVYCKNILALSMTKKFKTIIDAIGGNIIVNST | Inside |
| NO: 57 | (75% 3′) | | ILKKLSPYFRTHLRQKYTKNKDPVTWVCLDLDIHSL | Deletion |
| | | | TSIVIYSYTGKVYIDSHNVVNLLRASILTSVEFIIYTCI | |
| | | | NFILRDFRKEYCVECYMMGIEYGLSNLLCHTKNFIA | |
| | | | KHFLELEDDIIDNFDYLSMKLILESDELNVPDEDYV | |
| | | | VDFVIKWYIKRRNKLGNLLLLIKNVIRSNYLSPRGIN | |
| | | | NVKWILDCTKIFHCDKQPRKSYKYPFIEYPMNMDQI | |
| | | | IDIFHMCTSTHVGEVVYLIGGWMNNEIHNNAIAVN | |
| | | | YISNNWIPIPPMNSPRLYASGIPANNKLYVVGGLPNP | |
| | | | TSVERWFHGDAAWVNMPSLLKPRCNPAVASINNVI | |
| | | | YVMGGHSETDTTTEYLLPNHDQWQFGPSTYYPHY | |
| | | | KSCALVFGRRLFLVGRNAEFYCESSNTWTLIDDPIY | |
| | | | PRDNPELIIVDNKLLLIGGFYRESYIDTIEVYNHHTYS | |
| | | | WNIWDGK | |

TABLE 33
Examples of proteins encoded by Tian Tan Vaccinia genes
equivalent to those deleted in CopMD5p vector

| SEQ ID | | Protein | | |
|---|---|---|---|---|
| NO | Gene | Accession ID | AA Sequence | Location |
| SEQ ID | TC2L | AAF33878.1 | MESVIFSINGEIIQVNKEIITASPYNFFKRIQDHHLKD | Inside |
| NO: 58 | (26% 5′) | | EAIILNGINYHAFESLLDYIRWKKINITINNVEMILVA | Deletion |
| | | | AIIIDVPPVVDLCVKTMIHNINSTNCIRMFNFSKQYGI | |
| | | | KKLYNASMSEIINNITAVTSDPEFGKLSKDELTTILS | |
| | | | HEDVNVNHEDVTAMILLKWIHKNPNDVDIINILHPK | |
| | | | FMTNTMRNAISLLGLTISKSTKPVTRNGIKHNIVVIK | |
| | | | NSDYISTITHYSPRTEYWTIVGNTDRQFYNANVLHN | |
| | | | CLYIIGGMINNRHVYSVSRVDLETKKWKTVTNMSS | |
| | | | LKSEVSTCVNNGKLYVIGGLEFSISTGVAEYLKHGT | |
| | | | SKWIRLPNLITPRYSGASVFVNDDIYVMGGVYTTYE | |
| | | | KYVVLNDVECFTKNRWIKKSPMPRHHSIVYAVEYD | |
| | | | GDIYVITGITHETRNYLYKYIVKEDKWIELYMYFNH | |
| | | | VGKMFVCSCGDYILIIADAKYEYYPKSNTWNLFDM | |
| | | | STRNIEYYDMFTKDETPKCNVTHKSLPSFLSNCEKQ | |
| | | | FLQ | |
| SEQ ID | TC1L | AAF33879.1 | MVKNNKISNSCRMIMSTDPNNILMRHLKNLTDDEF | Inside |
| NO: 59 | | | KCIIHRSSDFLYLSDSDYTSITKETLVSEIVEEYPDDC | Deletion |
| | | | NKILAIIFLVLDKDIDVDIKTKLKPKPAVRFAILDKM | |
| | | | TEDIKLTDLVRHYFRYIEQDIPLGPLFKKIDSYRTRAI | |
| | | | NKYSKELGLATEYFNKYGHLMFYTLPIPYNRFFCRN | |
| | | | SIGFLAVLSPTIGHVKAFYKFIEYVSIDDRRKFKKEL | |
| | | | MSK | |
| SEQ ID | TN1L | AAF33880.1 | MRTLLIRYILWRNDNDQTYYNDDFKKLMLLDELVD | Inside |
| NO: 60 | | | DGDVCTLIKNMRMTLSDGPLLDRLNQPVNNIEDAK | Deletion |
| | | | RMIAISAKVARDIGERSEIRWEESFTILFRMIETYFDD | |
| | | | LMIDLYGEK | |
| SEQ ID | TN2L | AAF33881.1 | MTSSAMDNNEPKVLEMVYDATILPEGSSMDPYIMD | Inside |
| NO: 61 | | | CINRHINMCIQRTYSSSIIAILDRFLMMNKDELNNTQ | Deletion |
| | | | CHIIKNL | |
| SEQ ID | TM1L | AAF33882.1 | MIFVIESKLLQIYRNRNRNINFYTTMDNIMSAEYYLS | Inside |
| NO: 62 | | | LYAKYNSKNLDVFRNMLQAIEPSGNNYHILHAYCG | Deletion |
| | | | IKGLDERFVEELLHRGYSPNETDDDGNYPLHIASKIN | |
| | | | NNRIVAMLLTHGADPNACDKHNKTPLYYLSGTDDE | |
| | | | VIERINLLVQYGAKINNSVDEEGCGPLLACTDPSER | |
| | | | VFKKIMSIGFEARIVDKFGKNHIHRHLMSDNPKASTI | |
| | | | SWMMKLGISPSKPDHDGNTPLHIVCSKTVKNVDIID | |
| | | | LLLPSTDVNKQNKFGDSPLTLLIKTLSPAHLINKLLS | |
| | | | TSNVITDQTVNICIFYDRDDVLEIINDKGKQYDSTDF | |
| | | | KMAVEVGSIRCVKYLLDNDIICEDAMYYAVLSEYE | |
| | | | TMVDYLLFNHFSVDFVVNGHTCMSECVRLNNPVIL | |
| | | | SKLMLHNLTSETMYLTMKAIEKDRLDKSIIIPFIAYF | |
| | | | VLMHPDFCKNRRYFTSYKRFVTDYVHEGVSYEVFD | |
| | | | DYF | |
| SEQ ID | TM2L | AAF33883.1 | MSSSTRLPVLVLAAELTIGVNYDINSTIIGECHMSES | Inside |
| NO: 63 | | | YIDRNANIVLTGYGLEINMTIMDTDQRFVAAAEGV | Deletion |
| | | | GKDNKLSVLLFTTQRLDKVHHNISVTITCMEMNCG | |
| | | | TTKYDSDLPESIHKSSSCDITINGSCVTCVNLETDPT | |
| | | | KINPHYLHPKDKYLYHNSEYGMRGSYGVTFIDELN | |
| | | | QCLLDIKELSYDICYRE | |
| SEQ ID | TK1L | AAF33884.1 | MLQALFKYDINIYSANLENVLLDDAEIAKMIIEKHV | Inside |
| NO: 64 | | | EYKSDSYTKDLDIVKNNKLDEIISKNKELRLMYVNC | Deletion |
| | | | VKKN | |
| SEQ ID | TK2L | AAF33885.1 | MDLSRINTWKSKQLKSFLSSKDTFKADVHGHSALY | Inside |
| NO: 65 | | | YAIADNNVRLVCTLLNSGALKNLLENEFPLHQAAT | Deletion |
| | | | LEDTKIVKILLFSGLDDSQFDDKGNTALYYAVDSGN | |
| | | | MQTVKLFVKKNWRLMFYGKTGWKTSFYHAVMLN | |
| | | | DVSIVSYFLSEIPSTFDLAILLSCIHITIKNGHVDMMIL | |
| | | | LLDYMTVDKHQ | |
| SEQ ID | TK3L | AAF33886.1 | MIALLILSLACSASAYRLQGFTNAGIVAYKNIQDDNI | Inside |
| NO: 66 | | | VFSPFGYSFSMFMSLLPASGNTRIELLKTMDLRKRD | Deletion |
| | | | LGPAFTELISGLAKLKTSKYTYTDLTYQSFVDNTVCI | |
| | | | KPSYYQQYHRFGLYRLNFRRDAVNKINSIVERRSG | |
| | | | MSNVVDSNMLDNNTLWAIINTIYFKGTWQYPFDIT | |
| | | | KTRNASFTNKYGTKTVPMMNVVTKLQGNTITIDDE | |
| | | | EYDMVRLPYKDANISMYLAIGDNMTHFTDSITAAK | |
| | | | DYWSFQLGNKVYNLKLPKFSIENKRDIKSIAEMMAP | |
| | | | SMFNPDNASFKHMTRDPLYIYKMFQNAKIDVDEQG | |
| | | | TVAEASTIMVATARSSPEELEFNTPFVFIIRHDITGFIL | |
| | | | FMGKVESP | |
| SEQ ID | ORFR | AAF33887.1 | MGHIITYCQVHTNISILIRKAYHIIFFVIDCDCISLQFS | |
| NO: 67 | | | NYVHHGNRFRTVLISKTSIACFSDIKRILPCTFKIYSI | |
| | | | NDCP | |
| SEQ ID | TK4L | AAF33888.1 | MLAFCYSLPNAGDVIKGRVYEKDYALYIYLFDYPH | Inside |
| NO: 68 | | | SEAILAESVKMHMDRYVEYRDKLVGKTVKVKVIR | Deletion |
| | | | VDYTKGYIDVNYKRMCRHQ | |
| SEQ ID | TK6L | AAF33889.1 | MTLVQHVVTIKSTYWVIPWELASYDNPNLFTAMIL | Inside |
| NO: 69 | | | MSPLVNADAVSKLNLLAAKLMGTITLNAPVGKLCP | Deletion |
| | | | ESVSRDMDKVYKYQYDPLINHEKIKAGFASQVLKA | |
| | | | TNKVRKIISKINTPRLSYSREQTIRLAMF | |
| SEQ ID | TK8R | AAF33890.1 | MATKLDYEDAVFYFVDDDKICSRDSIIDLIDEYITW | Inside |
| NO: 70 | | | RNHVIVFNKDITSCGRLYKELMKFDDVAIRYYGIDK | Deletion |
| | | | INEIVEAMSEGDHYINFTKVHDQESLFATIGICAKITE | |
| | | | HWGYKKISESRFQSLGNITDLMTDDNINILILFLEKK | |
| | | | LN | |
| SEQ ID | TF1L | AAF33891.1 | MLSMFMCNNIVDYVDDIDNGIVQDIEDEASNNVDR | Inside |
| NO: 71 | | | DYVYPLPENMVYRFDKSTNILDYLSTERDHVMMA | Deletion |
| | | | VRYYMSKQRLDDLYRQLPTKTRSYIDIINIYCDKVS | |
| | | | NDYNRDMNIMYDMASTKSFTVYDINNEVNTILMD | |
| | | | NKGLGVRLATISFITELGRRCMNPVKTIKMFTLLSHT | |
| | | | ICDDCFVDYITDISPPDNTIPNTSTREYLKLIGITAIMF | |
| | | | ATYKTLKYMIG | |
| SEQ ID | TF2L | AAF33892.1 | MFNMNINSPVRFVKETNRAKSPTRQSPGAAGYDLY | Inside |
| NO: 72 | | | SAYDYTIPPGERQLIKTDISMSMPKICYGRIAPRSGLS | Deletion |
| | | | LKGIDIGGGVIDEDYRGNIGVILINNGKCTFNVNTGD | |
| | | | RIAQLIYQRIYYPELEEVQSLDSTDRGDQGFGSTGLR | |
| SEQ ID | TF3L | AAF33893.1 | MPIFVNTVYCKNILALSMTKKFKTIIDAIGGNIIVNST | Inside |
| NO: 73 | (75% 3′) | | ILKKLSPYFRTHLRQKYTKNKDPVTRVCLDLDIHSL | Deletion |
| | | | TSIVIYSYTGKVYIDSHNVVNLLRASILTSVEFIIYTCI | |
| | | | NFILRDFRKEYCVECYMMGIEYGLSNLLCHTKNFIA | |
| | | | KHFLELEDDIIDNFDYLSMKLILESDELNVPDEDYV | |
| | | | VDFVIKWYIKRRNKLGNLLLLIKNVIRSNYLSPRGIN | |
| | | | NVKWILDCTKIFHCDKQPRKSYKYPFIEYPMNMDQI | |
| | | | IDIFHMCTSTHVGEVVYLIGGWMNNEIHNNAIAVN | |
| | | | YISNNWIPIPPMNSPRLYASGIPANNKLYVVGGLPNP | |
| | | | TSVERWFHGDAAWVNMPSLLKPRCNPAVASINNVI | |
| | | | YVMGGHSETDTTTEYLLPNHDQWQFGPSTYYPHY | |
| | | | KSCALVFGRRLFLVGRNAEFYCESSNTWTLIDDPIY | |
| | | | PRDNPELIIVDNKLLLIGGFYRESYIDTIEVYNHHTYS | |
| | | | WNIWDGK | |
| | TK5L | | | |
| | ORFR | | | |
| | TK7L | | | |

TABLE 34
Examples of proteins encoded by Wyeth Vaccinia genes
equivalent to those deleted in CopMD5p vector

| | | Protein | | |
|---|---|---|---|---|
| SEQ ID | | Accession | | |
| NO | Gene | ID | Amino Acid Sequence | Location |
| SEQ ID | VAC_DPP20_035 | AEY74729.1 | MESVTFSINGEIIQVNKEIITASPYNFFKRIQEHHINDE | Inside |
| NO: 74 | (26% 5′) | | VIILNGINYHAFESLLDYMRWKKINITINNVEMILVA | Deletion |
| | | | AVIIDVTPVVDLCVKTMIHNINSTNCIRMFNFSKRYG | |
| | | | IKKLYNASMSEIINNITAVTSDPEFGKLSKDELTTILS | |
| | | | HEDVNVNHEDVTAMILLKWIHKNPNDVDIINILHPK | |
| | | | FMTNTMRNAISLLGLTISKSTKPVTRNGIKHNIVVIK | |
| | | | NSDYISTITHYSPRTEYWTIVGNTDRQFYNANVLHN | |
| | | | CLYIIGGMINNRHVYSVSRVDLETKKWKTVTNMSS | |
| | | | LKSEVSTCVNNGKLYVIGGLEFSISTGVAEYLKHGT | |
| | | | SKWIRLPNLITPRYSGASVFVNDDIYVMGGVYTTYE | |
| | | | KYVVLNDVECFTKNRWIKKSPMPRHHSIVYAVEYD | |
| | | | GDIYAITGITHETRNYLYKYIVKEDKWIELYMYFNH | |
| | | | VGKMFVCSCGDYILIIADAKYEYYPKSNTWNLFDM | |
| | | | STRNIEYYDMFTKDETHKSLPSFLSNCEKQFLQ | |
| SEQ ID | VAC_DPP10_036 | AEY74730.1 | MVKNNKISNSCRMIMSTNPNNILMRHLKNLTDDEF | Inside |
| NO: 75 | | | KCIIHRSSDFLYLSDRDYTSITKETLVSEIVEEYPDDC | Deletion |
| | | | NKILAIIFLVLDKDIDVDIKTKLKPKPAVRFAILDKM | |
| | | | TEDIKLTDLVRHYFRYIEQDIPLGPLFKKIDSYRTRAI | |
| | | | NKYSKELGLATEYFNKYGHLMFYTLPIPYNRFFCRN | |
| | | | SIGFLAVLSPTIGHVKAFYKFIEYVSIDDRRKFKKEL | |
| | | | MSK | |
| SEQ ID | N1L | AEY74731.1 | MRTLLIRYILWRNDNDQTYYNDDFKKLMLLDELVD | Inside |
| NO: 76 | | | DGDVCTLIKNMRMTLSDGPLLDRLNQPVNNIEDAK | Deletion |
| | | | RMIAISAKVARDIGERSEIRWEESFTILFRMIETYFDD | |
| | | | LMIDLYGEK | |
| SEQ ID | VAC_DPP11_038 | AEY74732.1 | MTSSAMDNNEPKVLEMVYDATILPEGSSMDPNIIDC | Inside |
| NO: 77 | | | INRHINMCIQRTYSSSIIAILDRFLTMNKDELNNTQC | Deletion |
| | | | HIIKEFMTYEQMAIDHYGGYVNAILYQIRKRPNQHH | |
| | | | TIDLFKKIKRTRYDTFKVDPVEFVKKVIGFVSILNKY | |
| | | | KPVYSYVLYENVLYDEFKCFIDYVETKYF | |
| SEQ ID | VAC_DPP11_039 | AEY74733.1 | MLHNPTSETMYLTMNAIKKDKLDKSIIIPFIAYFVLM | Not |
| NO: 78 | | | HPDFCKNRRYFTSYKRFVTDYVHEGVSYEVFDDYF | Present |
| SEQ ID | VAC_DPP12_040 | AEY74734.1 | MSIGFEARIVDKFGKNHIHRHLMSDNPKASTISWM | Inside |
| NO: 79 | | | MKLGISPSKPDHDGNTPLHIVCSKTVKYVDIIDLLLP | Deletion |
| | | | STDVNKQNKFGDSPLTLLIKTLSLAHINKLLSTSNV | |
| | | | ITDQTVNICIFYDRDDVLEIINDKGKQYDFKMAVEV | |
| | | | GSIKCVKYLLDNDIICEDAMYYAVLSEYKTMVDYL | |
| | | | LFNHFSVDSVVNGHTCMSECVKLNNRHFIEADVT | |
| SEQ ID | VAC_DPP12_041 | AEY74735.1 | MIFVLESKLLQIYRNRNRNINFYTTMDNIMSAEYYLS | Not |
| NO: 80 | | | LYAKYNSKNLDVFRNMLQAIEPSGNNYHILHAYCG | Present |
| | | | IKGLDERFVEELLHRGYSPNETDDDGNYPLHIASKIN | |
| | | | NNRIVAMLLTHGADPNACDKHNKTPLYYLSGTDDE | |
| | | | VIERINLLVQYGAKINN | |
| SEQ ID | VAC_DPP12_042 | AEY74736.1 | MVYKLVLLFCIASLGYSVEYKNTICPPRQDYRYWY | Inside |
| NO: 81 | | | FAAELTIGVNYDINSTIIGECHMSESYIDRNANIVLTG | Deletion |
| | | | YGLEINMTIMDTDQRFVAAAEGVGKDNKLSVLLFT | |
| | | | TQRLDKVHHNISVTITCMEMNCGTTKYDSDLPESIH | |
| | | | KSSSCDITINGSCVTCVNLETDPTKINPHYLHPKDKY | |
| | | | LYHNSEYGMRGSYGVTFIDELNQCLLDIKELSYDIC | |
| | | | YRE | |
| SEQ ID | VAC_DPP10_043 | AEY74737.1 | MDLSRINTWKSKQLKSFLSSKDTFKADVHGHSALY | Inside |
| NO: 82 | | | YAIADNNVRLVCTLLNAGALKNLLENEFPLHQAAT | Deletion |
| | | | LEDTKIVKILLFSGLDDSQFDDKGNTALYYAVDSGN | |
| | | | MQTVKLFVKKNWRLMFYGKTGWKTSFYHAVMLN | |
| | | | DVSIVSYFLSEIPSTFDLAILLSCIHITIKNGHVDMMIL | |
| | | | LLDYMTSTNTNNSLLFIPDIKLAIDNKDIEMLQALFK | |
| | | | YDINIYSANLENVLLDDAEIAKMIIEKHVEYKSDSYT | |
| | | | KDLDIVKNNKLDEIISKNKELRLMYVNCVKKN | |
| SEQ ID | VAC_DPP20_044 | AEY74738.1 | MIALLILSLTCSVSTYRLQGFTNAGIVAYKNIQDDNI | Inside |
| NO: 83 | | | VFSPFGYSFSMFMSLLPASGNTRIELLKTMDLRKRD | Deletion |
| | | | LGPAFTELISGLAKLKTSKYTYTDLTYQSFVDNTVCI | |
| | | | KPSYYQQYHRLNFRRDAVNKINSIVERRSGMSNVV | |
| | | | DSNMLDNNTLWAIINTIYFKGIWQYPFDITKTRNAS | |
| | | | FTNKYGTKTVPMMNVVTKLQGNTITIDDKEYDMV | |
| | | | RLPYKDANISMYLAIGDNMTHFTDSITAAKLDYWS | |
| | | | FQLGNKVYNLKLPKFSIENKRDIKSIAEMMAPSMFN | |
| | | | PDNASFKHMTRDPLYIYKMFQNAKIDVDEQGTVAE | |
| | | | ASTIMVATARSSPEKLEFNTPFVFIIRHDITGFILFMG | |
| | | | KVESP | |
| SEQ ID | VAC_DPP10_045 | AEY74739.1 | MLAFCYSLPNAGDVIKGRVYENDYALYIYLFDYPH | Inside |
| NO: 84 | | | FEAILAESVKMHMDRYVEYRDKLVGKTVKVKVIR | Deletion |
| | | | VDYTKGYIDVNYKRMCRHQ | |
| SEQ ID | K4L | AEY74740.1 | MNPDNTIAVITETIPIGMQFDKVYLSTFNMWREILSN | Inside |
| NO: 85 | | | TTKTLDISSFYWSLSDEVGTNFGTIILNEIVQLPKRG | Deletion |
| | | | VRVRVAVNKSNKPLKDVERLQMAGVEVRYIDITNI | |
| | | | LGGVLHTKFWISDNTHIYLGSANMDWRSLTQVKEL | |
| | | | GIAIFNNRNLAADLTQIFEVYWYLGVNNLPYNWKN | |
| | | | FYPSYYNTDHPLSINVSGVPHSVFIASAPQQLCTME | |
| | | | RTNDLTALLSCIRNASKFVYVSVMNFIPIIYSKAGKI | |
| | | | LFWPYIEDELRRSAIDRQVSVKLLISCWQRSSFIMRN | |
| | | | FLRSIAMLKSKNINIEVKLFIVPDADPPIPYSRVNHAK | |
| | | | YMVTDKTAYIGTSNWTGNYFTDTCGASINITPDDGL | |
| | | | GLRQQLEDIFMRDWNSKYSYELYDTSPTKRCKLLK | |
| | | | NMKQCTNDIYCDEIQPEKEIPEYSLE | |
| SEQ ID | VAC_DPP20_047 | AEY74741.1 | MGATISILASYDNPNLFTAMILMSPLVNADAVSKLN | Inside |
| NO: 86 | | | LLAAKLMGTITPNAPVGKLCPESVSRDMDKVYKYQ | Deletion |
| | | | YDPLINHEKIKAGFASQVLKATNKVRKIISKINTPPT | |
| | | | LILQGTNNEISDVLGAYYFMQHANCNREIKIYEGAK | |
| | | | HHLHKETDEVKKSVMKEIETWIFNRVK | |
| SEQ ID | List034/ | AEY74742.1 | MSANCMFNLDNDYIYWKPITYPKALVFISHGAGKH | Inside |
| NO: 87 | VAC_DPP20_048 | | SGRYDELAENISSLGILVFSHDHIGHGRSNGEKMMI | Deletion |
| | | | DDFGTARGNY | |
| SEQ ID | K7R/ | AEY74743.1 | MATKLDYEDAVFYFVDDDKICSRDSIIDLIDEYITW | Inside |
| NO: 88 | VAC_DPP20-49 | | RNHVIVFNKDITSCGRLYKELMKFDDVAIRYYGIDK | Deletion |
| | | | INEIVEAMSEGDHYINFTKVHDQESLFATIGICAKITE | |
| | | | HWGYKKISESRFQSLGNITDLMTDDNINILILFLEKK | |
| | | | LN | |
| SEQ ID | LIVPclone14_046/ | AEY74744.1 | MLSMFMCNNIVDYVDDIDNGIVQDIEDEASNNVDH | Inside |
| NO: 89 | VAC_DPP20_047 | | DYVYPLPENMVYRFDKSTNILDYLSTERDHVMMA | Deletion |
| | | | VRYYMSKQRLDDLYRQLPTKTRSYIDIINIYCDKVS | |
| | | | NDYNRDMNIMYDMASTKSFTVYDINNEVNTILMD | |
| | | | NKGLGVRLATISFITKLGRRCMNPVKTIKMFTLLSH | |
| | | | TICDDCFVDYITDISPPDNTIPNTSTREYLKLIGITAIM | |
| | | | FATYKTLKYMIG | |
| SEQ ID | F2L/ | AEY74745.1 | MFNMNINSPVRFVKETNRAKSPTRQSPGAAGYDLY | Inside |
| NO: 90 | VAC_DPP20_051 | | SAYDYTIPPGERQLIKTDISMSMPKICYGRIAPRSGLS | Deletion |
| | | | LKGIDIGGGVIDEDYRGNIGVILINNGKCTFNVNTGD | |
| | | | RIAQLIYQRIYYPELEEVQSLDSTNRGDQGFGSTGLR | |
| SEQ ID | F3L | AEY74746.1 | MPIFVNTVYCKNILALSMTKKFKTIIDAIGGNIIVNST | Inside |
| NO: 91 | (75% 3′) | | ILKKLSPYFRTHLRQKYTKNKDPVTRVCLDLDIHSL | Deletion |
| | | | TSIVIYSYTGKVYIDSHNVVNLLRASILTSVEFIIYTCI | |
| | | | NFILRDFRKEYCVECYMMGIEYGLSNLLCHTKNFIA | |
| | | | KHFLELEDDIIDNFDYLSMKLILESDELNVPDEDYV | |
| | | | VDFVIKWYIKRRNKLGNLLLLIKNVIRSNYLSPRGIN | |
| | | | NVKWILDCTKIFHCDKQPRKSYKYPFIEYPMNMDQI | |
| | | | IDIFHMCTSTHVGEVVYLIGGWMNNEIHNNAIAVN | |
| | | | YISNNWIPIPPMNSPRLYASGIPANNKLYVVGGLPNP | |
| | | | TSVERWFHGDAAWVNMPSLLKPRCNPAVASINNVI | |
| | | | YVMGGHSETDTTTEYLLPNHDQWQFGPSTYYPHY | |
| | | | KSCALVFGRRLFLVGRNAEFYCESSNTWTLIDDPIY | |
| | | | PRDNPELIIVDNKILLIGGFYRESYIDTIEVYNHHTYS | |
| | | | WNIWDGK | |

TABLE 35
Examples of proteins encoded by Lister Vaccinia genes equivalent to
those deleted in CopMD5p vector

| | | Protein | | |
|---|---|---|---|---|
| SEQ ID | | Accession | | |
| NO | Gene | ID | Amino Acid Sequence | Location |
| SEQ ID | List023 | ABD52473.1 | MESVIFSINGEIIQVNKEIITASPYNFFKRIQDHHLKD | Inside |
| NO: 92 | (26% 5′) | | EAIILNGINYHAFESLLDYMRWKKINITINNVEMILV | Deletion |
| | | | AAIIIDVPPVVDLCVKTMIHNINFTNCIRMFNFSKRY | |
| | | | GIKKLYNASMSEIINNITAVTSDPEFGKLSKDELTTIL | |
| | | | SHEDVNVNHEDVTAMILLKWIHKNPNDVDIINILHP | |
| | | | KFMTNTMRNAISLLGLTISKSTKPVTRNGIKHNIVVI | |
| | | | KNSDYISTITHYSPRTEYWTIVGNTDRQFYNANVLH | |
| | | | NCLYIIGGMINNRHVYSVSRVDLKTKKWKTVTNMS | |
| | | | SLKSEVSTCVNDGKLYVIGGLEFSISTGVAEYLKHG | |
| | | | TSKWIRLPNLITPRYSGASVFVNDDIYVMGGVYTTY | |
| | | | EKYVVLNDVECFTKNRWIKKSPMPRHHSIVYAVEY | |
| | | | DGDIYVITGITHETRNYLYKYIVKEDKWIELYMYFN | |
| | | | HVGKMFVCSCGDYILIIADAKYEYYPKSNTWNLFD | |
| | | | MSTRNIEYYDMFTKDETPKCNVTHKSLPSFLSNCEK | |
| | | | QFLQ | |
| SEQ ID | C1L/List024 | ABD52474.1 | MVKNNKISNSCRMIMSTNPNNILMRHLKNLTDDEF | Inside |
| NO: 93 | | | KCIIHRSSDFLYLSDSDYTSITKETLVSEIVEEYPDDC | Deletion |
| | | | NKILAIIFLVLDKDIDVDIETKLKPKPAVRFAILDKM | |
| | | | TADIKLTDLVRHYFRYIEQDIPLGPLFKKIDSYRTRAI | |
| | | | NKYSKELGLATEYFNKYGHLMFYTLPIPYNRFFCRN | |
| | | | SIGFLAVLSPTIGHVKAFYKFIEYVSIDDRRKFKKEL | |
| | | | MSK | |
| SEQ ID | N1L/List025 | ABD52475.1 | MRTLLIRYILWRNDNDQTYYNDDFKKLMLLDELVD | Inside |
| NO: 94 | | | DGDVCTLIKNMRMTLSDGPLLDRLNQPVNNIEDAK | Deletion |
| | | | RMIAISAKVARDIGERSEIRWEESFTILFRMIETYFDD | |
| | | | LMIDLYGEK | |
| SEQ ID | List026 | ABD52476.1 | MTSSAMDNNEPKVLEMVYDATILPEGSSMDPNIMD | Inside |
| NO: 95 | | | CINRHINMCIQRTYSSSIIAILDRFLTMNKDELNNTQ | Deletion |
| | | | CHIIKEFMTYEQMAIDHYGEYVNAILYQIRKRPNQH | |
| | | | HTIDLFKKIKRTRYDTFKVDPVEFVKKVIGFVSILNK | |
| | | | YKPVYSYVLYENVLYDEFKCFIDYVETKYF | |
| SEQ ID | List027 | ABD52477.1 | MIFVIESKLLQIYRNRNINFYTTMDNIMSAEYYLSLY | Inside |
| NO: 96 | | | AKYNSKNLDVFRNMLQAIEPSGNNYHILHAYCGIK | Deletion |
| | | | GLDERFVEELLHRGYSPNETDDDGNYPLHIASKINN | |
| | | | NRIVAMLLTHGADPNACDKQHKTPLYYLSGTDDEV | |
| | | | IERINLLVQYGAKINNSVDEEGCGPLLACTDPSERVF | |
| | | | KKIMSIGFEARIVDKFGKNHIHRHLMSDNPKASTIS | |
| | | | WMMKLGISPSKPDHDGNTPLHIVCSKTVKNVDIIDL | |
| | | | LLPSTDVNKQNKFGDSPLTLLIKTLSPAHLINKLLST | |
| | | | SNVITDQTVNICIFYDRDDVLEIINDKGKQYDSTDFK | |
| | | | MAVEVGSIRCVKYLLDNDIICEDAMYYAVLSEYET | |
| | | | MVDYLLFNHFSVDSVVNGHTCMSECVRLNNPVILS | |
| | | | KLMLHNPTSETMYLTMKAIEKDRLDKSIIIPFIAYFV | |
| | | | LMHPDFCKNRRYFTSYKRFVTDYVHEGVSYEVFDD | |
| | | | YF | |
| SEQ ID | List028 | ABD52478.1 | MVYKLVLLFCIASLGYSVEYKNTICPPRQDYRYWY | Inside |
| NO: 97 | | | FAAELTIGVNYDINSTIIGECHMSESYIDRNANIVLTG | Deletion |
| | | | YGLEINMTIMDTDQRFVAAAEGVGKDNKLSVLLFT | |
| | | | TQRLDKVHHNISVTITCMEMNCGTTKYDSDLPESIH | |
| | | | KSSSCDITINGSCVTCVNLETDPTKINPHYLHPKDKY | |
| | | | LYHNSEYGMRGSYGVTFIDELNQCLLDIKELSYDIC | |
| | | | YRE | |
| SEQ ID | K1L/List029 | ABD52479.1 | MDLSRINTWKSKQLKSFLSSKDAFKADINGHSALY | Inside |
| NO: 98 | | | YAIADNNVRLVCTLLNAGALKNLLENEFPLHQAAT | Deletion |
| | | | LEDTKIVKILLFSGLDDSQFDDKGNTALYYAVDSGN | |
| | | | MQTVKLFVKKNWRLMFYGKTGWKTSFYHAVMLN | |
| | | | DVSIVSYFLSEIPSTFDLAILLSCIHITIKNGHVDMMIL | |
| | | | LLDYMTSTNTNNSLLFIPDIKLAIDNKDIEMLQALFK | |
| | | | YDINIYSANLENVLLDDAEIAKMIIEKHVEYKSDSYT | |
| | | | KDLDIVKNNKLDEIISKNKELKLMYVNCVKKN | |
| SEQ ID | List030 | ABD52480.1 | IELLKTMDLRKRDLGPAFTELISGLAKLKTSKYTYT | Inside |
| NO: 99 | | | DLTYQSFVDNTVCIKPSYYQQYHRFGLYRLNFRRD | Deletion |
| | | | AVNKINSIVERRSGMSNVVDSNMLDNNTLWAIINTI | |
| | | | YFKGIWQYPFDITKTRNASFTNKYGTKTVPMMNVV | |
| | | | TKLQGNTITIDDEEYDMVRLPYKDANISMYLAIGDN | |
| | | | MTHFTDSITAAKLDYWSSQLGNKVYNLKLPKFSIEN | |
| | | | KRDIKSIAEMMAPSMFNPDNASFKHMTRDPLYIYK | |
| | | | MFQNAKIDVDEQGTVAEASTIMVATARSSPEKLEFN | |
| | | | TPFVFIIRHDITGFILFMGKVESP | |
| SEQ ID | K3L/List031 | ABD52483.1 | MLAFCYSLPNAGDVIKGRVYENDYALYIYLFDYPH | Inside |
| NO: 100 | | | SEAILAESVKMHMDRYVEYRDKLVGKTVKVKVIR | Deletion |
| | | | VDYTKGYIDVNYKRMCRHQ | |
| SEQ ID | K4L/List032 | ABD52484.1 | MNPDNTIAVITETIPIGMQFDKVYLSTFNMWREILSN | Inside |
| NO: 101 | | | TTKTLDISSFYWSLSDEVGTNFGTIILNEIVQLPKRG | Deletion |
| | | | VRVRVAVNKSNKPLKDVERLQMAGVEVRYIDITNI | |
| | | | LGGVLHTKFWISDNTHIYLGSANMDWRSLTQVKEL | |
| | | | GIAIFNNRNLAADLTQIFEVYWYLGVNNLPYNWKN | |
| | | | FYPSYYNTDHPLSINVSGVPHSVFIASAPQQLCTME | |
| | | | RTNDLTALLSCIRNASKFVYVSVMNFIPIIYSKAGKI | |
| | | | LFWPYIEDELRRSAIDRQVSVKLLISCWQRSSFIMRN | |
| | | | FLRSIAMLKSKNINIEVKLFIVPDADPPIPYSRVNHAK | |
| | | | YMVTDKTAYIGTSNWTGNYFTDTCGASINITPDDGL | |
| | | | GLRQQLEDIFMRDWNSKYSYELYDTSPTKRCKLLK | |
| | | | NMKQCTNDIYCDEIQPEKEIPEYSLE | |
| SEQ ID | List033 | ABD52485.1 | MGHSMGATISILASYDNPNLFTAMILMSPLVNADA | Outside |
| NO: 102 | | | VSRLNLLAAKLMGTITPNAPVGKLCPESVSRDMDK | Deletion |
| | | | VYKYQYDPLINHEKIKAGFASQVLKATNKVRKIISKI | |
| | | | NTPPTLILQGTNNKISDVLGAYYFMQHANCNREIKI | |
| | | | YEGAKHHLHKETDEVKKSVMKEIETWIFNRVK | |
| SEQ ID | List034 | ABD52486.1 | MSANCMFNLDNDYIYWKPITYPKALVFISHGAGKH | Inside |
| NO: 103 | | | SGRYDELAENISSLGILVFSHDHIGHGRSNGEKMMI | Deletion |
| | | | DDFGTARGNY | |
| SEQ ID | K7R/List035 | ABD52487.1 | MATKLDYEDAVFYFVDDDKICSRDSIIDLIDEYITW | Inside |
| NO: 104 | | | RNHVIVFNKDITSCGRLYKELMKFDDVAIRYYGIDK | Deletion |
| | | | INEIVEAMSEGDHYINFTKVHDQESLFATIGICAKITE | |
| | | | HWGYKKISESRFQSLGNITDLMTDDNINILILFLEKK | |
| | | | LN | |
| SEQ ID | F1L/List036 | ABD52489.1 | MLSMFMCNNIVDYVDDIDNGIVQDIEDEASNNVDH | Inside |
| NO: 105 | | | DYVYPLPENMVYRFDKSTNILDYLSTERDHVMMA | Deletion |
| | | | VRYYMSKQRLDDLYRQLPTKTRSYIDIINIYCDKVS | |
| | | | NDYNRDMNIMYDMASTKSFTVYDINNEVNTILMD | |
| | | | NKGLGVRLATISFITELGRRCMNPVKTIKMFTLLSHT | |
| | | | ICDDCFVDYITDISPPDNTIPNTSTREYLKLIGITAIMF | |
| | | | ATYKTLKYMIG | |
| SEQ ID | List037 | ABD52490.1 | MFNMNINSPVRFVKETNRAKSPTRQSPGAAGYDLY | Inside |
| NO: 106 | | | SAYDYTIPPGERQLIKTDISMSMPKFCYGRIAPRSGL | Deletion |
| | | | SLKGIDIGGGV1DEDYRGNIGVILINNGKCTFNVNTG | |
| | | | DRIAQLIYQRIYYPELEEVQSLDSTNRGDQGFGSTGLR | |
| SEQ ID | List038 | ABD52491.1 | MPIFVNTVYCKNILALSMTKKFKTIIDAIGGNIIVNST | Inside |
| NO: 107 | (75% 3′) | | ILKKLSPYFRTHLRQKYTKNKDPVTRVCLDLDIHSL | Deletion |
| | | | TSIVIYSTGKVYIDSHNVVNLLRASILTSVEFIIYTCI | |
| | | | NFILRDFRKEYCVECYMMGIEYGLSNLLCHTKNFIA | |
| | | | KHFLELEDDIIDNFDYLSIKLILESDELNVPDEDYVV | |
| | | | DFVIKWYIKRRNKLGNLLLLIKNVIRSNYLSPRGINN | |
| | | | VKWILDCTKIFHCDKQPRKSYKYPFIEYPMNMDQII | |
| | | | DIFHMCTSTHVGEVVYLIGGWMNNEIHNNAIAVNY | |
| | | | ISNNWIPIPPMNSPRLYASGIPANNKLYVVGGLPNPT | |
| | | | SVERWFHGDAAWVNMPSLLKPRCNPAVASINNVIY | |
| | | | VMGGHSETDTTTEYLLPNHDQWQFGPSTYYPHYKS | |
| | | | CALVFGRRLFLVGRNAEFYCESSNTWTLIDDPIYPR | |
| | | | DNPELIIVDNKLLLIGGFYRESYIDTIEVYNHHTYSW | |
| | | | NIWDGK | |

TABLE 36
Examples of proteins encoded by Copenhagen Vaccinia genes deleted in CopMD3p vector

| SEQ ID | | Protein | | |
|---|---|---|---|---|
| NO | Gene | Accession ID | Amino Acid Sequence | Location |
| SEQ ID | B14R | AAA49211.1 | MNHCLLAISAVYFKAKWLTPFEKEFTSDYPFYVSPT | Inside |
| NO: 108 | (41% 3′) | | EMVDVSMMSMYGELFNHASVKESFGNFSIIELPYV | Deletion |
| | | | GDTSMMVILPDKIDGLESIEQNLTDTNFKKWCNSLD | |
| | | | AMFIDVHIPKFKVTGSYNLVDTLVKSGLTEVFGSTG | |
| | | | DYSNMCNLDVSVDAMIHKTYIDVNEEYTEAAAATC | |
| | | | ALVSDCASTITNEFCVDHPFIYVIRHVDGKILFVGRY | |
| | | | CSPTTNC | |
| SEQ ID | B15R | AAA49212.1 | MTANFSTHVFSPQHCGCDRLTSIDDVKQCLTEYIYW | Inside |
| NO: 109 | | | SSYAYRNRQCAGQLYSTLLSFRDDAELVFIDIRELV | Deletion |
| | | | KNMPWDDVKDCTEIIRCYIPDEQKTIREISAIIGLCA | |
| | | | YAATYWGGEDHPTSNSLNALFVMLEMLNYVDYNII | |
| | | | FRRMN | |
| SEQ ID | B ORF E | AAA48213.1 | MYNSSIHTPEYDVIIHVIEHLKHHKQCVQTVTSGMV | Inside |
| NO: 110 | | | FTSPVSSSICTKSDDGRNLSDGFLLIRYITTDDFCTIF | Deletion |
| | | | DIIPRHIFYQLANVDEH | |
| SEQ ID | B16R | AAA48214.1 | MSILPVIFLPIFFYSSFVQTFNASECIDKGXYFASFME | Inside |
| NO: 111 | | | LENEPVILPCPQINTLSSGYNILDILWEKRGADNDRII | Deletion |
| | | | PIDNGSNMLILNPTQSDSGIYICITTNETYCDMMSLN | |
| | | | LTIVSVSESNIDFISYPQIVNERSTGEMVCPNINAFIAS | |
| | | | NVNADIIWSGHRRLRNKRLKQRTPGIITIEDVRKND | |
| | | | AGYYTCVLEYIYGGKTYNVTRIVKLEVRDKIIHPTM | |
| | | | QLPEGVVTSIGSNLTIACRVSLRPPTTDADVFWISNG | |
| | | | MYYEEDDGDGDGRISVANKIYMTDKRRVITSRLNI | |
| | | | NPVKEEDATTFTCMAFTIPSISKTVTVSIT | |
| SEQ ID | B ORF F | AAA48215.1 | MVIIPGVRCLSLLFLRRRCPLHIISAFTLLAINALILGH | Inside |
| NO: 112 | | | TISPVDLSFTICGYEIKSIFDSETDTIVKFNDIMSQ | Deletion |
| SEQ ID | B17L | AAA48216.1 | MSRKFMQVYEYDREQYLDEFIEDRYNDSFITSPEYY | Inside |
| NO: 113 | | | SAEKYMCRYTTLNHNCVNVRRCALDSKLLHDIITN | Deletion |
| | | | CKIYNNIELVRATKFVYYLDLIKCNWVSKVGDSVL | |
| | | | YPVIFITHTSTRNLDKVSVKTYKGVKVKKLNRCAD | |
| | | | HAIVINPFVKFKLTLPNKTSHAKVLVTFCKLRTDITP | |
| | | | VEAPLPGNVLVYTFPDINKRIPGYIHVNIEGCIDGMI | |
| | | | YINSSKFACVLKLHRSMYRIPPFPIDICSCCSQYTND | |
| | | | DIEIPIHDLIKDVAIFKNKETVYYLKLNNKTIARFTYF | |
| | | | NNIDTAITQEHEYVKIALGIVCKLMINNMHSIVGVN | |
| | | | HSNTFVNCLLEDNV | |
| SEQ ID | B18R | AAA48217.1 | MSRRLIYVLNINRKSTHKIQENEIYTYFSHCNIDHTS | Inside |
| NO: 114 | | | TELDFVVKNYDLNRRQHVTGYTALHCYLYNNYFT | Deletion |
| | | | NDVLKILLNHDVNVTMKTSSGRMPVYILLTRCCNIS | |
| | | | HDVVIDMIDKDKNHLSHRDYSNLLLEYIKSRYMLL | |
| | | | KEEDIDENIVSTLLDKGIDPNFKQDGYTALHYYYLC | |
| | | | LAHVYKPGECRKPITIKKAKRIISLFIQHGANLNALD | |
| | | | NCGNTPFHLYLSIEMCNNIHMTKMLLTFNPNFKICN | |
| | | | NHGLTPILCYITSDYIQHDILVMLIHHYETNVGEMPI | |
| | | | DERRMIVFEFIKTYSTRPADSITYLMNRFKNINIYTR | |
| | | | YEGKTLLHVACEYNNTQVIDYLIRINGDINALTDNN | |
| | | | KHATQLIIDNKENSPYTINCLLYILRYIVDKNVIRSLV | |
| | | | DQLPSLPIFDIKSFEKFISYCILLDDTFYDRHVKNRDS | |
| | | | KTYRYAFSKYMSFDKYDGIITKCHDETMLLKLSTVL | |
| | | | DTTLYAVLRCHNSRKLRRYLTELKKYNNDKSFKIY | |
| | | | SNIMNERYLNVYYKDMYVSKVYDKLFPVFTDKNC | |
| | | | LLTLLPSEIIYEILYMLTINDLYNISYPPTKV | |
| SEQ ID | B19R | AAA48218.1 | MTMKMMVHIYFVSLSLLLLLFHSYAIDIENEITEFFN | Inside |
| NO: 115 | | | KMRDTLPAKDSKWLNPACMFGGTMNDMATLGEPF | Deletion |
| | | | SAKCPPIEDSLLSHRYKDYVVKWERLEKNRRRQVS | |
| | | | NKRVKHGDLWIANYTSKFSNRRYLCTVTTKNGDC | |
| | | | VQGIVRSHIKKPPSCIPKTYELGTHDKYGIDLYCGIL | |
| | | | YAKHYNNITWYKDNKEINIDDIKYSQTGKELIIHNPE | |
| | | | LEDSGRYDCYVHYDDVRIKNDIVVSRCKILTVIPSQ | |
| | | | DHRFKLILDPKINVTIGEPANITCTAVSTSLLIDDVLI | |
| | | | EWENPSGWLIGFDFDVYSVLTSRGGITEATLYFENV | |
| | | | TEEYIGNTYKCRGHNYYFEKTLTTTVVLE | |
| SEQ ID | B20R | AAA48219.1 | MDEDTRLSRYLYLTDREHINVDSIKQLCKISDPNAC | Inside |
| NO: 116 | | | YRCGCTALHEYFYNYRSVNGKYKYRYNGYYQYYS | Deletion |
| | | | SSDYENYNEYYYDDYDRTGMNSESDSESDNISIKTE | |
| | | | YENEYEFYDETQDQSTQHNDL | |
| SEQ ID | B21R | AAA48220.1 | MSLESFIITTFNNNSSTNIDNMCHLYVKVCPSSLLFR | Inside |
| NO: 117 | | | LFVECCDINKLVEGTTPLHCYLMNEGFESSVLKNLL | Deletion |
| | | | KEYVMNTFNVHDIHYTNI | |
| SEQ ID | B22R | AAA48221.1 | MISLSFLIHNPLKKWKLKPSISINGYRSTFTMAFPCA | Inside |
| NO: 118 | | | QFRPCHCHATKDSLNTVADVRHCLTEYILWVSHRW | Deletion |
| | | | THRESAGSLYRLLISFRTDATELFGGELKDSLPWDNI | |
| | | | DNCVEIIKCFIRNDSMKTAEELRAIIGLCTQSAIVSGR | |
| | | | VFNDKYIDILLMLRKILNENDYLTLLDHIRTAKY | |
| SEQ ID | B23R | AAA48222.1 | MIAFIIFREIGIISTRIAMDYCGRECTILCRLLDEDVTY | Inside |
| NO: 119 | | | KKIKLEIETCHNLSKHIDRRGNNALHCYVSNKCDTD | Deletion |
| | | | IK1VRLLLSRGVERLCRNNEGLTPLGAYSKHRYVKS | |
| | | | QIVHLLISSYSNSSNELKSNINDFDLSSDNIDLRLLKY | |
| | | | LIVDKRIRPSKNTNYAINGLGLVDIYVTTPNPRPEVL | |
| | | | LWLLKSECYSTGYVFRTCMYNSDMCKNSLHYYISS | |
| | | | HRESQSLSKDVIKCLINNNVSIHGRDEGGSLPIQYY | |
| | | | WSFSTIDIEIVKLLLIKDVDTCRVYDVSPILEAYYLN | |
| | | | KRFRVTPYNVDMEIVNLLIERRHTLVDVMRSITSYD | |
| | | | SREYNHYIIDNILKRFRQQDESIVQAMLINYLHYGD | |
| | | | MVVRCMLDNGQQLSSARLLC | |
| SEQ ID | B24R | AAA48223.1 | MYGLILSRFNNCGYHCYETILIDVFDILSKYMDDID | Inside |
| NO: 120 | | | MIDNENKTLLYYAVDVNNIQFAKRLLEYGASVTTS | Deletion |
| | | | RSIINTAIQKSSYQRENKTRIVDLLLSYHPTLETMIDA | |
| | | | FNRDIRYLYPEPLFACIRYALILDDDFPSKVSMISPVII | |
| | | | RN | |
| SEQ ID | B ORF G | AAA48224.1 | MRRCIHIKERKIHMTNIVDRNVTFILTVVHKYVRYV | Inside |
| NO: 121 | | | PHTVANDAHNLVHLAHLIHFIIYFFIIRDVRKKKKKK | Deletion |
| | | | KKNRTIYFFSNVYARHIK | |
| SEQ ID | B25R | AAA48225.1 | MSRINITKKIYCSVFLFLFLFLSYISNYEKVNDEMYE | Inside |
| NO: 122 | | | MGEMDEIVSIVRDSMWYIPNVFMDDGKNEGHVSV | Deletion |
| | | | NNVCHMYFTFFDVDTSSHLFKLVIKHCDLNKRGNS | |
| | | | PLHCYTMNTRFNPSVLKILLHHGMRNFDSKDEKGH | |
| | | | HYLIHSLSIDNKIFDILTDTIDDFSKSSDLLLCYLRYK | |
| | | | FNGSLNYYVLYKGSDPNCADEDELTSLHYYCKHIST | |
| | | | FYKSNYYKLSHTKMRAEKRFIYAIIDYGANINAVTH | |
| | | | LPSTVYQT | |
| SEQ ID | B26R | AAA48226.1 | MEQTLTRLHTYLQQYTKHSPRVVYALLSRGYVIILI | Inside |
| NO: 123 | | | VHPSWNDCATGHILIMLLNWHEQKEEGQHLLYLFI | Deletion |
| | | | KHNQGYTLNILRYLLDRFDIQKDEYIYRLSKL | |
| SEQ ID | B27R | AAA48227.1 | MLPHTSDTTSTFRLKTVFDLVFENRNIIYKADVVNDI | Inside |
| NO: 124 | | | IHHRLKVSLPMIKSLFYKMSEFSPYDDYYVKKILAY | Deletion |
| | | | CLLRDESFAELHSKFCLNEDYKSVFMKNISFDKIDSII | |
| | | | VT | |
| SEQ ID | B28R | AAA48228.1 | MKSVLYSYILFLSCIIINGRDIAPHAPSDGKCKDNEY | Inside |
| NO: 125 | | | KRHNLCPGTYASRLCDSKTNTQCTPCGSGTFTSRNN | Deletion |
| | | | HLPACLSCNGRRDRVTLLTIESVNALPDIIVFSKDHP | |
| | | | DARHVFPKQNVE | |
| SEQ ID | C23L/B29R | AAA48229.1 | MHVPASLQQSSSSSSSCTEEENKHHMGIDVIIKVTK | Inside |
| NO: 126 | (44% 5′) | | QDQTPTNDKKQSVTEITESESDPDPEVESEDDSTSV | Deletion |
| | | | EDVDPPTTYYSIIGGGLRMNFGFTKCPQIKSISESAD | |
| | | | GNTVNARLSSVSPGQGKDSPAITREEALAMIKDCEV | |
| | | | SIDIRCSEEEKDSDIKTHPVLGSNISHKKVSYEDIIGST | |
| | | | IVDTKCVKNLEFSVRIGDMCKESSELEVKDGFKYVD | |
| | | | GSASEGATDDTSLIDSTKLKACV | |

TABLE 37
Examples of proteins encoded by Western Reserve Vaccinia genes
equivalent to those deleted in CopMD3p vector

| | | Protein | | |
|---|---|---|---|---|
| SEQ ID | | Accession | | |
| NO | Gene | ID | Amino Acid Sequence | Location |
| SEQ ID | SPI- | AAO89474.1 | MDIFREIASSMKGENVFISPASISSVLTILYYGANGST | Inside |
| NO: 127 | 2/B13R/ | | AEQLSKYVEKEENMDKVSAQNISFKSINKVYGRYS | Deletion |
| | VACWR195 | | AVFKDSFLRKIGDKFQTVDFTDCRTIDAINKCVDIFT | |
| | (26% 3′) | | EGKINPLLDEPLSPDTCLLAISAVYFKAKWLTPFEKE | |
| | | | FTSDYPFYVSPTEMVDVSMMSMYGKAFNHASVKE | |
| | | | SFGNFSIIELPYVGDTSMMVILPDKIDGLESIEQNLTD | |
| | | | TNFKKWCNSLEATFIDVHIPKFKVTGSYNLVDTLVK | |
| | | | SGLTEVFGSTGDYSNMCNSDVSVDAMIHKTYIDVN | |
| | | | EEYTEAAAATCALVSDCASTITNEFCVDHPFIYVIRH | |
| | | | VDGKILFVGRYCSPTTNC | |
| SEQ ID | VACWR196 | AAO89475.1 | MTANFSTHVFSPQHCGCDRLTSIDDVRQCLTEYIYW | Inside |
| NO: 128 | | | SSYAYRNRQCAGQLYSTLLSFRDDAELVFIDIRELV | Deletion |
| | | | KNMPWDDVKDCAEIIRCYIPDEQKTIREISAIIGLCA | |
| | | | YAATYWGGEDHPTSNSLNALFVMLEMLNYVDYNII | |
| | | | FRRMN | |
| SEQ ID | VACWR197 | AAO89476.1 | MSILPVIFLSIFFYSSFVQTFNAPECIDKGQYFASFME | Inside |
| NO: 129 | | | LENEPVILPCPQINTLSSGYNILDILWEKRGADNDRII | Deletion |
| | | | PIDNGSNMLILNPTQSDSGIYICITTNETYCDMMSLN | |
| | | | LTIVSVSESNIDLISYPQIVNERSTGEMVCPNINAFIAS | |
| | | | NVNADIIWSGHRRLRNKRLKQRTPGIITIEDVRKND | |
| | | | AGYYTCVLEYIYGGKTYNVTRIVKLEVRDKIIPSTM | |
| | | | QLPDGIVTSIGSNLTIACRVSLRPPTTDADVFWISNG | |
| | | | MYYEEDDGDGNGRISVANKIYMTDKRRVITSRLNI | |
| | | | NPVKEEDATTFTCMAFTIPSISKTVTVSIT | |
| SEQ ID | VACWR198 | AAO89477.1 | MSRKFMQVYEYDREQYLDEFIEDRYNDSFITSPEYY | Inside |
| NO: 130 | | | SAEKYMCRYTTLNHNCINVRRCALDSKLLHDIITNC | Deletion |
| | | | KIYNNIELVRATKFVYYLDLIKCNWVSKVGDSVLYP | |
| | | | VIFITHTSTRNLDKVSVKTYKGVKVKKLNRCADHAI | |
| | | | VINPFVKFKLTLPNKTSHAKVLVTFCKLKTDITPVEA | |
| | | | PLPGNVLVYTFPDINKRIPGYIHLNIEGCIDGMIYINS | |
| | | | SKFACVLKLHRSMYRIPPFPIDKSCCSQYINYDIEIPI | |
| | | | HDLIKDVAIFKNKETVYYLKLNNKTIARFTYFNNID | |
| | | | TAITQEHEYVKIALGIVCKLMINNMHSIVGVNHSNT | |
| | | | FVNCLLEDNV | |
| SEQ ID | VACWR199 | AAO89478.1 | MSRRLIYVLNINRESTHKIQENEIYTYFSHCNIDHTST | Inside |
| NO: 131 | | | ELDFVVKNYDLNRRQPVTGYTALHCYLYNNYFTN | Deletion |
| | | | DVLKILLNHGVDVTMKTSSGRMPVYILLTRCCNISH | |
| | | | DVVIDMIDKDKNHLLHRDYSNLLLEYIKSRYMLLK | |
| | | | EEDIDENIVSTLLDKGIDPNFKQDGYTALHYYYLCL | |
| | | | AHVYKPGECRKPITIKKAKRIISLFIQHGANLNALDN | |
| | | | CGNTPFHLYLSIEMCNNIHMTKMLLTFNPNFEICNN | |
| | | | HGLTPILCYITSDYIQHDILVMLIHHYETNVGEMPID | |
| | | | ERRIIVFEFIKTYSTRPADSITYLMNRFKNIDIYTRYE | |
| | | | GKTLLHVACEYNNTHVIDYLIRINGDINALTDNNKH | |
| | | | ATQLIIDNKENSPYTINCLLYILRYIVDKNVIRSLVDQ | |
| | | | LPSLPIFDIKSFEKFISYCILLDDTFYNRHVRNRDSKT | |
| | | | YRYAFSKYMSFDKYDGIITKCHKETILLKLSTVLDT | |
| | | | TLYAVLRCHNSKKLRRYLTELKKYNNDKSFKIYSNI | |
| | | | MNERYLNVYYKDMYVSKVYDKLFPVFTDKNCLLT | |
| | | | LLPSEIIYEILYMLTINDLYNISYPPTKV | |
| SEQ ID | B18R/ | AAO89479.1 | MTMKMMVHIYFVSLLLLLFHSYAIDIENEITEFFNK | Inside |
| NO: 132 | VACWR200 | | MRDTLPAKDSKWLNPACMFGGTMNDIAALGEPFS | Deletion |
| | | | AKCPPIEDSLLSHRYKDYVVKWERLEKNRRRQVSN | |
| | | | KRVKHGDLWIANYTSKFSNRRYLCTVTTKNGDCV | |
| | | | QGIVSHIRKPPSCIPKTYELGTHDKYGIDLYCGILY | |
| | | | AKHYNNITWYKDNKEINIDDIKYSQTGKELIIHNPEL | |
| | | | EDSGRYDCYVHYDDVRIKNDIVVSRCKILTVIPSQD | |
| | | | HRFKLILDPKINVTIGEPANITCTAVSTSLLIDDVLIE | |
| | | | WENPSGWLIGFDFDVYSVLTSRGGITEATLYFENVT | |
| | | | EEYIGNTYKCRGHNYYFEKTLTTTVVLE | |
| SEQ ID | VACWR201 | AAO89480.1 | MHVIDVDVRLYMSTFIIIDQSTENTSIDTTVTINIIYL | Not |
| NO: 133 | | | AIMKIIMNIIMMIMIELV | Present |
| SEQ ID | VACWR202 | AAO89481.1 | MNSESDNISIKTEYEFYDETQDQSTQLVGYDIKLKT | Not |
| NO: 134 | | | NEDDFMANIDQWVSMII | Present |
| SEQ ID | VACWR203 | AAO89482.1 | MEMYPRHRYSKHSVFKGFSDKVRKNDLDMNVVKE | Inside |
| NO: 135 | | | LLSNGASLTIKDSSNKDPITVYFRRTIMNLEMIDERK | Deletion |
| | | | YIVHSYLKNYKNFDYPFFRKLVLTNKHCLNNYYNIS | |
| | | | DSKYGTPLHILASNKKLITPNYMKLLVYNGNDINAR | |
| | | | GEDTQMRTPLHKYLCKFVYHNIEYGIRYYNEKIIDA | |
| | | | FIELGADLTIPNDDGMIPVVYCIHSNAEYGYNNITNI | |
| | | | KIIRKLLNLSRRASHNLFRDRVMHDYISNTYIDLECL | |
| | | | DIIRSLDGFDINGYFEGRTPLHCAIQHNFTQIAKYLL | |
| | | | DRGADIVVPNTLIIHQYIQ | |
| SEQ ID | VACWR204 | AAO89483.1 | MLNFSLCLYPVFILNKLVLRTQSIILHTINNASIKNR | Not |
| NOS 677 | | | \\\\\\ and \\\\\\ | Present |
| and 136 | | | MEEDTNISNKVIRYNTVNNIWETLPNFWTGTINPGV | |
| | | | VSHKDDIYVVCDIKDEKNVKTCIFRYNTNTYNGWE | |
| | | | LVTTTESRLSALHTILYNNTIMMLHCYESYMLQDTF | |
| | | | NVYTREWNHMCHQHSNSYIMYNILPIY | |
| SEQ ID | SPI-1/ | AAO89484.1 | MDIFKELILKHTDENVLISPVSILSTLSILNHGAAGST | Outside |
| NO: 137 | VACWR205 | | AEQLSKYIENMNENTPDDNNDMDVDIPYCATLATA | Deletion |
| | | | NKIYGSDSIEFHASFLQKIKDDFQTVNFNNANQTKE | |
| | | | LINEWVKTMTNGKINSLLTSPLSINTRMTVVSAVHF | |
| | | | KAMWKYPFSKHLTYTDKFYISKNIVTSVDMMVSTE | |
| | | | NNLQYVHINELFGGFSIIDIPYEGNSSMVIILPDDIEGI | |
| | | | YNIEKNITDEKFKKWCGMLSTKSIDLYMPKFKVEM | |
| | | | TEPYNLVPILENLGLTNIFGYYADFSKMCNETITVEK | |
| | | | FLHTTFIDVNEEYTEASAVTGVFMTNFSMVYRTKV | |
| | | | YINHPFMYMIKDNTGRILFIGKYCYPQ | |
| SEQ ID | C13L/ | AAO89485.1 | MMIYGLIACLIFVTSSIASPLYIPVIPPISEDKSFNSVE | Outside |
| NO: 138 | VACWR206 | | VLVSLFRDDQKDYTVTSQFNNYTIDTKDWTIGVLST | Deletion |
| | | | PDGLDIPLTNITYWSRFTIGRALFKSESEDIFQKKMSI | |
| | | | LGVSIECKKSSTLLTFLTVRKMTRVFNKFPDMAYYR | |
| | | | GDCLKAVYVTMTYKNTKTGETDYTYLSNGGLPAY | |
| | | | YRNGVDG | |
| SEQ ID | VACWR207 | AAO89486.1 | MKLFTQNDRYFGLLDSCTHIFCITCINIWHKTRRETG | Outside |
| NO: 139 | | | ASDNCPICRTRFRNITMSKFYKLVN | Deletion |
| SEQ ID | p28/ | AAO89487.1 | MEFDPAKINTSSIDHVTILQYIDEPNDIRLTVCIIRNIN | Outside |
| NO: 140 | VACWR208 | | NITYYINITKINTHLANQFRAWKKRIAGRDYMTNLS | Deletion |
| | | | RDTGIQQSKLTETIRNCQKNRNIYGLYIHYNLVINVV | |
| | | | IDWITDVIVQSILRGLVNWYIANNTYTPNTPNNTTTI | |
| | | | SELDIIKILDKYEDVYRVSKEKECGICYEVVYSKR | |
| SEQ ID | C10L/ | AAO89488.1 | MDIYDDKGLQTIKLFNNEFDCIRNDIRELFKHVTDS | Outside |
| NO: 141 | VACWR209 | | DSIQLPMEDNSDIIENIRKILYRRLKNVECVDIDSTIT | Deletion |
| | | | FMKYDPNDDNKRTCSNWVPLTNNYMEYCLVIYLE | |
| | | | TPICGGKIKLYHPTGNIKSDKDIMFAKTLDFKSKKVL | |
| | | | TGRKTIAVLDISVSYNRSMTTIHYNDDVDIDIHTDK | |
| | | | NGKELCYCYITIDDHYLVDVETIGVIVNRSGKCLLV | |
| | | | NNHLGIGIVKDKRISDSFGDVCMDTIFDFSEARELFS | |
| | | | LTNDDNRNIAWDTDKLDDDTDIWTPVTEDDYKFLS | |
| | | | RLVLYAKSQSDTVFDYYVLTGDTEPPTVFIFKVTRF | |
| | | | YFNMPK | |
| SEQ ID | VGF-1/ | AAO89489.1 | MSMKYLMLLFAAMIIRSFADSGNAIETTSPEITNATT | Outside |
| NO: 142 | VACWR210 | | DIPAIRLCGPEGDGYCLHGDCIHARDIDGMYCRCSH | Deletion |
| | | | GYTGIRCQHVVLVDYQRSENPNTTTSYIPSPGIMLV | |
| | | | LVGIIIITCCLLSVYRFTRRTKLPIQDMVVP | |
| SEQ ID | VACWR211 | AAO89490.1 | MDEIVRIVRDSMWYIPNVFMDDGKNEGHVSVNNV | Inside |
| NO: 143 | | | CHMYFTFFDVDTSSHLFKLVIKHCDLNKRGNSPLHC | Deletion |
| | | | YTMNTRFNPSVLKILLHHGMRNFDSKDEKGHHYQS | |
| | | | ITRSLIY | |
| SEQ ID | C20L/ | AAO89491.1 | MLFYLEEPIRGYVIILIVHPSWNDCATGHILIMLLNW | Inside |
| NO: 144 | VACWR212 | | HEQKEEGQHLLYLFIKHNQGYTLNILRYLLDRFDIQ | Deletion |
| | | | KDEYYNTAFQNCNNNVASYIGYDINLPTKDGIRLGV | |
| SEQ ID | VACWR213 | AAO89492.1 | MLPHTSDTTSTFRLKTVFDLVFENRNIIYKADVVNDI | Inside |
| NO: 145 | | | IHHRLKVSLPMIKSLFYKMSLPTTITT | Deletion |
| SEQ ID | VACWR214 | AAO89493.1 | MYDDLIEQCHLSMERKSKLVDKALNKLESTIGQSRL | Outside |
| NO: 146 | | | SYLPPEIMRNII | Deletion |
| SEQ ID | B28R/ | AAO89494.1 | MKSVLYSYILFLSCIIINGRDIAPHAPSDGKCKDNEY | Inside |
| NO: 147 | VACWR215 | | KRHNLCPGTYASRLCDSKTNTQCTPCGSGTFTSRNN | Deletion |
| | | | HLPACLSCNGRRDRVTRLTIESVNALPDIIVFSKDHP | |
| | | | DARHVFPKQNVE | |
| SEQ ID | VACWR216 | AAO89495.1 | MDSLRPVVVVNWIQINFHIDIVKGITGYGFAFICGRD | Outside |
| NO: 148 | | | GVRICSETTRRTDDVSGYSVSYSTFCLGNTCLASG | Deletion |
| SEQ ID | VACWR217 | AAO89496.1 | MWKLICIQLTTTTGLSESISTSELTITMNHKDCNPVF | Outside |
| NO: 149 | | | REEYFSVLNKVATSGFFTGERCAL | Deletion |
| SEQ ID | B29R/ | AAO89497.1 | MHVPASLQQSSSSSSSCTEEENKHHMGIDVIIKVTK | Inside |
| NO: 150 | VACWR218 | | QDQTPTNDKICQSVTEITESESDPDPEVESEDDSTSV | Deletion |
| | (44% 5′) | | EDVDPPTTYYSIIGGGLRMNFGFTKCPQIKSISESAD | |
| | | | GNTVNARLSSVSPGQGKDSPAITHEEALAMIKDCEV | |
| | | | SIDIRCSEEEKDSDIKTHPVLGSNISHKKVSYEDIIGST | |
| | | | IVDTKCVKNLEFSVRIGDMCKESSELEVKDGFKYVD | |
| | | | GSASEGATDDTSLIDSTKLKACV | |

TABLE 38
Examples of proteins encoded by Tian Tan Vaccinia genes
equivalent to those deleted in CopMD3p vector

| | | Protein | | |
|---|---|---|---|---|
| SEQ ID | | Accession | | |
| NO | Gene | ID | Amino Acid Sequence | Location |
| SEQ ID | TF3L | AAF34083.1 | MNHCLLAISAVYFKAKWLTPFEKEFTSDYPFYVSPT | Inside |
| NO: 151 | (41% 3′) | | EMVDVSMMSMYGKAFNHASVKESFGNFSIIELPYV | Deletion |
| | | | GDTSMMVILPDKIDGLESIEQNLTDTNFKKWCDFM | |
| | | | DAMFIDVHIPKFKVTGSYNLVDTLVKSGLTEVFGST | |
| | | | GDYSNMCNLDVSVDAMIHKTYIDVNEEYTEAAAA | |
| | | | TCALVSDCASTITNEFCVDHPFIYVIRHVDGKILFVG | |
| | | | RYCSPTTNC | |
| SEQ ID | TB15R | AAF34084.1 | MTANFSTHVFSPQHCGCDRLTSIDDVKQCLTEYIYW | Inside |
| NO: 152 | | | SSYAYRNRQCAGQLYSTLLSFRDDAELVFIDIRELV | Deletion |
| | | | KNMPWDDVKDCTEIIRCYIPDEQKTIREISAIIGLCA | |
| | | | YAATYWGGEDHPTSNSLNALFVMLEMLNYVDYNII | |
| | | | FRRMN | |
| SEQ ID | ORFL | AAF34085.1 | MYNSSIHTPEYDVIIHVIEHLKHHKQCVQTVTSGMV | |
| NO: 153 | | | FTSPVSSSICTKSDDGRNLSDGFLLIRYITTDDFCTIF | |
| | | | DIIPRHIFYQLANVDEH | |
| SEQ ID | TB16R | AAF34086.1 | MELENEPVILPCPQINTLSSGYNILDILWEKRGADND | Inside |
| NO: 154 | | | RIIPIDNGSNMLILNPTQSDSGIYICITTNETYCDMMS | Deletion |
| | | | LNLTIVSVLESNIDLISYPQIVNERSTGEMVCPNINAF | |
| | | | IASNVNADIIWSGHRRLRNKRLKQRTPGIITIEDVRK | |
| | | | NDAGYYTCVLEYIYRGKTYNVTRIVKLEVRDKIIPS | |
| | | | TMQLPDGIVTSIGSNLTIACRVSLRPPTTDADVFWIS | |
| | | | NGMYYEEDDGDGNGRISVANKIYMTDKRRVITSRL | |
| | | | NINPVKEEDATTFTCMAFTIPSISKTVTVSIT | |
| SEQ ID | ORFL | AAF34087.1 | MVIIPGVRCLSLLFLRRRCPLHIISAFTLLAINALILGH | |
| NO: 155 | | | TISPVDLSFTICGYEIRSIFDSKTDTIVKFNDIMSQ | |
| SEQ ID | TB17L | AAF34088.1 | MSRKFMQVYEYDREQYLDEFIEDRYNDSFITSPEYY | Inside |
| NO: 156 | | | SAEKYMCRYTTLNHNCVNVRRCALDSKLLHDIITN | Deletion |
| | | | CKIYNNIELVRATKFVYYLDLIKCNWVSKVGDSVL | |
| | | | YPVIFITHTSTRNLDKVSVKTYKGVKVKKLNRCAD | |
| | | | HAIVINPFVKFKLTLPNKTSHAKVLVTFCKLRTDITQ | |
| | | | IEAPLSGNVLVYTFPNINKRIPGYIHVNIEGCIDGMIY | |
| | | | INSSKFACVLKLHRSMYRIPPFPIDKSCCSQYTNGDI | |
| | | | EIPIHDLIKDVAIFKNKETVYYLKLNNKTIARFTYFN | |
| | | | NIDTAITQEHEYVKIALGIVCKLMINNMHSIVGVNH | |
| | | | SNTFVNCLLEDNV | |
| SEQ ID | TB18R | AAF34089.1 | MSRRLIYVLNINRESTHKIQENEIYTYFSHCNIDHTST | Inside |
| NO: 157 | | | ELDFVVKNYDLNRRHPVTGYTALHCYLYNNYFTN | Deletion |
| | | | DVLKILLNHGVDVTMKTSSGRMPVYILLTRCCNISH | |
| | | | DVVIDMIDKDKNHLLHRDYSNLLLEYIKSRYMLLK | |
| | | | EEDIDENIVSTLLDKGIDPNFKQDGYTALHYYYLCL | |
| | | | AHVYKPGECRKPITIKKAKRIISLFIQHGANLNALDN | |
| | | | CGNTPFHLYLSIEMCNNIHMTKMLLTFNPNFKICNN | |
| | | | HGLTPILCYITSDYIQHDILVMLIHHYETNVGEMPID | |
| | | | ERRIIVFEFIKTYSTRPADSITYLMNRFKNINIYTRYE | |
| | | | GKTLLHVACEYNNTQVIDYLIRINGDINALTDNNKH | |
| | | | ATQLIIDNKENSPYTINCLLYILRYIVDKNVIRSLVDQ | |
| | | | LPSLPIFDIKSFEKFISYCILLDDTFYDRHVKNRNSKT | |
| | | | YRYAFSKYMSFDKYDGIITKCHDETMLLKLSTVLDT | |
| | | | TLYAVLRCHNSRKLRRYLTELKKYNNDKSFKIYSNI | |
| | | | MNERYLNVYYKDMYVSKVYDKLFPVFTDKNCLLT | |
| | | | LLPSEIIYEILYMLTINDLYNISYPPTKV | |
| SEQ ID | TB19R | AAF34090.1 | MTMKMMVHIYFVSLSLLLLLFHSYAIDIENEITEFFN | Inside |
| NO: 158 | | | KMRDTLPAKDSKWLNPACMFGGTMNDIAALGEPF | Deletion |
| | | | SAKCPPIEDSLLSHRYKDYVVKWERLEKNRRRQVS | |
| | | | NKRVKHGDLWIANYTSKFSNRRYLCTVTTKNGDC | |
| | | | VQGIVRSHIKKPPSCIPKTYELGTHDKYGIDLYCGIL | |
| | | | YAKHYNNITWYKDNKEINIDDIKYSQTGKELIIHNPE | |
| | | | LEDSGRYDCYVHYDDVRIKNDIVVSRCKILTVIPSQ | |
| | | | DHRFKLILDPKINVTIGEPANITCTAVSTSLLIDDVLI | |
| | | | EWENPSGWLIGFDFDVYSVLTSRGGITEATLYFENV | |
| | | | TEEYIGNTYKCRGHNYYFEKTLTTTVVLE | |
| SEQ ID | ORFR | AAF34091.1 | MHVIDVDVRLYMSTFIIIDQSTENTSIDTTVTINIIYL | |
| NO: 159 | | | AIMKIIMNIIMMIMIELV | |
| SEQ ID | TB21R | AAF34092.1 | LKNVECVDIDSTITFMKYDPNDDNKRTCSNWVPLT | |
| NO: 160 | | | NNYMEYCLVIYLETPICGGKIKLYHPTGNIKSDKDI | |
| | | | MFAKTLDFKSTKVLTGRKTIAVLDISVSYNRSMTTI | |
| | | | HYNDDVDIDIHTDKNGKELCYCYITIDDHYLVDVET | |
| | | | IGVIVNRSGKCLLVNNHLGIGIVKDKRISDSFGDVC | |
| | | | MDTIFDFSEARELFSLTNDDNRNIAWDTDKLDDDT | |
| | | | DIWTPVTENDYKFLSRLVLYAKSQSDTVFDYYVLT | |
| | | | GDTEPPTVFIFKVTRFYFNMPK | |
| SEQ ID | TB22L | AAF34093.1 | MYCRCSHGYTGIRCQHVVLVDYQRSEKPNTTTSYIP | |
| NO: 161 | | | SPGIMLVLVGIIIITCCLLSVYRFTRRTKLPLQDMVVP | |
| SEQ ID | TB23R | AAF34094.1 | MHVPASLQQSSSSSSSCTEEENKHHMGIDVIIKVTK | Inside |
| NO: 162 | (44% 5′) | | QDQTPTNDKICQSVTEITESESDPDPEVESEDDSTSV | Deletion |
| | | | EDVDPPTTYYSIIGGGLRMNFGFTKCPQIKSISESAD | |
| | | | GNTVNARLSSVSPGQGKDSPAITHEEALAMIKDCEV | |
| | | | SIDIRCSEEEKDSDIKTHPVLGSNISHKKVSYEDIIGST | |
| | | | IVDTKCVKNLEFSVRIGDMCKESSELEVKDGFKYVD | |
| | | | GSASEGATDDTSLIDSTKLKACV | |
| | TB20R | | | |
| | ORFL | | | |

TABLE 39
Examples of proteins encoded by Wyeth Vaccinia genes equivalent to
those deleted in CopMD3p vector

| | | Protein | | |
|---|---|---|---|---|
| SEQ ID | | Accession | | |
| NO | Gene | ID | Amino Acid Sequence | Location |
| SEQ ID | VAC_DPP20_207 | AEY74905.1 | MNHCLLAISAVYFKAKWLTPFEKEFTSDYPFYVSPT | Inside |
| NO: 163 | | | EMVDVSMMSMYGKAFNHASVKESFGNFSIIELPYV | Deletion |
| | | | GDTSMMVILPDKIDGLESIEQNLTDTNFKKWCDFM | |
| | | | DAMFIDVHIPKFKVTGSYNLVDTLVKSGLTEVFGST | |
| | | | GDYSNMCNLDVSVDAMIHKTYIDVNEEYTEAAAA | |
| | | | TCALVSDCASTVTNEFCADHPFIYVIRHVDGKILFV | |
| | | | GRYCSPTTNC | |
| SEQ ID | VAC_DPP10_208 | AEY74906.1 | MTANFSTHVFSPQHCGCDRLTSIDDVKQCLTEYIYW | Inside |
| NO: 164 | | | SSYAYRNRQCAGQLYSTLLSFRDDAELVFIDIRELV | Deletion |
| | | | KHMPWDDVKDCAEIIRCYIPDEQKTIREISAIIGLCA | |
| | | | YAATYWGGEDHPTSNSLNALFVMLEMLNYVDYNII | |
| | | | FRRMN | |
| SEQ ID | VAC_DPP12_209 | AEY74907.1 | MSILPVIFLSIFFYSSFVQTFNASECIDKGQYFASFME | Inside |
| NO: 165 | | | LENEPVILPCPQINTLSSGYNILDILWEKRGADNDRII | Deletion |
| | | | PIDNGSNMLILNPTQSDSGIYICITTNETYCDMMSLN | |
| | | | LTIVSVSESNIDLISYPQIVNERSTGEMVCPNINAFIAS | |
| | | | NVNADIIWSGHRRLRNKRLKQRTPGIITIEDVRKND | |
| | | | AGYYTCVLEYIYGGKTYNVTRIVKLEVRDKIIPSTM | |
| | | | QLPDGIVTSIGSNLTIACRVSLRPPTTDTDVFWISNG | |
| | | | MYYEEDDGDGDGRISVANKIYMTDKRRVITSRLNI | |
| | | | NPVKEEDATTFTCMAFTIPSISKTVTVSIT | |
| SEQ ID | VAC_DPP20_210 | AEY4908.1 | MSRKFMQVYEYDREQYLDEFIEDRYNDSFITSPEYY | Inside |
| NO: 166 | | | SAEKYMCRYTTLNHNCVNVRRCALDSKLLHDIITN | Deletion |
| | | | CKIYNNIELVRATKFVYYLDLIKCNWVSKVGDSVL | |
| | | | YPVIFITHTSTRNLDKVSVKTYKGVKVKKLNRCAD | |
| | | | HAIVINPFVKFKLTLPNKTSHAKVLVTFCKLRTDITQ | |
| | | | IEAPLSGNVLVYTFPDINKRIPGYIHVNIEGCIDGMIY | |
| | | | INSSKFACVLKLHRSMYRIPPFPIDKSCCSQYTNDDI | |
| | | | EIPIHDLIKDVAIFKNKETVYYLKLNNKTIARFTYFN | |
| | | | NIDTAITQEHEYVKIALGIVCKLMINNMHSIVGVNH | |
| | | | SNTFVNCLLEDNV | |
| SEQ ID | VAC_DPP20_211 | AEY74909.1 | MSRRLIYVLNINRESTHKIQENEIYTYFSHCNIDHTST | Inside |
| NO: 167 | | | ELDFVVKNYDLNRRQPVTGYTALHCYLYNNYFTN | Deletion |
| | | | DVLKILLNHGVDVTMKTSSGRMPVYILLTRCCNISH | |
| | | | DVVIDMIDKDKNHLSHRDYSNLLLEYIKSRYMLLK | |
| | | | EEDIDENIVSTLLDKGIDPNFKQDGYTALHYYYLCL | |
| | | | AHVYKPGECRKPITIKKAKRIISLFIQHGANLNALDN | |
| | | | CGNTPFHLYLSIEMCNNIHMTKMLLTFNPNFKICNN | |
| | | | HGLTPILCYITSDYIQHDILVMLIHHYETNVGEMPID | |
| | | | ERRIIVFEFIKTYSTRPADSITYLMNRFKNINIYTRYE | |
| | | | GKTLLHVACEYNNTHVIDYLIRINGDINALTDNNKH | |
| | | | AIQLIIDNKENSPYTIDCLLYILRYIVDKNVIRSLVDQ | |
| | | | LPSLPIFDIKSFEKFISYCILLDDTFYNRHVRNRNSKT | |
| | | | YRYAFSKYMSFDKYDGIITKCHDETMLLKLSTVLDT | |
| | | | TLYAVLRCHNSKKLRRYLNELKKYNNDKSFKIYSNI | |
| | | | MNERYLNVYYKDMYVSKVYDKLFPVFTDKNCLLT | |
| | | | LLPSEIIYEILYMLTINDLYNISYPPTKV | |
| SEQ ID | VAC_DPP20_212 | AEY74910.1 | MTMKMMVHIYFVSLSLLLLLFHSYAIDIENEITEFFN | Inside |
| NO: 168 | | | KMRDTLPAKDSKWLNPACMFGGTMNDIATLGEPFS | Deletion |
| | | | AKCPPIEDSLLSHRYKDYVVKWERLEKNRRRQVSN | |
| | | | KRVKHGDLWIANYTSKFSNRRYLCTVTTKNGDCV | |
| | | | QGIVRSHIRKPPSCIPKTYELGTHDKYGIDLYCGILY | |
| | | | AKHYNNITWYKDNKEINIDDIKYSQTGKKLIIHNPEL | |
| | | | EDSGRYDCYVHYDDVRIKNDIVVSRCKILTVIPSQD | |
| | | | HRFKLKRNCGYASN | |
| SEQ ID | VAC_DPP10_217 | AEY74911.1 | MRQIKINGTDMLTVMYMLNKPTKKRYVNNPIFTD | Not |
| NO: 169 | | | WANKQYKFYNQIIYNANKLIEQSKKIDDMIEEVSID | Present |
| | | | DNRLSTLPLEIRHLIFSYAFL | |
| SEQ ID | VAC_DPP10_218 | AEY74912.1 | MSSKGGSGGMWSVFIHGHDGSNKGSKTYTSGGGG | Outside |
| NO: 170 | | | MWGGGSSSGVNGGVKSGTGKI | Deletion |
| SEQ ID | VAC_DPP10_219 | AEY74913.1 | MFDYLENEEVALDELKQMLRDRDPNDTRNQFKNN | Outside |
| NO: 171 | | | ALHAYLFNEHCNNVEVVKLLLDSGTNPLRKNWRQ | Deletion |
| | | | LPH | |
| SEQ ID | VAC_DPP10_220 | AEY74914.1 | MLKLKDIAMALLEATGFSNINDFNIFSYMKSKNVD | Outside |
| NO: 172 | | | VDLIKVLVEHGFDLSVKCENHRSVIENYVMTMILFI | Deletion |
| | | | ENGCSVLYEDEY | |
| SEQ ID | VAC_DPP10_221 | AEY74915.1 | MKGIDNTAYSYIDDLTCCTRGIMADYLNSDYRYNK | Outside |
| NO: 173 | | | DVDLVKLFLENGKPHGIMCSIVPLWRNDKETIFLILK | Deletion |
| | | | TMNSDVLQHILIEYMTFGDIPLVEYGTVVNKEAIHG | |
| | | | YFRNINIDSYTMKYLLKKEGRCHQLSRLDTYVNPT | |
| | | | MDVIISTLIHTKRVFVTCLMLAQFLVL | |
| SEQ ID | VAC_DPP10_222 | AEY74916.1 | MPSIISIGHLCKSNYGCYNFYTYTYKKGLCDMSYAC | Outside |
| NO: 174 | | | PILSTINICLPYLKDINMIDKRGETLLHKAVRYNKQS | Deletion |
| | | | LVSLLLESGSDVNIRSNNGYTCIAIAINESKNIELLKM | |
| | | | LLCHKPTLDYVIDSLREISNIVDNDYAIKQCIKYAMII | |
| | | | DDCTSSKIPEFISQRYNDYIDLCN | |
| SEQ ID | VAC_DPP10_223 | AEY49171.1 | MKKIMVGGNTMFSLIFTDHGAKIIHRYANNPELREY | Outside |
| NO: 175 | | | YELKQNKIYVEAYDIISNAIVKHDRIHKTIESVDDNT | Deletion |
| | | | YISNLPYTIKYKIFEQQ | |
| SEQ ID | VAC_DPP10_224 | AEY74918.1 | MRILFLIAFMYGCVHSYVNAVETKCSNLDIVTSSGE | Outside |
| NO: 176 | | | FHCSGCVEHMPNFSYMYWLAKDMRSDEDAKFIEH | Deletion |
| | | | LGEGIKEDETVRTIDGRIVTLQKVLHVTDTNKFAHY | |
| | | | RFTCVLTTIDGVSKKNIWLK | |
| SEQ ID | VAC_DPP10_225 | AEY74919.1 | MKLFTQNDRYFGLLDSCNHIFCITCINIWFIKTRRET | Outside |
| NO: 177 | | | GASDNCPICRTRFRNITMSKFYKLVN | Deletion |
| SEQ ID | VAC_DPP10_226 | AEY74920.1 | MHYPKYYINITKINPHLANQFRAWKKRIAGRDYMT | Outside |
| NO: 178 | | | NLSKDTGIQQSKLYVTVKKIETYMVYIYTTI | Deletion |
| SEQ ID | VAC_DPP20_227 | AEY74921.1 | MDIYDDKGLQTIKLFNNEFDCIRNDIRELFKHVTDS | Outside |
| NO: 179 | | | DSIQLPMEDNSDIIENIRKILYRRLKNVECVDIDNTIT | Deletion |
| | | | FMKYDPNDDNKRTCSNWVPLTNNYMEYCLVIYLE | |
| | | | TPICGGKIKLYHPTGNIKSDKDIMFAKTLDFKSKKVL | |
| | | | TGRKTIAVLDISVSYNRSITTIHYNDDVDIDIHTDKN | |
| | | | GKELCYCYITIDDHYLVDVETIGVIVNRSGKCLLVN | |
| | | | NHLGIGIVKDKRISDSFGDVCMDTIFDFSEARELFSL | |
| | | | TNDDNRNIAWDTDKLDDDTDIWTPVTENDYKFLSR | |
| | | | LVLYAKSQSDTVFDYYVLTGDTEPPTVFIFKVTRFY | |
| | | | FNMFK | |
| SEQ ID | VAC_DPP20_228 | AEY74922.1 | MLINYLMLLFAAMIIRSFADSGNAIETTLPEITNATT | Outside |
| NO: 180 | | | DIPAIRLCGPEGDGYCLHGDCIHARDIDGMYCRCSH | Deletion |
| | | | GYTGIRCQHVVLVDYQRSEKPNITTSYIPSPGIMLV | |
| | | | LVGIIIITCCLLSVYRFTRRTNKLPLQDMVVP | |
| SEQ ID | VAC_DPP20_229 | AEY74923.1 | MDIFKELIVKHPDENVLISPVSILSTLSILNHGAAGST | Outside |
| NO: 181 | | | AEQLSKYIENMNENTPDDKKDDNNDMDVDIPYCAT | Deletion |
| | | | LATANKIYGSDSIEFHASFLQKIKDDFQTVNFNNAN | |
| | | | QTKELINEWVKTMTNGKINSLLTSPLSINTRMTVVS | |
| | | | AVHFKAMWKYPFSKHLTYTDKFYISKNIVTSVDMM | |
| | | | VGTENNLQYVHINELFGGFSIIDIPYEGNSSMVIILPD | |
| | | | DIEGIYNIEKNITDEKFKKWCGMLSTKSIDLYMPKF | |
| | | | KVEMTEPYNLVPILENLGLTNIFGYYADFSKMCNET | |
| | | | ITVEKFLHTTFIDVNEEYTEASAVTGVFMTNFAMVY | |
| | | | RTKVYINHPFMYMIKDTTGRILFIGKYCYPQ | |
| SEQ ID | C13L/ | AEY74924.1 | MMIYGLIACLIFVTSSIASPLYIPVIPPITEDKSFNSVE | Outside |
| NO: 182 | VAC_DPP20_230 | | VLVSLFRDDQKDYTVISQFNNYTIDTKDWTIGVLST | Deletion |
| | | | PDGLDIPLTNITYWSRFTIGRALFKSESEDIFQKKMSI | |
| | | | LGVSIECKKSSTLLTFLTVRKMTRVFNKFPDMAYYR | |
| | | | GDCLKAVYVTMTYKNTKTGETDYTYLSNGGLPAY | |
| | | | YRNGVDG | |
| SEQ ID | VAC_DPP20_231 | AEY74925.1 | MNLQKLSLAIYLTATCSWCYETCIRKTALYHDIQLE | Outside |
| NO: 183 | | | HVEDNKDSVASLPYK | Deletion |
| SEQ ID | VAC_DPP20_232 | AEY74926.1 | MSLESFIITTFNNSSTNIDNMCHLYVKVCPSSLLFR | Inside |
| NO: 184 | | | LFVECCDINKLVEGTTPLHCYLMNEGFESSVLKNLL | Deletion |
| | | | KEYVMTSITQIFNS | |
| SEQ ID | VAC_DPP20_233 | AEY74927.1 | MISLSFLIHNPLKKWKLKPSISINGYRSTFTMAFPCA | Inside |
| NO: 185 | | | QFRPCHCHATKDSLNTVADVRHCLTEYILWVSHRW | Deletion |
| | | | THRETAGPLYRLLISFRTDATELFGGELKDSLPWDNI | |
| | | | DNCVEIIKCFIRNDSMKTAEELRAIIGLCTQSAIVSGR | |
| | | | VFNDKYIDILLMLRKILNENDYLTLLDHIRTAKY | |
| SEQ ID | VAC_DPP20_234 | AEY74928.1 | MIAFIIFREIGIISTRIAMDCTCILCRLLDEDVTYKKIK | Inside |
| NO: 186 | | | LEIETCHNLSKHIDRRGNNALHCYVFNKCDTDIKIV | Deletion |
| | | | RLLLSRGVERLCRNNEGLTPLGVYSKHRYVKSQIVH | |
| | | | LLISSYSNSSNELKSNINDFDLSSDNIDLRLLKYLIVD | |
| | | | KRIRPSKNTNYAINSLGLVDIYVTTPNPRPEVLLWLL | |
| | | | KSECYSTGYVFRTCMYNSDMCKNSLHYYISSHRES | |
| | | | QSLSKDVIKCLINNNVSIHGRDEGGSLPIQYYWSFST | |
| | | | IDIEIVKLLLIKDVDTCRVYDVSPILEAYYLNKRFRV | |
| | | | TPYNVDMEIVNLLIERRHTLVDVMRSITSYDSREYN | |
| | | | HYIIDNILKRFRQQDESIVQAMLINYLHYGDMVVRC | |
| | | | MLDNGQQLSSARLLC | |
| SEQ ID | VAC_DPP20_235 | AEY74929.1 | MYGLILSRFNNCGYHCYETILIDVFDILSKYMDNID | Inside |
| NO: 187 | | | MIDNENKTLLYYAVDVNNIQFAKRLLEYGASVTTS | Deletion |
| | | | RSIINTAIQKSSYRRENKTKLVDLLLSYHPTLETMID | |
| | | | AFNRDIRYLYPEPLFACIRYALILDDDFPSKVKYDIS | |
| | | | GRHKELKRYRVDINRMKNAYISGVSMFDILFKRSK | |
| | | | RHRLRYAKNPTSNGTKKN | |
| SEQ ID | VAC_DPP20_236 | AEY74930.1 | MSRINITKKIYCSVFLFLFLSYISNYEKVNDEMYEMG | Inside |
| NO: 188 | | | EMDEIVSIVRDSMWYIPNVFMDDGKNEGHVSVNNV | Deletion |
| | | | CHMYFTFFDVDTSSHLFKLVIKHCDLNKRGNSPLHC | |
| | | | YTMNTRFNPSVLKILLHHGMRNFDSKDDHYQSITRS | |
| | | | LIY | |
| SEQ ID | VAC_DPP20_237 | AEY74931.1 | MEQTLTRLHTYLQQYTKHSPRVVYALLSRGYVIILI | Inside |
| NO: 189 | | | VHPSWNDCATGHILIMLLNWHEQKEEGQHLLYLFI | Deletion |
| | | | KHNQGYTLNILRYLLDRFDIQKDEYYNTAFQNCNN | |
| | | | NVASYIGYDINLPTKDGIRLGV | |
| SEQ ID | VAC_DPP20_238 | AEY74932.1 | MLPHTSDTTSTFRLKTVFDLVFENRNIIYKADVVNDI | Inside |
| NO: 190 | | | IHHRLKVPMIKSLFYKMSEFSPYDDYYVKKILAYCL | Deletion |
| | | | LRDESFAELHSKFCLNEDYKSVFMKNISFDKIDSIIVT | |
| SEQ ID | VAC_DPP20_239 | AEY74933.1 | MHHPMESVKTTNTNAIICVREHTLPDYANTQCTPC | Inside |
| NO: 191 | | | GSGTFTSRNNHLPACLSCNGRRDRVTLLTIESVNAL | Deletion |
| | | | PDIIVFSKDHPDARHVFPKQNVE | |
| SEQ ID | VAC_DPP20-241 | AEY74934.1 | MHVPASLQQSSSSCTEEENKHHMGIDVIIKVTKQDQ | Inside |
| NO: 192 | (43% 5′) | | TPTNDKKQSVTEITESESDPDPEVESEDDSTSVEDV | Deletion |
| | | | DLPTTYYSIIGGGLRMNFGFTKCPQIKSISESADGNT | |
| | | | VNARLSSVSPGQGKDSPAITHEEALAMIKDCEVSIDI | |
| | | | RCSEEEKDSDIKTHPVLGSNISHKKVSYEDIIGSTIVD | |
| | | | TKCVKNLEFSVRIGDMCKESSELEVKDGFKYVDGS | |
| | | | ASEGATDDTSLIDSTKLKACV | |
| SEQ ID | VAC_DPP10_225 | AEY74919.1 | MKLFTQNDRYFGLLDSCNHIFCITCINIWHKTRRET | Outside |
| NO: 193 | | | GASDNCPICRTRFRNITMSKFYKLVN | Deletion |
| SEQ ID | VAC_DPP10_226 | AEY74920.1 | MHYPKYYINITKINPHLANQFRAWKKRIAGRDYMT | Outside |
| NO: 194 | | | NLSKDTGIQQSKLYVTVKKIETYMVYIYTTI | Deletion |
| SEQ ID | VAC_DPP20_207 | AEY74921.1 | MDIYDDKGLQTIKLFNNEFDCIRNDIRELFKHVTDS | Outside |
| NO: 195 | | | DSIQLPMEDNSDIIENIRKILYRRLKNVECVDIDNTIT | Deletion |
| | | | FMKYDPNDDNKRTCSNWVPLTNNYMEYCLVIYLE | |
| | | | TPICGGKIKLYHPTGNIKSDKDIMFAKTLDFKSKKVL | |
| | | | TGRKTIAVLDISVSYNRSITTIHYNDDVDIDIHTDKN | |
| | | | GKELCYCYITIDDHYLVDVETIGVIVNRSGKCLLVN | |
| | | | NHLGIGIVKDKRISDSFGDVCMDTIFDFSEARELFSL | |
| | | | TNDDNRNIAWDTDKLDDDTDIWTPVTENDYKFLSR | |
| | | | LVLYAKSQSDTVFDYYVLTGDTEPPTVFIFKVTRFY | |
| | | | FNMPK | |
| SEQ ID | VAC_DPP20:228 | AEY74922.1 | MLINYLMLLFAAMIIRSFADSGNAIETTLPEITNATT | Outside |
| NO: 196 | | | DIPAIRLCGPEGDGYCLHGDCIHARDIDGMYCRCSH | Deletion |
| | | | GYTGIRCQHVVLVDYQRSEKPNTTTSYIPSPGIMLV | |
| | | | LVGIIIITCCLLSVYRFTRRTNKLPLQDMVVP | |
| SEQ ID | VAC_DPP20_239 | AEY74933.1 | MHHPMESVKTTNTNAIICVREHTLPDYANTQCTPC | Inside |
| NO: 197 | | | GSGTFTSRNNHLPACLSCNGRRDRVTLLTIESVNAL | Deletion |
| | | | PDIIVFSKDHPDARHVFPKQNVE | |
| SEQ ID | VAC_DPP20-241 | AEY74934.1 | MHVPASLQQSSSSCTEEENKHHMGIDVIIKVTKQDQ | Inside |
| NO: 198 | (43% 5′) | | TPTNDKICQSVTEITESESDPDPEVESEDDSTSVEDV | Deletion |
| | | | DLPTTYYSIIGGGLRMNFGFTKCPQIKSISESADGNT | |
| | | | VNARLSSVSPGQGKDSPAITHEEALAMIKDCEVSIDI | |
| | | | RCSEEEKDSDIKTHPVLGSNISHKKVSYEDIIGSTIVD | |
| | | | TKCVKNLEFSVRIGDMCKESSELEVKDGFKYVDGS | |
| | | | ASEGATDDTSLIDSTKLKACV | |

TABLE 40
Examples of proteins encoded by Lister Vaccinia genes equivalent to
those deleted in CopMD3p vector

| | | Protein | | |
|---|---|---|---|---|
| SEQ ID | | Accession | | |
| NO | Gene | ID | Amino Acid Sequence | Location |
| SEQ ID | B15R/ | ABD52695.1 | MTANFSTHVFSPQHCGCDRLTSIDDVKQCLTEYIYW | Inside |
| NO: 200 | List191 | | SSYAYRNRQCAGQLYSTLLSFRDDAELVFIDIRELV | Deletion |
| | | | KNMPWDDVKDCTEIIRCYIPDEQKTIREISAIIGLCA | |
| | | | YAATYWGGEDHPTSNSLNALFVMLEMLNYVDYNII | |
| | | | FRRMN | |
| SEQ ID | List192 | ABD52696.1 | MSILPVIFLPIFFYSSFVQTFNAPECIDKGQYFASFME | Inside |
| NO: 201 | | | LENEPVILPCPQINTLSSGYNILDILWEKRGADNDRII | Deletion |
| | | | PIDNGSNMLILNPTQSDSGIYICITTNETYCDMMSLN | |
| | | | LTIVSVSESNIDLISYPQIVNERSTGEMVCPNINAFIAS | |
| | | | NVNADIIWSGHRRLRNKRLKQRTPGIITIEDVRKND | |
| | | | AGYYTCVLEYIYRGKTYNVTRIVKLEVRDKIIPSTM | |
| | | | QLPDGIVTSIGSNLTIACRVSLRPPTTDADVFWISNG | |
| | | | MYYEEDDGDGNGRISVANKIYMTDKRRVITSRLNI | |
| | | | NPVKEEDATTFTCMAFTIPSISKTVTVSIT | |
| SEQ ID | B17L/ | ABD52698.1 | MSRKFMQVYEYDREQYLDEFIEDRYNDSFITSPEYY | Inside |
| NO: 202 | List193 | | SAEKYMCRYTTLNHNCINVRRCALDSKLLHDIITNC | Deletion |
| | | | KIYNNIELVRATKFVYYLDLIKCNWVSKVGDSVLYP | |
| | | | VIFITHTSTRNLDKVSVKTYKGVKVKKLNRCADHAI | |
| | | | VINPFVKFKLTLPNKTSHAKVLVTFCKLRTDITQIEA | |
| | | | PLSGNVLVYTFPDINKRIPGYIHVNIEGCIDGMIYINS | |
| | | | SKFACVLKLHRSMYRIPPFPIDKSCCSQYTNDDIEIPI | |
| | | | HDLIKDVAIFKNKETVYYLKLNNKTIARFTYFNNID | |
| | | | TAITQEHEYVKIALGIVCKLMINNMHSIVGVNHSNT | |
| | | | FVNCLLEDNV | |
| SEQ ID | crmE/ | ABD52700.1 | MTKVIIILGFLIINTNSLSMKCEQGVSYYNSQELKCC | Not |
| NO: 203 | List195 | | KLCKPGTYSDHRCDKYSDTICGHCPSDTFTSIYNRSP | Present |
| | | | WCHSCRGPCGTNRVEVTPCTPTTNRICHCDSNSYCL | |
| | | | LKASDGNCVTCAPKTKCGRGYGKKGEDEMGNTIC | |
| | | | KKCRKGTYSDIVSDSDQCKPMTR | |
| SEQ ID | L6/ | ABD52701.1 | MAMPSLSACSSIEDDFNYGSSVASASVHIRMAFLRK | Not |
| NO: 204 | List196 | | VYGILCLQFLLTTATTAVFLYFDCMRTFIQGSPVLIL | Present |
| | | | ASMFGSIGLIFALTLHRHKHPLNLYLLCGFTLSESLT | |
| | | | LASVVTFYDVHVVMQAFMLTTAAFLALTTYTLQSK | |
| | | | RDFSKLGAGLFAALWILILSGLLGIFVQNETVKLVLS | |
| | | | AFGALVFCGFIIYDTHSLIHKLSPEEYVLASINLYLDII | |
| | | | NLFLHLLQLLEVSNKK | |
| SEQ ID | List197 | ABD52704.1 | MASPCAKFRPCHCHATKDSLNTVADVRHCLTEYIL | Inside |
| NO: 205 | | | WVSHRWTHRESAGSLYRLLISFRTDATELFGGELKD | Deletion |
| | | | SLPWDNCVEIIKCFIRNDSMKTAEELRAIIGLCTQSAI | |
| | | | VSGRVFNDKYIDILLMLRKILNENDYLTLLDHIRTA | |
| | | | KY | |
| SEQ ID | List199C | ABD52706.I | MEQTLTRLHTYLQQYTKHSPRVVYALLSRGYVIILI | Inside |
| NO: 206 | | | VHPSWNDCATGHILIMLLNWHEQKEEGQHLLYLFI | Deletion |
| | | | KHNQGYTLNILRYLLDRFDIQKDEYYNTAFQNCNN | |
| | | | NVASYIGYDINLPTKDGIRLGV | |
| SEQ ID | List199D | ABL63830.1 | MLPHTSDTTSTFRLKTVFDLVFENRNIIYKADVVNDI | Inside |
| NO: 207 | | | IHHRLKVSLPMIKSLFYKMSLPTTITT | Deletion |
| SEQ ID | C23L/ | ABL63827.1 | MKQYIVLACMCLAAAAMPASLQQSSSSSSSCTEEE | Inside |
| NO: 208 | List201 | | NKHHMGIDVIIKVTKQDQTPTNDKICQSVTEITESES | Deletion |
| | (47% 5′) | | DPDPEVESEDDSTSVEDVDPPTTYYSIIGGGLRMNF | |
| | | | GFTKCPQIKSISESADGNTVNARLSSVSPGQGKDSPA | |
| | | | ITHEEALAMIKDCEVSIDIRCSEEEKDSDIKTHPVLGS | |
| | | | NISHKKVSYEDIIGSTIVDTKCVKNLEFSVRIGDMCK | |
| | | | ESSELEVKDGFKYVDGSASEGATDDTSLIDSTKLKA | |
| | | | CV | |
| | List198A | | | |
| | List198B | | | |
| | List199A | | | |
| | List199B | | | |
| | List200 | | | |
| | List194 | | | |

### EXAMPLES

The following examples are put forth so as to provide those of ordinary skill in the art with a description of how the compositions and methods claimed herein are performed, made, and evaluated, and are intended to be purely exemplary of the invention and are not intended to limit the scope of what the inventor regards as her invention.

### Example 1—Creation of the CopMD5p3p “SKV-B8R+” Recombinant Orthopoxvirus

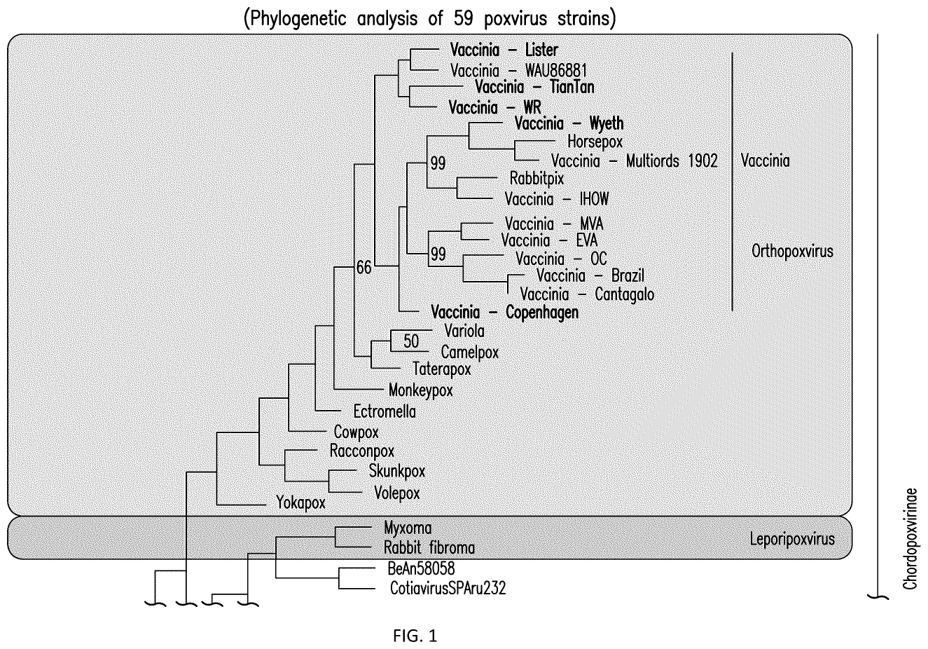

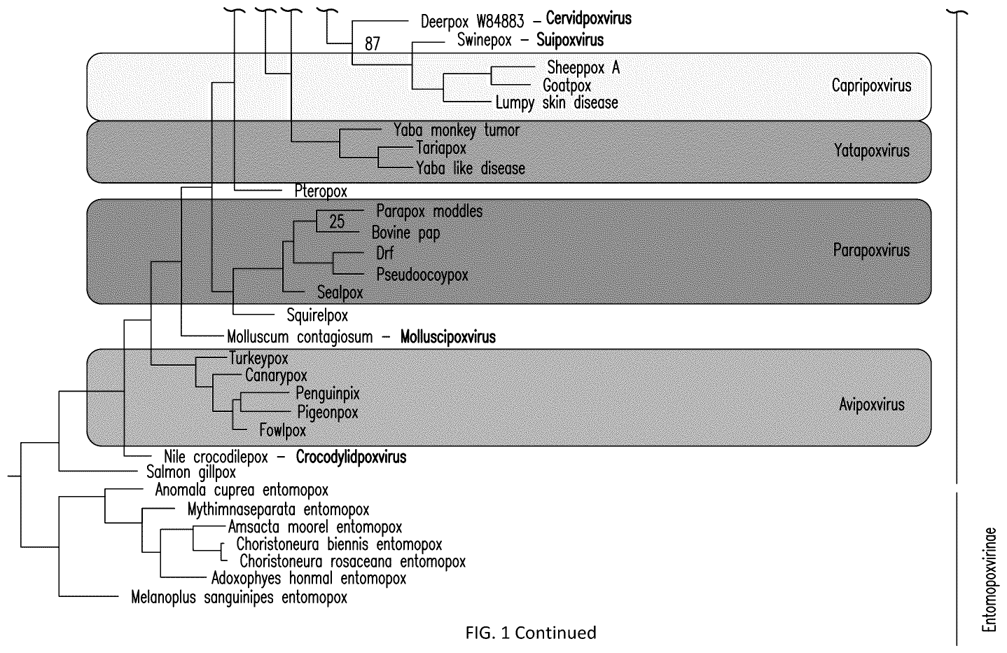

The open reading frames (ORFs) from 59 poxvirus strains were clustered into orthologs and aligned at the amino acid level (see FIG. 1 for phylogenetic analysis). Bayesian analysis was performed to determine relatedness of all strains. Poxviruses are very diverse in gene content and host range. There are several naturally occurring Vaccinia wild-type strains, which are different from one another.

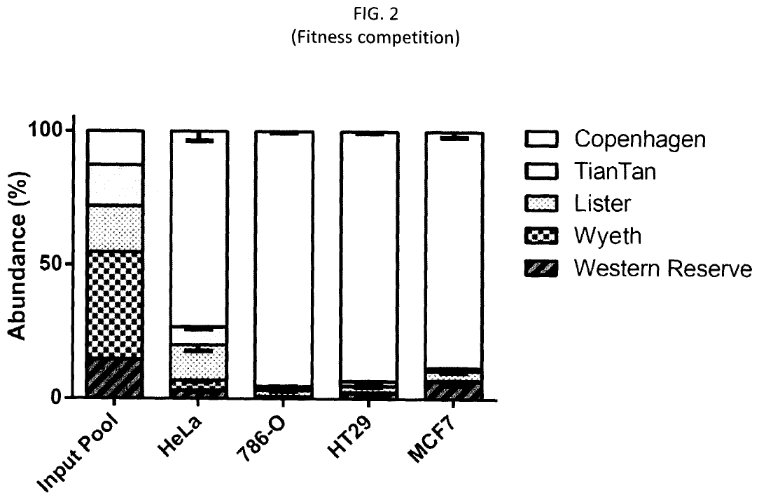

Five Vaccinia wild type strains (Copenhagen, TianTan, Lister, Wyeth, and Western Reserve) were mixed at equal plaque forming unit counts and sequenced with NGS (Input pool). The resulting mixture was passaged three times in different cancer cell lines (HeLa, 786-O, HT29, MCF7). The final population was sequenced with NGS illumina sequencing. Reads (short DNA fragments) were assigned to various strains based on sequence identity and used to calculate the percent of each strain in the final population. The relative abundance of the different viral strains was then quantified. As shown in FIG. 2, the Copenhagen strain was the most abundant vaccinia strain after three passages in any of the four cancer cell lines indicating that this strain was able to outgrow other strains and therefore replicates faster.

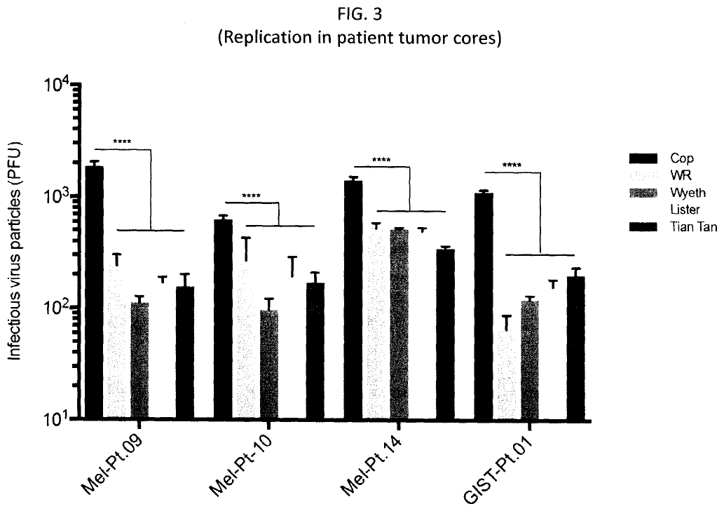

Different Vaccinia wild type strains were also used to infect at low PFU (1×10⁴) various patient tumor cores. Each strain infected on average 4 replicates each containing three 2×2 mm tumor cores. Replication was assessed through virus titering and is expressed as plaque forming units (PFU) as shown in FIG. 3. The Copenhagen strain grows to higher titers than other strains and therefore replicates faster in patient ex-vivo samples. Patient ex-vivo cores are a good mimic of a patient's 3D tumor.

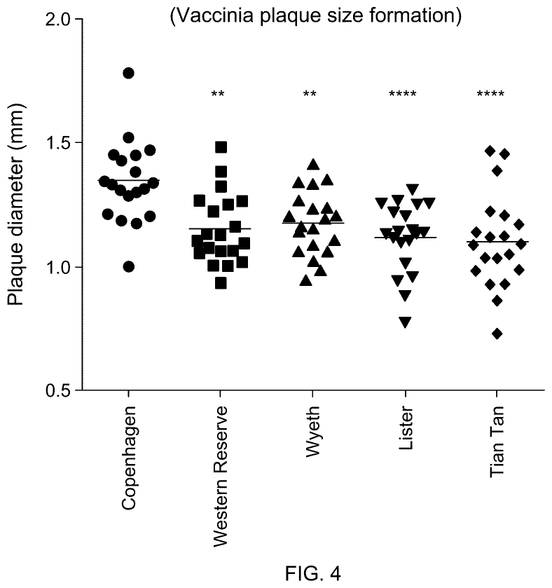

Vaccinia wild-type strains were then subjected to a plaque assay on U2-OS cells with a 3% CMC overlay. Two days past infection, 20-30 plaques for each strain were measured for their size. Plaque size measurements for Copenhagen, Western Reserve, Wyeth, Lister, and Tian Tan are shown in FIG. 4. Plaque formation is affected by the ability of the virus to replicate, spread, and kill. The larger plaque sizes observed for the Copenhagen strain suggest that this strain is superior in these abilities, which are important for the development of an oncolytic virus.

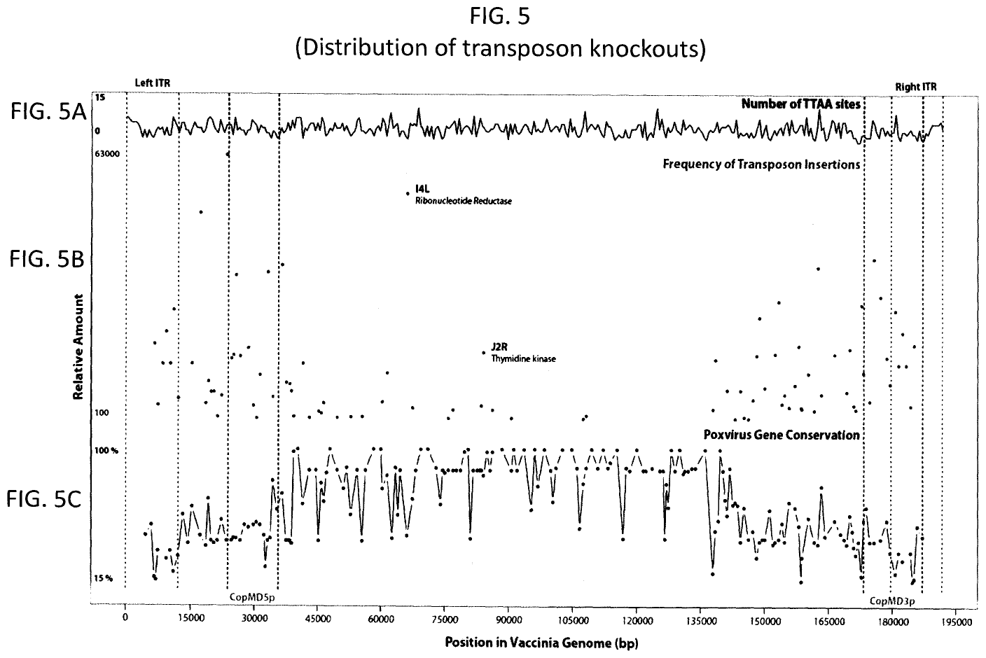

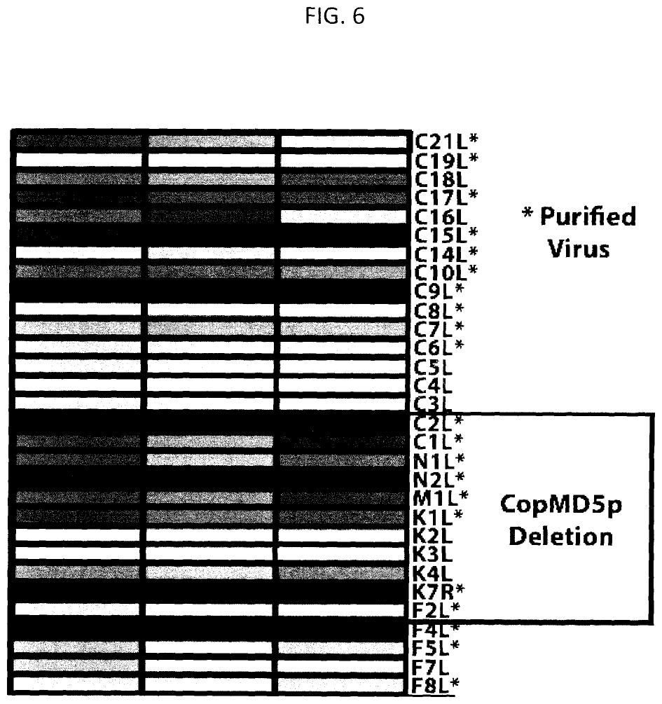

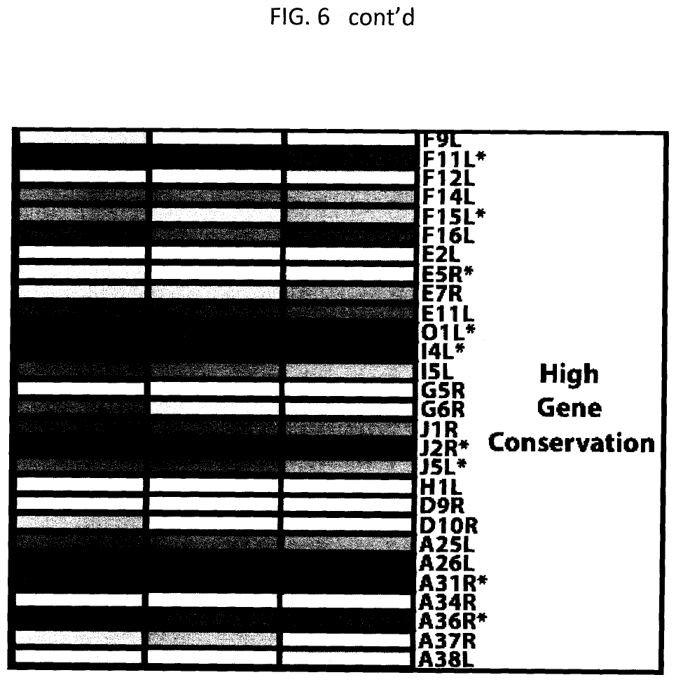

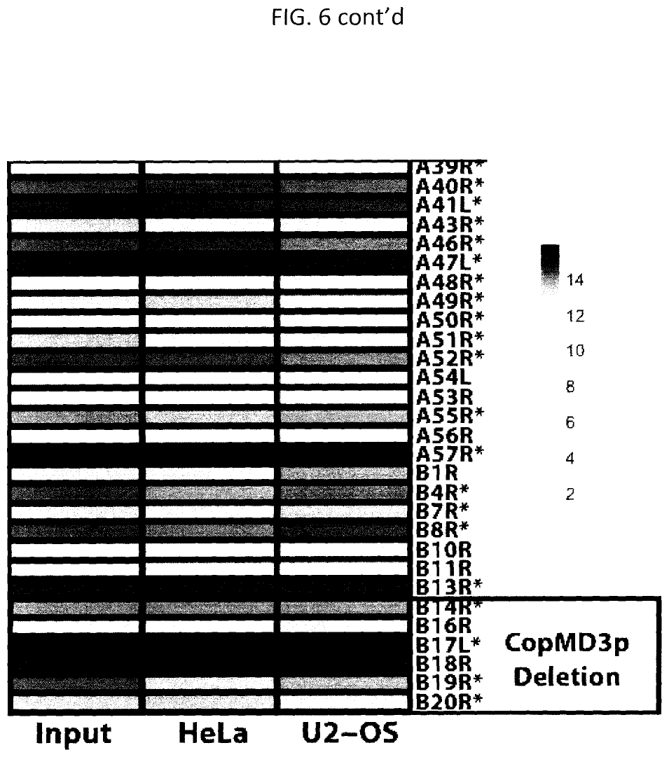

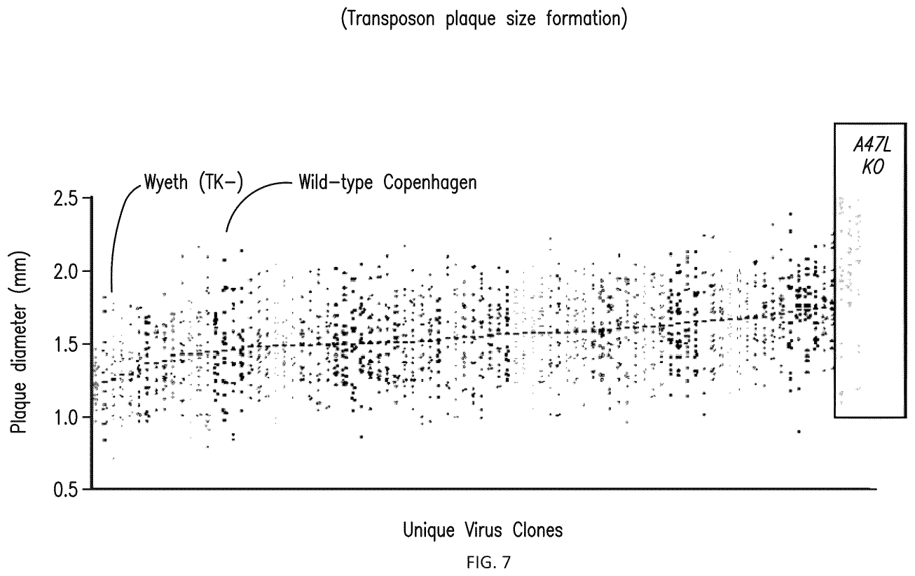

Then, the number of TTAA sites across 1 kb regions in Vaccinia Copenhagen genome were counted (see FIG. 5A). Ilumina NGS sequencing was combined and used to identify Transposon Insertion Sites (Tn-Seq). The input library was passaged three times in either HeLa or U2-OS cells after which frequencies of transposon knockouts were determined. The frequency of a knockout directly corresponds to the amount of reads supporting the event (see FIG. 5B and FIG. 6).

Finally, all 59 poxvirus genomes from FIG. 1 were used to find ORFs and clustered into orthologous groups. Groups containing Copenhagen genes were plotted based on location of the gene in the Copenhagen genome (x-axis) and size of the group (y-axis). When all 59 species share the same gene the conservation is considered to be 100%. The TTAA motif is required for a transposon insertion and this motif is ubiquitous along the genome, meaning transposons can insert anywhere in the genome. However, it was noted that transposons insert preferentially in areas of low poxvirus gene conservation. While sequencing transposon knockouts, major deletions were identified and labelled as CopMD5p and CopMD3p (see FIG. 5C and FIG. 6). Genes that are present in the middle of the genome and that have an elevated gene conservation (FIG. 5C) are important for viral replication. This is because knocking these genes out with transposon insertions causes a decrease in fitness (less frequency after passaging). Genes that are part of the major deletions CopMD5p and CopMD3p were found to be less important for viral replication as their deletion does not impact fitness.

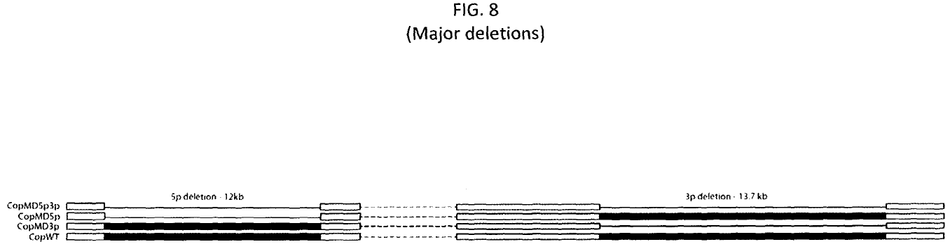

Illumina NGS deep sequencing revealed presence of major deletions during the plaque purification process. CopMD5p and CopMD3p represent clones, which were plaque purified and found to harbor major genomic deletions. These 2 clones were used to co-infect a monolayer of HeLa cells at a high MOI (MOI 10) to induce recombination. Random plaque picking and PCR revealed presence of a double deleted CopMD5p3p which contained both genome deletions (see FIG. 8). These 2 deletions were combined and purified to give a replicating virus, referred to herein as “CopMD5p3p”, that exhibits deletions in the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes, as well as single deletions in each of the ITR genes B21R, B22R, B23R, B24R, B25R, B26R, B27R, B28R, and B29R. As used herein, “CopWT” refers to wild-type Copenhagen vaccinia virus, “CopMD5p” refers to a Copenhagen vaccinia virus harboring deletions in representative 5′ genes (C2L, C1L, N1L, N2L, M1L, M2L, K1L, K2L, K3L, K4L, K5L, K6L, K7R, F1L, F2L, F3L), and “CopMD3p” refers to a Copenhagen vaccinia virus harboring deletions in representative 3′ genes (B14R, B15R, B16R, B17L, B18R, B19R, and B20R) as well as single deletions in each of the ITR genes B21R, B22R, B23R, B24R, B25R, B26R, B27R, B28R, and B29R.

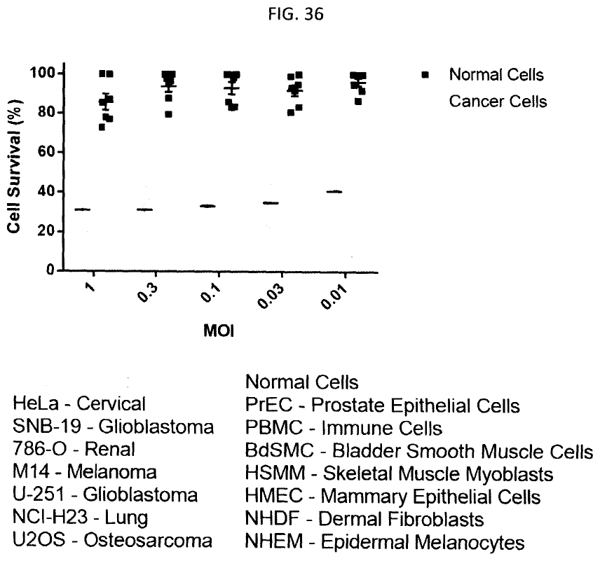

The 59 poxvirus genomes were then assessed for the presence of these 31 genes deleted in the CopMD5p3p. Homology searches were used to query poxviruses from other clades with amino acid sequences of Table 2 genes from the Copenhagen genome. As shown in FIG. 36, the percentage of these 32 genes present in various poxvirus strains decreases with increasing divergence from the Copenhagen strain (each dot on the plot represents one poxvirus genome). However, a majority of the members of the orthopox family, comprise at least 85% of the genes which are deleted in the CopMD5p3p recombinant vector.

### Example 2—Cancer Cell Death

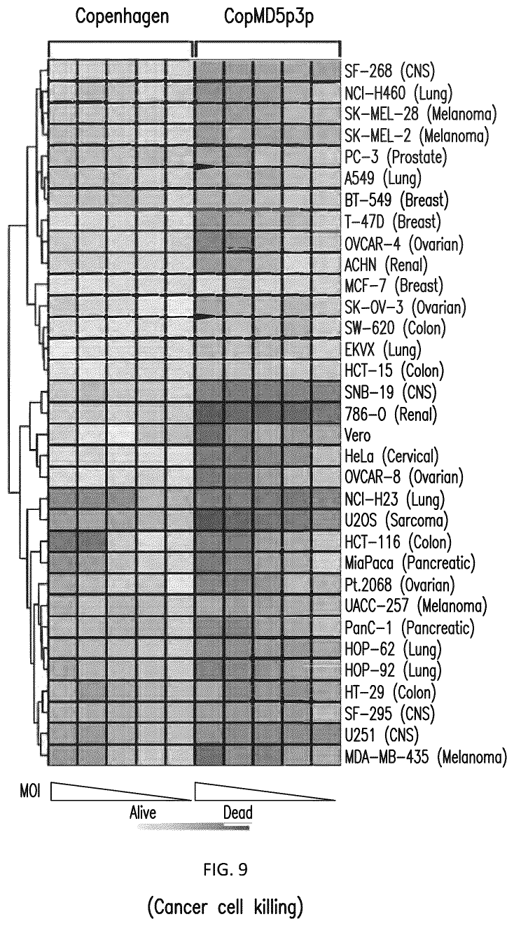

Cancer cells were infected with CopMD5p3p at a range of MOIs (1 to 0.01) in 24-well plates in 4 replicates. Two days post infection with virus, plates were stained with crystal violet. Crystal violet stain was dissolved into SDS and read by spectrophotometry. Data is represented as percent of non-infected cells (see FIG. 9). This data shows that the majority of cancer cell lines die faster when exposed to the CopMD5p3p virus.

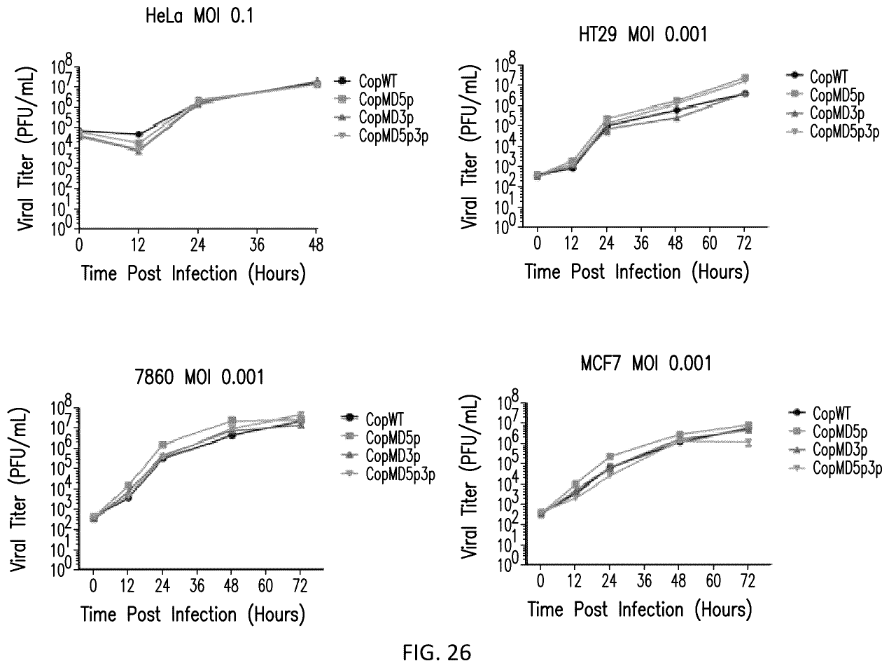

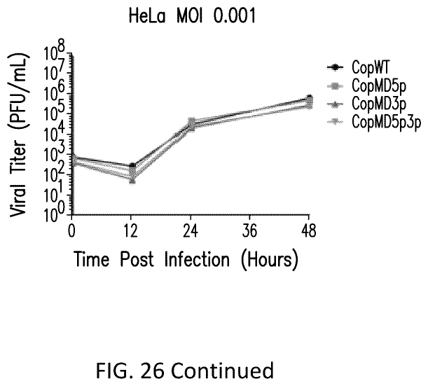

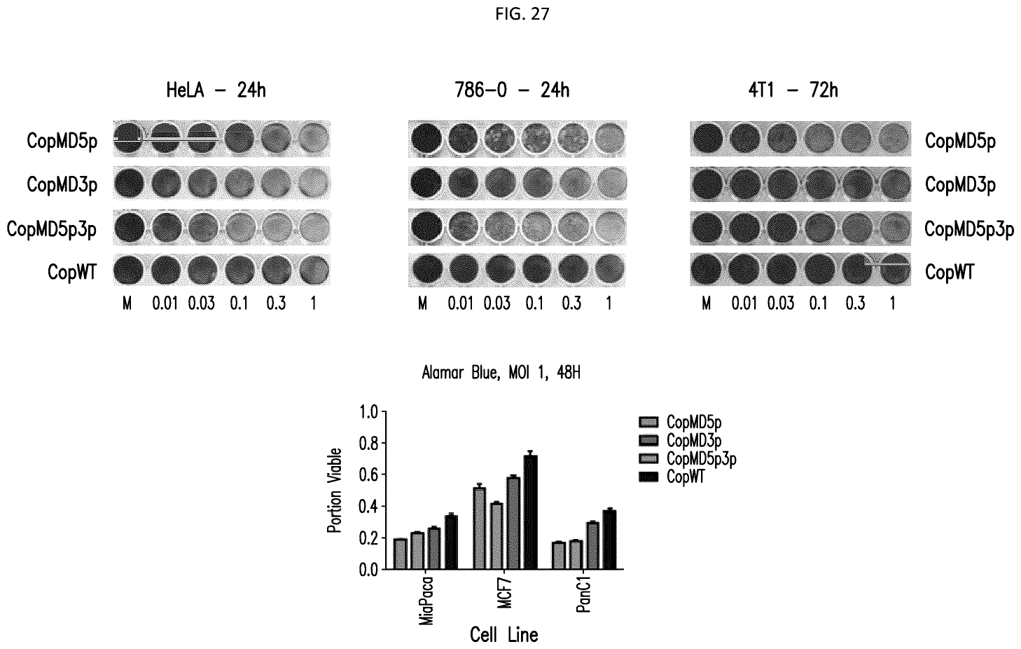

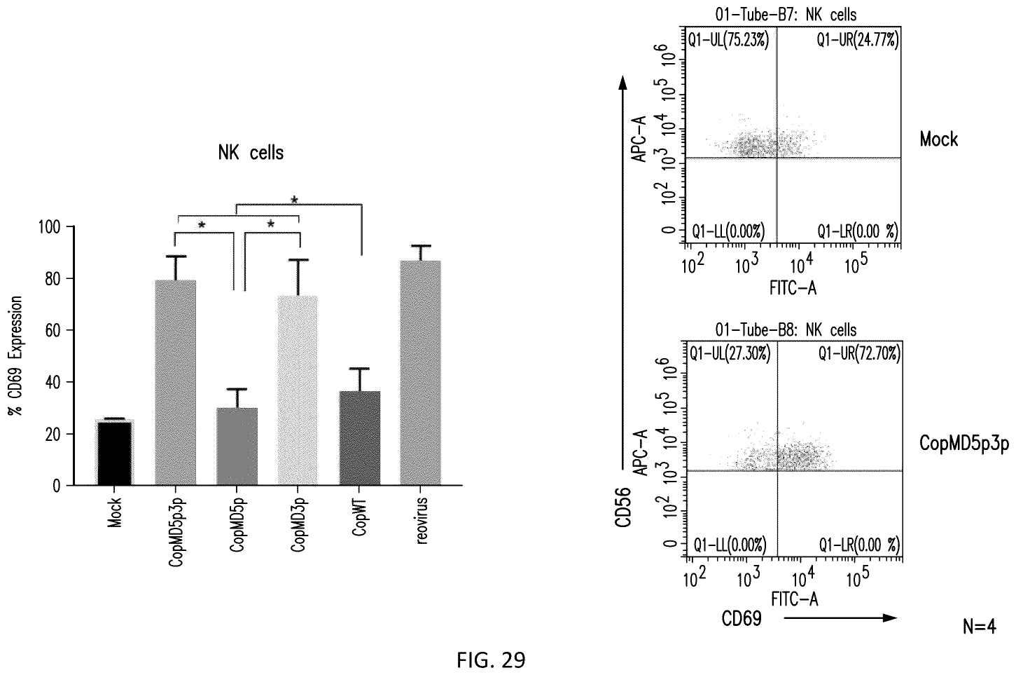

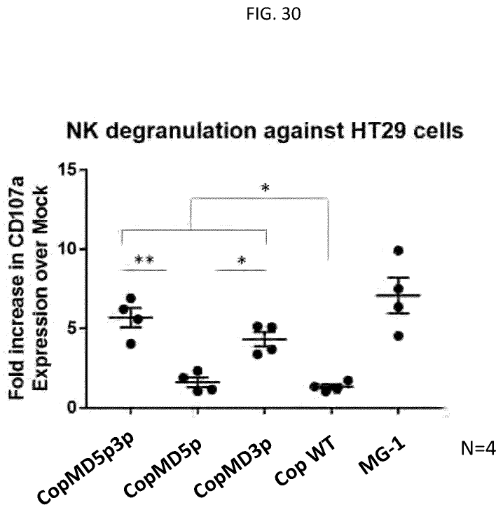

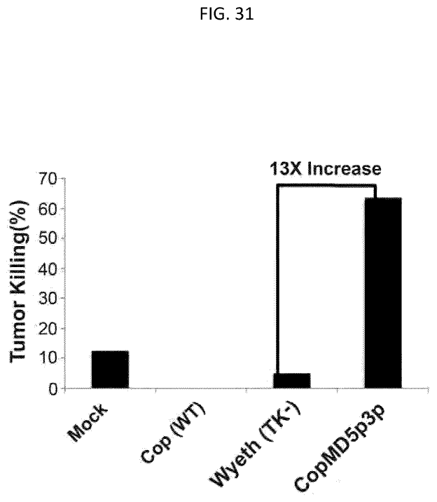

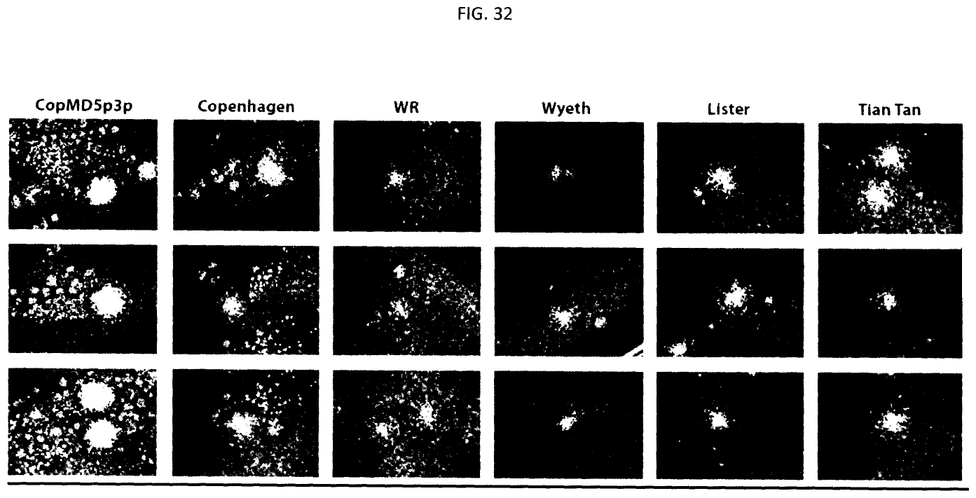

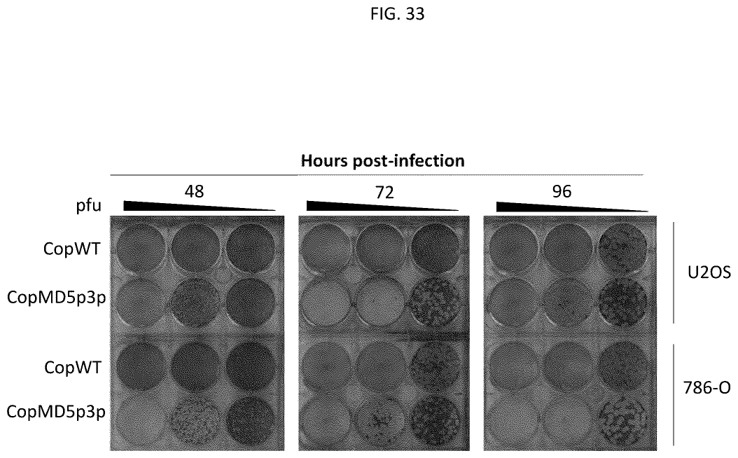

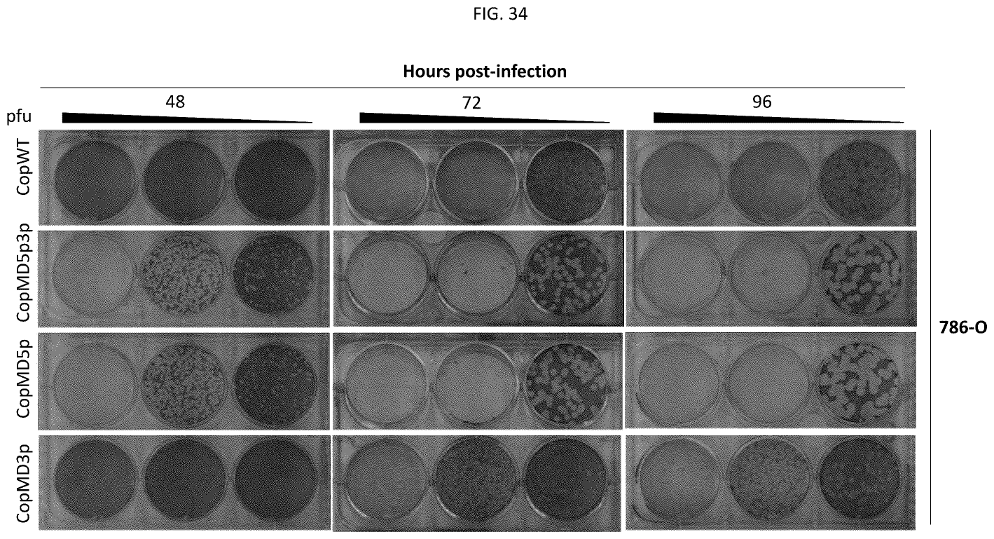

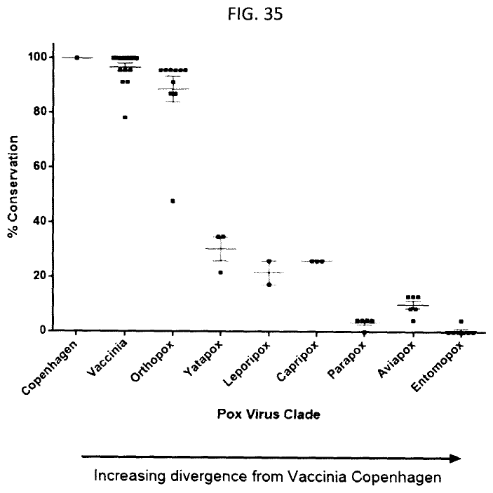

The ability of wild-type Copenhagen vaccinia virus and several modified Copenhagen vaccinia virions to induce an anti-tumor immune response and to propagate in various cancer cell lines is also shown in FIGS. 26, 27, and 29-35.

### Example 3—Growth in Cancer Cells

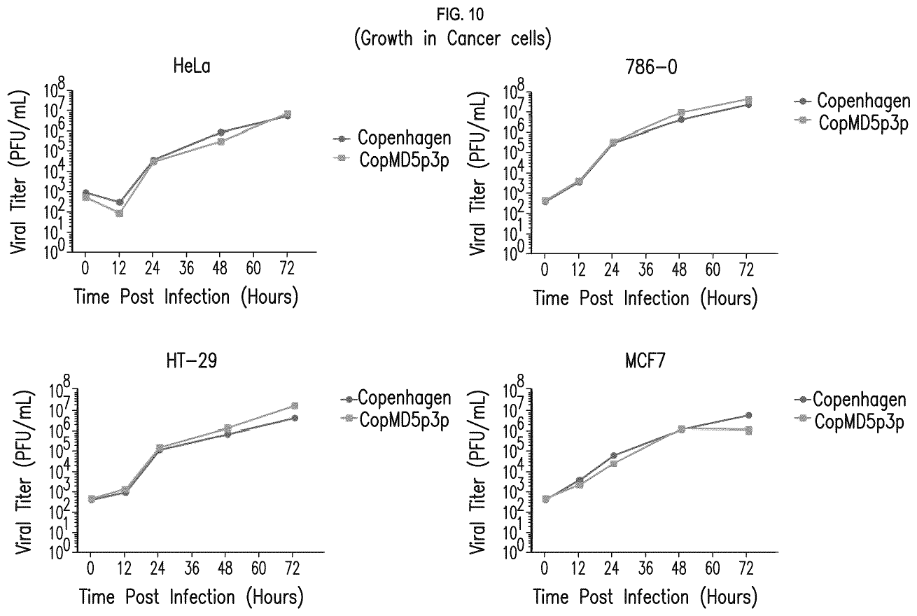

Four cancer cell lines were infected with CopMD5p3p at a low MOI (0.001) in 24-well plates in triplicates, and at different time points, the virus was collected and tittered. Time 0 h represents input. The growth curves of HeLa, 786-O, HT-29, and MCF7 are shown in FIG. 10. This data shows that the modified CopMD5p3p virus is not impaired in its ability to grow in vitro. This means that the virus is replication competent, even in presence of interferon response. The ability to replicate in mammalian cell lines provides another important advantage. As such, viruses may be manufactured with enhanced speed and efficiency.

### Example 4—Growth in Patient Tumor Samples

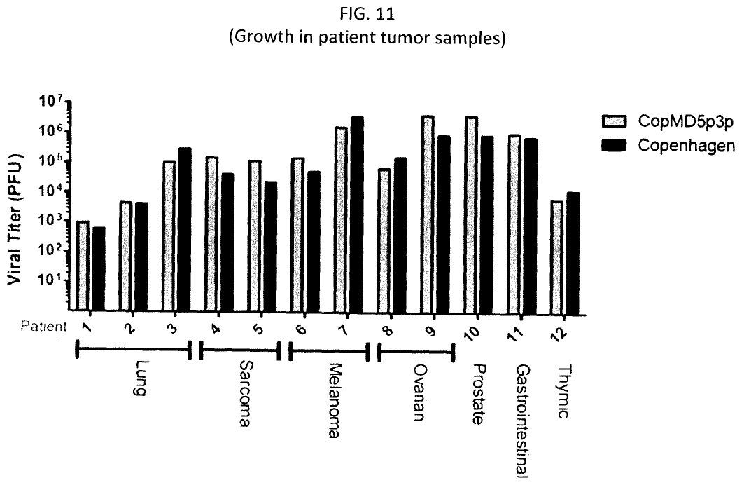

Patient tumor samples were obtained immediately after surgery and cut into 2 mm×2 mm cores. Three cores were infected with a small amount of virus (1×10⁴ PFU), either wild-type Copenhagen or CopMD5p3p. After 72 h virus output was assessed by plaque assay and final Viral Titer expressed as PFU (see FIG. 11). This data shows that the modified CopMD5p3p virus can replicate in fresh patient tumor samples. Replication in patient tumor samples is a good model of replication in a patient 3D tumor.

### Example 5—Syncytia in U2-OS Cells

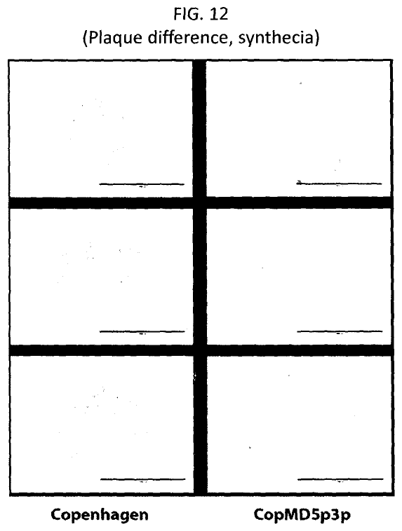

Monolayers of U2-OS cells were infected with either Copenhagen wild-type or CopMD5p3p virus. After 2 h, the media was changed for overlay media as done for a plaque assay. At 48 h post infection, pictures were taken with EVOS to assess plaque phenotype (see FIG. 12). Cell fusion, also known as syncytia, is thought to help the virus spread, since uninfected cells merge with infected cells. Additionally, it has been shown that fused cells are immunogenic and in the case of cancer cells can help initiate an anti-tumor immune response. See, e.g., http://cancerres.aacrjournals.org/content/62/22/6566.long.

### Example 6—Syncytia in 786-O Cells

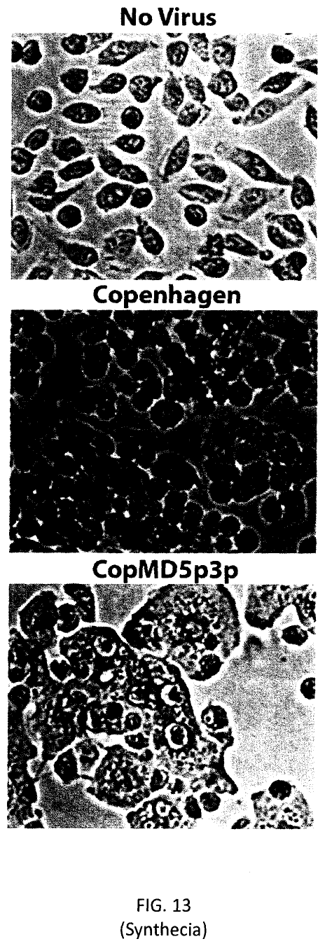

Monolayers of 786-O cells were infected with either Copenhagen wild-type or CopMD5p3p virus. After 24 h pictures were taken with EVOS at 10× magnification (see FIG. 13). This is additional evidence for the occurrence of syncytia. In FIG. 12, the phenotype of a plaque is shown. In the current experiment, monolayers of cells were infected without overlay. Most cells infected by the CopMD5p3p virus have fused.

### Example 7—Tumor Control and Weight Loss in Mouse Model

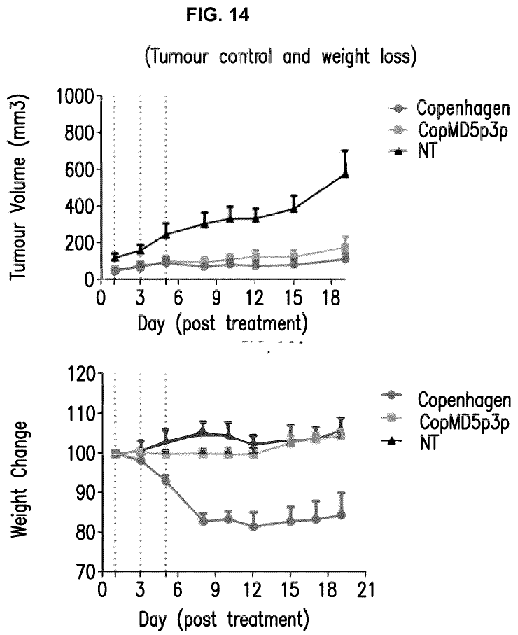

Nude CD-1 (Crl:CD1-Foxn1 nu) mice were seeded with HT-29 human colon cancer xenograft (5e6 cells). Once subcutaneous tumours have established an approximate 5 mm×5 mm size, mice were treated three times (dashed lines) 24 h apart with 1×10⁷ PFU of either vaccinia virus intravenously. Mice were measured approximately every other day for tumor size and weight loss (see FIG. 14). This experiment shows that CopMD5p3p is a much safer virus because it does not cause any weight loss or other signs of sickness in immunocompromised nude mice. This experiment also shows CopMD5p3p is able to control tumor growth similarly to the parental Copenhagen wild-type virus.

### Example 8—Pox Lesion Formation

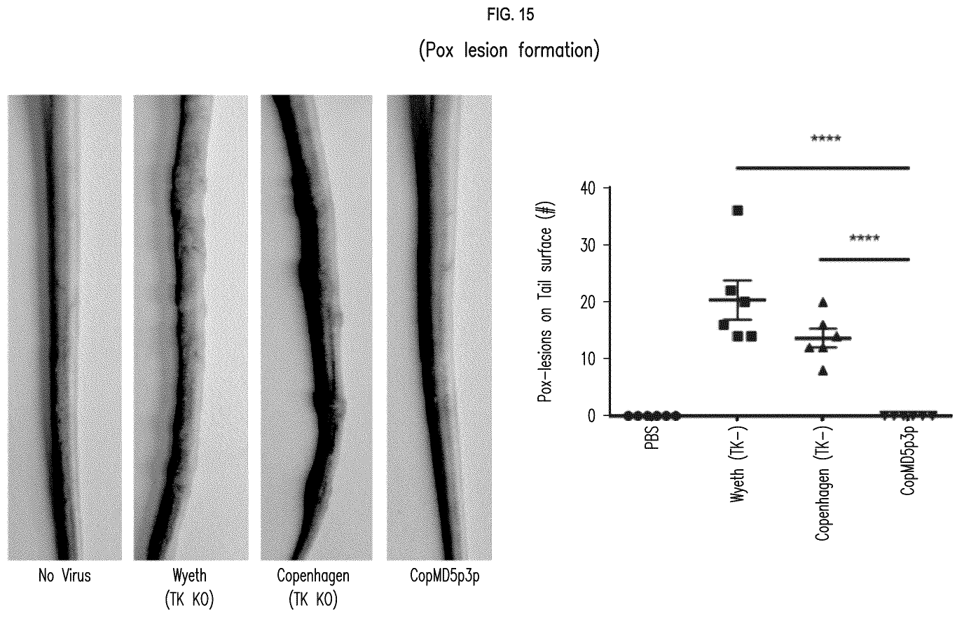

Nude CD-1 mice were treated once with 1×10⁷ PFU of either vaccinia virus intravenously, six mice per group. Two weeks post treatment, mice were sacrificed and pictures of tails were taken. Pox lesions on tails were counted manually on every mouse tail. Representative pictures shown in FIG. 15. This experiment shows that CopMD5p3p is a much safer virus because it does not cause any pox lesions in immunocompromised nude mice. This is important since prior Oncolytic Vaccinia clinical data has shown patients developing pox lesions upon treatment. Knockout of thymidine kinase (TK) is a popular way of increasing the safety of an OV (oncolytic virus), currently present in a Phase III Oncolytic Vaccinia and in FDA approved Oncolytic T-Vec. The data shows that deleting TK does not play a crucial role in this assay, where mice develop pox lesions when challenged with TK deleted viruses, but do not develop pox lesions with CopMD5p3p which has an intact TK.

### Example 9—IVIS Bio-Distribution of Vaccinia after Systemic Administration

Vaccinia viruses wild-type Wyeth, wild-type Copenhagen, and CopMD5p3p were engineered to express Firefly Luciferase (Flue) and YFP through transfection of infected cells with a pSEM1 plasmid replacing TK with Fluc and YFP. Viruses were plaque purified and expanded. All viruses are TK knockouts and encode functional Fluc in their TK locus.

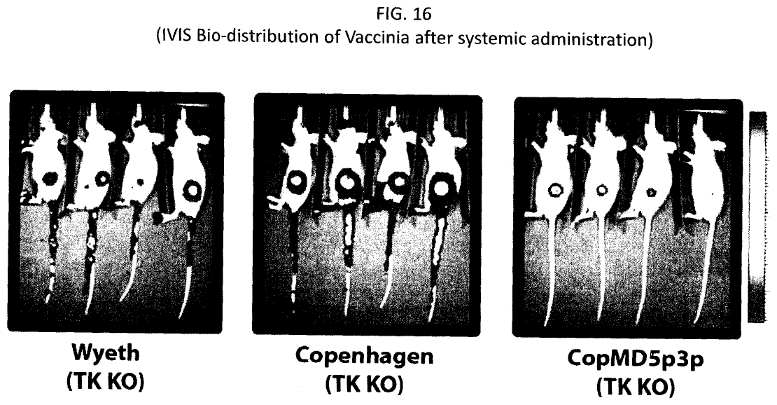

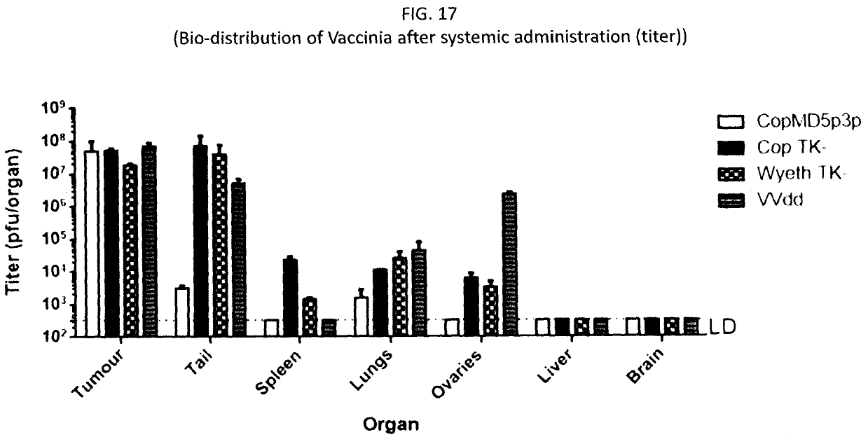

Nude CD-1 mice were then seeded with HT-29 human colon cancer xenograft. Once subcutaneous tumors have established an approximate 5 mm×5 mm size, mice were treated once with 1e7 PFU of either vaccinia Fluc encoding virus intravenously, four mice per group. Four days post treatment, mice were injected i.p. (intraperitoneal) with luciferin and imaged with IVIS for presence of virus (see FIG. 16). This experiment shows that CopMD5p3p is a much safer virus because it is more specific to the tumor. Other viruses show off target replication in the tail, muscle, paws and intra-nasal cavity. CopMD5p3p is only localized in the tumor. As shown in previous FIGS. 15 and 16, there is less detectable CopMD5p3p in the tail compared to the other strains. FIG. 17 shows that CopMD5p3p also has lower titers in other organs when compared to other oncolytic Vaccinia. Since the CopMD5p3p replicates at the same level as the other viruses in the tumor but less in off-target tissues, CopMD5p3p fits the profile of an oncolytic virus better.

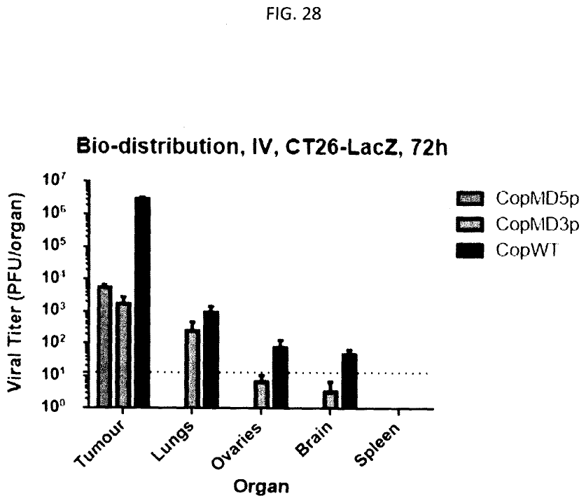

An additional example of the biodistribution of various vaccinia viral vectors, including the wild-type Copenhagen vaccinia virus and several modified Copenhagen vaccinia virions, is shown in FIG. 28.

### Example 10—Immunogenicity of Vaccinia in Human PBMCs

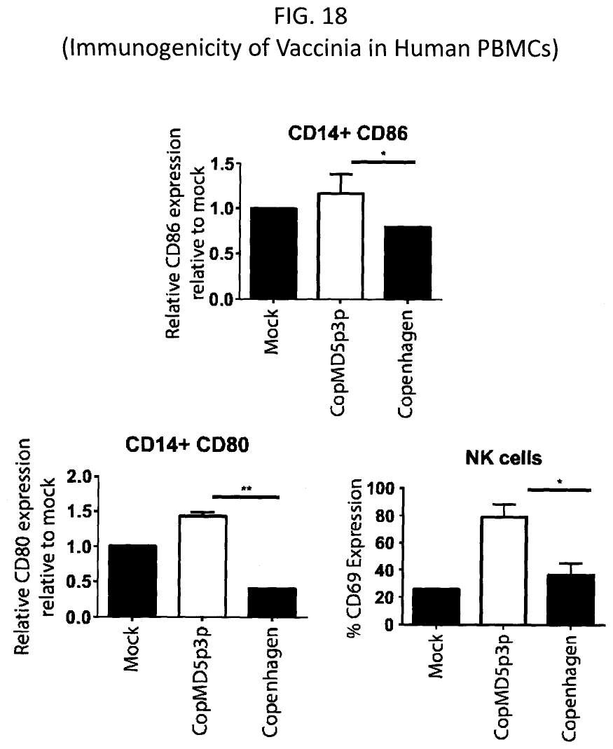

PBMCs were isolated from blood of healthy human donors (n=2). PBMCs were incubated with either Vaccinia for 24 h and checked for early activation markers using Flow Cytometry (see FIG. 18). This experiment shows that CopMD5p3p is more immunogenic and more readily detectable by immune cells. We believe that this is a desirable trait, since OVs replicating in tumor tissue need to activate immune cells for a successful anti-tumor immune response.

### Example 11—Immunogenicity of Vaccinia in Mouse Splenocytes

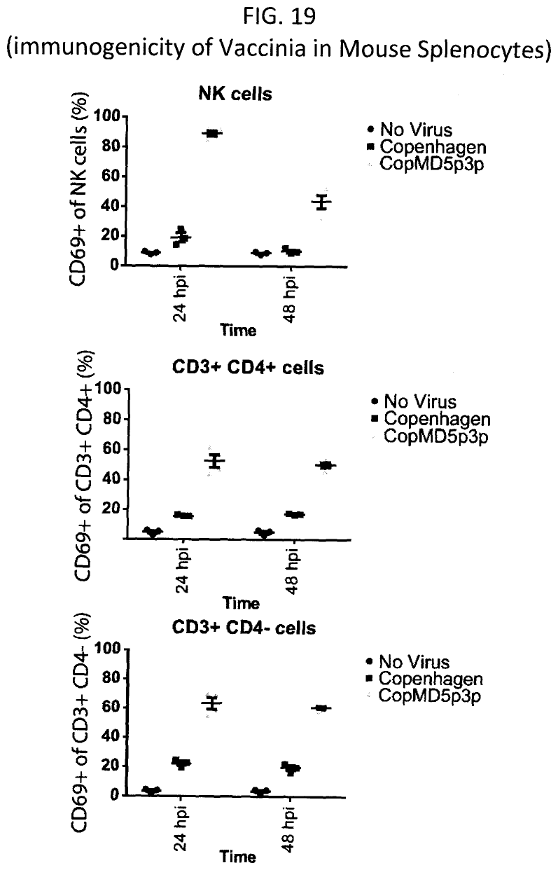

Immune competent Balb/C mice were injected with 1×10 7 Vaccinia PFU Vaccinia virus intravenously. After one or two days, mice were sacrificed, spleens were harvested and analyzed for immune activation using Flow Cytometry (see FIG. 19). This experiment shows that CopMD5p3p is more immunogenic and more readily detectable by mouse immune cells. This data complements nicely the previous FIG. 18, since most of the in vivo experiments are done in mice.

### Example 12—Immunogenicity of Vaccinia in Human Cells

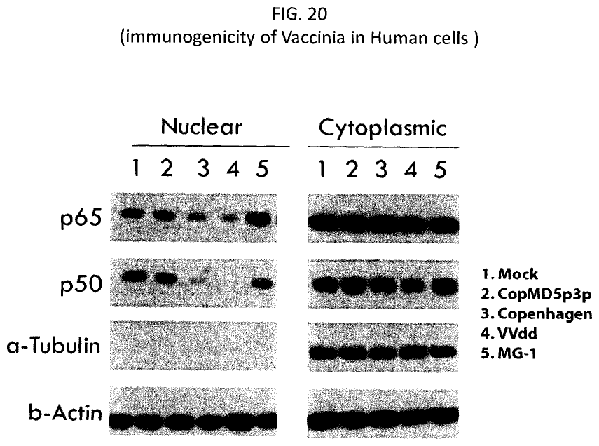

Human cancer cells 786-O were infected at an MOI of 0.01 with either virus. The next day, cells were harvested and nuclei and cytoplasm were separated by cell fractionation. Protein was extracted from each fraction and blotted for NF-kB subunits p65 and p50 (see FIG. 20). NF-kB immune transcription factor initiated an immune response once it's subunit p65 and p50 are translocated to the nucleus. Some viruses are immunosuppressive and block this translocation, preventing an immune response. Suppressing NF-kB function is counter-intuitive to the goal of using oncolytic viruses in combination with immunotherapeutic approaches. Thus, CopMD5p3p is a more advantageous virus as it behaves similarly to MG-1.

### Example 13—Synergy with Immune Checkpoint Inhibitor Anti-CTLA4 in Aggressive Melanoma Model

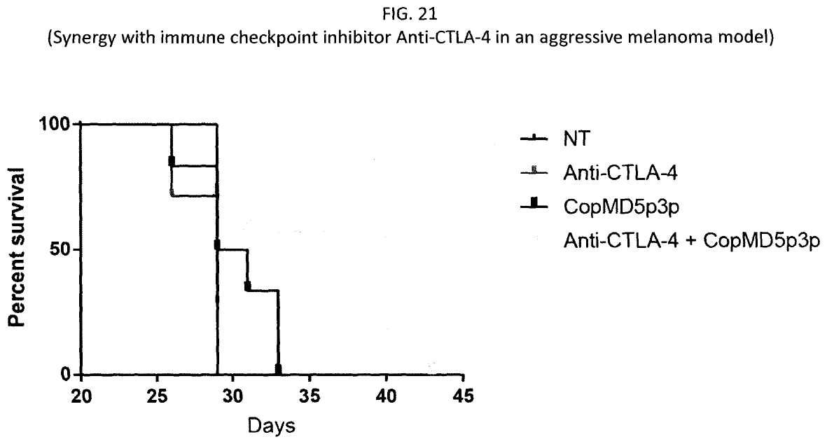

Immune competent C57BL/6 mice were seeded (5e5 cells) subcutaneously with B16-F10 melanoma tumors. Treatment began once subcutaneous tumors have established an approximate 5 mm×5 mm size. Mice treated with CopMD5p3p virus received three 1×10⁷ PFU doses into the tumor (intra-tumor) one day apart. Mice treated with anti-CTLA4 received five 100 μg doses of antibody i.p. one day apart. Survival were recorded every other day once treatment started (see FIG. 21). In this experiment, we tested if the oncolytic effect of our CopMD5p3p virus can synergize with blockade of a well-known immune checkpoint CTLA-4 in a very aggressive melanoma murine model. Surprisingly, the median survival of mice treated with virus and checkpoint was higher than any other group. This suggests that CopMD5p3p has some stimulating properties that synergize with checkpoint blockade immunotherapy.

### Example 14—Synergy with Immune Checkpoint Inhibitor Anti-CTLA4

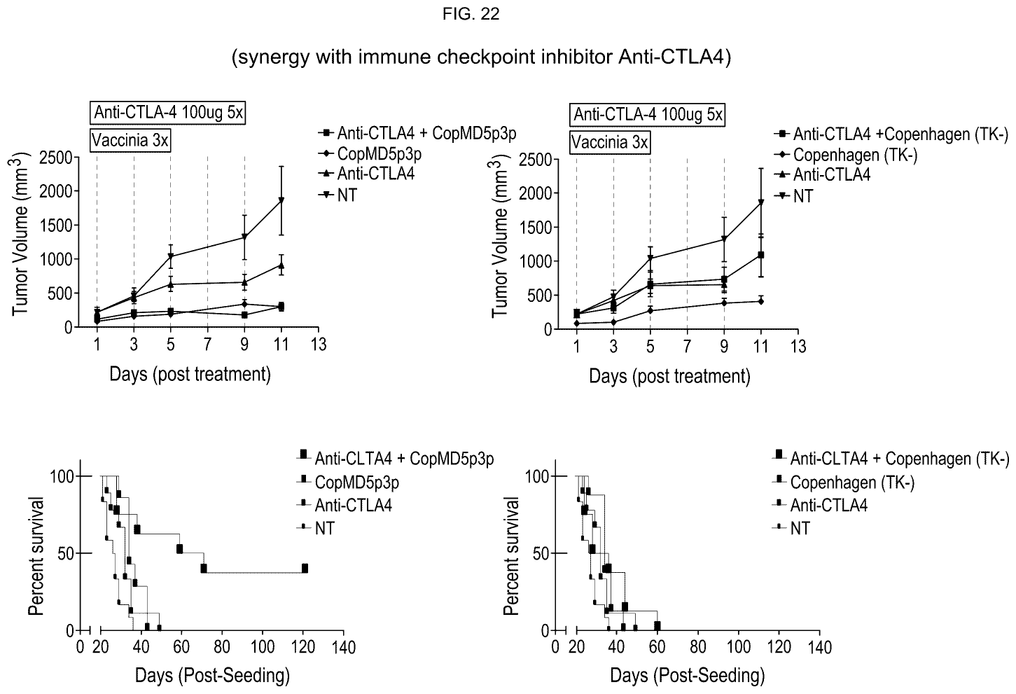

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1e7 PFU doses into the tumour (intra-tumour). Mice treated with Anti-CTLA4 received five (24 h apart, dashed lines) 100 μg doses of antibody i.p. Tumor size and survival were recorded every other day once treatment started (see FIG. 22). The data shows that a TK knockout Vaccinia virus does not work as well with Anti-CTLA4 as does CopMD5p3p. This suggests CopMD5p3p is more immunogenic and more capable of generating an anti-tumour immune response.

### Example 15—Synergy with Immune Checkpoint Inhibitor Anti-PD1

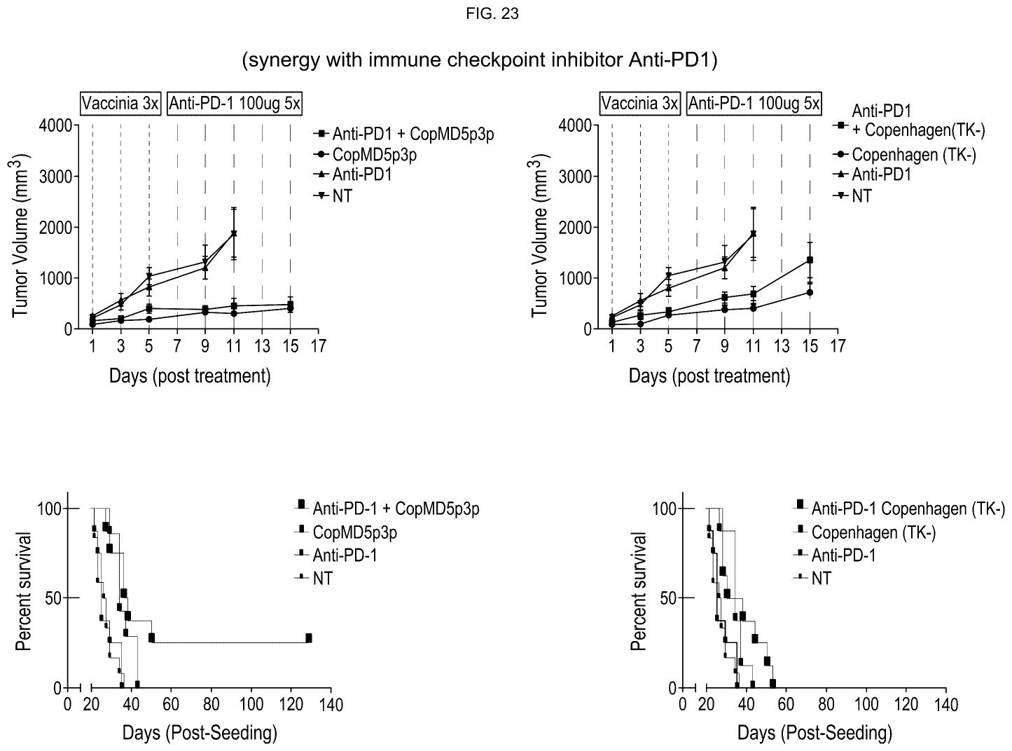

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1e7 PFU doses into the tumor (intra-tumor). Mice treated with Anti-PD1 received five (24 h apart, last five dashed lines) 100 μg doses of antibody i.p. 24 h after the last dose of Vaccinia virus. Tumor size and survival were recorded every other day once treatment started (see FIG. 23). The data shows that a TK knockout Vaccinia virus does not work as well with Anti-PD1 as does CopMD5p3p. This suggests CopMD5p3p is more immunogenic and more capable of generating an anti-tumor immune response.

### Example 16—Synergy with Immune Checkpoint Inhibitor Anti-PD1 and Anti-CDLA4

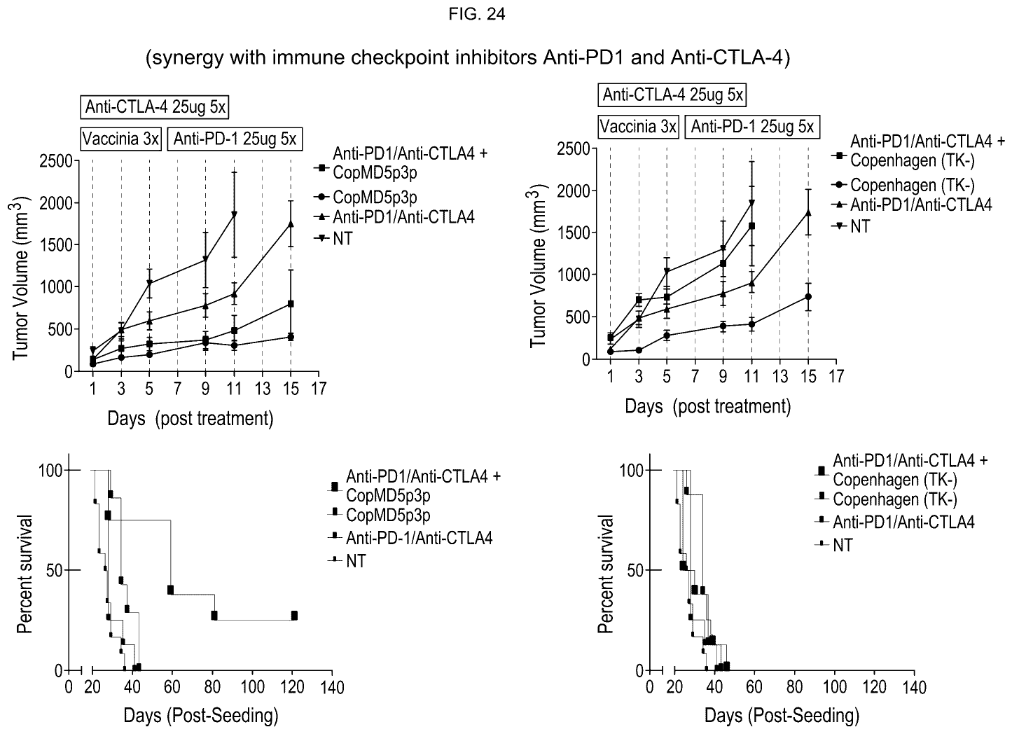

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1×10⁷ PFU doses into the tumor (intra-tumor). Mice treated with Anti-CTLA4 received five (24 h apart, first five dashed lines) 100 μg doses of antibody i.p. Mice treated with Anti-PD1 received five (24 h apart, last five dashed lines) 100 μg doses of antibody i.p. 24 h after the last dose of Vaccinia virus. Tumor size and survival were recorded every other day once treatment started (see FIG. 24). In this experiment we tested whether a lower dose (25 μg instead of 100 μg) of checkpoint inhibitor antibody could work if we blocked both checkpoints simultaneously. The CopMD5p3p still managed to achieve cures in this murine model with a lower dose (50 μg total) of both inhibitors of checkpoints. Since checkpoint inhibitors have dose dependent toxicity, it is advantageous that very small doses of checkpoint blockers can still achieve an observable phenotype. As in other experiments, the CopMD5p3p virus manages to cure established tumors, and this effect is not observed with wild-type virus lacking the corresponding deletions of CopMD5p3p.

### Example 17—Administration for the Treatment of a Subject

Using the methods described herein, a clinician of skill in the art can administer to a subject (e.g., a patient) a pharmaceutical composition containing a recombinant orthopoxvirus vector described herein to treat cancer or tumor cells. The cancer may be, for example, leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer, among others.

For instance, a clinician of skill in the art may assess that a patient is suffering from cancer or tumors and may administer to the patient a therapeutically effective amount (e.g., an amount sufficient to decrease the size of the tumor) of a pharmaceutical composition containing the recombinant orthopoxvirus vector disclosed herein. The pharmaceutical composition may be administered to the subject in one or more doses (e.g., 1, 2, 3, 4, 5, 6, 7, 8, 9, 10, 15, 20, 25, or more) per a specified time interval (e.g., weekly, daily, or hourly). The patient may be evaluated between doses to monitor the effectiveness of the therapy and to increase or decrease the dosage based on the patient's response. The pharmaceutical composition may be administered to the patient orally, parenterally (e.g., topically), intravenously, intramuscularly, subcutaneously, or intranasally. The treatment may involve a single dosing of the pharmaceutical composition. The treatment may involve continued dosing of the pharmaceutical composition (e.g., days, weeks, months, or years). The treatment may further involve the use of another therapeutic agent (e.g., an immune checkpoint inhibitor, such as an anti-PD-1 or anti-CTLA-4 antibody or antigen-binding fragment thereof, IL-12, FLT3L).

### Example 18—Targeted Deletions of CopMD5p and CopMD3p

The following protocol for producing modified vaccinia viral vectors utilizes techniques described, e.g., in Rintoul et al. PLoS One. 6(9): e24643 (2011), the disclosure of which is incorporated herein by reference.

Briefly, CopMD5p (Copenhagen vaccinia virus harboring deletions in 5′ genes: C2L, C1L, N1L, N2L, M1L, M2L, K1L, K2L, K3L, K4L, K5L, K6L, K7R, F1L, F2L, F3L) and CopMd3p (Copenhagen vaccinia virus harboring deletions in 3′ genes: (B14R, B15R, B16R, B17L, B18R, B19R, and B20R as well as single deletions in each of the ITR genes B21R, B22R, B23R, B24R, B25R, B26R, B27R, B28R, and B29R targeting recombinant constructs were synthesized by g-Block technology (IDT, Coralville Iowa). U2OS cells were infected with wildtype vaccinia virus (Wyeth, Western Reserve, Tian Tan, Lister) at an MOI of 0.01 in serum free DMEM for 1.5 hours. Viral supernatant was aspirated and U2OS cells were transfected with PCR amplified CopMD5p or CopMd3p targeting g-Blocks by Lipofectamine 2000 (Invitrogen) in OptiMEM (Gibco). DMEM supplemented with 10% FBS was added to cells 30 minutes after transfection and left overnight. The following day, transfection media was aspirated and fresh DMEM 10% FBS media was added to cells. 48 hours after infection transfection, U2OS cells were harvested and lysed by a single freeze thaw cycle. Serially diluted lysates were plated onto a confluent monolayer of U2OS cells and eGFP positive (CopMD5p targeted) or mCherry positive (CopMd3p targeted) plaques were isolated and purified through 5 rounds of plaque purifications.

Double major deleted vaccinia viruses were generated by co-infection of CopMD5p and CopMd3p deleted vaccinia viruses at an MOI of 5 for each virus in U2OS cells. Cells were harvested the next day and lysed by one round of freeze thaw. Lysates were serially diluted and plated onto a confluent monolayer of U2OS cells and selected for double positive plaques (eGFP+mCherry). Plaques were purified by 5 rounds of plaque purification.

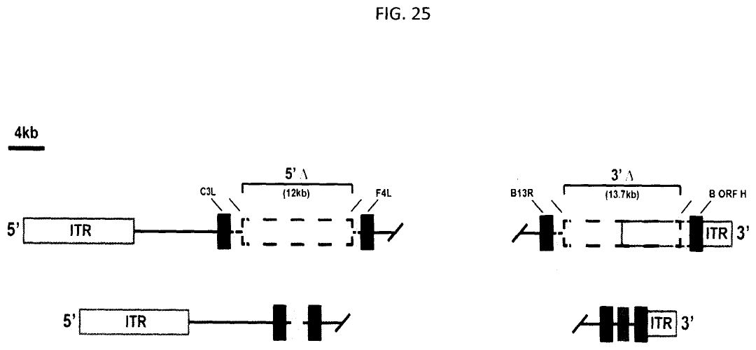

An exemplary scheme for the production of modified orthopoxvirus vectors (e.g., modified vaccinia viral vectors, such as modified Copenhagen vaccinia viral vectors) of the disclosure is shown in FIG. 25.

### Example 19—SKV-GFP (CopMD5p3p-B8R−) has Similar Efficacy in Tumour Control Compared to SKV (CopMD5p3p-B8R+)

The vaccinia virus (VV) B8R gene encodes a secreted protein with homology to gamma interferon receptor (IFN-γ). In vitro, the B8R protein binds to and neutralizes the antiviral activity of several species of gamma interferon including human and rat gamma interferon; it does not, however, bind significantly to murine IFN-γ. Here we describe the construction and characterization of recombinant VVs lacking the B8R gene. Homologous recombination between the targeting construct and the B8R locus resulted in the replacement of 75% of the B8R gene with the eGFP transgenes flanked by two loxP sites (SKV-GFP).

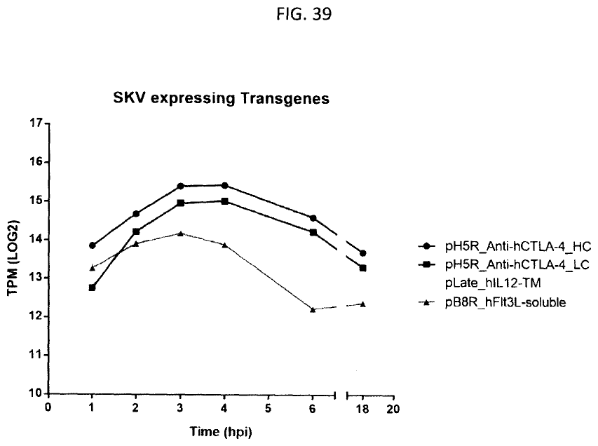

B8R− viruses showed similar efficacy to B8R+ viruses. FIG. 39. Survival of mice treated with either SKV or SKV-GFP was assessed. 5×10⁶ CT26-LacZ cells were seeded subcutaneously on day 0. On day 14, 16 and 18 tumours were treated at a dose of 10⁷ pfu with an intratumoural injection of either SKV or SKV-GFP. No significant decrease in efficacy was seen when the viruses injected had a deletion of the B8R locus.

### Example 20—Infection of Normal Versus Cancer Cell Lines of SKV (CopMD5p3p-B8R+) Virus

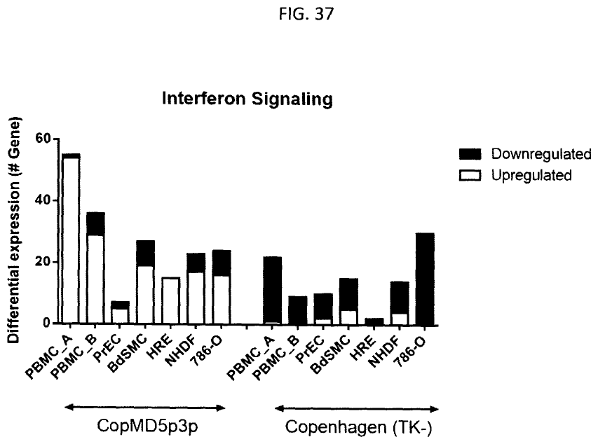

Primary health cell viability was compared to that of cancer cells. Confluent normal or cancer cells were infected at a range of MOI (pfu/cell) for 48 hrs, after which viability was quantified. As indicated in FIG. 37, SKV-B8R+ virus preferentially infects cancer cells.

### Example 21—SKV (CopMD5p3p-B8R+) does not Impair Interferon Signaling

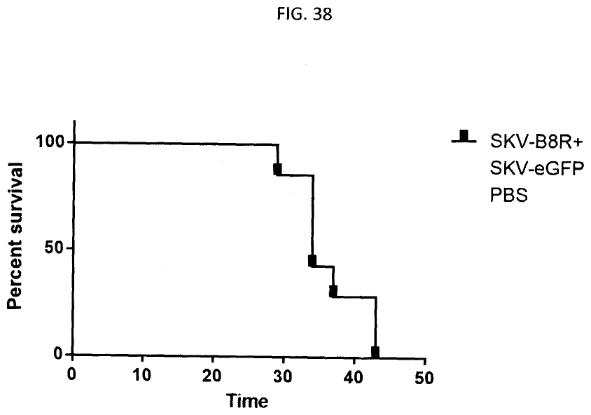

Interferon signaling was assessed by determining the number of genes in the interferon pathway that are upregulated (induced expression) or downregulated (repressed expression) in a variety of normal cell lines and one cancer cell line (786-O). FIG. 38 Confluent monolayers of 1 million cells were infected at an MOI of 3 (3×10⁶ PFU) for 18 h with either SKV (CopMD5p3p-B8R+) or the parental Copenhagen virus strain having the TK gene disabled. RNA was sequenced using RNA-seq and gene expression of interferon genes was determined after read mapping a expression normalization. While the SKV (CopMD5p3p-B8R+) virus mostly induces genes in the interferon pathway the parental Copenhagen represses genes. This suggests SKV (CopMD5p3p-B8R+) is able to induce Type I Interferon signaling which is critical in viral clearance of normal cells.

### Example 22—B8R Negative Vaccinia Virus Engineered to Express Flt3L, IL-12 TM and Anti-hCTLA-4

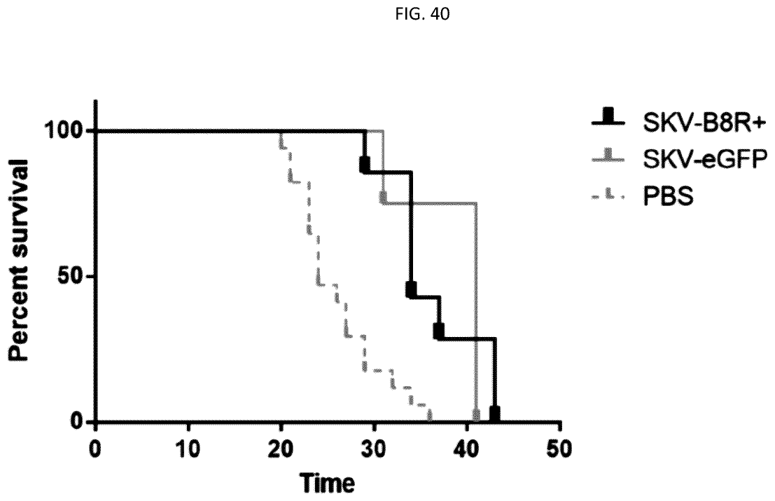

Modified vaccinia viruses containing both the CopMD5p3p and B8R deletions, as described above, were further engineered to express the immunotherapeutic transgenes. An SKV-123 virus 3 (CopMD5p3p-B8R+-IL12TM-FLT3-antiCLTA4) expressing three transgenes was evaluated in terms of transgene expression kinetics. Confluent monolayers of 786-O human adenocarcinoma cell lines were infected with SKV-123 virus at an MOI of 3 (3×10⁶ pfu). RNA was sequenced using RNA-seq and gene expression of inserted transgenes were determined after read mapping after expression normalization. Transgene expression peaked at 3-4 hours after cell infection. See FIG. 40.

### Example 23 SKV Expressing Murine IL-12 p35 Membrane Bound (SKV3) has Greater Efficacy in Controlling Murine Tumors

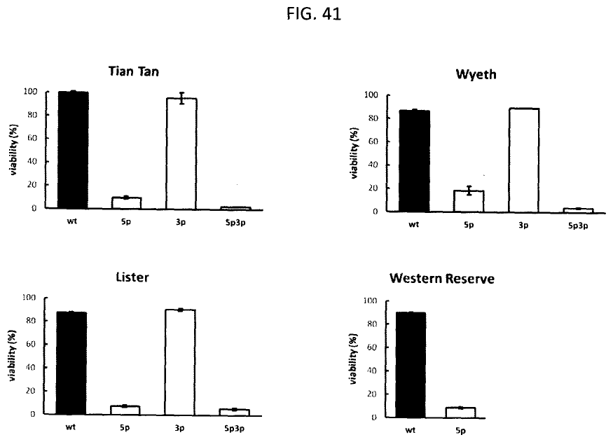

The survival of mice treated with either SKV (CopMD5p3p-B8R+) or SKV-3 (CopMD5p3p-B8R+-IL12TM) virus (expressing murine membrane bound p35 IL-12) was assessed. 5×10⁶ CT26-LacZ cells were seeded sub cutaneously on day 0. On day 14, 16 and 18 tumours were treated at a dose of 1e7 pfu with an intratumoural injection of either SKV or SKV-3. Although SKV virus extend survival of mice bearing CT26 colon tumours. SKV-3 expression of IL-12 is able to induce remissions that lead to durable cures. See FIG. 41.

### Example 24—Major Double Deletions in Engineered in Various Vaccinia Strains Enhance Cancer Cell Killing In Vitro

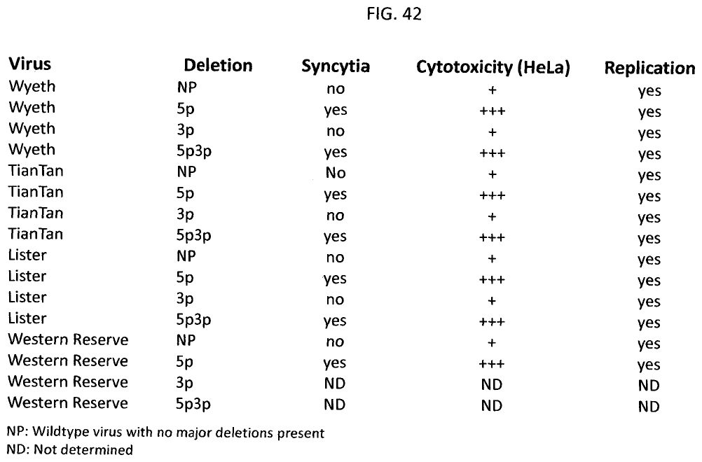

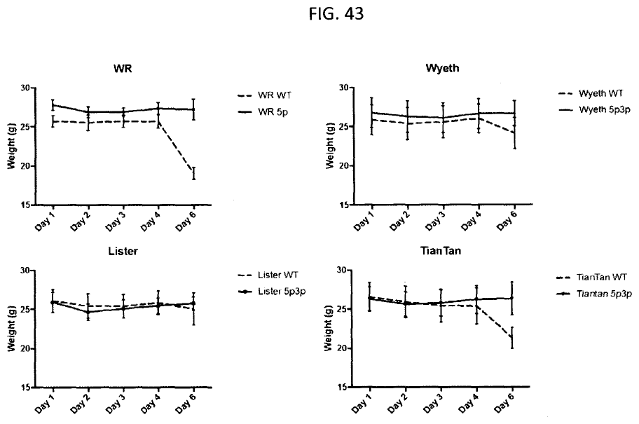

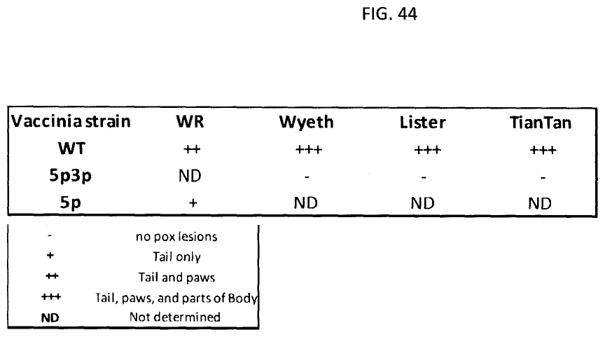

Hela cells were infected at an MOI of 0.1 with the following strains of engineered vaccinia viruses: (1) parental wildtype virus (wt); (2) 5 prime major deleted (5p), (3) 3 prime major deleted (3p), and (4) recombined 5 prime and 3 prime major double deleted (5p3p). Cell viability was quantified by alamar blue assay 72 hours post infection. Both 5p and 5p3p major double deleted vaccinia strains are more cytotoxic in HelLa cells when compared to their parental wildtype and 3p major deleted strains. See FIG. 42. FIG. 43 depicts a summary of the major deleted Vaccinia strains, and the effect of 5p, 3p and 5p3p deletions on syncytia, cytotoxicity and replication. CD-1 nude mice were treated with 1×10⁷ pfu via intravenously tail vein injection and measured at the indicated timepoints. 5p3p vaccinia strains did not induce weight loss compared to wildtype strains. FIG. 44. Mice were also examined for pox lesions 6 days post-injection. 5p3p vaccinia strains do not induce pox lesions compared to wildtype strains. FIG. 45.

### Some Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Some embodiments are within the claims.

1. A nucleic acid comprising a recombinant orthopoxvirus genome, wherein the genome comprises deletions in each of the following 23 genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R and further comprises deletions in each of the following 9 genes: B21R, B22R, B23R, B24R, B25R, B26R, B27R, B28R, and B29R; and
wherein the nucleic acid further comprises: (i) a transgene encoding a tumor-associated antigen; (ii) a transgene encoding an immune checkpoint inhibitor; (iii) a transgene encoding an interleukin (IL); (iv) a transgene encoding an interferon (IFN); (v) a transgene encoding a TNF superfamily member protein; (vi) a transgene encoding a cytokine; or (vii) a combination thereof.

2-79. (canceled)

80. A recombinant orthopoxvirus vector comprising the nucleic acid of claim 1.

81-164. (canceled)

165. A packaging cell line comprising the nucleic acid of claim 1.

166. A method of treating cancer in a mammalian patient, said method comprising administering a therapeutically effective amount of the nucleic acid of claim 1 to said mammalian patient.

167-185. (canceled)

186. A kit comprising the nucleic acid of claim 1 and (a) a package insert instructing a user of said kit to express said nucleic acid in a host cell, or (b) a package insert instructing a user to administer a therapeutically effective amount of said nucleic acid to a mammalian patient having cancer, thereby treating said cancer.

187-196. (canceled)

197. A nucleic acid comprising a recombinant orthopoxvirus genome, wherein the genome comprises:
a first set of deletions in each of the following genes: C2L, C1L, N1L, N2L, M1L, M2L, K1L, K2L, K3L, K4L, K5L, K6L, K7R, F1L, F2L, and F3L, and
a second set of deletions in each of the following genes: B14R, B15R, B16R, B17L, B18R, B19R, B20R, B21R, B22R, B23R, B24R, B25R, B26R, B27R, B28R, and B29R;
and wherein:
(i) the genome further comprises a deletion in the B8R gene; and/or
(ii) the genome does not comprise a thymidine kinase (TK) gene deletion.

198. The nucleic acid of claim 197, wherein the orthopoxvirus is a vaccinia virus and the vaccinia virus is a strain selected from the group consisting of Copenhagen, Western Reserve, Wyeth, Lister, EM63, ACAM2000, CV-1, modified vaccinia Ankara (MVA), Dairen I, GLV-1h68, IHD-J, L-IVP, LC16m8, LC16mO, Tashkent, Tian Tan, and WAU86/88-1.

199. The nucleic acid of claim 197, wherein the orthopoxvirus is a Copenhagen strain vaccinia virus.

200. The nucleic acid of claim 197, wherein each of the deletions is a deletion of the entire polynucleotide encoding the corresponding gene.

201. The nucleic acid of claim 197, wherein each of the deletions is a deletion of at least a portion of the polynucleotide encoding the corresponding gene that is sufficient to render the gene nonfunctional upon introduction into a host cell.

202. The nucleic acid of claim 197, wherein the genome comprises a deletion in the B8R gene.

203. The nucleic acid of claim 202, wherein the only deletions in the genome are deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, B20R, B21R, B22R, B23R, B24R, B25R, B26R, B27R, B28R, and B29R.

204. The nucleic acid of claim 197, wherein the genome does not comprise a TK gene deletion.

205. The nucleic acid of claim 202, wherein the genome does not comprise a TK gene deletion.

206. The nucleic acid of claim 197, wherein the genome does not comprise a ribonucleotide reductase gene deletion.

207. A recombinant orthopoxvirus vector comprising the nucleic acid of claim 197.

208. A packaging cell line comprising the nucleic acid of claim 197.

209. A pharmaceutical composition for treating a cancer in a mammalian patient, comprising a therapeutically effective amount of the nucleic acid of claim 197.

210. A kit comprising the nucleic acid of claim 197 and (a) a package insert instructing a user of the kit to express the nucleic acid in a host cell, or (b) a package insert instructing a user to administer a therapeutically effective amount of the nucleic acid to a mammalian patient having a cancer, thereby treating the cancer.

211. A method of treating cancer in a mammalian patient, said method comprising administering a therapeutically effective amount of the nucleic acid of claim 197 to said mammalian patient.


SEQUENCE LISTING (677 sequences)

SEQ ID NO: 1-22 - skipped (22 entries)

SEQ ID NO: 23 - protein, 512 aa, Vaccinia virus
MESVIFSINGEIIQVNKEIITASPYNFFKRIQDHHLKDEAIILNGINYHAFESLLDYIRWKKINITINNVEMILVAAIIIDVPPVVDLCVKTMIHNINSTNCIRMFNFSKRYGIKKLYNASMSEIINNITAVTSDPEFGKLSKDELTTILSHENVNVNHEDVTAMILLKWIHKNPNDVDIINILHPKFMTNTMRNAISLLGLTISKSTKPVTRNGIKHNIVVIKNSDYISTITHYSPRTEYWTIVGNTDRQFYNANVLHNCLYIIGGMINNRHVYSVSRVDLETKKWKTVTNMSSLKSEVSTCVNDGKLYVIGGLEFSISTGVAEYLKHGTSKWIRLPNLITPRYSGASVFVNDDIYVMGGVYTTYEKYVVLNDVECFTKNRWIKKSPMPRHHSIVYAVEYDGDIYVITGITHETRNYLYKYIVKEDKWIELYMYFNHVGKMFVCSCGDYILIIADAKYEYYPKSNTWNLFDMSTRNIEYYDMFTKDETPKCNVTHKSLPSFLSNCEKQFLQ

SEQ ID NO: 24 - protein, 224 aa, Vaccinia virus
MVKNNKISNSCRMIMSTNPNNILMRHLKNLTDDEFKCIIHRSSDFLYLSDSDYTSITKETLVSEIVEEYPDDCNKILAIIFLVLDKDIDVDIETKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 25 - protein, 117 aa, Vaccinia virus
MRTLLIRYILWRNDNDQTYYNDDFKKLMLLDELVDDGDVCTLIKNMRMTLSDGPLLDRLNQPVNNIEDAKRMIAISAKVARDIGERSEIRWEESFTILFRMIETYFDDLMIDLYGEK

SEQ ID NO: 26 - protein, 175 aa, Vaccinia virus
MTSSAMDNNEPKVLEMVYDATILPEGSSMDPNIMDCINRHINMCIQRTYSSSIIAILNRFLTMNKDELNNTQCHIIKEFMTYEQMAIDHYGEYVNAILYQIRKRPNQHHTIDLFKKIKRTPYDTFKVDPVEFVKKVIGFVSILNKYKPVYSYVLYENVLYDEFKCFINYVETKYF

SEQ ID NO: 27 - protein, 472 aa, Vaccinia virus
MIFVIESKLLQIYRNRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKHNKTPLYYLSGTDDEVIERINLLVQYGAKINNSVDEEGCGPLLACTDPSERVFKKIMSIGFEARIVDKFGKNHIHRHLMSDNPKASTISWMMKLGISPSKPDHDGNTPLHIVCSKTVKNVDIIDLLLPSTDVNKQNKFGDSPLTLLIKTLSPAHLINKLLSTSNVITDQTVNICIFYDRDDVLEIINDKGKQYDSTDFKMAVEVGSIRCVKYLLDNDIICEDAMYYAVLSEYETMVDYLLFNHFSVDSVVNGHTCMSECVRLNNPVILSKLMLHNPTSETMYLTMKAIEKDKLDKSIIIPFIAYFVLMHPDFCKNRRYFTSYKRFVTDYVHEGVSYEVFDDYF

SEQ ID NO: 28 - protein, 220 aa, Vaccinia virus
MVYKLVLLFCIASLGYSVEYKNTICPPRQDYRYWYFAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYGMRGSYGVTFIDELNQCLLDIKELSYDICYRE

SEQ ID NO: 29 - protein, 284 aa, Vaccinia virus
MDLSRINTWKSKQLKSFLSSKDTFKADVHGHSALYYAIADNNVRLVCTLLNAGALKNLLENEFPLHQAATLEDTKIVKILLFSGMDDSQFDDKGNTALYYAVDSGNMQTVKLFVKKNWRLMFYGKTGWKTSFYHAVMLNDVSIVSYFLSEIPSTFDLAILLSCIHTTIKNGHVDMMILLLDYMTSTNTNNSLLFIPDIKLAIDNKDIEMLQALFKYDINIYSVNLENVLLDDAEITKMIIEKHVEYKSDSYTKDLDIVKNNKLDEIISKNKELRLMYVNCVKKN

SEQ ID NO: 30 - protein, 369 aa, Vaccinia virus
MIALLILSLTCSVSTYRLQGFTNAGIVAYKNIQDDNIVFSPFGYSFSMFMSLLPASGNTRIELLKTMDLRKRDLGPAFTELISGLAKLKTSKYTYTDLTYQSFVDNTVCIKPLYYQQYHRFGLYRLNFRRDAVNKINSIVERRSGMSNVVDSNMLDNNTLWAIINTIYFKGTWQYPFDITKTRNASFTNKYGTKTVPMMNVVTKLQGNTITIDDEEYDMVRLPYKDANISMYLAIGDNMTHFTDSITAAKLDYWSFQLGNKVYNLKLPKFSIENKRDIKSIAEMMAPSMFNPDNASFKHMTRDPLYIYKMFQNAKIDVDEQGTVAEASTIMVATARSSPEKLEFNTPFVFIIRHDITGFILFMGKVESP

SEQ ID NO: 31 - protein, 81 aa, Vaccinia virus
MGHIITYCQVHTNISILIRKAHHIIFFVIDCDCISLQFSNYVHHGNRFRTVLISKTSIACFSDIKRILPCTFKIYSINDCP

SEQ ID NO: 32 - protein, 79 aa, Vaccinia virus
MGTVFVPYLLVKLALRVLVISNGYCHVPLKYIVLMIAHRVLLSSILESTTLDIPDLRSTIELILLTASRLKFNLYRPNL

SEQ ID NO: 33 - protein, 88 aa, Vaccinia virus
MLAFCYSLPNAGDVIKGRVYEKDYALYIYLFDYPHSEAILAESVKMHMDRYVEYRDKLVGKTVKVKVIRVDYTKGYIDVNYKRMCRHQ

SEQ ID NO: 34 - protein, 424 aa, Vaccinia virus
MNPDNTIAVITETIPIGMQFDKVYLSTFNMWREILSNTTKTLDISSFYWSLSDEVGTNFGTIILNEIVQLPKRGVRVRVAVNKSNKPLKDVERLQMAGVEVRYIDITNILGGVLHTKFWISDNTHIYLGSANMDWRSLTQVKELGIAIFNNRNLAADLTQIFEVYWYLGVNNLPYNWKNFYPSYYNTDHPLSINVSGVPHSVFIASAPQQLCTMERTNDLTALLSCIRNASKFVYVSVMNFIPIIYSKAGKILFWPYIEDELRRSAIDRQVSVKLLISCWQRSSFIMRNFLRSIAMLKSKNIDIEVKLFIVPDADPPIPYSRVNHAKYMVTDKTAYIGTSNWTGNYFTDTCGASINITPDDGLGLRQQLEDIFMRDWNSKYSYELYDTSPTKRCKLLKNMKQCTNDIYCDEIQPEKEIPEYSLE

SEQ ID NO: 35 - protein, 135 aa, Vaccinia virus
MGATISILASYDNPNLFTAMILMSPLVNADAVSRLNLLAAKLMGTITPNAPVGKLCPESVSRDMDKVYKYQYDPLINHEKIKAGFASQVLKATNKVRKIISKINTPRLSYSREQTMRLVMFQVHIISCNMQIVIE

SEQ ID NO: 36 - protein, 81 aa, Vaccinia virus
MSANCMFNLDNDYIYWKPITYPKALVFISHGAGKHSGRYDELAENISSLGILVFSHDHIGHGRSNGEKMMIDDFGTARGNY

SEQ ID NO: 37 - protein, 149 aa, Vaccinia virus
MATKLDYEDAVFYFVDDDKICSRDSIIDLIDEYITWRNHVIVFNKDITSCGRLYKELMKFDDVAIRYYGIDKINEIVEAMSEGDHYINFTKVHDQESLFATIGICAKITEHWGYKKISESRFQSLGNITDLMTDDNINILILFLEKKLN

SEQ ID NO: 38 - protein, 226 aa, Vaccinia virus
MLSMFMCNNIVDYVDDIDNGIVQDIEDEASNNVDHDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITELGRRCMNPVKTIKMFTLLSHTICDDCFVDYITDISPPDNTIPNTSTREYLKLIGITAIMFATYKTLKYMIG

SEQ ID NO: 39 - protein, 147 aa, Vaccinia virus
MFNMNINSPVRFVKETNRAKSPTRQSPGAAGYDLYSAYDYTIPPGERQLIKTDISMSMPKICYGRIAPRSGLSLKGIDIGGGVIDEDYRGNIGVILINNGKCTFNVNTGDRIAQLIYQRIYYPELEEVQSLDSTNRGDQGFGSTGLR

SEQ ID NO: 40 - protein, 480 aa, Vaccinia virus
MPIFVNTVYCKNILALSMTKKFKTIIDAIGGNIIVNSTILKKLSPYFRTHLRQKYTKNKDPVTRVCLDLDIHSLTSIVIYSYTGKVYIDSHNVVNLLRASILTSVEFIIYTCINFILRDFRKEYCVECYMMGIEYGLSNLLCHTKNFIAKHFLELEDDIIDNFDYLSMKLILESDELNVPDEDYVVDFVIKWYIKRRNKLGNLLLLIKNVIRSNYLSPRGINNVKWILDCTKIFHCDKQPRKSYKYPFIEYPMNMDQIIDIFHMCTSTHVGEVVYLIGGWMNNEIHNNAIAVNYISNNWIPIPPMNSPRLYATGIPANNKLYVVGGLPNPTSVERWFHGDAAWVNMPSLLKPRCNPAVASINNVIYVMGGHSETDTTTEYLLPNHDQWQFGPSTYYPHYKSCALVFGRRLFLVGRNAEFYCESSNTWTLIDDPIYPRDNPELIIVDNKLLLIGGFYRGSYIDTIEVYNHHTYSWNIWDGK

SEQ ID NO: 41 - protein, 512 aa, Vaccinia virus; identical to SEQ ID NO: 23

SEQ ID NO: 42 - protein, 229 aa, Vaccinia virus
MVKNNKIQKNKISNSCRMIMSTDPNNILMRHLKNLTDDEFKCIIHRSSDFLYLSDSDYTSITKETLVSEIVEEYPDDCNKILAIIFLVLDKDIDVDIKTKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 43 - protein, 117 aa, Vaccinia virus
MRTLLIRYILWRNDNDQTYYNDNFKKLMLLDELVDDGDVCTLIKNMRMTLSDGPLLDRLNQPVNNIEDAKRMIAISAKVARDIGERSEIRWEESFTILFRMIETYFDDLMIDLYGEK

SEQ ID NO: 44 - protein, 175 aa, Vaccinia virus
MTSSAMDNNEPKVLEMVYDATILPEGSSMDPNIMDCINRHINMCIQRTYSSSIIAILDRFLMMNKDELNNTQCHIIKEFMTYEQMAIDHYGGYVNAILYQIRKRPNQHHTIDLFKRIKRTRYDTFKVDPVEFVKKVIGFVSILNKYKPVYSYVLYENVLYDEFKCFINYVETKYF

SEQ ID NO: 45 - protein, 472 aa, Vaccinia virus
MIFVIESKLLQIYRNRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKHNKTPLYYLSGTDDEVIERINLLVQYGAKINNSVDEEGCGPLLACTDPSERVFKKIMSIGFEARIVDKFGKNHIHRHLMSDNPKASTISWMMKLGISPSKPDHDGNTPLHIVCSKTVKNVDIIDLLLPSTDVNKQNKFGDSPLTLLIKTLSPAHLINKLLSTSNVITDQTVNICIFYDRDDVLEIINDKGKQYDSTDFKMAVEVGSIRCVKYLLDNDIICEDAMYYAVLSEYETMVDYLLFNHFSVDFVVNGHTCMSECVRLNNPVILSKLMLHNPTSETMYLTMKAIEKDRLDKSIIIPFIAYFVLMHPDFCKNRRYFTSYKRFVTDYVHEGVSYEVFDDYF

SEQ ID NO: 46 - protein, 220 aa, Vaccinia virus
MVYKLVLLFCIASLGYSVEYKNTICPPRQDYRYWYFAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYSMRGSYGVTFIDELNQCLLDIKELSYDICYRE

SEQ ID NO: 47 - protein, 284 aa, Vaccinia virus
MDLSRINTWKSKQLKSFLSSKDAFKADVHGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELRLMYVNCVKKN

SEQ ID NO: 48 - protein, 369 aa, Vaccinia virus
MIALLILSLTCSVSTYRLQGFTNAGIVAYKNIQDDNIVFSPFGYSFSMFMSLLPASGNTRIELLKTMDLRKRDLGPAFTELISGLAKLKTSKYTYTDLTYQSFVDNTVCIKPSYYQQYHRFGLYRLNFRRDAVNKINSIVERRSGMSNVVDSNMLDNNTLWAIINTIYFKGIWQYPFDITKTRNASFTNKYGTKTVPMMNVVTKLQGNTITIDDEEYDMVRLPYKDANISMYLAIGDNMTHFTDSITAAKLDYWSFQLGNKVYNLKLPKFSIENKRDIKSIAEMMAPSMFNPDNASFKHMTRDPLYIYKMFQNAKIDVDEQGTVAEASTIMVATARSSPEKLEFNTPFVFIIRHDITGFILFMGKVESP

SEQ ID NO: 49 - protein, 88 aa, Vaccinia virus
MLAFCYSLPNAGDVIKGRVYEKDYALYIYLFDYPHFEAILAESVKMHMDRYVEYRDKLVGKTVKVKVIRVDYTKGYIDVNYKRMCRHQ

SEQ ID NO: 50 - protein, 424 aa, Vaccinia virus
MNPDNTIAVITETIPIGMQFDKVYLSTFNMWREILSNTTKTLDISSFYWSLSDEVGTNFGTIILNKIVQLPKRGVRVRVAVNKSNKPLKDVERLQMAGVEVRYIDITNILGGVLHTKFWISDNTHIYLGSANMDWRSLTQVKELGIAIFNNRNLAADLTQIFEVYWYLGVNNLPYNWKNFYPSYYNTDHPLSINVSGVPHSVFIASAPQQLCTMERTNDLTALLSCIRNASKFVYVSVMNFIPIIYSKAGNILFWPYIEDELRRAAIDRQVSVKLLISCWQRSSFIMRNFLRSIAMLKSKNINIEVKLFIVPDADPPIPYSRVNHAKYMVTDKTAYIGTSNWTGNYFTDTCGASINITPDDGLGLRQQLEDIFMRDWNSKYSYELYDTSPTKRCRLLKNMKQCTNDIYCDEIQPEKEIPEYSLE

SEQ ID NO: 51 - protein, 44 aa, Vaccinia virus
MQHANCNREIKIYEGAKHHLHKETDEVKKSVMKEIETWIFNRVK

SEQ ID NO: 52 - protein, 134 aa, Vaccinia virus
MTLVQHVVTIKSTYWVIPWELASYDNPNLFTAMILMSPLVNADAVSKLNLLAAKLMGTITLNAPVGKLCPESVSRDMDKVYKYQYDPLINHEKIKAGFASQVLKATNKVRKIISKINTPRLSYSREQTIRLAMF

SEQ ID NO: 53 - protein, 81 aa, Vaccinia virus; identical to SEQ ID NO: 36

SEQ ID NO: 54 - protein, 149 aa, Vaccinia virus; identical to SEQ ID NO: 37

SEQ ID NO: 55 - protein, 226 aa, Vaccinia virus
MLSMFMCNNIVDYVDDIDNGIVQDIEDEASNNVDHDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITELGRRCMNPVETIKMFTLLSHTICDDYFVDYITDISPPDNTIPNTSTREYLKLIGITAIMFATYKTLKYMIG

SEQ ID NO: 56 - protein, 147 aa, Vaccinia virus
MFNMNINSPVRFVKETNRAKSPTRQSPYAAGYDLYSAYDYTIPPGERQLIKTDISMSMPKFCYGRIAPRSGLSLKGIDIGGGVIDEDYRGNIGVILINNGKCTFNVNTGDRIAQLIYQRIYYPELEEVQSLDSTNRGDQGFGSTGLR

SEQ ID NO: 57 - protein, 480 aa, Vaccinia virus
MPIFVNTVYCKNILALSMTKKFKTIIDAIGGNIIVNSTILKKLSPYFRTHLRQKYTKNKDPVTWVCLDLDIHSLTSIVIYSYTGKVYIDSHNVVNLLRASILTSVEFIIYTCINFILRDFRKEYCVECYMMGIEYGLSNLLCHTKNFIAKHFLELEDDIIDNFDYLSMKLILESDELNVPDEDYVVDFVIKWYIKRRNKLGNLLLLIKNVIRSNYLSPRGINNVKWILDCTKIFHCDKQPRKSYKYPFIEYPMNMDQIIDIFHMCTSTHVGEVVYLIGGWMNNEIHNNAIAVNYISNNWIPIPPMNSPRLYASGIPANNKLYVVGGLPNPTSVERWFHGDAAWVNMPSLLKPRCNPAVASINNVIYVMGGHSETDTTTEYLLPNHDQWQFGPSTYYPHYKSCALVFGRRLFLVGRNAEFYCESSNTWTLIDDPIYPRDNPELIIVDNKLLLIGGFYRESYIDTIEVYNHHTYSWNIWDGK

SEQ ID NO: 58 - protein, 512 aa, Vaccinia virus
MESVIFSINGEIIQVNKEIITASPYNFFKRIQDHHLKDEAIILNGINYHAFESLLDYIRWKKINITINNVEMILVAAIIIDVPPVVDLCVKTMIHNINSTNCIRMFNFSKQYGIKKLYNASMSEIINNITAVTSDPEFGKLSKDELTTILSHEDVNVNHEDVTAMILLKWIHKNPNDVDIINILHPKFMTNTMRNAISLLGLTISKSTKPVTRNGIKHNIVVIKNSDYISTITHYSPRTEYWTIVGNTDRQFYNANVLHNCLYIIGGMINNRHVYSVSRVDLETKKWKTVTNMSSLKSEVSTCVNNGKLYVIGGLEFSISTGVAEYLKHGTSKWIRLPNLITPRYSGASVFVNDDIYVMGGVYTTYEKYVVLNDVECFTKNRWIKKSPMPRHHSIVYAVEYDGDIYVITGITHETRNYLYKYIVKEDKWIELYMYFNHVGKMFVCSCGDYILIIADAKYEYYPKSNTWNLFDMSTRNIEYYDMFTKDETPKCNVTHKSLPSFLSNCEKQFLQ

SEQ ID NO: 59 - protein, 224 aa, Vaccinia virus
MVKNNKISNSCRMIMSTDPNNILMRHLKNLTDDEFKCIIHRSSDFLYLSDSDYTSITKETLVSEIVEEYPDDCNKILAIIFLVLDKDIDVDIKTKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 60 - protein, 117 aa, Vaccinia virus; identical to SEQ ID NO: 25

SEQ ID NO: 61 - protein, 79 aa, Vaccinia virus
MTSSAMDNNEPKVLEMVYDATILPEGSSMDPYIMDCINRHINMCIQRTYSSSIIAILDRFLMMNKDELNNTQCHIIKNL

SEQ ID NO: 62 - protein, 472 aa, Vaccinia virus
MIFVIESKLLQIYRNRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKHNKTPLYYLSGTDDEVIERINLLVQYGAKINNSVDEEGCGPLLACTDPSERVFKKIMSIGFEARIVDKFGKNHIHRHLMSDNPKASTISWMMKLGISPSKPDHDGNTPLHIVCSKTVKNVDIIDLLLPSTDVNKQNKFGDSPLTLLIKTLSPAHLINKLLSTSNVITDQTVNICIFYDRDDVLEIINDKGKQYDSTDFKMAVEVGSIRCVKYLLDNDIICEDAMYYAVLSEYETMVDYLLFNHFSVDFVVNGHTCMSECVRLNNPVILSKLMLHNLTSETMYLTMKAIEKDRLDKSIIIPFIAYFVLMHPDFCKNRRYFTSYKRFVTDYVHEGVSYEVFDDYF

SEQ ID NO: 63 - protein, 196 aa, Vaccinia virus
MSSSTRLPVLVLAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYGMRGSYGVTFIDELNQCLLDIKELSYDICYRE

SEQ ID NO: 64 - protein, 76 aa, Vaccinia virus
MLQALFKYDINIYSANLENVLLDDAEIAKMIIEKHVEYKSDSYTKDLDIVKNNKLDEIISKNKELRLMYVNCVKKN

SEQ ID NO: 65 - protein, 189 aa, Vaccinia virus
MDLSRINTWKSKQLKSFLSSKDTFKADVHGHSALYYAIADNNVRLVCTLLNSGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTVDKHQ

SEQ ID NO: 66 - protein, 368 aa, Vaccinia virus
MIALLILSLACSASAYRLQGFTNAGIVAYKNIQDDNIVFSPFGYSFSMFMSLLPASGNTRIELLKTMDLRKRDLGPAFTELISGLAKLKTSKYTYTDLTYQSFVDNTVCIKPSYYQQYHRFGLYRLNFRRDAVNKINSIVERRSGMSNVVDSNMLDNNTLWAIINTIYFKGTWQYPFDITKTRNASFTNKYGTKTVPMMNVVTKLQGNTITIDDEEYDMVRLPYKDANISMYLAIGDNMTHFTDSITAAKDYWSFQLGNKVYNLKLPKFSIENKRDIKSIAEMMAPSMFNPDNASFKHMTRDPLYIYKMFQNAKIDVDEQGTVAEASTIMVATARSSPEELEFNTPFVFIIRHDITGFILFMGKVESP

SEQ ID NO: 67 - protein, 81 aa, Vaccinia virus
MGHIITYCQVHTNISILIRKAYHIIFFVIDCDCISLQFSNYVHHGNRFRTVLISKTSIACFSDIKRILPCTFKIYSINDCP

SEQ ID NO: 68 - protein, 88 aa, Vaccinia virus; identical to SEQ ID NO: 33

SEQ ID NO: 69 - protein, 134 aa, Vaccinia virus; identical to SEQ ID NO: 52

SEQ ID NO: 70 - protein, 149 aa, Vaccinia virus; identical to SEQ ID NO: 37

SEQ ID NO: 71 - protein, 226 aa, Vaccinia virus
MLSMFMCNNIVDYVDDIDNGIVQDIEDEASNNVDRDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITELGRRCMNPVKTIKMFTLLSHTICDDCFVDYITDISPPDNTIPNTSTREYLKLIGITAIMFATYKTLKYMIG

SEQ ID NO: 72 - protein, 147 aa, Vaccinia virus
MFNMNINSPVRFVKETNRAKSPTRQSPGAAGYDLYSAYDYTIPPGERQLIKTDISMSMPKICYGRIAPRSGLSLKGIDIGGGVIDEDYRGNIGVILINNGKCTFNVNTGDRIAQLIYQRIYYPELEEVQSLDSTDRGDQGFGSTGLR

SEQ ID NO: 73 - protein, 480 aa, Vaccinia virus
MPIFVNTVYCKNILALSMTKKFKTIIDAIGGNIIVNSTILKKLSPYFRTHLRQKYTKNKDPVTRVCLDLDIHSLTSIVIYSYTGKVYIDSHNVVNLLRASILTSVEFIIYTCINFILRDFRKEYCVECYMMGIEYGLSNLLCHTKNFIAKHFLELEDDIIDNFDYLSMKLILESDELNVPDEDYVVDFVIKWYIKRRNKLGNLLLLIKNVIRSNYLSPRGINNVKWILDCTKIFHCDKQPRKSYKYPFIEYPMNMDQIIDIFHMCTSTHVGEVVYLIGGWMNNEIHNNAIAVNYISNNWIPIPPMNSPRLYASGIPANNKLYVVGGLPNPTSVERWFHGDAAWVNMPSLLKPRCNPAVASINNVIYVMGGHSETDTTTEYLLPNHDQWQFGPSTYYPHYKSCALVFGRRLFLVGRNAEFYCESSNTWTLIDDPIYPRDNPELIIVDNKLLLIGGFYRESYIDTIEVYNHHTYSWNIWDGK

SEQ ID NO: 74 - protein, 506 aa, Vaccinia virus
MESVTFSINGEIIQVNKEIITASPYNFFKRIQEHHINDEVIILNGINYHAFESLLDYMRWKKINITINNVEMILVAAVIIDVTPVVDLCVKTMIHNINSTNCIRMFNFSKRYGIKKLYNASMSEIINNITAVTSDPEFGKLSKDELTTILSHEDVNVNHEDVTAMILLKWIHKNPNDVDIINILHPKFMTNTMRNAISLLGLTISKSTKPVTRNGIKHNIVVIKNSDYISTITHYSPRTEYWTIVGNTDRQFYNANVLHNCLYIIGGMINNRHVYSVSRVDLETKKWKTVTNMSSLKSEVSTCVNNGKLYVIGGLEFSISTGVAEYLKHGTSKWIRLPNLITPRYSGASVFVNDDIYVMGGVYTTYEKYVVLNDVECFTKNRWIKKSPMPRHHSIVYAVEYDGDIYAITGITHETRNYLYKYIVKEDKWIELYMYFNHVGKMFVCSCGDYILIIADAKYEYYPKSNTWNLFDMSTRNIEYYDMFTKDETHKSLPSFLSNCEKQFLQ

SEQ ID NO: 75 - protein, 224 aa, Vaccinia virus
MVKNNKISNSCRMIMSTNPNNILMRHLKNLTDDEFKCIIHRSSDFLYLSDRDYTSITKETLVSEIVEEYPDDCNKILAIIFLVLDKDIDVDIKTKLKPKPAVRFAILDKMTEDIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 76 - protein, 117 aa, Vaccinia virus; identical to SEQ ID NO: 25

SEQ ID NO: 77 - protein, 175 aa, Vaccinia virus
MTSSAMDNNEPKVLEMVYDATILPEGSSMDPNIIDCINRHINMCIQRTYSSSIIAILDRFLTMNKDELNNTQCHIIKEFMTYEQMAIDHYGGYVNAILYQIRKRPNQHHTIDLFKKIKRTRYDTFKVDPVEFVKKVIGFVSILNKYKPVYSYVLYENVLYDEFKCFIDYVETKYF

SEQ ID NO: 78 - protein, 72 aa, Vaccinia virus
MLHNPTSETMYLTMNAIKKDKLDKSIIIPFIAYFVLMHPDFCKNRRYFTSYKRFVTDYVHEGVSYEVFDDYF

SEQ ID NO: 79 - protein, 214 aa, Vaccinia virus
MSIGFEARIVDKFGKNHIHRHLMSDNPKASTISWMMKLGISPSKPDHDGNTPLHIVCSKTVKYVDIIDLLLPSTDVNKQNKFGDSPLTLLIKTLSLAHLINKLLSTSNVITDQTVNICIFYDRDDVLEIINDKGKQYDFKMAVEVGSIKCVKYLLDNDIICEDAMYYAVLSEYKTMVDYLLFNHFSVDSVVNGHTCMSECVKLNNRHFIEADVT

SEQ ID NO: 80 - protein, 161 aa, Vaccinia virus
MIFVIESKLLQIYRNRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKHNKTPLYYLSGTDDEVIERINLLVQYGAKINN

SEQ ID NO: 81 - protein, 220 aa, Vaccinia virus; identical to SEQ ID NO: 28

SEQ ID NO: 82 - protein, 284 aa, Vaccinia virus
MDLSRINTWKSKQLKSFLSSKDTFKADVHGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELRLMYVNCVKKN

SEQ ID NO: 83 - protein, 364 aa, Vaccinia virus
MIALLILSLTCSVSTYRLQGFTNAGIVAYKNIQDDNIVFSPFGYSFSMFMSLLPASGNTRIELLKTMDLRKRDLGPAFTELISGLAKLKTSKYTYTDLTYQSFVDNTVCIKPSYYQQYHRLNFRRDAVNKINSIVERRSGMSNVVDSNMLDNNTLWAIINTIYFKGIWQYPFDITKTRNASFTNKYGTKTVPMMNVVTKLQGNTITIDDKEYDMVRLPYKDANISMYLAIGDNMTHFTDSITAAKLDYWSFQLGNKVYNLKLPKFSIENKRDIKSIAEMMAPSMFNPDNASFKHMTRDPLYIYKMFQNAKIDVDEQGTVAEASTIMVATARSSPEKLEFNTPFVFIIRHDITGFILFMGKVESP

SEQ ID NO: 84 - protein, 88 aa, Vaccinia virus
MLAFCYSLPNAGDVIKGRVYENDYALYIYLFDYPHFEAILAESVKMHMDRYVEYRDKLVGKTVKVKVIRVDYTKGYIDVNYKRMCRHQ

SEQ ID NO: 85 - protein, 424 aa, Vaccinia virus
MNPDNTIAVITETIPIGMQFDKVYLSTFNMWREILSNTTKTLDISSFYWSLSDEVGTNFGTIILNEIVQLPKRGVRVRVAVNKSNKPLKDVERLQMAGVEVRYIDITNILGGVLHTKFWISDNTHIYLGSANMDWRSLTQVKELGIAIFNNRNLAADLTQIFEVYWYLGVNNLPYNWKNFYPSYYNTDHPLSINVSGVPHSVFIASAPQQLCTMERTNDLTALLSCIRNASKFVYVSVMNFIPIIYSKAGKILFWPYIEDELRRSAIDRQVSVKLLISCWQRSSFIMRNFLRSIAMLKSKNINIEVKLFIVPDADPPIPYSRVNHAKYMVTDKTAYIGTSNWTGNYFTDTCGASINITPDDGLGLRQQLEDIFMRDWNSKYSYELYDTSPTKRCKLLKNMKQCTNDIYCDEIQPEKEIPEYSLE

SEQ ID NO: 86 - protein, 171 aa, Vaccinia virus
MGATISILASYDNPNLFTAMILMSPLVNADAVSKLNLLAAKLMGTITPNAPVGKLCPESVSRDMDKVYKYQYDPLINHEKIKAGFASQVLKATNKVRKIISKINTPPTLILQGTNNEISDVLGAYYFMQHANCNREIKIYEGAKHHLHKETDEVKKSVMKEIETWIFNRVK

SEQ ID NO: 87 - protein, 81 aa, Vaccinia virus; identical to SEQ ID NO: 36

SEQ ID NO: 88 - protein, 149 aa, Vaccinia virus; identical to SEQ ID NO: 37

SEQ ID NO: 89 - protein, 226 aa, Vaccinia virus
MLSMFMCNNIVDYVDDIDNGIVQDIEDEASNNVDHDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITKLGRRCMNPVKTIKMFTLLSHTICDDCFVDYITDISPPDNTIPNTSTREYLKLIGITAIMFATYKTLKYMIG

SEQ ID NO: 90 - protein, 147 aa, Vaccinia virus; identical to SEQ ID NO: 39

SEQ ID NO: 91 - protein, 480 aa, Vaccinia virus; identical to SEQ ID NO: 73

SEQ ID NO: 92 - protein, 512 aa, Vaccinia virus
MESVIFSINGEIIQVNKEIITASPYNFFKRIQDHHLKDEAIILNGINYHAFESLLDYMRWKKINITINNVEMILVAAIIIDVPPVVDLCVKTMIHNINFTNCIRMFNFSKRYGIKKLYNASMSEIINNITAVTSDPEFGKLSKDELTTILSHEDVNVNHEDVTAMILLKWIHKNPNDVDIINILHPKFMTNTMRNAISLLGLTISKSTKPVTRNGIKHNIVVIKNSDYISTITHYSPRTEYWTIVGNTDRQFYNANVLHNCLYIIGGMINNRHVYSVSRVDLKTKKWKTVTNMSSLKSEVSTCVNDGKLYVIGGLEFSISTGVAEYLKHGTSKWIRLPNLITPRYSGASVFVNDDIYVMGGVYTTYEKYVVLNDVECFTKNRWIKKSPMPRHHSIVYAVEYDGDIYVITGITHETRNYLYKYIVKEDKWIELYMYFNHVGKMFVCSCGDYILIIADAKYEYYPKSNTWNLFDMSTRNIEYYDMFTKDETPKCNVTHKSLPSFLSNCEKQFLQ

SEQ ID NO: 93 - protein, 224 aa, Vaccinia virus
MVKNNKISNSCRMIMSTNPNNILMRHLKNLTDDEFKCIIHRSSDFLYLSDSDYTSITKETLVSEIVEEYPDDCNKILAIIFLVLDKDIDVDIETKLKPKPAVRFAILDKMTADIKLTDLVRHYFRYIEQDIPLGPLFKKIDSYRTRAINKYSKELGLATEYFNKYGHLMFYTLPIPYNRFFCRNSIGFLAVLSPTIGHVKAFYKFIEYVSIDDRRKFKKELMSK

SEQ ID NO: 94 - protein, 117 aa, Vaccinia virus; identical to SEQ ID NO: 25

SEQ ID NO: 95 - protein, 175 aa, Vaccinia virus
MTSSAMDNNEPKVLEMVYDATILPEGSSMDPNIMDCINRHINMCIQRTYSSSIIAILDRFLTMNKDELNNTQCHIIKEFMTYEQMAIDHYGEYVNAILYQIRKRPNQHHTIDLFKKIKRTRYDTFKVDPVEFVKKVIGFVSILNKYKPVYSYVLYENVLYDEFKCFIDYVETKYF

SEQ ID NO: 96 - protein, 470 aa, Vaccinia virus
MIFVIESKLLQIYRNRNINFYTTMDNIMSAEYYLSLYAKYNSKNLDVFRNMLQAIEPSGNNYHILHAYCGIKGLDERFVEELLHRGYSPNETDDDGNYPLHIASKINNNRIVAMLLTHGADPNACDKQHKTPLYYLSGTDDEVIERINLLVQYGAKINNSVDEEGCGPLLACTDPSERVFKKIMSIGFEARIVDKFGKNHIHRHLMSDNPKASTISWMMKLGISPSKPDHDGNTPLHIVCSKTVKNVDIIDLLLPSTDVNKQNKFGDSPLTLLIKTLSPAHLINKLLSTSNVITDQTVNICIFYDRDDVLEIINDKGKQYDSTDFKMAVEVGSIRCVKYLLDNDIICEDAMYYAVLSEYETMVDYLLFNHFSVDSVVNGHTCMSECVRLNNPVILSKLMLHNPTSETMYLTMKAIEKDRLDKSIIIPFIAYFVLMHPDFCKNRRYFTSYKRFVTDYVHEGVSYEVFDDYF

SEQ ID NO: 97 - protein, 220 aa, Vaccinia virus; identical to SEQ ID NO: 28

SEQ ID NO: 98 - protein, 284 aa, Vaccinia virus
MDLSRINTWKSKQLKSFLSSKDAFKADINGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELKLMYVNCVKKN

SEQ ID NO: 99 - protein, 309 aa, Vaccinia virus
IELLKTMDLRKRDLGPAFTELISGLAKLKTSKYTYTDLTYQSFVDNTVCIKPSYYQQYHRFGLYRLNFRRDAVNKINSIVERRSGMSNVVDSNMLDNNTLWAIINTIYFKGIWQYPFDITKTRNASFTNKYGTKTVPMMNVVTKLQGNTITIDDEEYDMVRLPYKDANISMYLAIGDNMTHFTDSITAAKLDYWSSQLGNKVYNLKLPKFSIENKRDIKSIAEMMAPSMFNPDNASFKHMTRDPLYIYKMFQNAKIDVDEQGTVAEASTIMVATARSSPEKLEFNTPFVFIIRHDITGFILFMGKVESP

SEQ ID NO: 100 - protein, 88 aa, Vaccinia virus
MLAFCYSLPNAGDVIKGRVYENDYALYIYLFDYPHSEAILAESVKMHMDRYVEYRDKLVGKTVKVKVIRVDYTKGYIDVNYKRMCRHQ

SEQ ID NO: 101 - protein, 424 aa, Vaccinia virus; identical to SEQ ID NO: 85

SEQ ID NO: 102 - protein, 175 aa, Vaccinia virus
MGHSMGATISILASYDNPNLFTAMILMSPLVNADAVSRLNLLAAKLMGTITPNAPVGKLCPESVSRDMDKVYKYQYDPLINHEKIKAGFASQVLKATNKVRKIISKINTPPTLILQGTNNKISDVLGAYYFMQHANCNREIKIYEGAKHHLHKETDEVKKSVMKEIETWIFNRVK

SEQ ID NO: 103 - protein, 81 aa, Vaccinia virus; identical to SEQ ID NO: 36

SEQ ID NO: 104 - protein, 149 aa, Vaccinia virus; identical to SEQ ID NO: 37

SEQ ID NO: 105 - protein, 226 aa, Vaccinia virus; identical to SEQ ID NO: 38

SEQ ID NO: 106 - protein, 147 aa, Vaccinia virus
MFNMNINSPVRFVKETNRAKSPTRQSPGAAGYDLYSAYDYTIPPGERQLIKTDISMSMPKFCYGRIAPRSGLSLKGIDIGGGVIDEDYRGNIGVILINNGKCTFNVNTGDRIAQLIYQRIYYPELEEVQSLDSTNRGDQGFGSTGLR

SEQ ID NO: 107 - protein, 480 aa, Vaccinia virus
MPIFVNTVYCKNILALSMTKKFKTIIDAIGGNIIVNSTILKKLSPYFRTHLRQKYTKNKDPVTRVCLDLDIHSLTSIVIYSYTGKVYIDSHNVVNLLRASILTSVEFIIYTCINFILRDFRKEYCVECYMMGIEYGLSNLLCHTKNFIAKHFLELEDDIIDNFDYLSIKLILESDELNVPDEDYVVDFVIKWYIKRRNKLGNLLLLIKNVIRSNYLSPRGINNVKWILDCTKIFHCDKQPRKSYKYPFIEYPMNMDQIIDIFHMCTSTHVGEVVYLIGGWMNNEIHNNAIAVNYISNNWIPIPPMNSPRLYASGIPANNKLYVVGGLPNPTSVERWFHGDAAWVNMPSLLKPRCNPAVASINNVIYVMGGHSETDTTTEYLLPNHDQWQFGPSTYYPHYKSCALVFGRRLFLVGRNAEFYCESSNTWTLIDDPIYPRDNPELIIVDNKLLLIGGFYRESYIDTIEVYNHHTYSWNIWDGK

SEQ ID NO: 108 - protein, 222 aa, Vaccinia virus
MNHCLLAISAVYFKAKWLTPFEKEFTSDYPFYVSPTEMVDVSMMSMYGELFNHASVKESFGNFSIIELPYVGDTSMMVILPDKIDGLESIEQNLTDTNFKKWCNSLDAMFIDVHIPKFKVTGSYNLVDTLVKSGLTEVFGSTGDYSNMCNLDVSVDAMIHKTYIDVNEEYTEAAAATCALVSDCASTITNEFCVDHPFIYVIRHVDGKILFVGRYCSPTTNC

SEQ ID NO: 109 - protein, 149 aa, Vaccinia virus
MTANFSTHVFSPQHCGCDRLTSIDDVKQCLTEYIYWSSYAYRNRQCAGQLYSTLLSFRDDAELVFIDIRELVKNMPWDDVKDCTEIIRCYIPDEQKTIREISAIIGLCAYAATYWGGEDHPTSNSLNALFVMLEMLNYVDYNIIFRRMN

SEQ ID NO: 110 - protein, 91 aa, Vaccinia virus
MYNSSIHTPEYDVIIHVIEHLKHHKQCVQTVTSGMVFTSPVSSSICTKSDDGRNLSDGFLLIRYITTDDFCTIFDIIPRHIFYQLANVDEH

SEQ ID NO: 111 - protein, 326 aa, Vaccinia virus
  VARIANT 31: note = any amino acid
MSILPVIFLPIFFYSSFVQTFNASECIDKGXYFASFMELENEPVILPCPQINTLSSGYNILDILWEKRGADNDRIIPIDNGSNMLILNPTQSDSGIYICITTNETYCDMMSLNLTIVSVSESNIDFISYPQIVNERSTGEMVCPNINAFIASNVNADIIWSGHRRLRNKRLKQRTPGIITIEDVRKNDAGYYTCVLEYIYGGKTYNVTRIVKLEVRDKIIHPTMQLPEGVVTSIGSNLTIACRVSLRPPTTDADVFWISNGMYYEEDDGDGDGRISVANKIYMTDKRRVITSRLNINPVKEEDATTFTCMAFTIPSISKTVTVSIT

SEQ ID NO: 112 - protein, 75 aa, Vaccinia virus
MVIIPGVRCLSLLFLRRRCPLHIISAFTLLAINALILGHTISPVDLSFTICGYEIKSIFDSETDTIVKFNDIMSQ

SEQ ID NO: 113 - protein, 340 aa, Vaccinia virus
MSRKFMQVYEYDREQYLDEFIEDRYNDSFITSPEYYSAEKYMCRYTTLNHNCVNVRRCALDSKLLHDIITNCKIYNNIELVRATKFVYYLDLIKCNWVSKVGDSVLYPVIFITHTSTRNLDKVSVKTYKGVKVKKLNRCADHAIVINPFVKFKLTLPNKTSHAKVLVTFCKLRTDITPVEAPLPGNVLVYTFPDINKRIPGYIHVNIEGCIDGMIYINSSKFACVLKLHRSMYRIPPFPIDICSCCSQYTNDDIEIPIHDLIKDVAIFKNKETVYYLKLNNKTIARFTYFNNIDTAITQEHEYVKIALGIVCKLMINNMHSIVGVNHSNTFVNCLLEDNV

SEQ ID NO: 114 - protein, 574 aa, Vaccinia virus
MSRRLIYVLNINRKSTHKIQENEIYTYFSHCNIDHTSTELDFVVKNYDLNRRQHVTGYTALHCYLYNNYFTNDVLKILLNHDVNVTMKTSSGRMPVYILLTRCCNISHDVVIDMIDKDKNHLSHRDYSNLLLEYIKSRYMLLKEEDIDENIVSTLLDKGIDPNFKQDGYTALHYYYLCLAHVYKPGECRKPITIKKAKRIISLFIQHGANLNALDNCGNTPFHLYLSIEMCNNIHMTKMLLTFNPNFKICNNHGLTPILCYITSDYIQHDILVMLIHHYETNVGEMPIDERRMIVFEFIKTYSTRPADSITYLMNRFKNINIYTRYEGKTLLHVACEYNNTQVIDYLIRINGDINALTDNNKHATQLIIDNKENSPYTINCLLYILRYIVDKNVIRSLVDQLPSLPIFDIKSFEKFISYCILLDDTFYDRHVKNRDSKTYRYAFSKYMSFDKYDGIITKCHDETMLLKLSTVLDTTLYAVLRCHNSRKLRRYLTELKKYNNDKSFKIYSNIMNERYLNVYYKDMYVSKVYDKLFPVFTDKNCLLTLLPSEIIYEILYMLTINDLYNISYPPTKV

SEQ ID NO: 115 - protein, 353 aa, Vaccinia virus
MTMKMMVHIYFVSLSLLLLLFHSYAIDIENEITEFFNKMRDTLPAKDSKWLNPACMFGGTMNDMATLGEPFSAKCPPIEDSLLSHRYKDYVVKWERLEKNRRRQVSNKRVKHGDLWIANYTSKFSNRRYLCTVTTKNGDCVQGIVRSHIKKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 116 - protein, 127 aa, Vaccinia virus
MDEDTRLSRYLYLTDREHINVDSIKQLCKISDPNACYRCGCTALHEYFYNYRSVNGKYKYRYNGYYQYYSSSDYENYNEYYYDDYDRTGMNSESDSESDNISIKTEYENEYEFYDETQDQSTQHNDL

SEQ ID NO: 117 - protein, 91 aa, Vaccinia virus
MSLESFIITTFNNNSSTNIDNMCHLYVKVCPSSLLFRLFVECCDINKLVEGTTPLHCYLMNEGFESSVLKNLLKEYVMNTFNVHDIHYTNI

SEQ ID NO: 118 - protein, 181 aa, Vaccinia virus
MISLSFLIHNPLKKWKLKPSISINGYRSTFTMAFPCAQFRPCHCHATKDSLNTVADVRHCLTEYILWVSHRWTHRESAGSLYRLLISFRTDATELFGGELKDSLPWDNIDNCVEIIKCFIRNDSMKTAEELRAIIGLCTQSAIVSGRVFNDKYIDILLMLRKILNENDYLTLLDHIRTAKY

SEQ ID NO: 119 - protein, 386 aa, Vaccinia virus
MIAFIIFREIGIISTRIAMDYCGRECTILCRLLDEDVTYKKIKLEIETCHNLSKHIDRRGNNALHCYVSNKCDTDIKIVRLLLSRGVERLCRNNEGLTPLGAYSKHRYVKSQIVHLLISSYSNSSNELKSNINDFDLSSDNIDLRLLKYLIVDKRIRPSKNTNYAINGLGLVDIYVTTPNPRPEVLLWLLKSECYSTGYVFRTCMYNSDMCKNSLHYYISSHRESQSLSKDVIKCLINNNVSIHGRDEGGSLPIQYYWSFSTIDIEIVKLLLIKDVDTCRVYDVSPILEAYYLNKRFRVTPYNVDMEIVNLLIERRHTLVDVMRSITSYDSREYNHYIIDNILKRFRQQDESIVQAMLINYLHYGDMVVRCMLDNGQQLSSARLLC

SEQ ID NO: 120 - protein, 150 aa, Vaccinia virus
MYGLILSRFNNCGYHCYETILIDVFDILSKYMDDIDMIDNENKTLLYYAVDVNNIQFAKRLLEYGASVTTSRSIINTAIQKSSYQRENKTRIVDLLLSYHPTLETMIDAFNRDIRYLYPEPLFACIRYALILDDDFPSKVSMISPVIIRN

SEQ ID NO: 121 - protein, 91 aa, Vaccinia virus
MRRCIHIKERKIHMTNIVDRNVTFILTVVHKYVRYVPHTVANDAHNLVHLAHLIHFIIYFFIIRDVRKKKKKKKKNRTIYFFSNVYARHIK

SEQ ID NO: 122 - protein, 259 aa, Vaccinia virus
MSRINITKKIYCSVFLFLFLFLSYISNYEKVNDEMYEMGEMDEIVSIVRDSMWYIPNVFMDDGKNEGHVSVNNVCHMYFTFFDVDTSSHLFKLVIKHCDLNKRGNSPLHCYTMNTRFNPSVLKILLHHGMRNFDSKDEKGHHYLIHSLSIDNKIFDILTDTIDDFSKSSDLLLCYLRYKFNGSLNYYVLYKGSDPNCADEDELTSLHYYCKHISTFYKSNYYKLSHTKMRAEKRFIYAIIDYGANINAVTHLPSTVYQT

SEQ ID NO: 123 - protein, 103 aa, Vaccinia virus
MEQTLTRLHTYLQQYTKHSPRVVYALLSRGYVIILIVHPSWNDCATGHILIMLLNWHEQKEEGQHLLYLFIKHNQGYTLNILRYLLDRFDIQKDEYIYRLSKL

SEQ ID NO: 124 - protein, 113 aa, Vaccinia virus
MLPHTSDTTSTFRLKTVFDLVFENRNIIYKADVVNDIIHHRLKVSLPMIKSLFYKMSEFSPYDDYYVKKILAYCLLRDESFAELHSKFCLNEDYKSVFMKNISFDKIDSIIVT

SEQ ID NO: 125 - protein, 122 aa, Vaccinia virus
MKSVLYSYILFLSCIIINGRDIAPHAPSDGKCKDNEYKRHNLCPGTYASRLCDSKTNTQCTPCGSGTFTSRNNHLPACLSCNGRRDRVTLLTIESVNALPDIIVFSKDHPDARHVFPKQNVE

SEQ ID NO: 126 - protein, 244 aa, Vaccinia virus
MHVPASLQQSSSSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITREEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV

SEQ ID NO: 127 - protein, 345 aa, Vaccinia virus
MDIFREIASSMKGENVFISPASISSVLTILYYGANGSTAEQLSKYVEKEENMDKVSAQNISFKSINKVYGRYSAVFKDSFLRKIGDKFQTVDFTDCRTIDAINKCVDIFTEGKINPLLDEPLSPDTCLLAISAVYFKAKWLTPFEKEFTSDYPFYVSPTEMVDVSMMSMYGKAFNHASVKESFGNFSIIELPYVGDTSMMVILPDKIDGLESIEQNLTDTNFKKWCNSLEATFIDVHIPKFKVTGSYNLVDTLVKSGLTEVFGSTGDYSNMCNSDVSVDAMIHKTYIDVNEEYTEAAAATCALVSDCASTITNEFCVDHPFIYVIRHVDGKILFVGRYCSPTTNC

SEQ ID NO: 128 - protein, 149 aa, Vaccinia virus
MTANFSTHVFSPQHCGCDRLTSIDDVRQCLTEYIYWSSYAYRNRQCAGQLYSTLLSFRDDAELVFIDIRELVKNMPWDDVKDCAEIIRCYIPDEQKTIREISAIIGLCAYAATYWGGEDHPTSNSLNALFVMLEMLNYVDYNIIFRRMN

SEQ ID NO: 129 - protein, 326 aa, Vaccinia virus
MSILPVIFLSIFFYSSFVQTFNAPECIDKGQYFASFMELENEPVILPCPQINTLSSGYNILDILWEKRGADNDRIIPIDNGSNMLILNPTQSDSGIYICITTNETYCDMMSLNLTIVSVSESNIDLISYPQIVNERSTGEMVCPNINAFIASNVNADIIWSGHRRLRNKRLKQRTPGIITIEDVRKNDAGYYTCVLEYIYGGKTYNVTRIVKLEVRDKIIPSTMQLPDGIVTSIGSNLTIACRVSLRPPTTDADVFWISNGMYYEEDDGDGNGRISVANKIYMTDKRRVITSRLNINPVKEEDATTFTCMAFTIPSISKTVTVSIT

SEQ ID NO: 130 - protein, 340 aa, Vaccinia virus
MSRKFMQVYEYDREQYLDEFIEDRYNDSFITSPEYYSAEKYMCRYTTLNHNCINVRRCALDSKLLHDIITNCKIYNNIELVRATKFVYYLDLIKCNWVSKVGDSVLYPVIFITHTSTRNLDKVSVKTYKGVKVKKLNRCADHAIVINPFVKFKLTLPNKTSHAKVLVTFCKLKTDITPVEAPLPGNVLVYTFPDINKRIPGYIHLNIEGCIDGMIYINSSKFACVLKLHRSMYRIPPFPIDICSCCSQYINYDIEIPIHDLIKDVAIFKNKETVYYLKLNNKTIARFTYFNNIDTAITQEHEYVKIALGIVCKLMINNMHSIVGVNHSNTFVNCLLEDNV

SEQ ID NO: 131 - protein, 574 aa, Vaccinia virus
MSRRLIYVLNINRESTHKIQENEIYTYFSHCNIDHTSTELDFVVKNYDLNRRQPVTGYTALHCYLYNNYFTNDVLKILLNHGVDVTMKTSSGRMPVYILLTRCCNISHDVVIDMIDKDKNHLLHRDYSNLLLEYIKSRYMLLKEEDIDENIVSTLLDKGIDPNFKQDGYTALHYYYLCLAHVYKPGECRKPITIKKAKRIISLFIQHGANLNALDNCGNTPFHLYLSIEMCNNIHMTKMLLTFNPNFEICNNHGLTPILCYITSDYIQHDILVMLIHHYETNVGEMPIDERRIIVFEFIKTYSTRPADSITYLMNRFKNIDIYTRYEGKTLLHVACEYNNTHVIDYLIRINGDINALTDNNKHATQLIIDNKENSPYTINCLLYILRYIVDKNVIRSLVDQLPSLPIFDIKSFEKFISYCILLDDTFYNRHVRNRDSKTYRYAFSKYMSFDKYDGIITKCHKETILLKLSTVLDTTLYAVLRCHNSKKLRRYLTELKKYNNDKSFKIYSNIMNERYLNVYYKDMYVSKVYDKLFPVFTDKNCLLTLLPSEIIYEILYMLTINDLYNISYPPTKV

SEQ ID NO: 132 - protein, 351 aa, Vaccinia virus
MTMKMMVHIYFVSLLLLLFHSYAIDIENEITEFFNKMRDTLPAKDSKWLNPACMFGGTMNDIAALGEPFSAKCPPIEDSLLSHRYKDYVVKWERLEKNRRRQVSNKRVKHGDLWIANYTSKFSNRRYLCTVTTKNGDCVQGIVRSHIRKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 133 - protein, 56 aa, Vaccinia virus
MHVIDVDVRLYMSTFIIIDQSTENTSIDTTVTINIIYLAIMKIIMNIIMMIMIELV

SEQ ID NO: 134 - protein, 53 aa, Vaccinia virus
MNSESDNISIKTEYEFYDETQDQSTQLVGYDIKLKTNEDDFMAMIDQWVSMII

SEQ ID NO: 135 - protein, 309 aa, Vaccinia virus
MEMYPRHRYSKHSVFKGFSDKVRKNDLDMNVVKELLSNGASLTIKDSSNKDPITVYFRRTIMNLEMIDERKYIVHSYLKNYKNFDYPFFRKLVLTNKHCLNNYYNISDSKYGTPLHILASNKKLITPNYMKLLVYNGNDINARGEDTQMRTPLHKYLCKFVYHNIEYGIRYYNEKIIDAFIELGADLTIPNDDGMIPVVYCIHSNAEYGYNNITNIKIIRKLLNLSRRASHNLFRDRVMHDYISNTYIDLECLDIIRSLDGFDINGYFEGRTPLHCAIQHNFTQIAKYLLDRGADIVVPNTLIIHQYIQ

SEQ ID NO: 136 - protein, 134 aa, Vaccinia virus
MEEDTNISNKVIRYNTVNNIWETLPNFWTGTINPGVVSHKDDIYVVCDIKDEKNVKTCIFRYNTNTYNGWELVTTTESRLSALHTILYNNTIMMLHCYESYMLQDTFNVYTREWNHMCHQHSNSYIMYNILPIY

SEQ ID NO: 137 - protein, 353 aa, Vaccinia virus
MDIFKELILKHTDENVLISPVSILSTLSILNHGAAGSTAEQLSKYIENMNENTPDDNNDMDVDIPYCATLATANKIYGSDSIEFHASFLQKIKDDFQTVNFNNANQTKELINEWVKTMTNGKINSLLTSPLSINTRMTVVSAVHFKAMWKYPFSKHLTYTDKFYISKNIVTSVDMMVSTENNLQYVHINELFGGFSIIDIPYEGNSSMVIILPDDIEGIYNIEKNITDEKFKKWCGMLSTKSIDLYMPKFKVEMTEPYNLVPILENLGLTNIFGYYADFSKMCNETITVEKFLHTTFIDVNEEYTEASAVTGVFMTNFSMVYRTKVYINHPFMYMIKDNTGRILFIGKYCYPQ

SEQ ID NO: 138 - protein, 190 aa, Vaccinia virus
MMIYGLIACLIFVTSSIASPLYIPVIPPISEDKSFNSVEVLVSLFRDDQKDYTVTSQFNNYTIDTKDWTIGVLSTPDGLDIPLTNITYWSRFTIGRALFKSESEDIFQKKMSILGVSIECKKSSTLLTFLTVRKMTRVFNKFPDMAYYRGDCLKAVYVTMTYKNTKTGETDYTYLSNGGLPAYYRNGVDG

SEQ ID NO: 139 - protein, 62 aa, Vaccinia virus
MKLFTQNDRYFGLLDSCTHIFCITCINIWHKTRRETGASDNCPICRTRFRNITMSKFYKLVN

SEQ ID NO: 140 - protein, 184 aa, Vaccinia virus
MEFDPAKINTSSIDHVTILQYIDEPNDIRLTVCIIRNINNITYYINITKINTHLANQFRAWKKRIAGRDYMTNLSRDTGIQQSKLTETIRNCQKNRNIYGLYIHYNLVINVVIDWITDVIVQSILRGLVNWYIANNTYTPNTPNNTTTISELDIIKILDKYEDVYRVSKEKECGICYEVVYSKR

SEQ ID NO: 141 - protein, 331 aa, Vaccinia virus
MDIYDDKGLQTIKLFNNEFDCIRNDIRELFKHVTDSDSIQLPMEDNSDIIENIRKILYRRLKNVECVDIDSTITFMKYDPNDDNKRTCSNWVPLTNNYMEYCLVIYLETPICGGKIKLYHPTGNIKSDKDIMFAKTLDFKSKKVLTGRKTIAVLDISVSYNRSMTTIHYNDDVDIDIHTDKNGKELCYCYITIDDHYLVDVETIGVIVNRSGKCLLVNNHLGIGIVKDKRISDSFGDVCMDTIFDFSEARELFSLTNDDNRNIAWDTDKLDDDTDIWTPVTEDDYKFLSRLVLYAKSQSDTVFDYYVLTGDTEPPTVFIFKVTRFYFNMPK

SEQ ID NO: 142 - protein, 140 aa, Vaccinia virus
MSMKYLMLLFAAMIIRSFADSGNAIETTSPEITNATTDIPAIRLCGPEGDGYCLHGDCIHARDIDGMYCRCSHGYTGIRCQHVVLVDYQRSENPNTTTSYIPSPGIMLVLVGIIIITCCLLSVYRFTRRTKLPIQDMVVP

SEQ ID NO: 143 - protein, 112 aa, Vaccinia virus
MDEIVRIVRDSMWYIPNVFMDDGKNEGHVSVNNVCHMYFTFFDVDTSSHLFKLVIKHCDLNKRGNSPLHCYTMNTRFNPSVLKILLHHGMRNFDSKDEKGHHYQSITRSLIY

SEQ ID NO: 144 - protein, 109 aa, Vaccinia virus
MLFYLEEPIRGYVIILIVHPSWNDCATGHILIMLLNWHEQKEEGQHLLYLFIKHNQGYTLNILRYLLDRFDIQKDEYYNTAFQNCNNNVASYIGYDINLPTKDGIRLGV

SEQ ID NO: 145 - protein, 64 aa, Vaccinia virus
MLPHTSDTTSTFRLKTVFDLVFENRNIIYKADVVNDIIHHRLKVSLPMIKSLFYKMSLPTTITT

SEQ ID NO: 146 - protein, 48 aa, Vaccinia virus
MYDDLIEQCHLSMERKSKLVDKALNKLESTIGQSRLSYLPPEIMRNII

SEQ ID NO: 147 - protein, 122 aa, Vaccinia virus
MKSVLYSYILFLSCIIINGRDIAPHAPSDGKCKDNEYKRHNLCPGTYASRLCDSKTNTQCTPCGSGTFTSRNNHLPACLSCNGRRDRVTRLTIESVNALPDIIVFSKDHPDARHVFPKQNVE

SEQ ID NO: 148 - protein, 72 aa, Vaccinia virus
MDSLRPVVVVNWIQINFHIDIVKGITGYGFAFICGRDGVRICSETTRRTDDVSGYSVSYSTFCLGNTCLASG

SEQ ID NO: 149 - protein, 61 aa, Vaccinia virus
MWKLICIQLTTTTGLSESISTSELTITMNHKDCNPVFREEYFSVLNKVATSGFFTGERCAL

SEQ ID NO: 150 - protein, 244 aa, Vaccinia virus
MHVPASLQQSSSSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITHEEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV

SEQ ID NO: 151 - protein, 222 aa, Vaccinia virus
MNHCLLAISAVYFKAKWLTPFEKEFTSDYPFYVSPTEMVDVSMMSMYGKAFNHASVKESFGNFSIIELPYVGDTSMMVILPDKIDGLESIEQNLTDTNFKKWCDFMDAMFIDVHIPKFKVTGSYNLVDTLVKSGLTEVFGSTGDYSNMCNLDVSVDAMIHKTYIDVNEEYTEAAAATCALVSDCASTITNEFCVDHPFIYVIRHVDGKILFVGRYCSPTTNC

SEQ ID NO: 152 - protein, 149 aa, Vaccinia virus; identical to SEQ ID NO: 109

SEQ ID NO: 153 - protein, 91 aa, Vaccinia virus; identical to SEQ ID NO: 110

SEQ ID NO: 154 - protein, 290 aa, Vaccinia virus
MELENEPVILPCPQINTLSSGYNILDILWEKRGADNDRIIPIDNGSNMLILNPTQSDSGIYICITTNETYCDMMSLNLTIVSVLESNIDLISYPQIVNERSTGEMVCPNINAFIASNVNADIIWSGHRRLRNKRLKQRTPGIITIEDVRKNDAGYYTCVLEYIYRGKTYNVTRIVKLEVRDKIIPSTMQLPDGIVTSIGSNLTIACRVSLRPPTTDADVFWISNGMYYEEDDGDGNGRISVANKIYMTDKRRVITSRLNINPVKEEDATTFTCMAFTIPSISKTVTVSIT

SEQ ID NO: 155 - protein, 75 aa, Vaccinia virus
MVIIPGVRCLSLLFLRRRCPLHIISAFTLLAINALILGHTISPVDLSFTICGYEIRSIFDSKTDTIVKFNDIMSQ

SEQ ID NO: 156 - protein, 340 aa, Vaccinia virus
MSRKFMQVYEYDREQYLDEFIEDRYNDSFITSPEYYSAEKYMCRYTTLNHNCVNVRRCALDSKLLHDIITNCKIYNNIELVRATKFVYYLDLIKCNWVSKVGDSVLYPVIFITHTSTRNLDKVSVKTYKGVKVKKLNRCADHAIVINPFVKFKLTLPNKTSHAKVLVTFCKLRTDITQIEAPLSGNVLVYTFPNINKRIPGYIHVNIEGCIDGMIYINSSKFACVLKLHRSMYRIPPFPIDICSCCSQYTNGDIEIPIHDLIKDVAIFKNKETVYYLKLNNKTIARFTYFNNIDTAITQEHEYVKIALGIVCKLMINNMHSIVGVNHSNTFVNCLLEDNV

SEQ ID NO: 157 - protein, 574 aa, Vaccinia virus
MSRRLIYVLNINRESTHKIQENEIYTYFSHCNIDHTSTELDFVVKNYDLNRRHPVTGYTALHCYLYNNYFTNDVLKILLNHGVDVTMKTSSGRMPVYILLTRCCNISHDVVIDMIDKDKNHLLHRDYSNLLLEYIKSRYMLLKEEDIDENIVSTLLDKGIDPNFKQDGYTALHYYYLCLAHVYKPGECRKPITIKKAKRIISLFIQHGANLNALDNCGNTPFHLYLSIEMCNNIHMTKMLLTFNPNFKICNNHGLTPILCYITSDYIQHDILVMLIHHYETNVGEMPIDERRIIVFEFIKTYSTRPADSITYLMNRFKNINIYTRYEGKTLLHVACEYNNTQVIDYLIRINGDINALTDNNKHATQLIIDNKENSPYTINCLLYILRYIVDKNVIRSLVDQLPSLPIFDIKSFEKFISYCILLDDTFYDRHVKNRNSKTYRYAFSKYMSFDKYDGIITKCHDETMLLKLSTVLDTTLYAVLRCHNSRKLRRYLTELKKYNNDKSFKIYSNIMNERYLNVYYKDMYVSKVYDKLFPVFTDKNCLLTLLPSEIIYEILYMLTINDLYNISYPPTKV

SEQ ID NO: 158 - protein, 353 aa, Vaccinia virus
MTMKMMVHIYFVSLSLLLLLFHSYAIDIENEITEFFNKMRDTLPAKDSKWLNPACMFGGTMNDIAALGEPFSAKCPPIEDSLLSHRYKDYVVKWERLEKNRRRQVSNKRVKHGDLWIANYTSKFSNRRYLCTVTTKNGDCVQGIVRSHIKKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 159 - protein, 56 aa, Vaccinia virus; identical to SEQ ID NO: 133

SEQ ID NO: 160 - protein, 271 aa, Vaccinia virus
LKNVECVDIDSTITFMKYDPNDDNKRTCSNWVPLTNNYMEYCLVIYLETPICGGKIKLYHPTGNIKSDKDIMFAKTLDFKSTKVLTGRKTIAVLDISVSYNRSMTTIHYNDDVDIDIHTDKNGKELCYCYITIDDHYLVDVETIGVIVNRSGKCLLVNNHLGIGIVKDKRISDSFGDVCMDTIFDFSEARELFSLTNDDNRNIAWDTDKLDDDTDIWTPVTENDYKFLSRLVLYAKSQSDTVFDYYVLTGDTEPPTVFIFKVTRFYFNMPK

SEQ ID NO: 161 - protein, 74 aa, Vaccinia virus
MYCRCSHGYTGIRCQHVVLVDYQRSEKPNTTTSYIPSPGIMLVLVGIIIITCCLLSVYRFTRRTKLPLQDMVVP

SEQ ID NO: 162 - protein, 244 aa, Vaccinia virus; identical to SEQ ID NO: 150

SEQ ID NO: 163 - protein, 222 aa, Vaccinia virus
MNHCLLAISAVYFKAKWLTPFEKEFTSDYPFYVSPTEMVDVSMMSMYGKAFNHASVKESFGNFSIIELPYVGDTSMMVILPDKIDGLESIEQNLTDTNFKKWCDFMDAMFIDVHIPKFKVTGSYNLVDTLVKSGLTEVFGSTGDYSNMCNLDVSVDAMIHKTYIDVNEEYTEAAAATCALVSDCASTVTNEFCADHPFIYVIRHVDGKILFVGRYCSPTTNC

SEQ ID NO: 164 - protein, 149 aa, Vaccinia virus
MTANFSTHVFSPQHCGCDRLTSIDDVKQCLTEYIYWSSYAYRNRQCAGQLYSTLLSFRDDAELVFIDIRELVKHMPWDDVKDCAEIIRCYIPDEQKTIREISAIIGLCAYAATYWGGEDHPTSNSLNALFVMLEMLNYVDYNIIFRRMN

SEQ ID NO: 165 - protein, 326 aa, Vaccinia virus
MSILPVIFLSIFFYSSFVQTFNASECIDKGQYFASFMELENEPVILPCPQINTLSSGYNILDILWEKRGADNDRIIPIDNGSNMLILNPTQSDSGIYICITTNETYCDMMSLNLTIVSVSESNIDLISYPQIVNERSTGEMVCPNINAFIASNVNADIIWSGHRRLRNKRLKQRTPGIITIEDVRKNDAGYYTCVLEYIYGGKTYNVTRIVKLEVRDKIIPSTMQLPDGIVTSIGSNLTIACRVSLRPPTTDTDVFWISNGMYYEEDDGDGDGRISVANKIYMTDKRRVITSRLNINPVKEEDATTFTCMAFTIPSISKTVTVSIT

SEQ ID NO: 166 - protein, 340 aa, Vaccinia virus
MSRKFMQVYEYDREQYLDEFIEDRYNDSFITSPEYYSAEKYMCRYTTLNHNCVNVRRCALDSKLLHDIITNCKIYNNIELVRATKFVYYLDLIKCNWVSKVGDSVLYPVIFITHTSTRNLDKVSVKTYKGVKVKKLNRCADHAIVINPFVKFKLTLPNKTSHAKVLVTFCKLRTDITQIEAPLSGNVLVYTFPDINKRIPGYIHVNIEGCIDGMIYINSSKFACVLKLHRSMYRIPPFPIDICSCCSQYTNDDIEIPIHDLIKDVAIFKNKETVYYLKLNNKTIARFTYFNNIDTAITQEHEYVKIALGIVCKLMINNMHSIVGVNHSNTFVNCLLEDNV

SEQ ID NO: 167 - protein, 574 aa, Vaccinia virus
MSRRLIYVLNINRESTHKIQENEIYTYFSHCNIDHTSTELDFVVKNYDLNRRQPVTGYTALHCYLYNNYFTNDVLKILLNHGVDVTMKTSSGRMPVYILLTRCCNISHDVVIDMIDKDKNHLSHRDYSNLLLEYIKSRYMLLKEEDIDENIVSTLLDKGIDPNFKQDGYTALHYYYLCLAHVYKPGECRKPITIKKAKRIISLFIQHGANLNALDNCGNTPFHLYLSIEMCNNIHMTKMLLTFNPNFKICNNHGLTPILCYITSDYIQHDILVMLIHHYETNVGEMPIDERRIIVFEFIKTYSTRPADSITYLMNRFKNINIYTRYEGKTLLHVACEYNNTHVIDYLIRINGDINALTDNNKHAIQLIIDNKENSPYTIDCLLYILRYIVDKNVIRSLVDQLPSLPIFDIKSFEKFISYCILLDDTFYNRHVRNRNSKTYRYAFSKYMSFDKYDGIITKCHDETMLLKLSTVLDTTLYAVLRCHNSKKLRRYLNELKKYNNDKSFKIYSNIMNERYLNVYYKDMYVSKVYDKLFPVFTDKNCLLTLLPSEIIYEILYMLTINDLYNISYPPTKV

SEQ ID NO: 168 - protein, 265 aa, Vaccinia virus
MTMKMMVHIYFVSLSLLLLLFHSYAIDIENEITEFFNKMRDTLPAKDSKWLNPACMFGGTMNDIATLGEPFSAKCPPIEDSLLSHRYKDYVVKWERLEKNRRRQVSNKRVKHGDLWIANYTSKFSNRRYLCTVTTKNGDCVQGIVRSHIRKPPSCIPKTYELGTHDKYGIDLYCGILYAKHYNNITWYKDNKEINIDDIKYSQTGKKLIIHNPELEDSGRYDCYVHYDDVRIKNDIVVSRCKILTVIPSQDHRFKLKRNCGYASN

SEQ ID NO: 169 - protein, 91 aa, Vaccinia virus
MRQIKINGTDMLTVMYMLNKPTKKRYVNNPIFTDWANKQYKFYNQIIYNANKLIEQSKKIDDMIEEVSIDDNRLSTLPLEIRHLIFSYAFL

SEQ ID NO: 170 - protein, 55 aa, Vaccinia virus
MSSKGGSGGMWSVFIHGHDGSNKGSKTYTSGGGGMWGGGSSSGVNGGVKSGTGKI

SEQ ID NO: 171 - protein, 71 aa, Vaccinia virus
MFDYLENEEVALDELKQMLRDRDPNDTRNQFKNNALHAYLFNEHCNNVEVVKLLLDSGTNPLRKNWRQLPH

SEQ ID NO: 172 - protein, 83 aa, Vaccinia virus
MLKLKDIAMALLEATGFSNINDFNIFSYMKSKNVDVDLIKVLVEHGFDLSVKCENHRSVIENYVMTMILFIENGCSVLYEDEY

SEQ ID NO: 173 - protein, 170 aa, Vaccinia virus
MKGIDNTAYSYIDDLTCCTRGIMADYLNSDYRYNKDVDLVKLFLENGKPHGIMCSIVPLWRNDKETIFLILKTMNSDVLQHILIEYMTFGDIPLVEYGTVVNKEAIHGYFRNINIDSYTMKYLLKKEGRCHQLSRLDTYVNPTMDVIISTLIHTKRVFVTCLMLAQFLVL

SEQ ID NO: 174 - protein, 173 aa, Vaccinia virus
MPSIISIGHLCKSNYGCYNFYTYTYKKGLCDMSYACPILSTINICLPYLKDINMIDKRGETLLHKAVRYNKQSLVSLLLESGSDVNIRSNNGYTCIAIAINESKNIELLKMLLCHKPTLDYVIDSLREISNIVDNDYAIKQCIKYAMIIDDCTSSKIPEFISQRYNDYIDLCN

SEQ ID NO: 175 - protein, 90 aa, Vaccinia virus
MKKIMVGGNTMFSLIFTDHGAKIIHRYANNPELREYYELKQNKIYVEAYDIISNAIVKHDRIHKTIESVDDNTYISNLPYTIKYKIFEQQ

SEQ ID NO: 176 - protein, 126 aa, Vaccinia virus
MRILFLIAFMYGCVHSYVNAVETKCSNLDIVTSSGEFHCSGCVEHMPNFSYMYWLAKDMRSDEDAKFIEHLGEGIKEDETVRTIDGRIVTLQKVLHVTDTNKFAHYRFTCVLTTIDGVSKKNIWLK

SEQ ID NO: 177 - protein, 62 aa, Vaccinia virus
MKLFTQNDRYFGLLDSCNHIFCITCINIWHKTRRETGASDNCPICRTRFRNITMSKFYKLVN

SEQ ID NO: 178 - protein, 66 aa, Vaccinia virus
MHYPKYYINITKINPHLANQFRAWKKRIAGRDYMTNLSKDTGIQQSKLYVTVKKIETYMVYIYTTI

SEQ ID NO: 179 - protein, 331 aa, Vaccinia virus
MDIYDDKGLQTIKLFNNEFDCIRNDIRELFKHVTDSDSIQLPMEDNSDIIENIRKILYRRLKNVECVDIDNTITFMKYDPNDDNKRTCSNWVPLTNNYMEYCLVIYLETPICGGKIKLYHPTGNIKSDKDIMFAKTLDFKSKKVLTGRKTIAVLDISVSYNRSITTIHYNDDVDIDIHTDKNGKELCYCYITIDDHYLVDVETIGVIVNRSGKCLLVNNHLGIGIVKDKRISDSFGDVCMDTIFDFSEARELFSLTNDDNRNIAWDTDKLDDDTDIWTPVTENDYKFLSRLVLYAKSQSDTVFDYYVLTGDTEPPTVFIFKVTRFYFNMPK

SEQ ID NO: 180 - protein, 141 aa, Vaccinia virus
MLINYLMLLFAAMIIRSFADSGNAIETTLPEITNATTDIPAIRLCGPEGDGYCLHGDCIHARDIDGMYCRCSHGYTGIRCQHVVLVDYQRSEKPNTTTSYIPSPGIMLVLVGIIIITCCLLSVYRFTRRTNKLPLQDMVVP

SEQ ID NO: 181 - protein, 357 aa, Vaccinia virus
MDIFKELIVKHPDENVLISPVSILSTLSILNHGAAGSTAEQLSKYIENMNENTPDDKKDDNNDMDVDIPYCATLATANKIYGSDSIEFHASFLQKIKDDFQTVNFNNANQTKELINEWVKTMTNGKINSLLTSPLSINTRMTVVSAVHFKAMWKYPFSKHLTYTDKFYISKNIVTSVDMMVGTENNLQYVHINELFGGFSIIDIPYEGNSSMVIILPDDIEGIYNIEKNITDEKFKKWCGMLSTKSIDLYMPKFKVEMTEPYNLVPILENLGLTNIFGYYADFSKMCNETITVEKFLHTTFIDVNEEYTEASAVTGVFMTNFAMVYRTKVYINHPFMYMIKDTTGRILFIGKYCYPQ

SEQ ID NO: 182 - protein, 190 aa, Vaccinia virus
MMIYGLIACLIFVTSSIASPLYIPVIPPITEDKSFNSVEVLVSLFRDDQKDYTVTSQFNNYTIDTKDWTIGVLSTPDGLDIPLTNITYWSRFTIGRALFKSESEDIFQKKMSILGVSIECKKSSTLLTFLTVRKMTRVFNKFPDMAYYRGDCLKAVYVTMTYKNTKTGETDYTYLSNGGLPAYYRNGVDG

SEQ ID NO: 183 - protein, 51 aa, Vaccinia virus
MNLQKLSLAIYLTATCSWCYETCIRKTALYHDIQLEHVEDNKDSVASLPYK

SEQ ID NO: 184 - protein, 87 aa, Vaccinia virus
MSLESFIITTFNNNSSTNIDNMCHLYVKVCPSSLLFRLFVECCDINKLVEGTTPLHCYLMNEGFESSVLKNLLKEYVMTSITQIFNS

SEQ ID NO: 185 - protein, 181 aa, Vaccinia virus
MISLSFLIHNPLKKWKLKPSISINGYRSTFTMAFPCAQFRPCHCHATKDSLNTVADVRHCLTEYILWVSHRWTHRETAGPLYRLLISFRTDATELFGGELKDSLPWDNIDNCVEIIKCFIRNDSMKTAEELRAIIGLCTQSAIVSGRVFNDKYIDILLMLRKILNENDYLTLLDHIRTAKY

SEQ ID NO: 186 - protein, 382 aa, Vaccinia virus
MIAFIIFREIGIISTRIAMDCTCILCRLLDEDVTYKKIKLEIETCHNLSKHIDRRGNNALHCYVFNKCDTDIKIVRLLLSRGVERLCRNNEGLTPLGVYSKHRYVKSQIVHLLISSYSNSSNELKSNINDFDLSSDNIDLRLLKYLIVDKRIRPSKNTNYAINSLGLVDIYVTTPNPRPEVLLWLLKSECYSTGYVFRTCMYNSDMCKNSLHYYISSHRESQSLSKDVIKCLINNNVSIHGRDEGGSLPIQYYWSFSTIDIEIVKLLLIKDVDTCRVYDVSPILEAYYLNKRFRVTPYNVDMEIVNLLIERRHTLVDVMRSITSYDSREYNHYIIDNILKRFRQQDESIVQAMLINYLHYGDMVVRCMLDNGQQLSSARLLC

SEQ ID NO: 187 - protein, 198 aa, Vaccinia virus
MYGLILSRFNNCGYHCYETILIDVFDILSKYMDNIDMIDNENKTLLYYAVDVNNIQFAKRLLEYGASVTTSRSIINTAIQKSSYRRENKTKLVDLLLSYHPTLETMIDAFNRDIRYLYPEPLFACIRYALILDDDFPSKVKYDISGRHKELKRYRVDINRMKNAYISGVSMFDILFKRSKRHRLRYAKNPTSNGTKKN

SEQ ID NO: 188 - protein, 147 aa, Vaccinia virus
MSRINITKKIYCSVFLFLFLSYISNYEKVNDEMYEMGEMDEIVSIVRDSMWYIPNVFMDDGKNEGHVSVNNVCHMYFTFFDVDTSSHLFKLVIKHCDLNKRGNSPLHCYTMNTRFNPSVLKILLHHGMRNFDSKDDHYQSITRSLIY

SEQ ID NO: 189 - protein, 128 aa, Vaccinia virus
MEQTLTRLHTYLQQYTKHSPRVVYALLSRGYVIILIVHPSWNDCATGHILIMLLNWHEQKEEGQHLLYLFIKHNQGYTLNILRYLLDRFDIQKDEYYNTAFQNCNNNVASYIGYDINLPTKDGIRLGV

SEQ ID NO: 190 - protein, 111 aa, Vaccinia virus
MLPHTSDTTSTFRLKTVFDLVFENRNIIYKADVVNDIIHHRLKVPMIKSLFYKMSEFSPYDDYYVKKILAYCLLRDESFAELHSKFCLNEDYKSVFMKNISFDKIDSIIVT

SEQ ID NO: 191 - protein, 94 aa, Vaccinia virus
MHHPMESVKTTNTNAIICVREHTLPDYANTQCTPCGSGTFTSRNNHLPACLSCNGRRDRVTLLTIESVNALPDIIVFSKDHPDARHVFPKQNVE

SEQ ID NO: 192 - protein, 241 aa, Vaccinia virus
MHVPASLQQSSSSCTEEENKHHMGIDVIIKVTKQDQTPTNDKICQSVTEITESESDPDPEVESEDDSTSVEDVDLPTTYYSIIGGGLRMNFGFTKCPQIKSISESADGNTVNARLSSVSPGQGKDSPAITHEEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV

SEQ ID NO: 193 - protein, 62 aa, Vaccinia virus; identical to SEQ ID NO: 177

SEQ ID NO: 194 - protein, 66 aa, Vaccinia virus; identical to SEQ ID NO: 178

SEQ ID NO: 195 - protein, 331 aa, Vaccinia virus; identical to SEQ ID NO: 179

SEQ ID NO: 196 - protein, 141 aa, Vaccinia virus; identical to SEQ ID NO: 180

SEQ ID NO: 197 - protein, 94 aa, Vaccinia virus; identical to SEQ ID NO: 191

SEQ ID NO: 198 - protein, 241 aa, Vaccinia virus; identical to SEQ ID NO: 192

SEQ ID NO: 199 - skipped

SEQ ID NO: 200 - protein, 149 aa, Vaccinia virus; identical to SEQ ID NO: 109

SEQ ID NO: 201 - protein, 326 aa, Vaccinia virus
MSILPVIFLPIFFYSSFVQTFNAPECIDKGQYFASFMELENEPVILPCPQINTLSSGYNILDILWEKRGADNDRIIPIDNGSNMLILNPTQSDSGIYICITTNETYCDMMSLNLTIVSVSESNIDLISYPQIVNERSTGEMVCPNINAFIASNVNADIIWSGHRRLRNKRLKQRTPGIITIEDVRKNDAGYYTCVLEYIYRGKTYNVTRIVKLEVRDKIIPSTMQLPDGIVTSIGSNLTIACRVSLRPPTTDADVFWISNGMYYEEDDGDGNGRISVANKIYMTDKRRVITSRLNINPVKEEDATTFTCMAFTIPSISKTVTVSIT

SEQ ID NO: 202 - protein, 340 aa, Vaccinia virus
MSRKFMQVYEYDREQYLDEFIEDRYNDSFITSPEYYSAEKYMCRYTTLNHNCINVRRCALDSKLLHDIITNCKIYNNIELVRATKFVYYLDLIKCNWVSKVGDSVLYPVIFITHTSTRNLDKVSVKTYKGVKVKKLNRCADHAIVINPFVKFKLTLPNKTSHAKVLVTFCKLRTDITQIEAPLSGNVLVYTFPDINKRIPGYIHVNIEGCIDGMIYINSSKFACVLKLHRSMYRIPPFPIDICSCCSQYTNDDIEIPIHDLIKDVAIFKNKETVYYLKLNNKTIARFTYFNNIDTAITQEHEYVKIALGIVCKLMINNMHSIVGVNHSNTFVNCLLEDNV

SEQ ID NO: 203 - protein, 167 aa, Vaccinia virus
MTKVIIILGFLIINTNSLSMKCEQGVSYYNSQELKCCKLCKPGTYSDHRCDKYSDTICGHCPSDTFTSIYNRSPWCHSCRGPCGTNRVEVTPCTPTTNRICHCDSNSYCLLKASDGNCVTCAPKTKCGRGYGKKGEDEMGNTICKKCRKGTYSDIVSDSDQCKPMTR

SEQ ID NO: 204 - protein, 237 aa, Vaccinia virus
MAMPSLSACSSIEDDFNYGSSVASASVHIRMAFLRKVYGILCLQFLLTTATTAVFLYFDCMRTFIQGSPVLILASMFGSIGLIFALTLHRHKHPLNLYLLCGFTLSESLTLASVVTFYDVHVVMQAFMLTTAAFLALTTYTLQSKRDFSKLGAGLFAALWILILSGLLGIFVQNETVKLVLSAFGALVFCGFIIYDTHSLIHKLSPEEYVLASINLYLDIINLFLHLLQLLEVSNKK

SEQ ID NO: 205 - protein, 147 aa, Vaccinia virus
MASPCAKFRPCHCHATKDSLNTVADVRHCLTEYILWVSHRWTHRESAGSLYRLLISFRTDATELFGGELKDSLPWDNCVEIIKCFIRNDSMKTAEELRAIIGLCTQSAIVSGRVFNDKYIDILLMLRKILNENDYLTLLDHIRTAKY

SEQ ID NO: 206 - protein, 128 aa, Vaccinia virus; identical to SEQ ID NO: 189

SEQ ID NO: 207 - protein, 64 aa, Vaccinia virus; identical to SEQ ID NO: 145

SEQ ID NO: 208 - protein, 258 aa, Vaccinia virus
MKQYIVLACMCLAAAAMPASLQQSSSSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITHEEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV

SEQ ID NO: 209 - protein, 271 aa, Vaccinia virus
MRYIIILAVLFINSIHAKITSYKFESVNFDSKIEWTGDGLYNISLKNYGIKTWQTMYTNVPEGTYDISAFPKNDFVSFWVKFEQGDYKVEEYCTGLCVEVKIGPPTVTLTEYDDHINLYIEHPYATRGSKKIPIYKRGDMCDIYLLYTANFTFGDSEEPVTYDIDDYDCTSTGCSIDFATTEKVCVTAQGATEGFLEKITPWSSEVCLTPKKNVYTCAIRKEDVPNFKDKMARVIKRKFNKQSQSYLTKFLGSTSNDVTTFLSMLNLTKYS

SEQ ID NO: 210 - protein, 9 aa, unidentified
  source 1..9: note = Description of Unknown: immunogenic epitope
FLGYLILGV

SEQ ID NO: 211 - protein, 15 aa, unidentified
  source 1..15: note = Description of Unknown: immunogenic epitope
SVSESDTIRSISIAS

SEQ ID NO: 212 - protein, 15 aa, unidentified
  source 1..15: note = Description of Unknown: immunogenic epitope
LLANGRMPTVLQCVN

SEQ ID NO: 213 - protein, 15 aa, unidentified
  source 1..15: note = Description of Unknown: immunogenic epitope
RMPTVLQCVNVSVVS

SEQ ID NO: 214 - protein, 12 aa, unidentified
  source 1..12: note = Description of Unknown: immunogenic epitope
CTACRWKKACQR

SEQ ID NO: 215 - protein, 9 aa, unidentified
  source 1..9: note = Description of Unknown: immunogenic epitope
VLDGLDVLL

SEQ ID NO: 216 - protein, 10 aa, unidentified
  source 1..10: note = Description of Unknown: immunogenic epitope
SLYSFPEPEA

SEQ ID NO: 217 - protein, 10 aa, unidentified
  source 1..10: note = Description of Unknown: immunogenic epitope
ALYVDSLFFL

SEQ ID NO: 218 - protein, 9 aa, unidentified
  source 1..9: note = Description of Unknown: immunogenic epitope
SLLQHLIGL

SEQ ID NO: 219 - protein, 9 aa, unidentified
  source 1..9: note = Description of Unknown: immunogenic epitope
LYVDSLFFL

SEQ ID NO: 220 - protein, 11 aa, unidentified
  source 1..11: note = Description of Unknown: immunogenic epitope
TSEKRPFMCAY

SEQ ID NO: 221 - protein, 9 aa, unidentified
  source 1..9: note = Description of Unknown: immunogenic epitope
CMTWNQMNL

SEQ ID NO: 222 - protein, 11 aa, unidentified
  source 1..11: note = Description of Unknown: immunogenic epitope
LSHLQMHSRKH

SEQ ID NO: 223 - protein, 16 aa, unidentified
  source 1..16: note = Description of Unknown: immunogenic epitope
KRYFKLSHLQMHSRKH

SEQ ID NO: 224 - protein, 8 aa, unidentified
  source 1..8: note = Description of Unknown: immunogenic epitope
CYMEAVAL

SEQ ID NO: 225 - protein, 9 aa, unidentified
  source 1..9: note = Description of Unknown: immunogenic epitope
SLLFLLFSL

SEQ ID NO: 226 - protein, 9 aa, unidentified
  source 1..9: note = Description of Unknown: immunogenic epitope
VLPLTVAEV

SEQ ID NO: 227 - protein, 9 aa, unidentified
  source 1..9: note = Description of Unknown: immunogenic epitope
ALQGGGPPY

SEQ ID NO: 228 - protein, 9 aa, unidentified
  source 1..9: note = Description of Unknown: immunogenic epitope
LYPKARLAF

SEQ ID NO: 229 - protein, 9 aa, unidentified
  source 1..9: note = Description of Unknown: immunogenic epitope
AFLPWHRLF

SEQ ID NO: 230 - protein, 9 aa, unidentified
  source 1..9: note = Description of Unknown: immunogenic epitope
SLLMWITQC

SEQ ID NO: 231 - protein, 9 aa, unidentified
  source 1..9: note = Description of Unknown: immunogenic epitope
LAMPFATPM

SEQ ID NO: 232 - protein, 9 aa, unidentified
  source 1..9: note = Description of Unknown: immunogenic epitope
ARGPESRLL

SEQ ID NO: 233 - protein, 11 aa, unidentified
  source 1..11: note = Description of Unknown: immunogenic epitope
MLMAQEALAFL

SEQ ID NO: 234 - protein, 11 aa, unidentified
  source 1..11: note = Description of Unknown: immunogenic epitope
YLAMPFATPME

SEQ ID NO: 235 - protein, 10 aa, unidentified
  source 1..10: note = Description of Unknown: immunogenic epitope
ASGPGGGAPR

SEQ ID NO: 236 - protein, 10 aa, unidentified
  source 1..10: note = Description of Unknown: immunogenic epitope
LAAQERRVPR

SEQ ID NO: 237 - protein, 10 aa, unidentified
  source 1..10: note = Description of Unknown: immunogenic epitope
TVSGNILTIR

SEQ ID NO: 238 - protein, 13 aa, unidentified
  source 1..13: note = Description of Unknown: immunogenic epitope
APRGPHGGAASGL

SEQ ID NO: 239 - protein, 11 aa, unidentified
  source 1..11: note = Description of Unknown: immunogenic epitope
MPFATPMEAEL

SEQ ID NO: 240 - protein, 12 aa, unidentified
  source 1..12: note = Description of Unknown: immunogenic epitope
KEFTVSGNILTI

SEQ ID NO: 241 - protein, 9 aa, unidentified
  source 1..9: note = Description of Unknown: immunogenic epitope
MPFATPMEA

SEQ ID NO: 242 - protein, 9 aa, unidentified
  source 1..9: note = Description of Unknown: immunogenic epitope
FATPMEAEL

SEQ ID NO: 243 - protein, 11 aa, unidentified
  source 1..11: note = Description of Unknown: immunogenic epitope
FATPMEAELAR

SEQ ID NO: 244 - protein, 14 aa, unidentified
  source 1..14: note = Description of Unknown: immunogenic epitope
SLLMWITQCFLPVF

SEQ ID NO: 245 - protein, 25 aa, unidentified
  source 1..25: note = Description of Unknown: immunogenic epitope
LLEFYLAMPFATPMEAELARRSLAQ

SEQ ID NO: 246 - protein, 12 aa, unidentified
  source 1..12: note = Description of Unknown: immunogenic epitope
EFYLAMPFATPM

SEQ ID NO: 247 - protein, 25 aa, unidentified
  source 1..25: note = Description of Unknown: immunogenic epitope
PGVLLKEFTVSGNILTIRLTAADHR

SEQ ID NO: 248 - protein, 12 aa, unidentified
  source 1..12: note = Description of Unknown: immunogenic epitope
RLLEFYLAMPFA

SEQ ID NO: 249 - protein, 20 aa, unidentified
  source 1..20: note = Description of Unknown: immunogenic epitope
QGAMLAAQERRVPRAAEVPR

SEQ ID NO: 250 - protein, 13 aa, unidentified
  source 1..13: note = Description of Unknown: immunogenic epitope
PFATPMEAELARR

SEQ ID NO: 251 - protein, 20 aa, unidentified
  source 1..20: note = Description of Unknown: immunogenic epitope
PGVLLKEFTVSGNILTIRLT

SEQ ID NO: 252 - protein, 10 aa, unidentified
  source 1..10: note = Description of Unknown: immunogenic epitope
VLLKEFTVSG

SEQ ID NO: 253 - protein, 18 aa, unidentified
  source 1..18: note = Description of Unknown: immunogenic epitope
AADHRQLQLSISSCLQQL

SEQ ID NO: 254 - protein, 15 aa, unidentified
  source 1..15: note = Description of Unknown: immunogenic epitope
LKEFTVSGNILTIRL

SEQ ID NO: 255 - protein, 11 aa, unidentified
  source 1..11: note = Description of Unknown: immunogenic epitope
KEFTVSGNILT

SEQ ID NO: 256 - protein, 14 aa, unidentified
  source 1..14: note = Description of Unknown: immunogenic epitope
LLEFYLAMPFATPM

SEQ ID NO: 257 - protein, 14 aa, unidentified
  source 1..14: note = Description of Unknown: immunogenic epitope
AGATGGRGPRGAGA

SEQ ID NO: 258 - protein, 13 aa, unidentified
  source 1..13: note = Description of Unknown: immunogenic epitope
TYYRPGVNLSLSC

SEQ ID NO: 259 - protein, 13 aa, unidentified
  source 1..13: note = Description of Unknown: immunogenic epitope
EIIYPNASLLIQN

SEQ ID NO: 260 - protein, 15 aa, unidentified
  source 1..15: note = Description of Unknown: immunogenic epitope
YACFVSNLATGRNNS

SEQ ID NO: 261 - protein, 13 aa, unidentified
  source 1..13: note = Description of Unknown: immunogenic epitope
LWWVNNQSLPVSP

SEQ ID NO: 262 - protein, 13 aa, unidentified
  source 1..13: note = Description of Unknown: immunogenic epitope
NSIVKSITVSASG

SEQ ID NO: 263 - protein, 9 aa, unidentified
  source 1..9: note = Description of Unknown: immunogenic epitope
KTWGQYWQV

SEQ ID NO: 264 - protein, 10 aa, unidentified
  source 1..10: note = Description of Unknown: immunogenic epitope
AMLGTHTMEV

SEQ ID NO: 265 - protein, 9 aa, unidentified
  source 1..9: note = Description of Unknown: immunogenic epitope
ITDQVPFSV

SEQ ID NO: 266 - protein, 9 aa, unidentified
  source 1..9: note = Description of Unknown: immunogenic epitope
YLEPGPVTA

SEQ ID NO: 267 - protein, 10 aa, unidentified
  source 1..10: note = Description of Unknown: immunogenic epitope
LLDGTATLRL

SEQ ID NO: 268 - protein, 10 aa, unidentified
  source 1..10: note = Description of Unknown: immunogenic epitope
VLYRYGSFSV

SEQ ID NO: 269 - protein, 10 aa, unidentified
  source 1..10: note = Description of Unknown: immunogenic epitope
SLADTNSLAV

SEQ ID NO: 270 - protein, 9 aa, unidentified
  source 1..9: note = Description of Unknown: immunogenic epitope
RLMKQDFSV

SEQ ID NO: 271 - protein, 9 aa, unidentified
  source 1..9: note = Description of Unknown: immunogenic epitope
RLPRIFCSC

SEQ ID NO: 272 - protein, 9 aa, unidentified
  source 1..9: note = Description of Unknown: immunogenic epitope
LIYRRRLMK

SEQ ID NO: 273 - protein, 9 aa, unidentified
  source 1..9: note = Description of Unknown: immunogenic epitope
ALLAVGATK

SEQ ID NO: 274 - protein, 10 aa, unidentified
  source 1..10: note = Description of Unknown: immunogenic epitope
IALNFPGSQK

SEQ ID NO: 275 - protein, 9 aa, unidentified
  source 1..9: note = Description of Unknown: immunogenic epitope
RSYVPLAHR

SEQ ID NO: 276 - protein, 9 aa, unidentified
  source 1..9: note = Description of Unknown: immunogenic epitope
VVPCEPPEV

SEQ ID NO: 277 - protein, 9 aa, unidentified
  source 1..9: note = Description of Unknown: immunogenic epitope
HLYQGCQVV

SEQ ID NO: 278 - protein, 10 aa, unidentified
  source 1..10: note = Description of Unknown: immunogenic epitope
YLVPQQGFFC

SEQ ID NO: 279 - protein, 9 aa, unidentified
  source 1..9: note = Description of Unknown: immunogenic epitope
PLQPEQLQV

SEQ ID NO: 280 - protein, 9 aa, unidentified
  source 1..9: note = Description of Unknown: immunogenic epitope
TLEEITGYL

SEQ ID NO: 281 - protein, 9 aa, unidentified
  source 1..9: note = Description of Unknown: immunogenic epitope
ALIHHNTHL

SEQ ID NO: 282 - protein, 9 aa, unidentified
  source 1..9: note = Description of Unknown: immunogenic epitope
PLTSIISAV

SEQ ID NO: 283 - protein, 9 aa, unidentified
  source 1..9: note = Description of Unknown: immunogenic epitope
VLRENTSPK

SEQ ID NO: 284 - protein, 9 aa, unidentified
  source 1..9: note = Description of Unknown: immunogenic epitope
TYLPTNASL

SEQ ID NO: 285 - protein, 9 aa, unidentified
  source 1..9: note = Description of Unknown: immunogenic epitope
RYQLDPKFI

SEQ ID NO: 286 - protein, 9 aa, unidentified
  source 1..9: note = Description of Unknown: immunogenic epitope
ILFTINFTI

SEQ ID NO: 287 - protein, 9 aa, unidentified
  source 1..9: note = Description of Unknown: immunogenic epitope
VLFTINFTI

SEQ ID NO: 288 - protein, 9 aa, unidentified
  source 1..9: note = Description of Unknown: immunogenic epitope
TLNFTITNL

SEQ ID NO: 289 - protein, 9 aa, unidentified
  source 1..9: note = Description of Unknown: immunogenic epitope
VLQGLLKPL

SEQ ID NO: 290 - protein, 9 aa, unidentified
  source 1..9: note = Description of Unknown: immunogenic epitope
VLQGLLRPV

SEQ ID NO: 291 - protein, 9 aa, unidentified
  source 1..9: note = Description of Unknown: immunogenic epitope
RLDPKSPGV

SEQ ID NO: 292 - protein, 9 aa, unidentified
  source 1..9: note = Description of Unknown: immunogenic epitope
QLYWELSKL

SEQ ID NO: 293 - protein, 9 aa, unidentified
  source 1..9: note = Description of Unknown: immunogenic epitope
KLTRGIVEL

SEQ ID NO: 294 - protein, 9 aa, unidentified
  source 1..9: note = Description of Unknown: immunogenic epitope
QLTNGITEL

SEQ ID NO: 295 - protein, 9 aa, unidentified
  source 1..9: note = Description of Unknown: immunogenic epitope
QLTHNITEL

SEQ ID NO: 296 - protein, 9 aa, unidentified
  source 1..9: note = Description of Unknown: immunogenic epitope
TLDRNSLYV

SEQ ID NO: 297 - protein, 9 aa, unidentified
  source 1..9: note = Description of Unknown: immunogenic epitope
FLLSLALML

SEQ ID NO: 298 - protein, 9 aa, unidentified
  source 1..9: note = Description of Unknown: immunogenic epitope
NLGPWIQQV

SEQ ID NO: 299 - protein, 9 aa, unidentified
  source 1..9: note = Description of Unknown: immunogenic epitope
ILDSSEEDK

SEQ ID NO: 300 - protein, 10 aa, unidentified
  source 1..10: note = Description of Unknown: immunogenic epitope
YLPKSWTIQV

SEQ ID NO: 301 - protein, 12 aa, unidentified
  source 1..12: note = Description of Unknown: immunogenic epitope
WIHEQMERDLKT

SEQ ID NO: 302 - protein, 9 aa, unidentified
  source 1..9: note = Description of Unknown: immunogenic epitope
LLANAYIYV

SEQ ID NO: 303 - protein, 9 aa, unidentified
  source 1..9: note = Description of Unknown: immunogenic epitope
YLLCKAFGA

SEQ ID NO: 304 - protein, 9 aa, unidentified
  source 1..9: note = Description of Unknown: immunogenic epitope
KLSPYVHYT

SEQ ID NO: 305 - protein, 20 aa, unidentified
  source 1..20: note = Description of Unknown: immunogenic epitope
PDTRPAPGSTAPPAHGVTSA

SEQ ID NO: 306 - protein, 9 aa, unidentified
  source 1..9: note = Description of Unknown: immunogenic epitope
STAPPVHNV

SEQ ID NO: 307 - protein, 9 aa, unidentified
  source 1..9: note = Description of Unknown: immunogenic epitope
LLLLTVLTV

SEQ ID NO: 308 - protein, 12 aa, unidentified
  source 1..12: note = Description of Unknown: immunogenic epitope
PGSTAPPAHGVT

SEQ ID NO: 309 - protein, 9 aa, unidentified
  source 1..9: note = Description of Unknown: immunogenic epitope
MLAVISCAV

SEQ ID NO: 310 - protein, 9 aa, unidentified
  source 1..9: note = Description of Unknown: immunogenic epitope
RQKRILVNL

SEQ ID NO: 311 - protein, 9 aa, unidentified
  source 1..9: note = Description of Unknown: immunogenic epitope
NYNNFYRFL

SEQ ID NO: 312 - protein, 10 aa, unidentified
  source 1..10: note = Description of Unknown: immunogenic epitope
EYSKECLKEF

SEQ ID NO: 313 - protein, 9 aa, unidentified
  source 1..9: note = Description of Unknown: immunogenic epitope
EYLSLSDKI

SEQ ID NO: 314 - protein, 9 aa, unidentified
  source 1..9: note = Description of Unknown: immunogenic epitope
ELVRRILSR

SEQ ID NO: 315 - protein, 9 aa, unidentified
  source 1..9: note = Description of Unknown: immunogenic epitope
APRGVRMAV

SEQ ID NO: 316 - protein, 22 aa, unidentified
  source 1..22: note = Description of Unknown: immunogenic epitope
CLSRRPWKRSWSAGSCPGMPHL

SEQ ID NO: 317 - protein, 13 aa, unidentified
  source 1..13: note = Description of Unknown: immunogenic epitope
ILSRDAAPLPRPG

SEQ ID NO: 318 - protein, 9 aa, unidentified
  source 1..9: note = Description of Unknown: immunogenic epitope
EVDPASNTY

SEQ ID NO: 319 - protein, 10 aa, unidentified
  source 1..10: note = Description of Unknown: immunogenic epitope
GVYDGREHTV

SEQ ID NO: 320 - protein, 9 aa, unidentified
  source 1..9: note = Description of Unknown: immunogenic epitope
NYKRCFPVI

SEQ ID NO: 321 - protein, 8 aa, unidentified
  source 1..8: note = Description of Unknown: immunogenic epitope
SESLKMIF

SEQ ID NO: 322 - protein, 20 aa, unidentified
  source 1..20: note = Description of Unknown: immunogenic epitope
INKTSGPKRGKHAWTHRLRE

SEQ ID NO: 323 - protein, 20 aa, unidentified
  source 1..20: note = Description of Unknown: immunogenic epitope
YFSKKEWEKMKSSEKIVYVY

SEQ ID NO: 324 - protein, 20 aa, unidentified
  source 1..20: note = Description of Unknown: immunogenic epitope
MKLNYEVMTKLGFKVTLPPF

SEQ ID NO: 325 - protein, 20 aa, unidentified
  source 1..20: note = Description of Unknown: immunogenic epitope
KHAWTHRLRERKQLVVYEEI

SEQ ID NO: 326 - protein, 20 aa, unidentified
  source 1..20: note = Description of Unknown: immunogenic epitope
LGFKVTLPPFMRSKRAADFH

SEQ ID NO: 327 - protein, 20 aa, unidentified
  source 1..20: note = Description of Unknown: immunogenic epitope
KSSEKIVYVYMKLNYEVMTK

SEQ ID NO: 328 - protein, 9 aa, unidentified
  source 1..9: note = Description of Unknown: immunogenic epitope
SLGWLFLLL

SEQ ID NO: 329 - protein, 9 aa, unidentified
  source 1..9: note = Description of Unknown: immunogenic epitope
LSRLSNRLL

SEQ ID NO: 330 - protein, 9 aa, unidentified
  source 1..9: note = Description of Unknown: immunogenic epitope
TMNGSKSPV

SEQ ID NO: 331 - protein, 9 aa, unidentified
  source 1..9: note = Description of Unknown: immunogenic epitope
PLLENVISK

SEQ ID NO: 332 - protein, 9 aa, unidentified
  source 1..9: note = Description of Unknown: immunogenic epitope
SLSKILDTV

SEQ ID NO: 333 - protein, 9 aa, unidentified
  source 1..9: note = Description of Unknown: immunogenic epitope
AARAVFLAL

SEQ ID NO: 334 - protein, 9 aa, unidentified
  source 1..9: note = Description of Unknown: immunogenic epitope
EADPTGHSY

SEQ ID NO: 335 - protein, 9 aa, unidentified
  source 1..9: note = Description of Unknown: immunogenic epitope
KVLEYVIKV

SEQ ID NO: 336 - protein, 9 aa, unidentified
  source 1..9: note = Description of Unknown: immunogenic epitope
SLFRAVITK

SEQ ID NO: 337 - protein, 10 aa, unidentified
  source 1..10: note = Description of Unknown: immunogenic epitope
EVYDGREHSA

SEQ ID NO: 338 - protein, 9 aa, unidentified
  source 1..9: note = Description of Unknown: immunogenic epitope
RVRFFFPSL

SEQ ID NO: 339 - protein, 10 aa, unidentified
  source 1..10: note = Description of Unknown: immunogenic epitope
REPVTKAEML

SEQ ID NO: 340 - protein, 10 aa, unidentified
  source 1..10: note = Description of Unknown: immunogenic epitope
KEADPTGHSY

SEQ ID NO: 341 - protein, 9 aa, unidentified
  source 1..9: note = Description of Unknown: immunogenic epitope
DPARYEFLW

SEQ ID NO: 342 - protein, 11 aa, unidentified
  source 1..11: note = Description of Unknown: immunogenic epitope
ITKKVADLVGF

SEQ ID NO: 343 - protein, 9 aa, unidentified
  source 1..9: note = Description of Unknown: immunogenic epitope
SAFPTTINF

SEQ ID NO: 344 - protein, 9 aa, unidentified
  source 1..9: note = Description of Unknown: immunogenic epitope
SAYGEPRKL

SEQ ID NO: 345 - protein, 15 aa, unidentified
  source 1..15: note = Description of Unknown: immunogenic epitope
TSCILESLFRAVITK

SEQ ID NO: 346 - protein, 15 aa, unidentified
  source 1..15: note = Description of Unknown: immunogenic epitope
PRALAETSYVKVLEY

SEQ ID NO: 347 - protein, 16 aa, unidentified
  source 1..16: note = Description of Unknown: immunogenic epitope
FLLLKYRAREPVTKAE

SEQ ID NO: 348 - protein, 12 aa, unidentified
  source 1..12: note = Description of Unknown: immunogenic epitope
EYVIKVSARVRF

SEQ ID NO: 349 - protein, 10 aa, unidentified
  source 1..10: note = Description of Unknown: immunogenic epitope
YLQLVFGIEV

SEQ ID NO: 350 - protein, 9 aa, unidentified
  source 1..9: note = Description of Unknown: immunogenic epitope
EYLQLVFGI

SEQ ID NO: 351 - protein, 9 aa, unidentified
  source 1..9: note = Description of Unknown: immunogenic epitope
EGDCAPEEK

SEQ ID NO: 352 - protein, 14 aa, unidentified
  source 1..14: note = Description of Unknown: immunogenic epitope
LLKYRAREPVTKAE

SEQ ID NO: 353 - protein, 9 aa, unidentified
  source 1..9: note = Description of Unknown: immunogenic epitope
KASEKIFYV

SEQ ID NO: 354 - protein, 16 aa, unidentified
  source 1..16: note = Description of Unknown: immunogenic epitope
EKIQKAFDDIAKYFSK

SEQ ID NO: 355 - protein, 11 aa, unidentified
  source 1..11: note = Description of Unknown: immunogenic epitope
FGRLQGISPKI

SEQ ID NO: 356 - protein, 18 aa, unidentified
  source 1..18: note = Description of Unknown: immunogenic epitope
WEKMKASEKIFYVYMKRK

SEQ ID NO: 357 - protein, 15 aa, unidentified
  source 1..15: note = Description of Unknown: immunogenic epitope
KIFYVYMKRKYEAMT

SEQ ID NO: 358 - protein, 14 aa, unidentified
  source 1..14: note = Description of Unknown: immunogenic epitope
KIFYVYMKRKYEAM

SEQ ID NO: 359 - protein, 15 aa, unidentified
  source 1..15: note = Description of Unknown: immunogenic epitope
CEFHACWPAFTVLGE

SEQ ID NO: 360 - protein, 9 aa, unidentified
  source 1..9: note = Description of Unknown: immunogenic epitope
KFLDALISL

SEQ ID NO: 361 - protein, 10 aa, unidentified
  source 1..10: note = Description of Unknown: immunogenic epitope
ALGGHPLLGV

SEQ ID NO: 362 - protein, 9 aa, unidentified
  source 1..9: note = Description of Unknown: immunogenic epitope
LPRWPPPQL

SEQ ID NO: 363 - protein, 9 aa, unidentified
  source 1..9: note = Description of Unknown: immunogenic epitope
ILAKFLHWL

SEQ ID NO: 364 - protein, 9 aa, unidentified
  source 1..9: note = Description of Unknown: immunogenic epitope
RLVDDFLLV

SEQ ID NO: 365 - protein, 15 aa, unidentified
  source 1..15: note = Description of Unknown: immunogenic epitope
RPGLLGASVLGLDDI

SEQ ID NO: 366 - protein, 15 aa, unidentified
  source 1..15: note = Description of Unknown: immunogenic epitope
LTDLQPYMRQFVAHL

SEQ ID NO: 367 - protein, 9 aa, unidentified
  source 1..9: note = Description of Unknown: immunogenic epitope
VVVGAVGVG

SEQ ID NO: 368 - protein, 9 aa, unidentified
  source 1..9: note = Description of Unknown: immunogenic epitope
HLSTAFARV

SEQ ID NO: 369 - protein, 9 aa, unidentified
  source 1..9: note = Description of Unknown: immunogenic epitope
KIFGSLAFL

SEQ ID NO: 370 - protein, 9 aa, unidentified
  source 1..9: note = Description of Unknown: immunogenic epitope
IISAVVGIL

SEQ ID NO: 371 - protein, 9 aa, unidentified
  source 1..9: note = Description of Unknown: immunogenic epitope
ALCRWGLLL

SEQ ID NO: 372 - protein, 9 aa, unidentified
  source 1..9: note = Description of Unknown: immunogenic epitope
ILHNGAYSL

SEQ ID NO: 373 - protein, 9 aa, unidentified
  source 1..9: note = Description of Unknown: immunogenic epitope
RLLQETELV

SEQ ID NO: 374 - protein, 9 aa, unidentified
  source 1..9: note = Description of Unknown: immunogenic epitope
VVKGVVFGI

SEQ ID NO: 375 - protein, 10 aa, unidentified
  source 1..10: note = Description of Unknown: immunogenic epitope
YMIMVKCWMI

SEQ ID NO: 376 - protein, 9 aa, unidentified
  source 1..9: note = Description of Unknown: immunogenic epitope
SLLSGDWVL

SEQ ID NO: 377 - protein, 9 aa, unidentified
  source 1..9: note = Description of Unknown: immunogenic epitope
GLQLGVQAV

SEQ ID NO: 378 - protein, 9 aa, unidentified
  source 1..9: note = Description of Unknown: immunogenic epitope
PLTEYIQPV

SEQ ID NO: 379 - protein, 9 aa, unidentified
  source 1..9: note = Description of Unknown: immunogenic epitope
SPRWWPTCL

SEQ ID NO: 380 - protein, 9 aa, unidentified
  source 1..9: note = Description of Unknown: immunogenic epitope
GVALQTMKQ

SEQ ID NO: 381 - protein, 9 aa, unidentified
  source 1..9: note = Description of Unknown: immunogenic epitope
FMNKFIYEI

SEQ ID NO: 382 - protein, 10 aa, unidentified
  source 1..10: note = Description of Unknown: immunogenic epitope
QLAVSVILRV

SEQ ID NO: 383 - protein, 14 aa, unidentified
  source 1..14: note = Description of Unknown: immunogenic epitope
LPAVVGLSPGEQEY

SEQ ID NO: 384 - protein, 9 aa, unidentified
  source 1..9: note = Description of Unknown: immunogenic epitope
NYARTEDFF

SEQ ID NO: 385 - protein, 9 aa, unidentified
  source 1..9: note = Description of Unknown: immunogenic epitope
FLIIWQNTM

SEQ ID NO: 386 - protein, 14 aa, unidentified
  source 1..14: note = Description of Unknown: immunogenic epitope
TLYQDDTLTLQAAG

SEQ ID NO: 387 - protein, 10 aa, unidentified
  source 1..10: note = Description of Unknown: immunogenic epitope
SLYKFSPFPL

SEQ ID NO: 388 - protein, 9 aa, unidentified
  source 1..9: note = Description of Unknown: immunogenic epitope
KELEGILLL

SEQ ID NO: 389 - protein, 9 aa, unidentified
  source 1..9: note = Description of Unknown: immunogenic epitope
RLSSCVPVA

SEQ ID NO: 390 - protein, 10 aa, unidentified
  source 1..10: note = Description of Unknown: immunogenic epitope
VLLQAGSLHA

SEQ ID NO: 391 - protein, 9 aa, unidentified
  source 1..9: note = Description of Unknown: immunogenic epitope
LLNAFTVTV

SEQ ID NO: 392 - protein, 8 aa, unidentified
  source 1..8: note = Description of Unknown: immunogenic epitope
VLFYLGQY

SEQ ID NO: 393 - protein, 10 aa, unidentified
  source 1..10: note = Description of Unknown: immunogenic epitope
FIASNGVKLV

SEQ ID NO: 394 - protein, 9 aa, unidentified
  source 1..9: note = Description of Unknown: immunogenic epitope
ETVSEQSNV

SEQ ID NO: 395 - protein, 9 aa, unidentified
  source 1..9: note = Description of Unknown: immunogenic epitope
FLDEFMEGV

SEQ ID NO: 396 - protein, 8 aa, unidentified
  source 1..8: note = Description of Unknown: immunogenic epitope
QQITKTEV

SEQ ID NO: 397 - protein, 8 aa, unidentified
  source 1..8: note = Description of Unknown: immunogenic epitope
YRPRPRRY

SEQ ID NO: 398 - protein, 9 aa, unidentified
  source 1..9: note = Description of Unknown: immunogenic epitope
MVKISGGPR

SEQ ID NO: 399 - protein, 9 aa, unidentified
  source 1..9: note = Description of Unknown: immunogenic epitope
EVDPIGHVY

SEQ ID NO: 400 - protein, 9 aa, unidentified
  source 1..9: note = Description of Unknown: immunogenic epitope
ISGGPRISY

SEQ ID NO: 401 - protein, 9 aa, unidentified
  source 1..9: note = Description of Unknown: immunogenic epitope
RQKKIRIQL

SEQ ID NO: 402 - protein, 16 aa, unidentified
  source 1..16: note = Description of Unknown: immunogenic epitope
HLGSRQKKIRIQLRSQ

SEQ ID NO: 403 - protein, 17 aa, unidentified
  source 1..17: note = Description of Unknown: immunogenic epitope
CATWKVICKSCISQTPG

SEQ ID NO: 404 - protein, 9 aa, unidentified
  source 1..9: note = Description of Unknown: immunogenic epitope
TYLPTNASL

SEQ ID NO: 405 - protein, 9 aa, unidentified
  source 1..9: note = Description of Unknown: immunogenic epitope
MIAVFLPIV

SEQ ID NO: 406 - protein, 15 aa, unidentified
  source 1..15: note = Description of Unknown: immunogenic epitope
HQQYFYKIPILVINK

SEQ ID NO: 407 - protein, 9 aa, unidentified
  source 1..9: note = Description of Unknown: immunogenic epitope
FLFLLFFWL

SEQ ID NO: 408 - protein, 9 aa, unidentified
  source 1..9: note = Description of Unknown: immunogenic epitope
TLMSAMTNL

SEQ ID NO: 409 - protein, 9 aa, unidentified
  source 1..9: note = Description of Unknown: immunogenic epitope
ALDVYNGLL

SEQ ID NO: 410 - protein, 10 aa, unidentified
  source 1..10: note = Description of Unknown: immunogenic epitope
FLTPKKLQCV

SEQ ID NO: 411 - protein, 10 aa, unidentified
  source 1..10: note = Description of Unknown: immunogenic epitope
VISNDVCAQV

SEQ ID NO: 412 - protein, 9 aa, unidentified
  source 1..9: note = Description of Unknown: immunogenic epitope
YYWPRPRRY

SEQ ID NO: 413 - protein, 9 aa, unidentified
  source 1..9: note = Description of Unknown: immunogenic epitope
NTYASPRFK

SEQ ID NO: 414 - protein, 10 aa, unidentified
  source 1..10: note = Description of Unknown: immunogenic epitope
SLFEGIDIYT

SEQ ID NO: 415 - protein, 9 aa, unidentified
  source 1..9: note = Description of Unknown: immunogenic epitope
ALSVMGVYV

SEQ ID NO: 416 - protein, 8 aa, unidentified
  source 1..8: note = Description of Unknown: immunogenic epitope
GLQLGVQA

SEQ ID NO: 417 - protein, 15 aa, unidentified
  source 1..15: note = Description of Unknown: immunogenic epitope
YSVYFNLPADTIYTN

SEQ ID NO: 418 - protein, 29 aa, unidentified
  source 1..29: note = Description of Unknown: immunogenic epitope
EDLTVKIGDFGLATEKSRWSGSHQFEQLS

SEQ ID NO: 419 - protein, 9 aa, unidentified
  source 1..9: note = Description of Unknown: immunogenic epitope
SYLDSGIHF

SEQ ID NO: 420 - protein, 12 aa, unidentified
  source 1..12: note = Description of Unknown: immunogenic epitope
FSWAMDLDPKGA

SEQ ID NO: 421 - protein, 10 aa, unidentified
  source 1..10: note = Description of Unknown: immunogenic epitope
ACDPHSGHFV

SEQ ID NO: 422 - protein, 9 aa, unidentified
  source 1..9: note = Description of Unknown: immunogenic epitope
CILGKLFTK

SEQ ID NO: 423 - protein, 9 aa, unidentified
  source 1..9: note = Description of Unknown: immunogenic epitope
AVCPWTWLR

SEQ ID NO: 424 - protein, 9 aa, unidentified
  source 1..9: note = Description of Unknown: immunogenic epitope
ILDKVLVHL

SEQ ID NO: 425 - protein, 9 aa, unidentified
  source 1..9: note = Description of Unknown: immunogenic epitope
GLFGDIYLA

SEQ ID NO: 426 - protein, 14 aa, unidentified
  source 1..14: note = Description of Unknown: immunogenic epitope
MIFEKHGFRRTTPP

SEQ ID NO: 427 - protein, 10 aa, unidentified
  source 1..10: note = Description of Unknown: immunogenic epitope
SLADEAEVYL

SEQ ID NO: 428 - protein, 10 aa, unidentified
  source 1..10: note = Description of Unknown: immunogenic epitope
TLDWLLQTPK

SEQ ID NO: 429 - protein, 9 aa, unidentified
  source 1..9: note = Description of Unknown: immunogenic epitope
ILNAMIAKI

SEQ ID NO: 430 - protein, 9 aa, unidentified
  source 1..9: note = Description of Unknown: immunogenic epitope
WRRAPAPGA

SEQ ID NO: 431 - protein, 9 aa, unidentified
  source 1..9: note = Description of Unknown: immunogenic epitope
PVTWRRAPA

SEQ ID NO: 432 - protein, 11 aa, unidentified
  source 1..11: note = Description of Unknown: immunogenic epitope
FLEGNEVGKTY

SEQ ID NO: 433 - protein, 9 aa, unidentified
  source 1..9: note = Description of Unknown: immunogenic epitope
KTLTSVFQK

SEQ ID NO: 434 - protein, 9 aa, unidentified
  source 1..9: note = Description of Unknown: immunogenic epitope
EEKLIVVLF

SEQ ID NO: 435 - protein, 11 aa, unidentified
  source 1..11: note = Description of Unknown: immunogenic epitope
SELFRSGLDSY

SEQ ID NO: 436 - protein, 9 aa, unidentified
  source 1..9: note = Description of Unknown: immunogenic epitope
FRSGLDSYV

SEQ ID NO: 437 - protein, 9 aa, unidentified
  source 1..9: note = Description of Unknown: immunogenic epitope
EAFIQPITR

SEQ ID NO: 438 - protein, 11 aa, unidentified
  source 1..11: note = Description of Unknown: immunogenic epitope
RVIKNSIRLTL

SEQ ID NO: 439 - protein, 9 aa, unidentified
  source 1..9: note = Description of Unknown: immunogenic epitope
KINKNPKYK

SEQ ID NO: 440 - protein, 9 aa, unidentified
  source 1..9: note = Description of Unknown: immunogenic epitope
YTDFHCQYV

SEQ ID NO: 441 - protein, 10 aa, unidentified
  source 1..10: note = Description of Unknown: immunogenic epitope
LLLDDLLVSI

SEQ ID NO: 442 - protein, 16 aa, unidentified
  source 1..16: note = Description of Unknown: immunogenic epitope
PYYFAAELPPRNLPEP

SEQ ID NO: 443 - protein, 10 aa, unidentified
  source 1..10: note = Description of Unknown: immunogenic epitope
ILDTAGREEY

SEQ ID NO: 444 - protein, 9 aa, unidentified
  source 1..9: note = Description of Unknown: immunogenic epitope
RPHVPESAF

SEQ ID NO: 445 - protein, 9 aa, unidentified
  source 1..9: note = Description of Unknown: immunogenic epitope
KIFSEVTLK

SEQ ID NO: 446 - protein, 9 aa, unidentified
  source 1..9: note = Description of Unknown: immunogenic epitope
SHETVIIEL

SEQ ID NO: 447 - protein, 15 aa, unidentified
  source 1..15: note = Description of Unknown: immunogenic epitope
GELIGILNAAKVPAD

SEQ ID NO: 448 - protein, 9 aa, unidentified
  source 1..9: note = Description of Unknown: immunogenic epitope
LYSACFWWL

SEQ ID NO: 449 - protein, 9 aa, unidentified
  source 1..9: note = Description of Unknown: immunogenic epitope
VLHWDPETV

SEQ ID NO: 450 - protein, 9 aa, unidentified
  source 1..9: note = Description of Unknown: immunogenic epitope
MSLQRQFLR

SEQ ID NO: 451 - protein, 20 aa, unidentified
  source 1..20: note = Description of Unknown: immunogenic epitope
ISPNSVFSQWRVVCDSLEDY

SEQ ID NO: 452 - protein, 10 aa, unidentified
  source 1..10: note = Description of Unknown: immunogenic epitope
SLPYWNFATG

SEQ ID NO: 453 - protein, 15 aa, unidentified
  source 1..15: note = Description of Unknown: immunogenic epitope
SQWRVVCDSLEDYDT

SEQ ID NO: 454 - protein, 9 aa, unidentified
  source 1..9: note = Description of Unknown: immunogenic epitope
SVYDFFVWL

SEQ ID NO: 455 - protein, 9 aa, unidentified
  source 1..9: note = Description of Unknown: immunogenic epitope
TLDSQVMSL

SEQ ID NO: 456 - protein, 9 aa, unidentified
  source 1..9: note = Description of Unknown: immunogenic epitope
LLGPGRPYR

SEQ ID NO: 457 - protein, 9 aa, unidentified
  source 1..9: note = Description of Unknown: immunogenic epitope
ANDPIFVVL

SEQ ID NO: 458 - protein, 15 aa, unidentified
  source 1..15: note = Description of Unknown: immunogenic epitope
QCTEVRADTRPWSGP

SEQ ID NO: 459 - protein, 10 aa, unidentified
  source 1..10: note = Description of Unknown: immunogenic epitope
ALPYWNFATG

SEQ ID NO: 460 - protein, 9 aa, unidentified
  source 1..9: note = Description of Unknown: immunogenic epitope
KCDICTDEY

SEQ ID NO: 461 - protein, 11 aa, unidentified
  source 1..11: note = Description of Unknown: immunogenic epitope
SSDYVIPIGTY

SEQ ID NO: 462 - protein, 9 aa, unidentified
  source 1..9: note = Description of Unknown: immunogenic epitope
MLLAVLYCL

SEQ ID NO: 463 - protein, 10 aa, unidentified
  source 1..10: note = Description of Unknown: immunogenic epitope
CLLWSFQTSA

SEQ ID NO: 464 - protein, 9 aa, unidentified
  source 1..9: note = Description of Unknown: immunogenic epitope
YMDGTMSQV

SEQ ID NO: 465 - protein, 11 aa, unidentified
  source 1..11: note = Description of Unknown: immunogenic epitope
IYMDGTADFSF

SEQ ID NO: 466 - protein, 9 aa, unidentified
  source 1..9: note = Description of Unknown: immunogenic epitope
QCSGNFMGF

SEQ ID NO: 467 - protein, 12 aa, unidentified
  source 1..12: note = Description of Unknown: immunogenic epitope
TPRLPSSADVEF

SEQ ID NO: 468 - protein, 9 aa, unidentified
  source 1..9: note = Description of Unknown: immunogenic epitope
LPSSADVEF

SEQ ID NO: 469 - protein, 10 aa, unidentified
  source 1..10: note = Description of Unknown: immunogenic epitope
LHHAFVDSIF

SEQ ID NO: 470 - protein, 9 aa, unidentified
  source 1..9: note = Description of Unknown: immunogenic epitope
SEIWRDIDF

SEQ ID NO: 471 - protein, 15 aa, unidentified
  source 1..15: note = Description of Unknown: immunogenic epitope
QNILLSNAPLGPQFP

SEQ ID NO: 472 - protein, 21 aa, unidentified [sequence omitted]

SEQ ID NO: 473 - protein, 30 aa, unidentified
  source 1..30: note = Description of Unknown: immunogenic epitope
YTTAEEAAGIGILTVILGVLLLIGCWYCRR

SEQ ID NO: 474 - protein, 9 aa, unidentified
  source 1..9: note = Description of Unknown: immunogenic epitope
ALNFPGSQK

SEQ ID NO: 475 - protein, 9 aa, unidentified
  source 1..9: note = Description of Unknown: immunogenic epitope
VYFFLPDHL

SEQ ID NO: 476 - protein, 9 aa, unidentified
  source 1..9: note = Description of Unknown: immunogenic epitope
RTKQLYPEW

SEQ ID NO: 477 - protein, 10 aa, unidentified
  source 1..10: note = Description of Unknown: immunogenic epitope
HTMEVTVYHR

SEQ ID NO: 478 - protein, 9 aa, unidentified
  source 1..9: note = Description of Unknown: immunogenic epitope
SSPGCQPPA

SEQ ID NO: 479 - protein, 10 aa, unidentified
  source 1..10: note = Description of Unknown: immunogenic epitope
VPLDCVLYRY

SEQ ID NO: 480 - protein, 9 aa, unidentified
  source 1..9: note = Description of Unknown: immunogenic epitope
LPHSSSHWL

SEQ ID NO: 481 - protein, 8 aa, unidentified
  source 1..8: note = Description of Unknown: immunogenic epitope
SNDGPTLI

SEQ ID NO: 482 - protein, 15 aa, unidentified
  source 1..15: note = Description of Unknown: immunogenic epitope
GRAMLGTHTMEVTVY

SEQ ID NO: 483 - protein, 16 aa, unidentified
  source 1..16: note = Description of Unknown: immunogenic epitope
WNRQLYPEWTEAQRLD

SEQ ID NO: 484 - protein, 18 aa, unidentified
  source 1..18: note = Description of Unknown: immunogenic epitope
TTEWVETTARELPIPEPE

SEQ ID NO: 485 - protein, 17 aa, unidentified
  source 1..17: note = Description of Unknown: immunogenic epitope
TGRAMLGTHTMEVTVYH

SEQ ID NO: 486 - protein, 10 aa, unidentified
  source 1..10: note = Description of Unknown: immunogenic epitope
VLPDVFIRCV

SEQ ID NO: 487 - protein, 10 aa, unidentified
  source 1..10: note = Description of Unknown: immunogenic epitope
RYCNLEGPPI

SEQ ID NO: 488 - protein, 24 aa, unidentified
  source 1..24: note = Description of Unknown: immunogenic epitope
KWTEPYCVIAAVKIFPRFFMVAKQ

SEQ ID NO: 489 - protein, 20 aa, unidentified
  source 1..20: note = Description of Unknown: immunogenic epitope
KCCKIRYCNLEGPPINSSVF

SEQ ID NO: 490 - protein, 9 aa, unidentified
  source 1..9: note = Description of Unknown: immunogenic epitope
GLYDGMEHL

SEQ ID NO: 491 - protein, 9 aa, unidentified
  source 1..9: note = Description of Unknown: immunogenic epitope
FLWGPRALV

SEQ ID NO: 492 - protein, 9 aa, unidentified
  source 1..9: note = Description of Unknown: immunogenic epitope
VRIGHLYIL

SEQ ID NO: 493 - protein, 15 aa, unidentified
  source 1..15: note = Description of Unknown: immunogenic epitope
REPFTKAEMLGSVIR

SEQ ID NO: 494 - protein, 14 aa, unidentified
  source 1..14: note = Description of Unknown: immunogenic epitope
AELVHFLLLKYRAR

SEQ ID NO: 495 - protein, 10 aa, unidentified
  source 1..10: note = Description of Unknown: immunogenic epitope
LLFGLALIEV

SEQ ID NO: 496 - protein, 9 aa, unidentified
  source 1..9: note = Description of Unknown: immunogenic epitope
ALKDVEERV

SEQ ID NO: 497 - protein, 9 aa, unidentified
  source 1..9: note = Description of Unknown: immunogenic epitope
SESIKKKVL

SEQ ID NO: 498 - protein, 9 aa, unidentified
  source 1..9: note = Description of Unknown: immunogenic epitope
ASSTLYLVF

SEQ ID NO: 499 - protein, 15 aa, unidentified
  source 1..15: note = Description of Unknown: immunogenic epitope
SSTLYLVFSPSSFST

SEQ ID NO: 500 - protein, 9 aa, unidentified
  source 1..9: note = Description of Unknown: immunogenic epitope
QGQHFLQKV

SEQ ID NO: 501 - protein, 10 aa, unidentified
  source 1..10: note = Description of Unknown: immunogenic epitope
EVISCKLIKR

SEQ ID NO: 502 - protein, 9 aa, unidentified
  source 1..9: note = Description of Unknown: immunogenic epitope
FPSDSWCYF

SEQ ID NO: 503 - protein, 9 aa, unidentified
  source 1..9: note = Description of Unknown: immunogenic epitope
LYATVIHDI

SEQ ID NO: 504 - protein, 9 aa, unidentified
  source 1..9: note = Description of Unknown: immunogenic epitope
SSKALQRPV

SEQ ID NO: 505 - protein, 9 aa, unidentified
  source 1..9: note = Description of Unknown: immunogenic epitope
GFKQSSKAL

SEQ ID NO: 506 - protein, 17 aa, unidentified
  source 1..17: note = Description of Unknown: immunogenic epitope
ATGFKQSSKALQRPVAS

SEQ ID NO: 507 - protein, 16 aa, unidentified
  source 1..16: note = Description of Unknown: immunogenic epitope
TMKQICKKEIRRLHQY

SEQ ID NO: 508 - protein, 10 aa, unidentified
  source 1..10: note = Description of Unknown: immunogenic epitope
KILDAVVAQK

SEQ ID NO: 509 - protein, 9 aa, unidentified
  source 1..9: note = Description of Unknown: immunogenic epitope
RIAECILGM

SEQ ID NO: 510 - protein, 15 aa, unidentified
  source 1..15: note = Description of Unknown: immunogenic epitope
IGRIAECILGMNPSR

SEQ ID NO: 511 - protein, 10 aa, unidentified
  source 1..10: note = Description of Unknown: immunogenic epitope
YVDFREYEYY

SEQ ID NO: 512 - protein, 9 aa, unidentified
  source 1..9: note = Description of Unknown: immunogenic epitope
FLDRFLSCM

SEQ ID NO: 513 - protein, 11 aa, unidentified
  source 1..11: note = Description of Unknown: immunogenic epitope
SLIAAAAFCLA

SEQ ID NO: 514 - protein, 9 aa, unidentified
  source 1..9: note = Description of Unknown: immunogenic epitope
VVMSWAPPV

SEQ ID NO: 515 - protein, 25 aa, unidentified
  source 1..25: note = Description of Unknown: immunogenic epitope
NSNHVASGAGEAAIETQSSSSEEIV

SEQ ID NO: 516 - protein, 10 aa, unidentified
  source 1..10: note = Description of Unknown: immunogenic epitope
ILFGISLREV

SEQ ID NO: 517 - protein, 9 aa, unidentified
  source 1..9: note = Description of Unknown: immunogenic epitope
KVVEFLAML

SEQ ID NO: 518 - protein, 13 aa, unidentified
  source 1..13: note = Description of Unknown: immunogenic epitope
SSALLSIFQSSPE

SEQ ID NO: 519 - protein, 9 aa, unidentified
  source 1..9: note = Description of Unknown: immunogenic epitope
SFSYTLLSL

SEQ ID NO: 520 - protein, 9 aa, unidentified
  source 1..9: note = Description of Unknown: immunogenic epitope
VSSFFSYTL

SEQ ID NO: 521 - protein, 15 aa, unidentified
  source 1..15: note = Description of Unknown: immunogenic epitope
KPLFRRMSSLELVIA

SEQ ID NO: 522 - protein, 512 aa, Vaccinia virus; identical to SEQ ID NO: 23

SEQ ID NO: 523 - protein, 512 aa, Vaccinia virus; identical to SEQ ID NO: 23

SEQ ID NO: 524 - protein, 512 aa, Vaccinia virus; identical to SEQ ID NO: 58

SEQ ID NO: 525 - protein, 506 aa, Vaccinia virus; identical to SEQ ID NO: 74

SEQ ID NO: 526 - protein, 512 aa, Vaccinia virus; identical to SEQ ID NO: 92

SEQ ID NO: 527 - protein, 224 aa, Vaccinia virus; identical to SEQ ID NO: 24

SEQ ID NO: 528 - protein, 229 aa, Vaccinia virus; identical to SEQ ID NO: 42

SEQ ID NO: 529 - protein, 224 aa, Vaccinia virus; identical to SEQ ID NO: 59

SEQ ID NO: 530 - protein, 224 aa, Vaccinia virus; identical to SEQ ID NO: 75

SEQ ID NO: 531 - protein, 224 aa, Vaccinia virus; identical to SEQ ID NO: 93

SEQ ID NO: 532 - protein, 117 aa, Vaccinia virus; identical to SEQ ID NO: 25

SEQ ID NO: 533 - protein, 117 aa, Vaccinia virus; identical to SEQ ID NO: 43

SEQ ID NO: 534 - protein, 117 aa, Vaccinia virus; identical to SEQ ID NO: 25

SEQ ID NO: 535 - protein, 117 aa, Vaccinia virus; identical to SEQ ID NO: 25

SEQ ID NO: 536 - protein, 117 aa, Vaccinia virus; identical to SEQ ID NO: 25

SEQ ID NO: 537 - protein, 175 aa, Vaccinia virus; identical to SEQ ID NO: 26

SEQ ID NO: 538 - protein, 175 aa, Vaccinia virus; identical to SEQ ID NO: 44

SEQ ID NO: 539 - protein, 79 aa, Vaccinia virus; identical to SEQ ID NO: 61

SEQ ID NO: 540 - protein, 175 aa, Vaccinia virus; identical to SEQ ID NO: 77

SEQ ID NO: 541 - protein, 175 aa, Vaccinia virus; identical to SEQ ID NO: 95

SEQ ID NO: 542 - protein, 472 aa, Vaccinia virus; identical to SEQ ID NO: 27

SEQ ID NO: 543 - protein, 472 aa, Vaccinia virus; identical to SEQ ID NO: 45

SEQ ID NO: 544 - protein, 472 aa, Vaccinia virus; identical to SEQ ID NO: 62

SEQ ID NO: 545 - protein, 161 aa, Vaccinia virus; identical to SEQ ID NO: 80

SEQ ID NO: 546 - protein, 470 aa, Vaccinia virus; identical to SEQ ID NO: 96

SEQ ID NO: 547 - protein, 220 aa, Vaccinia virus; identical to SEQ ID NO: 28

SEQ ID NO: 548 - protein, 220 aa, Vaccinia virus; identical to SEQ ID NO: 46

SEQ ID NO: 549 - protein, 196 aa, Vaccinia virus; identical to SEQ ID NO: 63

SEQ ID NO: 550 - protein, 220 aa, Vaccinia virus; identical to SEQ ID NO: 28

SEQ ID NO: 551 - protein, 220 aa, Vaccinia virus; identical to SEQ ID NO: 28

SEQ ID NO: 552 - protein, 284 aa, Vaccinia virus; identical to SEQ ID NO: 29

SEQ ID NO: 553 - protein, 284 aa, Vaccinia virus; identical to SEQ ID NO: 47

SEQ ID NO: 554 - protein, 189 aa, Vaccinia virus; identical to SEQ ID NO: 65

SEQ ID NO: 555 - protein, 284 aa, Vaccinia virus; identical to SEQ ID NO: 82

SEQ ID NO: 556 - protein, 284 aa, Vaccinia virus; identical to SEQ ID NO: 98

SEQ ID NO: 557 - protein, 369 aa, Vaccinia virus; identical to SEQ ID NO: 30

SEQ ID NO: 558 - protein, 369 aa, Vaccinia virus; identical to SEQ ID NO: 48

SEQ ID NO: 559 - protein, 368 aa, Vaccinia virus; identical to SEQ ID NO: 66

SEQ ID NO: 560 - protein, 364 aa, Vaccinia virus; identical to SEQ ID NO: 83

SEQ ID NO: 561 - protein, 309 aa, Vaccinia virus; identical to SEQ ID NO: 99

SEQ ID NO: 562 - protein, 81 aa, Vaccinia virus; identical to SEQ ID NO: 31

SEQ ID NO: 563 - protein, 81 aa, Vaccinia virus; identical to SEQ ID NO: 67

SEQ ID NO: 564 - protein, 88 aa, Vaccinia virus; identical to SEQ ID NO: 33

SEQ ID NO: 565 - protein, 88 aa, Vaccinia virus; identical to SEQ ID NO: 49

SEQ ID NO: 566 - protein, 88 aa, Vaccinia virus; identical to SEQ ID NO: 33

SEQ ID NO: 567 - protein, 88 aa, Vaccinia virus; identical to SEQ ID NO: 84

SEQ ID NO: 568 - protein, 88 aa, Vaccinia virus; identical to SEQ ID NO: 100

SEQ ID NO: 569 - protein, 424 aa, Vaccinia virus; identical to SEQ ID NO: 34

SEQ ID NO: 570 - protein, 424 aa, Vaccinia virus; identical to SEQ ID NO: 50

SEQ ID NO: 571 - protein, 424 aa, Vaccinia virus; identical to SEQ ID NO: 85

SEQ ID NO: 572 - protein, 424 aa, Vaccinia virus; identical to SEQ ID NO: 85

SEQ ID NO: 573 - protein, 135 aa, Vaccinia virus; identical to SEQ ID NO: 35

SEQ ID NO: 574 - protein, 134 aa, Vaccinia virus; identical to SEQ ID NO: 52

SEQ ID NO: 575 - protein, 134 aa, Vaccinia virus; identical to SEQ ID NO: 52

SEQ ID NO: 576 - protein, 171 aa, Vaccinia virus; identical to SEQ ID NO: 86

SEQ ID NO: 577 - protein, 175 aa, Vaccinia virus; identical to SEQ ID NO: 102

SEQ ID NO: 578 - protein, 81 aa, Vaccinia virus; identical to SEQ ID NO: 36

SEQ ID NO: 579 - protein, 81 aa, Vaccinia virus; identical to SEQ ID NO: 36

SEQ ID NO: 580 - protein, 81 aa, Vaccinia virus; identical to SEQ ID NO: 36

SEQ ID NO: 581 - protein, 81 aa, Vaccinia virus; identical to SEQ ID NO: 36

SEQ ID NO: 582 - protein, 149 aa, Vaccinia virus; identical to SEQ ID NO: 37

SEQ ID NO: 583 - protein, 149 aa, Vaccinia virus; identical to SEQ ID NO: 37

SEQ ID NO: 584 - protein, 149 aa, Vaccinia virus; identical to SEQ ID NO: 37

SEQ ID NO: 585 - protein, 149 aa, Vaccinia virus; identical to SEQ ID NO: 37

SEQ ID NO: 586 - protein, 149 aa, Vaccinia virus; identical to SEQ ID NO: 37

SEQ ID NO: 587 - protein, 226 aa, Vaccinia virus; identical to SEQ ID NO: 38

SEQ ID NO: 588 - protein, 226 aa, Vaccinia virus; identical to SEQ ID NO: 55

SEQ ID NO: 589 - protein, 226 aa, Vaccinia virus; identical to SEQ ID NO: 71

SEQ ID NO: 590 - protein, 226 aa, Vaccinia virus; identical to SEQ ID NO: 89

SEQ ID NO: 591 - protein, 226 aa, Vaccinia virus; identical to SEQ ID NO: 38

SEQ ID NO: 592 - protein, 147 aa, Vaccinia virus; identical to SEQ ID NO: 39

SEQ ID NO: 593 - protein, 147 aa, Vaccinia virus; identical to SEQ ID NO: 56

SEQ ID NO: 594 - protein, 147 aa, Vaccinia virus; identical to SEQ ID NO: 72

SEQ ID NO: 595 - protein, 147 aa, Vaccinia virus; identical to SEQ ID NO: 39

SEQ ID NO: 596 - protein, 147 aa, Vaccinia virus; identical to SEQ ID NO: 106

SEQ ID NO: 597 - protein, 480 aa, Vaccinia virus; identical to SEQ ID NO: 40

SEQ ID NO: 598 - protein, 480 aa, Vaccinia virus; identical to SEQ ID NO: 57

SEQ ID NO: 599 - protein, 480 aa, Vaccinia virus; identical to SEQ ID NO: 73

SEQ ID NO: 600 - protein, 480 aa, Vaccinia virus; identical to SEQ ID NO: 73

SEQ ID NO: 601 - protein, 480 aa, Vaccinia virus; identical to SEQ ID NO: 107

SEQ ID NO: 602 - protein, 222 aa, Vaccinia virus; identical to SEQ ID NO: 108

SEQ ID NO: 603 - protein, 345 aa, Vaccinia virus; identical to SEQ ID NO: 127

SEQ ID NO: 604 - protein, 222 aa, Vaccinia virus; identical to SEQ ID NO: 151

SEQ ID NO: 605 - protein, 222 aa, Vaccinia virus; identical to SEQ ID NO: 163

SEQ ID NO: 606 - protein, 149 aa, Vaccinia virus; identical to SEQ ID NO: 109

SEQ ID NO: 607 - protein, 149 aa, Vaccinia virus; identical to SEQ ID NO: 128

SEQ ID NO: 608 - protein, 149 aa, Vaccinia virus; identical to SEQ ID NO: 109

SEQ ID NO: 609 - protein, 149 aa, Vaccinia virus; identical to SEQ ID NO: 164

SEQ ID NO: 610 - protein, 149 aa, Vaccinia virus; identical to SEQ ID NO: 109

SEQ ID NO: 611 - protein, 91 aa, Vaccinia virus; identical to SEQ ID NO: 110

SEQ ID NO: 612 - protein, 91 aa, Vaccinia virus; identical to SEQ ID NO: 110

SEQ ID NO: 613 - protein, 326 aa, Vaccinia virus; identical to SEQ ID NO: 111
  VARIANT 31: note = Any amino acid

SEQ ID NO: 614 - protein, 326 aa, Vaccinia virus; identical to SEQ ID NO: 129

SEQ ID NO: 615 - protein, 289 aa, Vaccinia virus
MELENEPVILPCPQINTLSSGYNILDILWEKRGADNDRIIPIDNGSNMLILNPTQSDSGIYICITTNETYCDMMSLNLTIVSVLESNIDLISYPQIVNERSTGEMVCPNINAFIASNVNADIIWSGHRRLRNKRLKQRTPGIITIEDVRKNDAGYYTCVLEYIYRGKTYNVTRIVKLEVRDKIIPSTMQLPDGIVTSIGSNLTIACRVSLRPPTTDADVFWISNGMYYEEDDGDGNGRISVANKIYMTDKRRVITSRLNINPVKEEDATTFTCMAFTIPSISKTVTVSI

SEQ ID NO: 616 - protein, 326 aa, Vaccinia virus; identical to SEQ ID NO: 165

SEQ ID NO: 617 - protein, 326 aa, Vaccinia virus; identical to SEQ ID NO: 201

SEQ ID NO: 618 - protein, 75 aa, Vaccinia virus; identical to SEQ ID NO: 112

SEQ ID NO: 619 - protein, 75 aa, Vaccinia virus; identical to SEQ ID NO: 155

SEQ ID NO: 620 - protein, 340 aa, Vaccinia virus; identical to SEQ ID NO: 113

SEQ ID NO: 621 - protein, 340 aa, Vaccinia virus; identical to SEQ ID NO: 130

SEQ ID NO: 622 - protein, 340 aa, Vaccinia virus; identical to SEQ ID NO: 156

SEQ ID NO: 623 - protein, 340 aa, Vaccinia virus; identical to SEQ ID NO: 166

SEQ ID NO: 624 - protein, 340 aa, Vaccinia virus; identical to SEQ ID NO: 202

SEQ ID NO: 625 - protein, 574 aa, Vaccinia virus; identical to SEQ ID NO: 114

SEQ ID NO: 626 - protein, 574 aa, Vaccinia virus; identical to SEQ ID NO: 131

SEQ ID NO: 627 - protein, 574 aa, Vaccinia virus; identical to SEQ ID NO: 157

SEQ ID NO: 628 - protein, 574 aa, Vaccinia virus; identical to SEQ ID NO: 167

SEQ ID NO: 629 - protein, 353 aa, Vaccinia virus; identical to SEQ ID NO: 115

SEQ ID NO: 630 - protein, 351 aa, Vaccinia virus; identical to SEQ ID NO: 132

SEQ ID NO: 631 - protein, 353 aa, Vaccinia virus; identical to SEQ ID NO: 158

SEQ ID NO: 632 - protein, 265 aa, Vaccinia virus; identical to SEQ ID NO: 168

SEQ ID NO: 633 - protein, 91 aa, Vaccinia virus; identical to SEQ ID NO: 117

SEQ ID NO: 634 - protein, 87 aa, Vaccinia virus; identical to SEQ ID NO: 184

SEQ ID NO: 635 - protein, 181 aa, Vaccinia virus; identical to SEQ ID NO: 118

SEQ ID NO: 636 - protein, 181 aa, Vaccinia virus; identical to SEQ ID NO: 185

SEQ ID NO: 637 - protein, 147 aa, Vaccinia virus; identical to SEQ ID NO: 205

SEQ ID NO: 638 - protein, 386 aa, Vaccinia virus; identical to SEQ ID NO: 119

SEQ ID NO: 639 - protein, 382 aa, Vaccinia virus; identical to SEQ ID NO: 186

SEQ ID NO: 640 - protein, 150 aa, Vaccinia virus; identical to SEQ ID NO: 120

SEQ ID NO: 641 - protein, 198 aa, Vaccinia virus; identical to SEQ ID NO: 187

SEQ ID NO: 642 - protein, 259 aa, Vaccinia virus; identical to SEQ ID NO: 122

SEQ ID NO: 643 - protein, 112 aa, Vaccinia virus; identical to SEQ ID NO: 143

SEQ ID NO: 644 - protein, 147 aa, Vaccinia virus; identical to SEQ ID NO: 188

SEQ ID NO: 645 - protein, 103 aa, Vaccinia virus; identical to SEQ ID NO: 123

SEQ ID NO: 646 - protein, 109 aa, Vaccinia virus; identical to SEQ ID NO: 144

SEQ ID NO: 647 - protein, 128 aa, Vaccinia virus; identical to SEQ ID NO: 189

SEQ ID NO: 648 - protein, 128 aa, Vaccinia virus; identical to SEQ ID NO: 189

SEQ ID NO: 649 - protein, 113 aa, Vaccinia virus; identical to SEQ ID NO: 124

SEQ ID NO: 650 - protein, 64 aa, Vaccinia virus; identical to SEQ ID NO: 145

SEQ ID NO: 651 - protein, 111 aa, Vaccinia virus; identical to SEQ ID NO: 190

SEQ ID NO: 652 - protein, 64 aa, Vaccinia virus; identical to SEQ ID NO: 145

SEQ ID NO: 653 - protein, 122 aa, Vaccinia virus; identical to SEQ ID NO: 125

SEQ ID NO: 654 - protein, 122 aa, Vaccinia virus; identical to SEQ ID NO: 147

SEQ ID NO: 655 - protein, 93 aa, Vaccinia virus
MHHPMESVKTTNTNAIICVREHTLPDYANTQCTPCGSGTFTSRNNHLPACLSCNGRRDRVTLLTIESVNALPDIIVFSKDHPDARHVFPKQNV

SEQ ID NO: 656 - protein, 244 aa, Vaccinia virus; identical to SEQ ID NO: 126

SEQ ID NO: 657 - protein, 244 aa, Vaccinia virus; identical to SEQ ID NO: 150

SEQ ID NO: 658 - protein, 244 aa, Vaccinia virus; identical to SEQ ID NO: 150

SEQ ID NO: 659 - protein, 241 aa, Vaccinia virus; identical to SEQ ID NO: 192

SEQ ID NO: 660 - protein, 258 aa, Vaccinia virus; identical to SEQ ID NO: 208

SEQ ID NO: 661 - protein, 13 aa, unidentified
  source 1..13: note = Description of Unknown: immunogenic epitope
SYLQDSDPDSFQD

SEQ ID NO: 662 - protein, 9 aa, unidentified
  source 1..9: note = Description of Unknown: immunogenic epitope
EVDPIGHLY

SEQ ID NO: 663 - protein, 9 aa, unidentified
  source 1..9: note = Description of Unknown: immunogenic epitope
KVAELVHFL

SEQ ID NO: 664 - protein, 9 aa, unidentified
  source 1..9: note = Description of Unknown: immunogenic epitope
TFPDLESEF

SEQ ID NO: 665 - protein, 9 aa, unidentified
  source 1..9: note = Description of Unknown: immunogenic epitope
VAELVHFLL

SEQ ID NO: 666 - protein, 10 aa, unidentified
  source 1..10: note = Description of Unknown: immunogenic epitope
MEVDPIGHLY

SEQ ID NO: 667 - protein, 9 aa, unidentified
  source 1..9: note = Description of Unknown: immunogenic epitope
AELVHFLLL

SEQ ID NO: 668 - protein, 9 aa, unidentified
  source 1..9: note = Description of Unknown: immunogenic epitope
WQYFFPVIF

SEQ ID NO: 669 - protein, 16 aa, unidentified
  source 1..16: note = Description of Unknown: immunogenic epitope
KKLLTQHFVQENYLEY

SEQ ID NO: 670 - protein, 15 aa, unidentified
  source 1..15: note = Description of Unknown: immunogenic epitope
RKVAELVHFLLLKYR

SEQ ID NO: 671 - protein, 16 aa, unidentified
  source 1..16: note = Description of Unknown: immunogenic epitope
ACYEFLWGPRALVETS

SEQ ID NO: 672 - protein, 12 aa, unidentified
  source 1..12: note = Description of Unknown: immunogenic epitope
VIFSKASSSLQL

SEQ ID NO: 673 - protein, 15 aa, unidentified
  source 1..15: note = Description of Unknown: immunogenic epitope
VFGIELMEVDPIGHL

SEQ ID NO: 674 - protein, 15 aa, unidentified
  source 1..15: note = Description of Unknown: immunogenic epitope
GDNQIMPKAGLLIIV

SEQ ID NO: 675 - protein, 15 aa, unidentified
  source 1..15: note = Description of Unknown: immunogenic epitope
TSYVKVLHHMVKISG

SEQ ID NO: 676 - protein, 16 aa, unidentified
  source 1..16: note = Description of Unknown: immunogenic epitope
RKVAELVHFLLLKYRA

SEQ ID NO: 677 - protein, 37 aa, Vaccinia virus
MLNFSLCLYPVFILNKLVLRTQSIILHTINNASIKNR